SEQUENCE LISTING (91 sequences)

SEQ ID NO: 1 - DNA, 756 bp, Synthetic construct
  source 1..756: mol_type = other DNA
atggagctcaaccactgggcgcggcgctacagcgacgccgaggacacaaagaagggccccgctcccgccgcggcccggccccgcagcagctccgccggtcccgaagggcctcagctagacgtgcctcgcgagttcctgccgaggaccctcaccggctacgtgcctgaggacatctggaggaaggctgaagaggccgtgaatgaggtgaagcggcaggccatgtcggagctgcagaaagccgtgtcggacgcggagcgcaaagcgcacgagctcatcaccacggagcgtgccaagatggagcgggccctggccgaggcgaagcggcaggcctccgaggacgccctgacggtcatcaaccagcaggaggactccagcgaggacttccagccactgcgctatttggatggtgtccccagctccttccagttcttcctgcccctcggctccgggggggccctgcacctgcctgcctcctccttccttacccctcccaaggacaagtgcctctcgccagacctgcccctgcccaagcagctggtgtgtcgctgggccaagtgtaaccagctctttgagctcctgcaagacctggtggaccatgtcaacgattaccatgtcaagcccgagaaggatgcggggtactgctgccactgggagggctgcgcccgccatggccgaggtttcaacgccaggtacaagatgctcatccacatccgcacacacaccaacgagaagccacaccgctgtccgacctgctga

SEQ ID NO: 2 - DNA, 2634 bp, Synthetic construct
  source 1..2634: mol_type = other DNA
atgccggactccccagcggaggtgaagacgcagccccggtccacaccccccagcatgccgcccccaccgcctgccgcatcccagggggccacacgccccccctccttcacgccacacacactgatgaacggcagcagccactcaccaacagccatcaatggtgcaccgtgcacacccaacggcttcagcaatggcccggccacctcgtccacagcctccttgtccacacagcacctgcccccagcctgcggggcccggcagctcagcaagctcaagcgcttcctcaccacactgcagcagtttggcagcgacatctccccagagattggggagcgcgtgcgcacactggtgctgggcctggtgaactcgacattgacgatcgaggagtttcattccaagcttcaggaggccaccaacttccctctgcggccgtttgtcattcccttcctgaaggcaaacctgcccttgctgcagcgggagctcctgcactgtgcacgcctggccaagcagacgcccgcccagtacttggcccagcatgagcagctcctgctggacgccagcgcctcctcccccatcgactcctcagagctgctactggaagtcaacgagaacggcaagaggaggacgcccgacaggaccaaagagaacgggtcagaccgcgacccgctgcaccccgagcacctcagcaaacggccatgcaccctgaaccctgcccagcgctacagccccagcaacgggccaccgcagcccacaccgccgccgcactaccgcctggaggacatagccatggcccaccacttccgagatgcctaccgccacccagacccccgggagctacgagagcgccatcggccgcttgtggtgcctgggtcccggcaggaagaagtgatcgaccacaagctcacagagcgtgagtgggcagaagagtggaagcacctcaacaacctcctgaactgcatcatggacatggtggagaagacgcggcgctcgctcacggtgctgcgcaggtgccaggaggccgaccgcgaggagctcaaccactgggcgcggcgctacagcgacgccgaggacacaaagaagggccccgctcccgccgcggcccggccccgcagcagctccgccggtcccgaagggcctcagctagacgtgcctcgcgagttcctgccgaggaccctcaccggctacgtgcctgaggacatctggaggaaggctgaagaggccgtgaatgaggtgaagcggcaggccatgtcggagctgcagaaagccgtgtcggacgcggagcgcaaagcgcacgagctcatcaccacggagcgtgccaagatggagcgggccctggccgaggcgaagcggcaggcctccgaggacgccctgacggtcatcaaccagcaggaggactccagcgaggacttccagccactgcgctatttggatggtgtccccagctccttccagttcttcctgcccctcggctccgggggggccctgcacctgcctgcctcctccttccttacccctcccaaggacaagtgcctctcgccagacctgcccctgcccaagcagctggtgtgtcgctgggccaagtgtaaccagctctttgagctcctgcaagacctggtggaccatgtcaacgattaccatgtcaagcccgagaaggatgcggggtactgctgccactgggagggctgcgcccgccatggccgaggtttcaacgccaggtacaagatgctcatccacatccgcacacacaccaacgagaagccacaccgctgtccgacctgcagcaagagcttctcccgcctggagaacctgaagatccacaaccggtcgcacacaggtgagaagccctacgtctgcccctacgagggctgcaacaagcgctattccaactccagtgaccgctttaagcacacgcgcacccactacgtggacaagccctactactgcaagatgcccggctgccacaagcgctacacggaccccagctcactgcgcaagcacatcaaggcccatggccactttgtgtcccacgagcagcaagagctcctgcagctgcgcccaccccccaagccgccactgcccgcccccgacggcggcccctatgtcagtggggcccagatcatcatccccaacccagctgccctctttggaggccctggcctgcccggcttacccctacccctggcccccggcccccctgacctcagtgccctggcctgtggcaacggtgggggcagtgggggtggggggggcatgggccctgggctgccaggccccgtcctgcctctcaatctggccaagaacccgctgctgccctcgccctttggggctggcggactgggcttgcctgtggtctccctccttgctggcgcagctggtggcaaggccgagggggagaaggggcgtgggtcggtgcccaccagggccctgggcatggagggccacaagacgccccttgaaaggacggagagcagctgctcccggccaagccccgatggactccccctgctgccaggcaccgtgctggacctgtccacgggcgtcaactcagctgccagcagcccagaggcgttggcccctggctgggtggtcatcccgccgggctcggtgctgctcaaaccggctgtggtgaactga

SEQ ID NO: 3 - DNA, 93 bp, Synthetic construct
  source 1..93: mol_type = other DNA
ctggaaggagctggggacaccatccaaatagcgcagtagctggaaggtcctcgctagagtcctcctgctggttggtggccgtcagggcgtcct

SEQ ID NO: 4 - DNA, 351 bp, Synthetic construct
  source 1..351: mol_type = other DNA
cttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttca

SEQ ID NO: 5 - DNA, 495 bp, Synthetic construct
  source 1..495: mol_type = other DNA
cttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcagga

SEQ ID NO: 6 - DNA, 264 bp, Synthetic construct
  source 1..264: mol_type = other DNA
aggaggaggcaggcaggtgcagggcccacagaagcaccatcagggcttctggagccgaggggcaggaagaactggaagatgacgcaggaccaccgcgtcggggacaccatccaaatagcgcagtagctggaaggtcctcgctagagtcctcctgctggttggtggccgagacatgaggatcacccatgtgcgtcctcggaggcctgccgcttcgccaagggtggaggaacaccccaccctcagggcccgctccatcttggcacg

SEQ ID NO: 7 - DNA, 38 bp, Synthetic construct
  source 1..38: mol_type = other DNA
agatggccaacatgaggatcacccatgtctgcagggcc

SEQ ID NO: 8 - DNA, 3394 bp, Synthetic construct
  source 1..3394: mol_type = other DNA
caaataatgattttattttgactgatagtgacctgttcgttgcaacaaattgatgagcaatgcttttttataatgccaactttgtacaaaaaagcaggctccgaattgcccttactagtgccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccaattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaaccttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataaatgtcaggctcccttatacacagccagtctgcaggtcgacggccggccaggctcgggccagtgtactaattatgctctcttgaaattggctggagatgttgagagcaacccaggtcccttaattaaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagggcgaattcgacccagctttcttgtacaaagttggcattataaaaaataattgctcatcaatttgttgcaacgaacaggtcactatcagtcaaaataaaatcattatttg

SEQ ID NO: 9 - DNA, 3423 bp, Synthetic construct
  source 1..3423: mol_type = other DNA
caaataatgattttattttgactgatagtgacctgttcgttgcaacaaattgatgagcaatgcttttttataatgccaactttgtacaaaaaagcaggctccgaattgcccttactagtgccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccaattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaaccttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataaatgtcaggctcccttatacacagccagtctgcaggtcgacggccggccaggctcgggccagtgtactaattatgctctcttgaaattggctggagatgttgagagcaacccaggtcccttaattaaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaaaagggcggatccggtctccagatagatctcaattggaattcgacccagctttcttgtacaaagttggcattataaaaaataattgctcatcaatttgttgcaacgaacaggtcactatcagtcaaaataaaatcattatttg

SEQ ID NO: 10 - DNA, 3765 bp, Synthetic construct
  source 1..3765: mol_type = other DNA
caaataatgattttattttgactgatagtgacctgttcgttgcaacaaattgatgagcaatgcttttttataatgccaactttgtacaaaaaagcaggctccgaattgcccttactagtgccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccaattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaaccttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataaatgtcaggctcccttatacacagccagtctgcaggtcgacggccggccaggctcgggccagtgtactaattatgctctcttgaaattggctggagatgttgagagcaacccaggtcccttaattaaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattggaattcgacccagctttcttgtacaaagttggcattataaaaaataattgctcatcaatttgttgcaacgaacaggtcactatcagtcaaaataaaatcattatttg

SEQ ID NO: 11 - DNA, 3612 bp, Synthetic construct
  source 1..3612: mol_type = other DNA
caaataatgattttattttgactgatagtgacctgttcgttgcaacaaattgatgagcaatgcttttttataatgccaactttgtacaaaaaagcaggctccgaattgcccttactagtgccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccaattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaaccttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataaatgtcaggctcccttatacacagccagtctgcaggtcgacggccggccaggctcgggccagggagggccgtcatctggtgctcctcctccgtcaggtggctcacctgctggttccccgacatcaactgaggaaggaactagcgaaagtgcgacgcctgagagtggtcccggtactagcactgaaccgtcagaggggagtgcaccaggcagccccgccggctctccaacttccacggaggaggggacatctactgagccttctgagggttccgcacctggaaccagtactgagccctccgagcctaggttaattaaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctagtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaaaagggcggatccggtctccagatagatctcaattggaattcgacccagctttcttgtacaaagttggcattataaaaaataattgctcatcaatttgttgcaacgaacaggtcactatcagtcaaaataaaatcattatttg

SEQ ID NO: 12 - DNA, 3954 bp, Synthetic construct
  source 1..3954: mol_type = other DNA
caaataatgattttattttgactgatagtgacctgttcgttgcaacaaattgatgagcaatgcttttttataatgccaactttgtacaaaaaagcaggctccgaattgcccttactagtgccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccaattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaaccttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtgtccgttatcggggaagaagtggctgatctcagccaccgcgaaaatgacatcaaaaacgccattaacctgatgttctggggaatataaatgtcaggctcccttatacacagccagtctgcaggtcgacggccggccaggctcgggccagggagggccgtcatctggtgctcctcctccgtcaggtggctcacctgctggttccccgacatcaactgaggaaggaactagcgaaagtgcgacgcctgagagtggtcccggtactagcactgaaccgtcagaggggagtgcaccaggcagccccgccggctctccaacttccacggaggaggggacatctactgagccttctgagggttccgcacctggaaccagtactgagccctccgagcctaggttaattaaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattggaattcgacccagctttcttgtacaaagttggcattataaaaaataattgctcatcaatttgttgcaacgaacaggtcactatcagtcaaaataaaatcattatttg

SEQ ID NO: 13 - protein, 22 aa, Synthetic construct
GSGATNFSLLKQAGDVEENPGP

SEQ ID NO: 14 - protein, 23 aa, Synthetic construct
GSGQCTNYALLKLAGDVESNPGP

SEQ ID NO: 15 - protein, 80 aa, Synthetic construct
GGPSSGAPPPSGGSPAGSPTSTEEGTSESATPESGPGTSTEPSEGSAPGSPAGSPTSTEEGTSTEPSEGSAPGTSTEPSE

SEQ ID NO: 16 - protein, 561 aa, Synthetic construct
MNVGGGGSGGGGSGGGGSGRAMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGRGGGGSGGGGSGGGGSGPAQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTNV

SEQ ID NO: 17 - protein, 579 aa, Synthetic construct
MNVGGGGSGGGGSGGGGSGRAMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGRGGGGSGGGGSGGGGSGPALQLPPLERLTLGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTNV

SEQ ID NO: 18 - protein, 217 aa, Synthetic construct
VDIISVALKRHSTKAFDASKKLTPEQAEQIKTLLQYSPSSQNSQPWHFIVASTEEGKARVAKSAAGNYVFSERKMLDASHVVVFCAKTAMDDVWLKLVVDQEDADGRFATPEAKAANDKGRKFTADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 19 - DNA, 2713 bp, Synthetic construct
  source 1..2713: mol_type = other DNA
gccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccacttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcaggaggccggccaggctcgggccagggagggccgtcatctggtgctcctcctccgtcaggtggctcacctgctggttccccgacatcaactgaggaaggaactagcgaaagtgcgacgcctgagagtggtcccggtactagcactgaaccgtcagaggggagtgcaccaggcagccccgccggctctccaacttccacggaggaggggacatctactgagccttctgagggttccgcacctggaaccagtactgagccctccgagcctaggttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 20 - DNA, 501 bp, Synthetic construct
  source 1..501: mol_type = other DNA
tcgttgggcattccggacacctggccttcatacacctccccaaaggcgccatggcccagaccccgaatgagggtgatgtttttccgcggcacctccttcaggtcactgatggaggaggtcttgccagcaaagcagtggttggggttgtggtcggtcatgatggtcgaggtgcggagcttgctcagcttgtactcagggctctgcagctccatctgcatggcttgcagctcctagtgcttccggcggtacacttgggtcctttcccaggtgtaggctctacagtggttttgctccatatacgcatggctccacctgagtctccagtacgaacatctccattccccaagaaggctaaacactgcacaaattttggcttttcatatttcccaaaaattccctgttttcttgttagtgaattgccgctccaggtccagaagaaaatatgagatttaccgcaggtacttatggtatttgcatctgttgggtgaaactccacagcca

SEQ ID NO: 21 - DNA, 2530 bp, Synthetic construct
  source 1..2530: mol_type = other DNA
gccgccaccatggtgcggggttctcatcatcatcatcatcatggtatggctagcatgactggtggacagcaaatgggtcgggatctgtacgacgatgacgataaggatccgatggtgtctaagggcgaagagctgatcaaggaaaatatgcgtatgaaggtggtcatggaaggttcggtcaacggccaccaattcaaatgcacaggtgaaggagaaggcaatccgtacatgggaactcaaaccatgaggatcaaagtcatcgagggaggacccctgccatttgcctttgacattcttgccacgtcgttcatgtatggcagccgtacttttatcaagtacccgaaaggcattcctgatttctttaaacagtcctttcctgagggttttacttgggaaagagttacgagatacgaagatggtggagtcgtcaccgtcatgcaggacaccagccttgaggatggctgtctcgtttaccacgtccaagtcagaggggtaaactttccctccaatggtcccgtgatgcagaagaagaccaagggttgggagcctaatacagagatgatgtatccagcagatggtggtctgaggggatacactcatatggcactgaaagttgatggtggtggccatctgtcttgctctttcgtaacaacttacaggtcaaaaaagaccgtcgggaacatcaagatgcccggtatccatgccgttgatcaccgcctggaaaggttagaggaaagtgacaatgaaatgttcgtagtacaacgcgaacacgcagttgccaagttcgccgggcttggtggtgggatggacgagctgtacaagactagtggcagcggcgccacaaacttctctctgctaaagcaagcaggtgatgttgaagaaaaccccgggcctggcgcgccatcgttgggcattccggacacctggccttcatacacctccccaaaggcgccatggcccagaccccgaatgagggtgatgtttttccgcggcacctccttcaggtcactgatggaggaggtcttgccagcaaagcagtggttggggttgtggtcggtcatgatggtcgaggtgcggagcttgctcagcttgtactcagggctctgcagctccatctgcatggcttgcagctcctagtgcttccggcggtacacttgggtcctttcccaggtgtaggctctacagtggttttgctccatatacgcatggctccacctgagtctccagtacgaacatctccattccccaagaaggctaaacactgcacaaattttggcttttcatatttcccaaaaattccctgttttcttgttagtgaattgccgctccaggtccagaagaaaatatgagatttaccgcaggtacttatggtatttgcatctgttgggtgaaactccacagccaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 22 - protein, 535 aa, Synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGRGGGGSGGGGSGGGGSQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 23 - protein, 535 aa, Synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGRGGGGSGGGGSGGGGSQLHLPQVLADAVSRLVLGKFGDLTDNFSSPHARRKVLAGVVMTTGTDVKDAKVISVSTGTKFINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 24 - protein, 217 aa, Synthetic construct
DPDDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSLCEGTLLLWCDIIGQTTYRDLKL

SEQ ID NO: 25 - protein, 191 aa, Synthetic construct
DGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 26 - DNA, 90 bp, Synthetic construct
  source 1..90: mol_type = other DNA
gaaggagctggggacaccatccaaatagcgcagtagctggaaggtcctcgctagagtcctcctgctggttggtggccgtcagggcgtcct

SEQ ID NO: 27 - DNA, 150 bp, Synthetic construct
  source 1..150: mol_type = other DNA
ccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccaggg

SEQ ID NO: 28 - DNA, 90 bp, Synthetic construct
  source 1..90: mol_type = other DNA
tccatctgcatggcttgcagctcctagtgcttccggcggtacacttgggtcctttcccaggtgtaggctctacagtggttttgctccata

SEQ ID NO: 29 - DNA, 150 bp, Synthetic construct
  source 1..150: mol_type = other DNA
ttgctcagcttgtactcagggctctgcagctccatctgcatggcttgcagctcctagtgcttccggcggtacacttgggtcctttcccaggtgtaggctctacagtggttttgctccatatacgcatggctccacctgagtctccagtac

SEQ ID NO: 30 - DNA, 501 bp, Synthetic construct
  source 1..501: mol_type = other DNA
catagaagccatcccggcagtcctttcctacaagctcgtgggggtgaggccggtgaggagggtccttggtggccagggagatgcgcactgtccctggtcctgtgtagccattggtcttggtggtggggtgggtcttggtggtatctgtgctcctctcgcctgggatgctgcccgcggagcgcccctcgcacttgtagcggaagcgcatgccccgctgcttaggctgctcaatggtctccacagcggaggcccgagtcccggctctttccggtcaggtcctagggggcgaggtcatcgcctggtggggactgggtgagctcagacagcgcctcgggctggctcctccaggcctgcagcagggcactgcgctgggggccgctgagccccagggagccagggtgcacctccagcacgtgggcacggaggtcgtccaggtgcaggctgggcagtgcccggccacaggccaggcacaccagccggttcccccgcgggtcatggtcc

SEQ ID NO: 31 - DNA, 150 bp, Synthetic construct
  source 1..150: mol_type = other DNA
agcgcccctcgcacttgtagcggaagcgcatgccccgctgcttaggctgctcaatggtctccacagcggaggcccgagtcccggctctttccggtcaggtcctagggggcgaggtcatcgcctggtggggactgggtgagctcagacagc

SEQ ID NO: 32 - DNA, 90 bp, Synthetic construct
  source 1..90: mol_type = other DNA
tgccccgctgcttaggctgctcaatggtctccacagcggaggcccgagtcccggctctttccggtcaggtcctagggggcgaggtcatcg

SEQ ID NO: 33 - DNA, 501 bp, Synthetic construct
  source 1..501: mol_type = other DNA
gccacctcatcggggtccgtcattttgaactccccgttggtcccctcccaggtgatacagctggcgttggcgctgtcggagagcagctccaggaggaattgccacagctggatctgcccgcttccagggttggctaggcgactgctggtcgggcccaggatctggtacggatctggctagggccgttgctctgtattcttactgatcgtttgtgcccctccaaggggaggacttttgttgaggccagaattcatgttattgccccaagctcctcttctgactgagtcatacgaagggttctgctgcccgtggctgctgctctgttagctatattggcttggagcttggctgtgggatccagtttggggtgggtacctagtgggaggctgctgcccatggctgctttgttggccatggctactctgctgtccatggctgctcggttgcccatgggtgttctgctgggagtacctgctctggtcatacctagtcggctgtgta

SEQ ID NO: 34 - DNA, 501 bp, Synthetic construct
  source 1..501: mol_type = other DNA
atcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtacaaagaccatgaaataacagacaatggactcccttggcgggccaggttgtaggccgggtccaggggccattccaaaggggagacgactcaatggtgtaagacgacattgtggaatagcaagggcagttcctcgccttgggttgtaaagggaggtcttactacctccatatacgaacacaccggcgacccaagttccttcgtcggtagtcctttctacgtgactcctagccaggagagctcttacaccttctgcaatgttctcaaatttcgggttggaacctccttgaccacgaggctttcc

SEQ ID NO: 35 - DNA, 501 bp, Synthetic construct
  source 1..501: mol_type = other DNA
tgttgggagtgtgatgggccggaaaggtggaggcccattagggtttgcacttggcgctgtaggtctactcttgacaaagatctaagcattgacattagggcatccacgtcagtgggacccagtaggtctaagttttccatacagtacacccagtgtaagaatgtctgtggtgtgctgcgagaccctatagtgtccttgcttaaaaatatcaaagacctaatatccctcgcacacagctccccgtctacgtagagaacagtgagctggtacgggctgaaatacctcattgtgcccgctaggtggcgctctaaaaaacgcgggtctaagtggagcaggtcgcgcaagaggtctctgcgacctgcacgaaacagacattccgctaacaggggaaacgttaacctgccctcctcctttaaagctctaagagctccaattaattgggccagtgtgggttggggtaggaacacgtttaggaggaacaataccacttccctgtcatcc

SEQ ID NO: 36 - DNA, 2911 bp, Synthetic construct
  source 1..2911: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccagaaggagctggggacaccatccaaatagcgcagtagctggaaggtcctcgctagagtcctcctgctggttggtggccgtcagggcgtcctggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 37 - DNA, 2971 bp, Synthetic construct
  source 1..2971: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccaccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 38 - DNA, 3316 bp, Synthetic construct
  source 1..3316: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcaggaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 39 - DNA, 3316 bp, Synthetic construct
  source 1..3316: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcaggaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 40 - DNA, 2911 bp, Synthetic construct
  source 1..2911: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccatccatctgcatggcttgcagctcctagtgcttccggcggtacacttgggtcctttcccaggtgtaggctctacagtggttttgctccataggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 41 - DNA, 2911 bp, Synthetic construct; identical to SEQ ID NO: 40
  source 1..2911: mol_type = other DNA

SEQ ID NO: 42 - DNA, 2971 bp, Synthetic construct
  source 1..2971: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccattgctcagcttgtactcagggctctgcagctccatctgcatggcttgcagctcctagtgcttccggcggtacacttgggtcctttcccaggtgtaggctctacagtggttttgctccatatacgcatggctccacctgagtctccagtacggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 43 - DNA, 2971 bp, Synthetic construct; identical to SEQ ID NO: 42
  source 1..2971: mol_type = other DNA

SEQ ID NO: 44 - DNA, 3322 bp, Synthetic construct
  source 1..3322: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccatcgttgggcattccggacacctggccttcatacacctccccaaaggcgccatggcccagaccccgaatgagggtgatgtttttccgcggcacctccttcaggtcactgatggaggaggtcttgccagcaaagcagtggttggggttgtggtcggtcatgatggtcgaggtgcggagcttgctcagcttgtactcagggctctgcagctccatctgcatggcttgcagctcctagtgcttccggcggtacacttgggtcctttcccaggtgtaggctctacagtggttttgctccatatacgcatggctccacctgagtctccagtacgaacatctccattccccaagaaggctaaacactgcacaaattttggcttttcatatttcccaaaaattccctgttttcttgttagtgaattgccgctccaggtccagaagaaaatatgagatttaccgcaggtacttatggtatttgcatctgttgggtgaaactccacagccaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 45 - DNA, 3322 bp, Synthetic construct
  source 1..3322: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacatagaagccatcccggcagtcctttcctacaagctcgtgggggtgaggccggtgaggagggtccttggtggccagggagatgcgcactgtccctggtcctgtgtagccattggtcttggtggtggggtgggtcttggtggtatctgtgctcctctcgcctgggatgctgcccgcggagcgcccctcgcacttgtagcggaagcgcatgccccgctgcttaggctgctcaatggtctccacagcggaggcccgagtcccggctctttccggtcaggtcctagggggcgaggtcatcgcctggtggggactgggtgagctcagacagcgcctcgggctggctcctccaggcctgcagcagggcactgcgctgggggccgctgagccccagggagccagggtgcacctccagcacgtgggcacggaggtcgtccaggtgcaggctgggcagtgcccggccacaggccaggcacaccagccggttcccccgcgggtcatggtccggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 46 - DNA, 2971 bp, Synthetic construct
  source 1..2971: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccaagcgcccctcgcacttgtagcggaagcgcatgccccgctgcttaggctgctcaatggtctccacagcggaggcccgagtcccggctctttccggtcaggtcctagggggcgaggtcatcgcctggtggggactgggtgagctcagacagcggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 47 - DNA, 2911 bp, Synthetic construct
  source 1..2911: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccatgccccgctgcttaggctgctcaatggtctccacagcggaggcccgagtcccggctctttccggtcaggtcctagggggcgaggtcatcgggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 48 - DNA, 3322 bp, Synthetic construct
  source 1..3322: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccagccacctcatcggggtccgtcattttgaactccccgttggtcccctcccaggtgatacagctggcgttggcgctgtcggagagcagctccaggaggaattgccacagctggatctgcccgcttccagggttggctaggcgactgctggtcgggcccaggatctggtacggatctggctagggccgttgctctgtattcttactgatcgtttgtgcccctccaaggggaggacttttgttgaggccagaattcatgttattgccccaagctcctcttctgactgagtcatacgaagggttctgctgcccgtggctgctgctctgttagctatattggcttggagcttggctgtgggatccagtttggggtgggtacctagtgggaggctgctgcccatggctgctttgttggccatggctactctgctgtccatggctgctcggttgcccatgggtgttctgctgggagtacctgctctggtcatacctagtcggctgtgtaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 49 - DNA, 3316 bp, Synthetic construct
  source 1..3316: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcaggaggccggccaggctcgggccagtgtactaattatgctctcttgaaattggctggagatgttgagagcaacccaggtcccttaattaaggaccctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 50 - DNA, 3238 bp, Synthetic construct
  source 1..3238: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcaggaggccggccaggctcgggccagtgtactaattatgctctcttgaaattggctggagatgttgagagcaacccaggtcccttaattaaggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 51 - DNA, 3427 bp, Synthetic construct
  source 1..3427: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacttctcgggcttgacagggtaatcgttgacagggtccaccaggtcttgcaggagctcaaagagctggttacacttggcccagcgacacaccagctgcttgggcaggggcaggtctggcgagaggcacttgtccttgggaggggtaaggaaggaggaggcaggcaggtgcagggcccccccggagccgaggggcaggaagaactggaaggagctggggacaccatccaaatagcgcagtagctggaaggtcctcgctagagtcctcctgctggttggtggccgtcagggcgtcctcggaggcctgccgcttcgcctcggccagggcccgctccatcttggcacgctccgtggtggtgggctcgtgcgctttgcgctccgcgtccgacacggctttctgcagctccgacatggcctgccgcttcacctcattcacggcctcttcagccttcctccagatgtcctcaggcacgtagccggtgggggtcctcggcaggaggccggccaggctcgggccagggagggccgtcatctggtgctcctcctccgtcaggtggctcacctgctggttccccgacatcaactgaggaaggaactagcgaaagtgcgacgcctgagagtggtcccggtactagcactgaaccgtcagaggggagtgcaccaggcagccccgccggctctccaacttccacggaggaggggacatctactgagccttctgagggttccgcacctggaaccagtactgagccctccgagcctaggttaattaaggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 52 - DNA, 3511 bp, Synthetic construct
  source 1..3511: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccaatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtacaaagaccatgaaataacagacaatggactcccttggcgggccaggttgtaggccgggtccaggggccattccaaaggggagacgactcaatggtgtaagacgacattgtggaatagcaagggcagttcctcgccttgggttgtaaagggaggtcttactacctccatatacgaacacaccggcgacccaagttccttcgtcggtagtcctttctacgtgactcctagccaggagagctcttacaccttctgcaatgttctcaaatttcgggttggaacctccttgaccacgaggctttccggccggccaggctcgggccagggagggccgtcatctggtgctcctcctccgtcaggtggctcacctgctggttccccgacatcaactgaggaaggaactagcgaaagtgcgacgcctgagagtggtcccggtactagcactgaaccgtcagaggggagtgcaccaggcagccccgccggctctccaacttccacggaggaggggacatctactgagccttctgagggttccgcacctggaaccagtactgagccctccgagcctaggttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 53 - DNA, 3511 bp, Synthetic construct
  source 1..3511: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatgtattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccatgttgggagtgtgatgggccggaaaggtggaggcccattagggtttgcacttggcgctgtaggtctactcttgacaaagatctaagcattgacattagggcatccacgtcagtgggacccagtaggtctaagttttccatacagtacacccagtgtaagaatgtctgtggtgtgctgcgagaccctatagtgtccttgcttaaaaatatcaaagacctaatatccctcgcacacagctccccgtctacgtagagaacagtgagctggtacgggctgaaatacctcattgtgcccgctaggtggcgctctaaaaaacgcgggtctaagtggagcaggtcgcgcaagaggtctctgcgacctgcacgaaacagacattccgctaacaggggaaacgttaacctgccctcctcctttaaagctctaagagctccaattaattgggccagtgtgggttggggtaggaacacgtttaggaggaacaataccacttccctgtcatccggccggccaggctcgggccagggagggccgtcatctggtgctcctcctccgtcaggtggctcacctgctggttccccgacatcaactgaggaaggaactagcgaaagtgcgacgcctgagagtggtcccggtactagcactgaaccgtcagaggggagtgcaccaggcagccccgccggctctccaacttccacggaggaggggacatctactgagccttctgagggttccgcacctggaaccagtactgagccctccgagcctaggttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 54 - DNA, 756 bp, Synthetic construct
  source 1..756: mol_type = other DNA
atgtcaaaagcagattcaggtgttcatttatgtattattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgctga

SEQ ID NO: 55 - DNA, 756 bp, Synthetic construct
  source 1..756: mol_type = other DNA
atgcggcgtctgcgcctctcagcctcccggagggccgggggcagcagggggctgggggcccggcgcctggagaggaggctgaaggagtccctgcagaactggttccgggccgagtgtctcatggactatgacccgcgggggaaccggctggtgtgcatggcctgtggccgggcactgcccagcctgcacctggacgacatccgtgcccacgtgctggaggtgcaccctggctccctggggctcagcggcccccagcgcagtgccctgctgcaggcctgggggggccagcccgaggcgctgtctgagctcacccagtccccaccaggcgatgacctcgccccccaggacctgaccggaaagagccgggactcggcctccgctgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttccttga

SEQ ID NO: 56 - DNA, 756 bp, Synthetic construct
  source 1..756: mol_type = other DNA
atgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactattga

SEQ ID NO: 57 - DNA, 1926 bp, Synthetic construct
  source 1..1926: mol_type = other DNA
atgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtga

SEQ ID NO: 58 - DNA, 633 bp, Synthetic construct
  source 1..633: mol_type = other DNA
atggccacttacgaggttctctgtgaggtggcgcggaaactgggcacggatgacagggaagtggtattgttcctcctaaacgtgttcatacctcaacccacactggcccaattaattggagctcttagagctttaaaggaggagggcaggttaacgtttcccctgttagcggaatgtctgtttcgtgcaggtcgcagagacctcttgcgcgacctgcttcacttagacccgcgttttttagagcgccacctagcgggcacaatgagttatttcagcccttatcagctcactgttctccacgtagacggggagctgtgtgcgagggatattaggtctttgatatttttaagcaaggacactatagggtctcgcagcacaccacagacattcttacactgggtgtactgtatggaaaacttagacctactgggtcccactgacgtggatgccctaatgtcaatgcttagatctttgtcaagagtagacctacagcgccaagtgcaaaccctaatgggccttcacctttccggcccatcacactcccaacactatcgccatacaccagactacaaagaccatgacggtgattataaagatcatgatatcgattacaaggatgacgatgacaagtag

SEQ ID NO: 59 - DNA, 3319 bp, Synthetic construct
  source 1..3319: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccagcatcggggagcagggtgtgctggagcagcggggcttctctgcctccgcctttcccggggagttggcctcagccccagggaagggcaaggtggtcagggtacttctggactctccattactatcgacttccattttacgcaccagtctgcacaatccagactaaatcccagaagatgtcactgtacaatttccaataggtcccaagggaacacagacataataatttttctgttctactccaggtaatttttcattatatctggtacctgtagcaggcaagttctgttctctttcagcatcagactctctggtacataactttccatagtaattccagccctggaggcactagatacaatggcagtcagggcaatttgggaaggtgtgtatacaaggtaagcatccgtcaatgcaattctattaagaaagtcatcagctgttttcctcaaaatctctggattctccaatatgggatagcgggtctttacgtcgatgagggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 60 - DNA, 3322 bp, Synthetic construct
  source 1..3322: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccaccttttagatgtgtgttttcagttctgacagctttatatttcatatctgagaccttttgttttgctaacattttttcttttatttctaattccatttttaactgtttttccagaagggcagctttctttgtgacagttgctatagtgatctccttctggtggagctcttctgttaggtcagagatttcattcctcattctttcctgctcagagttatggtactcttccacctgttggagttagcttcttatttgcccttagtgggtcttaaaaggaacttccagtaagagggtctcaaagtctgaacttccatggcagctgcctggaagcaaattgctgtggagatggaatagatggtggtatctgcatgaggaaaatagggaaccttccctgcttcaatgcctttggaatacattgtctctaccaatttggacgctaaatacatggaaattgttgtgcttttataaaacatcattgatatacctgccaaaaatccagctaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 61 - DNA, 3322 bp, Synthetic construct
  source 1..3322: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccatattcaggtctccatgcttcatgtattcaaagaccatggtgggggggtccccatcgccgcacactccatagaacttggcaatgtgctcatgctgcaggttggtgggcagctcggcctccctctggaaatccttccgggcagccagggtgggatccttcagggccttcacagccacaagcatcttgtccttagtcgggctggggttgtagcactcggccaggaagacctttccaaaggctccctcacccagttctcgcttcagcacgatgtctctcctcttactgtgctgcacatctgctattctcccaataggcatggcgtgctcttcaggcggggagacagagaccatggtgtggttcatgtaagccaggtcttcccgatgagagaggttggtgggcttcccttccctatgcagcccgtcctcggagagcctggactgtttggaatccacggagtgccgggggttcaggatcagagggtgcatggtggggctgggcatcaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 62 - DNA, 3085 bp, Synthetic construct
  source 1..3085: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccattggaagtctgtccatggtcgctggaggaggacgcggtcatctctgtcttagccaggtgtggcgttccgtgggcacactcaaacaacgactagtcctcactcacaactgataaggcttctgagttcaaagcatcttgctgttatcaacagcatcgagtaaggataggtatctagaatgttcaatatgacctgccgcgctccaggcggcgctccccgcccctcgccctccgcctccgcctccgcctcctgcttagctcgcgcctaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 63 - DNA, 3022 bp, Synthetic construct
  source 1..3022: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccacttctgggtatcataggtaagggtagtattgggtggcaatgttctgtttctgttaattgtgttcacagcaaatctgaatgcaagttcctcagctcccattaggccagattccacatattcaaaaataccaccggcaggcggaactccagatgctcctgcgccaggaggagcagatacctgttaagggtacacgacagcgccggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 64 - DNA, 3319 bp, Synthetic construct
  source 1..3319: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccagtctcctcccagcagctcggtcaccatctccagctggtcggccgtggagaagctcccgccaccgccgtcgttgtctccccgaagggagaagggtgtgaccgcaacgtaggagggcgagcagagcccggagcggcggctaggggacaggggcggggtgggcagcagctcgaatttcttccagatatcctcgctgggcgccgggggctgcagctcgctctgctgctgctgctggtagaagttctcctcctcgtcgcagtagaaatacggctgcaccgagtcgtagtcgaggtcatagttcctgttggtgaagctaacgttgaggggcatcgtcgcgggaggctgtagagggcagatctggccgtgtctccacaggtcacagggaccgccaacatcctttccgcaaggaaatccactggaaggtgccgggggtcccggggcacatctttgctcgcagctcgtcgtcgcccctcctcgtcccggccgccccgagcccgcccgggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 65 - DNA, 2932 bp, Synthetic construct
  source 1..2932: mol_type = other DNA
gccgccaccatggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgctggcggccgcgggggaggcggttccggtggcggcggaagcggaggtggaggatcacagctgcatttaccgcaggttttagctgacgctgtctcacgcctggtcctgggtaagtttggtgacctgaccgacaacttctcctcccctcacgctcgcagaaaagtgctggctggagtcgtcatgacaacaggcacagatgttaaagatgccaaggtgataagtgtttctacaggaacaaaatttattaatggtgaatacatgagtgatcgtggccttgcattaaatgactgccatgcagaaataatatctcggagatccttgctcagatttctttatacacaacttgagctttacttaaataacaaagatgatcaaaaaagatccatctttcagaaatcagagcgaggggggtttaggctgaaggagaatgtccagtttcatctgtacatcagcacctctccctgtggagatgccagaatcttctcaccacatgagccaatcctggaagaaccagcagatagacacccaaatcgtaaagcaagaggacagctacggaccaaaatagagtctggtcaggggacgattccagtgcgctccaatgcgagcatccaaacgtgggacggggtgctgcaaggggagcggctgctcaccatgtcctgcagtgacaagattgcacgctggaacgtggtgggcatccagggatcactgctcagcattttcgtggagcccatttacttctcgagcatcatcctgggcagcctttaccacggggaccacctttccagggccatgtaccagcggatctccaacatagaggacctgccacctctctacaccctcaacaagcctttgctcagtggcatcagcaatgcagaagcacggcagccagggaaggcccccaacttcagtgtcaactggacggtaggcgactccgctattgaggtcatcaacgccacgactgggaaggatgagctgggccgcgcgtcccgcctgtgtaagcacgcgttgtactgtcgctggatgcgtgtgcacggcaaggttccctcccacttactacgctccaagattaccaagcccaacgtgtaccatgagtccaagctggcggcaaaggagtaccaggccgccaaggcgcgtctgttcacagccttcatcaaggcggggctgggggcctgggtggagaagcccaccgagcaggaccagttctcactcacgactagtggcagcggcgccacaaacttctctctgctaaagcaagcaggtgatgttgaagaaaaccccgggcctggcgcgccatccacctgccttggcctggagtcttccagtgtgatgatggtgaggatgggcctctagttcaggccgcccaggcaggaactgttacacaggtggttgtggtggagggtggtaggccggccaggctcgggccagtgtactaattatgctctcttgaaattggctggagatgttgagagcaacccaggtcccttaattaaggtggacatcatcagcgtggctctgaagaggcactccaccaaggctttcgacgcttccaagaaactgacccctgaacaggccgagcagatcaagaccctgctccagtacagccctagctcccagaacagccagccttggcacttcatcgtggctagcaccgaggaaggcaaagctagggtggctaagagcgccgctggcaactacgtgttcagcgagaggaagatgctggatgctagccacgtggtggtgttctgcgctaagaccgccatggacgatgtgtggctgaagctggtggtggatcaggaagatgctgatggcaggttcgctacccctgaagctaaggccgctaacgacaagggcaggaagttcactgccgacatgcacaggaaggatctgcacgatgatgctgagtggatggccaagcaggtgtacctgaacgtgggcaacttcctgctcggcgtggctgccctgggcctcgatgctgtgcccatcgaaggcttcgatgctgctatcctggatgccgagttcggcctgaaggagaaaggctacaccagcctggtggtggtgcctgtgggccaccacagcgtggaggacttcaacgctaccctgcctaagagcaggctgccccagaacatcaccctgaccgaggtgtgattaattaaaagggcggatccggtctccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatggccaacatgaggatcacccatgtctgcagggccagatagatctcaattg

SEQ ID NO: 66 - DNA, 527 bp, Synthetic construct
  source 1..527: mol_type = other DNA
gccgccaccatgctcatcgacttaaagacccgctatcccatattggagaatccagagattttgaggaaaacagctgatgactttcttaatagaattgcattgacggatgcttaccttttatacacaccttcccaaattgccctgactgccattttatctagtgcctccagggctggaattactatggaaagttatttatcagagagtctgatgctgaaagagaacagaacttgcctgtcacagttactagatataatgaaaatacctggagtagaacagaaaaattattatgtctgtgttcccttgggactcattggaaattgtacagtgacatcttctgggatttagtctggattgtgcagactggtgcttaaaatggaagtcgatattaatggagagtctagaagtaccctgaccaccttgcccttccctggggctgaggccaactccccgggaaaggcggaggcagagaagccccgctgctccagcacaccctgctccccgatgcggtgattaattaagaattc

SEQ ID NO: 67 - DNA, 776 bp, Synthetic construct
  source 1..776: mol_type = other DNA
gccgccaccatgtacaataaagaaaacttccagcttggagcttttcttggctcttttgttagtatatacaagggtactagttgcttcctgcgctggatcagaaacttagatgatgaactacatgctattatagctggatttttggcaggtatatcaatgatgttttataaaagcacaacaatttccatgtatttagcgtccaaattggtagagacaatgtatttcaaaggcattgaagcagggaaggttccctattttcctcatgcagatactatcatctattccatctctacagcaatttgcttccaggcagctgtcatggaagttcagactttgagaccatcttactggaagttccttttaagactcaccaagggcaaataagaagccaactccaacaggtggaagagtaccataactctgagcaggaaagaatgaggaatgaaatctctgacctaacagaagagcttcatcagaaggagatcactatagcaactgtcacaaagaaagctgcccttctggaaaaacagttaaaaatggaattagaaataaaagaaaaaatgttagcaaaacaaaaggtctcagatatgaaatataaagctgtcagaactgaaaacacacatctaaaaggaatgatgggagatttagaccccggagaatacatgagtatggacttcactaacagggaacagtcaaggcatacatctattaataaactgcaatatgagaatgaaaggctccgaaatgatcttgcaaaacttcttaattaagaattc

SEQ ID NO: 68 - DNA, 779 bp, Synthetic construct
  source 1..779: mol_type = other DNA
gccgccaccatgaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcagatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtgattaattaagaattc

SEQ ID NO: 69 - DNA, 922 bp, Synthetic construct
  source 1..922: mol_type = other DNA
accggtgagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggtgattaattaagaattc

SEQ ID NO: 70 - DNA, 520 bp, Synthetic construct
  source 1..520: mol_type = other DNA
accggtgggcagctgcaatggcgctgtcgtgtacccttaacaggtatctgctcctcatggcgcaggagcatctggagttccgcctgccggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgttaattaagaattc

SEQ ID NO: 71 - DNA, 1990 bp, Synthetic construct
  source 1..1990: mol_type = other DNA
accggtggccgggcgggctcggggcggccgggacgaggaggggcgacgacgagctgcgagcaaagatgtgccccgggacccccggcaccttccagtggatttccttgcggaaaggatgttggcggtccctgtgacctgtggagacacggccagatctgccctccacagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaattaattaagaattc

SEQ ID NO: 72 - DNA, 1044 bp, Homo sapiens
  source 1..1044: mol_type = genomic DNA
gccgccaccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccaggccaaggcaggtggatcacctgaggtcaggagttcaagacagcctggccaacatagcgaaatcccatctctactaaaaatacaaaaattagcctggcagtggtggcccacgcctgtaatcccagctactaagggggctgaggcaggaggatctcttaaccagggaggaggaggttgcagtgagcagagattgtgccactgcactccagcctgtgcaacagagtgagactctgtctctgattaattaagaattc

SEQ ID NO: 73 - DNA, 1044 bp, Synthetic construct
  source 1..1044: mol_type = other DNA
gccgccaccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccagaggcccatcctcaccatcatcacactggaagactccaggccaaggcaggtggatcacctgaggtcaggagttcaagacagcctggccaacatagcgaaatcccatctctactaaaaatacaaaaattagcctggcagtggtggcccacgcctgtaatcccagctactaagggggctgaggcaggaggatctcttaaccagggaggaggaggttgcagtgagcagagattgtgccactgcactccagcctgtgcaacagagtgagactctgtctctgattaattaagaattc

SEQ ID NO: 74 - DNA, 3894 bp, Synthetic construct
  source 1..3894: mol_type = other DNA
ggcgcggcgctcgcggctgctgcctgggagggaggccgggcaggcggctgagcggcgcggctctcaacgtgacggggaagtggttcgggcggccgcggcttactaccccagggcgaacggacggacgacggaggcgggagccggtagccgagccgggcgacctagagaacgagcgggtcaggctcagcgtcggccactctgtcggtccgctgaatgaagtgcccgcccctctaagcccggagcccggcgctttccccgcaagatggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 75 - DNA, 3606 bp, Synthetic construct
  source 1..3606: mol_type = other DNA
gccgagcgagcagcctgccgtccgtgcggccggccgccccgtgcatgcgccccgcgccccggggccccgcgcagcgagcgctccgcgcgggctgccgccggccccgcggccccgcgccccgccgccccggggccccgctccgcagcgcagcgcatggagcccggcggggaccaccggagccggagcagcggcggcaggggcggccccgggccagcagtggcctcggcacggggccgacggctgccgcccgccggatcgagcggcagcgcggagccagaggaagacgaaggcgggcaagatcttcagctggaagggggtgccttggggtcctgggggagtgcccccctgccctcctccagggccaggggaccagcatcttcaggcaggaaatattcagaccactgtgaggcccgggcctcgaggcctggaaagagccgcatccctggccgtgaccaccggcgctactaccacgaccactggcggctggagtacctgatggacttcaaccctgcccggcacggcatggtgtgcatggtgtgcggcagctccctggccaccctcaagctcagcaccatcaagcgccacatccgccaaaagcacccctactccttgcattggagtccccgggagaaggaagtcatcagcaacagctgggatgcacacctggggctgggggcctgcggagaggccgagggcctgggggtccagggggctgaggaggaggaggaggaggaagaagaggaggaggaggagggggccggtgtcccagcttgcccgcccaagggcccaggcaaagccccagctggtgggggctgccggcgccagcggcgagggggcccagtggcaccccgggctcggcgtctgcgcctctcagcctcccggagggccgggggcagcagggggctgggggcccggcgcctggagaggaggctgaaggagtccctgcagaactggttccgggccgagtgtctcatggactatgacccgcgggggaaccggctggtgtgcatggcctgtggccgggcactgcccagcctgcacctggacgacatccgtgcccacgtgctggaggtgcaccctggctccctggggctcagcggcccccagcgcagtgccctgctgcaggcctgggggggccagcccgaggcgctgtctgagctcacccagtccccaccaggcgatgacctcgccccccaggacctgaccggaaagagccgggactcggcctccgctgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctgg

SEQ ID NO: 76 - DNA, 3845 bp, Synthetic construct
  source 1..3845: mol_type = other DNA
gcagtgcggcgcctagagggaaagcgagagggagacggacgttgagagaacgaggaggaaggagagaaaatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagaacccttcttatgactcagtcagaagaggagcttggggcaataacatgaattctggcctcaacaaaagtcctccccttggaggggcacaaacgatcagtaagaatacagagcaacggccccagccagatccgtatcagatcctgggcccgaccagcagtcgcctagccaaccctggaagcgggcagatccagctgtggcaattcctcctggagctgctctccgacagcgccaacgccagctgtatcacctgggaggggaccaacggggagttcaaaatgacggaccccgatgaggtggccaggcgctggggcgagcggaaaagcaagcccaacatgaattacgacaagctgagccgggccctccgttattactatgataaaaacattatgaccaaagtgcacggcaaaagatatgcttacaaatttgacttccacggcattgcccaggctctgcagccacatccgaccgagtcgtccatgtacaagtacccttctgacatctcctacatgccttcctaccatgcccaccagcagaaggtgaactttgtccctccccatccatcctccatgcctgtcacttcctccagcttctttggagccgcatcacaatactggacctcccccacggggggaatctaccccaaccccaacgtcccccgccatcctaacacccacgtgccttcacacttaggcagctactactagaagcttactcatcagtggccttctagctgaagcccatcctgcacacttactggatgctttggactcaacaggacatatgtggccttgaagggaagacaaaactggatgttctttcttgttggatagaacctttgtatttgttctttaaaaacattttttttaatgttggtaacttttgcttcctctacctgaacaaagagatgaataattccatgggccagtatgccagtttgaattctcagtctcctagcatcttgtgagttgcatattaagattactggaatggttaagtcatggttctgagaaagaagctgtacgttttctttatgtttttatgaccaaagcagtttcttgtcaatacacggggttcagtatgacacagaatcatggacttaacccgtcatgttctggtttgagatttagtgacaaatagaggtgggaagcttataatctaattttaggaggaccaaattcagtggatggcaactggaacattgattgtaaggccagtgaagttttcacccaactggaatttgatggaaagaaggtttgtgtgtttaagacgccaagggcattgcagaatccctctcagtggacagtatgcactcagctgaccactctctctagaaatagtcaagatatgaactaagaaattttaatgcaaatacatacattcctgaaagacggggaattaaattactaatttttttttttttttaaatgatgacagtggtcccagaacttggaaaagttgtagggatttctaaactcaagcagattcgcaagtgctgtgcgcttgtcagaccatcagaccagggccaaccaatcagaaggcaacttactgtataaattatgcagagttattttcctatatctcacagtattaaaaataaataattaaaaattaagaataaataaacgagttgacctcggtcacaaaagcagttttactatcgaatcaatcgctgttattttttttaatgtaatttgtacatcttttttcaatctgtacatttgggctgtctgtatgtttttatagctggtttttaaaaagcataatatgcctatagctgaaaaggaaacagggctgtttaagtcactgacttatgagaaagcaaagcactggtacagttatttaacaggcatacacaagcagggaaaagataatccatttagatctttaatgctttggaaatgcgtgtaacagtactgcaataatcacagctctgggaaaaacaacgaaactttcccttgtggagaggagggattttcctgctctatataagcaacatatttttagacattaaaatatatataattttgcaggtaattgttgacttttttaactatattaagtgttaagctgacaactgtcaaagaagaccatgttgtaaaataatttgactaaataaatggttccttctctcagtgctgaggacagttttcttatttaccgcccccgttaggtcaaagggttttccctggggaactttcctatttacttcttgcactatcaagaatttttcgaatgtacctactgcagtacagcagaaggtaaaaaatcagtgtggtttttcattgttgttgatgatgtttgtagtgtttttgtgtgtgttatttaaatcttcctccagcctaaaagggttttataaaacagcagctaaggccatggataaacctgtatgtaaggactggagcaaagcgagctggtctatccagactggtctgtgagatttaactctgcagcctcccctgggcacttcagacccagacggccaccttctgccactccagcaaagaataagcgccctgcttccttcaggtctcagaccaggactttatggctcatgcagatttttaaggtcatttttcttcccaaggaagaaacttgcctccagttccttcactgttaggtagcttattttcattttctctattttacaatgaaaagagtgagacctgggaagtccttgatttgcaaggaattagactcacagcattggtaaccctagaaccttcttagggtaacactaagtaccttctagacaacatgtctacctaaatgaaatgggatgtgtttcggaacatttgtctccagtttttttttaatcttgcaccctgccatttaaaaagatgtgtaaagcacatattctcaacatatgcacattgatttataaatcatatatacaaactgttacattattcttcatattagaaaacaaatacaaaatagaacattttaaatggtgatataaaaataaattgaaactgaaattcta

SEQ ID NO: 77 - DNA, 3750 bp, Synthetic construct
  source 1..3750: mol_type = other DNA
agcgcgccagcgccggctagccggacgccctaggcttccgcgagatcttcggtgggggtacgggtgttttacgccaggacgctgatgcgtttgggttctcgtctgcagaccctctggacctggtcacgattccataatgtaccacaacagtagtcagaagcggcactggaccttctccagcgaggagcagctggcaagactgcgggctgacgccaaccgcaaattcagatgcaaagccgtggccaacgggaaggttcttccgaatgatccagtctttcttgagcctcatgaagaaatgacactctgcaaatactatgagaaaaggttattggaattctgttcggtgtttaagccagcaatgccaagatctgttgtgggtacggcttgtatgtatttcaaacgtttttatcttaataactcagtaatggaatatcaccccaggataataatgctcacttgtgcatttttggcctgcaaagtagatgaattcaatgtatctagtcctcagtttgttggaaacctccgggagagtcctcttggacaggagaaggcacttgaacagatactggaatatgaactacttcttatacagcaacttaatttccaccttattgtccacaatccttacagaccatttgagggcttcctcatcgacttaaagacccgctatcccatattggagaatccagagattttgaggaaaacagctgatgactttcttaatagaattgcattgacggatgcttaccttttatacacaccttcccaaattgccctgactgccattttatctagtgcctccagggctggaattactatggaaagttatttatcagagagtctgatgctgaaagagaacagaacttgcctgtcacagttactagatataatgaaaatacctggagtagaacagaaaaattattatgtctgtgttcccttgggactcattggaaattgtacagtgacatcttctgggatttagtctggattgtgcagactggtgcttaaaatggaagtcgatattaatggagagtctagaagtaccctgaccaccttgcccttccctggggctgaggccaactccccgggaaaggcggaggcagagaagccccgctgctccagcacaccctgctccccgatgcggaggaccgtgtcaggctaccagatcctacacatggactctaactatttggttggcttcacgactggcgaggaactcctgaagttagctcagaagtgcacaggaggtgaagagagcaaagcagaagccatgccatccttacgctccaaacagctagatgcaggacttgcccgttcctctcgtttgtataaaaccagaagtaggtactaccagccatacgagattccagctgtcaatggcaggaggcgaaggcggatgccaagctcaggagacaagtgcactaaatctttaccttatgaaccttacaaggccctccatgggcctctgcctctttgtcttcttaaaggtaagagggctcactccaaatctctggactacctcaatctagataaaatgatcaaggagccagctgatacagaagtgctacagtaccagcttcaacacctaaccctccgaggggaccgtgtgtttgctaggaataatacatgaatgacttggagagagcttaaaccaatttaggtcagcctacgcttggctagaaaaaacccactgctgtactctgtacatgactcttcacactatagatggttatatcagctaagtgttcctggaacataaaaattgtttgggtcaaatttgaatacaggaatgaaatcacaggtacttgggggggggatatcattctagagcacgcaactgcaaagaaaacagaatgttgactgttagtttgtatagctttttagctaggaaaaaaaggctttggtacagtaatttcatctttatgattctgacactcaaattgggaatgttacctgcttggttgttgctgacttgatatgattcattagaaatttatatcttcagtactcaagtacttcttgaatctctgtattttactataaaatgtatgtaatgatttgttttatgaaatttagaacttgaacattgctgaattggaccactttttatttttaaatattgagtttaaatattttataactggttttgcactgaaaaaattaacatttcagattgacaagagagtaatctttcttcacttgcctcaataatgttattgagcaatgaattttttatttccgcatggaaagttattgatctctatggctgtaaaatatttctttatagcgttattaaagtgtgtcttaataaaattaaatttgggatacaaagtatttattttacaatgggtgggcgggggaaacttttccagaaagtttccaatatgacgttttcataagttgaaaaaactctccttagtgcttattttctaacttaaaattcacctggaactttaaatgggaaaggattcttttaattgtggattataggcataatactgtttgcatctgaattttctgtaagtgaataatagtttaatagaggaactcatgatttgtactattgaatgattaaactaagtatgaagtgataccattcagcatggcatcaggtcattgcagttttagttctgtgtaacacaagcactcactgaaattccagtttctaggattagtgtaggagcctaacgtgcttctactgttttaatgggttaatcctggattacttaacaatttatgtcaattgcactggtttaatttgttgctaaagaaataatgccctgggtttagtaacaaatacagctcaactattcttgaatatattttgaaaaaaaaatgtatgtaacttaccttttgtaaacgttccatttcttttttccctcatttttgactcttaaaggtgcaatttattactgaattgggatttctggcagcacagaactgctttttattttggggtctgtgagtttcttaggtattagcaatcttgcttataaaataagaacaccttttaattaatgagtgggtcattcctggtgcaattgtgatttttctttagccagaatgaatggcaaactctatttagagcaaagtaagtattagaaaaccctaggaactcttaatcaacgtttattacactttcattaaggcaaactacgtgaaagagccttggggaagttggcccatatcttactaagttgatcagatttctcgttgggctggaaatgtttcgctgttgtatattttaaagtaaattgcacctttgtaacatattgtattgacgaatgatcactaagattagctatatctatacagtcattagtttgacaagaaatagaatcctgtcagatgccaaagagtgggatttttatgtttaatgattaaacaccattatttattgacaatttaccctgtggaactgtattatttctaactatgaaataaaggggtgatgtaaacacacattgttgtgtggtgctttaaactaggtccactatcaacaggctacttactgttcaagaattccactgaagcacttattttaaggccctatttttcttaaacaaaacagtgacaacaacaatcaaaccatttacttttgatgctcattggcattttatgataaaagatgtattcatggcaatgatatgtattcaccctattaggaaacacaactggttacctatgagacctgttctgtccgtgtgcctacgttccttaataatagctaaataaaaatttgtagctttt

SEQ ID NO: 78 - DNA, 2881 bp, Synthetic construct
  source 1..2881: mol_type = other DNA
attccgcacctccgagtgctggccgggcgagaggctggcggctgggctctcgcgcccctccctgcagggctcaggctctccccctcctgtcttctccgcgctgttcctcgtcatggcggccctcagcaagtccatccctcataactgctatgagatcggccacacttggcacccttcctgccgggtctccttcctgcagatcaccgggggcgccctggaggagtccctgaagatctatgctcctctgtacttgattgcagcaattctccggaaacggaaattagactattatttacacaaactactccctgagatcctacaatccgcttcatttctaactgctaatggggccttgtatatggctttcttttgcattttaaggaagatacttggaaaattctactcatggactcctggctttggtgccgctctgccagcatcttatgtggccattctcattgaaagaaaaagcaggagagggctgctcacaatttatatggccaacttggccacagaaacactattcagaatgggtgtagcaagaggaaccatcacaacattaagaaatggagaagtccttttgttttgcatcacagctgccatgtacatgttctttttcaggtgcaaggatggcttgaaaggatttacattttctgcacttaggttcattgtagggaaggaagaaattcccacacattctttttcaccagaggcagcatatgcaaaagtggaacaaaagagagagcaacatgaggaaaaacccggaagaatgaatatgattggtctagtcaggaaatttgtggattcaatatgcaaacatggaccaaggcatagatgttgcaaacattatgaagataattgcatctcttattgcattaaaggtttcatcagaatgtttagcgtggggtacttgatccagtgctgcctccgaatcccttctgcatttaggcatctgtttacacagccatctcggctactttctctcttctacaataaagaaaacttccagcttggagcttttcttggctcttttgttagtatatacaagggtactagttgcttcctgcgctggatcagaaacttagatgatgaactacatgctattatagctggatttttggcaggtatatcaatgatgttttataaaagcacaacaatttccatgtatttagcgtccaaattggtagagacaatgtatttcaaaggcattgaagcagggaaggttccctattttcctcatgcagatactatcatctattccatctctacagcaatttgcttccaggcagctgtcatggaagttcagactttgagaccatcttactggaagttccttttaagactcaccaagggcaaataagaagccaactccaacaggtggaagagtaccataactctgagcaggaaagaatgaggaatgaaatctctgacctaacagaagagcttcatcagaaggagatcactatagcaactgtcacaaagaaagctgcccttctggaaaaacagttaaaaatggaattagaaataaaagaaaaaatgttagcaaaacaaaaggtctcagatatgaaatataaagctgtcagaactgaaaacacacatctaaaaggaatgatgggagatttagaccccggagaatacatgagtatggacttcactaacagggaacagtcaaggcatacatctattaataaactgcaatatgagaatgaaaggctccgaaatgatcttgcaaaacttcatgtcaatggaaaatcaacctggactaatcaaaacacctatgaagaaacaggaagatatgcctatcaaagccaaataaaagtggaacaaaatgaagagagacttagtcatgactgtgagccaaacagaagtacaatgcctcccttgccaccttcgacatttcaagccaaagaaatgacaagtcctttggttagtgatgatgatgtattcccactgtctcccccagatatgtccttcccagcatctttggctgcacagcatttccttctggaagaagagaaacgagcaaaagaacttgaaaaacttctaaatacacatattgatgaactgcaaagacacacagaatttactcttaataaatactccaagctaaaacaaaatagacacatatgagcttttaaacttttttatttgcttcccccccccacccccgccaagaaaaaaagctctggcaaaatatttacaaagctgattaatgaaaccaagaaattctgttctgtttccttgagtaaataatttccgtaaggcagctagaaaatagtaagtattttgttctataaagctgttcacatttctgcattaacatgctaaattgtcctgctgtagagttactataaaataaacatgactattccaaaagagatttttttccagtctaaggaatattttggaatcaaatatgcagttttttttccccacttgaatcatgtatactgaaaacccatttcctcagcaactacacaaaataagagaaagggaagtgctatattccagatacaaatgactcaaaggcagctcagttgtactggttttgaaaactcaaggattttataaataaaaatatttaagtatttccagttatgtaaacactctaaaattctataattttttggaaaaaaaaagctatagctttattgtttttacattcacctttataatgtctgtctgtttcaacacaatgaaggtatatggaatgtgcaatttacaaacctaaaatatatgtgccaaaaattaacgcttttttgttagtactaattacttcatggacacaaacaagataaaggaaatataaagcattactgttgctacattttcctttgtaatcacttctgtttttaatacaataaaggaatgtaaaattcaa

SEQ ID NO: 79 - DNA, 19582 bp, Synthetic construct
  source 1..19582: mol_type = other DNA
acgtcagtttctgcactgaaactctcaagatcaatgagcaaagagctttctcagttctgcctttcagtttctctcttccaggaaggaaaacattcgagagagcgagggagagccgcgggagggcggggggcgggggcgccggctgcgggtgggaggagagaccgggaggccggccgggctgcgtcccgggtccccgcgccgcgccgcgacctgcagaccccgccgccgcgctcgggcccgtctcccacgcccccgccgccccgcgcgcccaactccgccggccgccccgccccgccccgcgcgctccagacccccggggcggctgccgggagagatgctggaagaaacttcttaaatgaccgcgtctggctggccgtggagcctttctgggttggggagaggaaaggaaagtggaaaaaacctgagaacttcctgatctctctcgctgtgagacatgtctgagactcctgctcagtgtagcattaagcaggaacgaatttcatatacacctccagagagcccagtgccgagttacgcttcctcgacgccacttcatgttccagtgcctcgagcgctcaggatggaggaagactcgatccgcctgcctgcgcacctgcgcttgcagccaatttactggagcagggatgacgtagcccagtggctcaagtgggctgaaaatgagttttctttaaggccaattgacagcaacacgtttgaaatgaatggcaaagctctcctgctgctgaccaaagaggactttcgctatcgatctcctcattcaggtgatgtgctctatgaactccttcagcatattctgaagcagaggaaacctcggattcttttttcaccattcttccaccctggaaactctatacacacacagccggaggtcatactgcatcagaaccatgaagaagataactgtgtccagaggacccccaggccatccgtggataatgtgcaccataaccctcccaccattgaactgttgcaccgctccaggtcacctatcacgacaaatcaccggccttctcctgaccccgagcagcggcccctccggtcccccctggacaacatgatccgccgcctctccccggctgagagagctcagggacccaggccgcaccaggagaacaaccaccaggagtcctaccctctgtcagtgtctcccatggagaataatcactgcccagcgtcctccgagtcccacccgaagccatccagcccccggcaggagagcacacgcgtgatccagctgatgcccagccccatcatgcaccctctgatcctgaacccccggcactccgtggatttcaaacagtccaggctctccgaggacgggctgcatagggaagggaagcccatcaacctctctcatcgggaagacctggcttacatgaaccacatcatggtctctgtctccccgcctgaagagcacgccatgcccattgggagaatagcagatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattcatactctgttgcctcctctctccctgcctcacatctcccttccacctcacaactccttccatccttgactgaagcgaacatcttcatataaactcaagtgcctgctacacatacaacactgaaaaaaggaaaaaaaaagaaagaaaaaaaaaccctgtaaggcagtttggcaaatatatatatatatatatatatttatatatctaactatctatcaatctatatctacagagagttaccttctctagtacacagagaagctgctgatacagaaaaccacaagactttaacaactcagaaactctaaaatattaataatacaaaggaaaattccctttgacttaagctgtggcccatgcttctaatgctacggctctttggagcagaagacctggaccatgcagagagacaatctttgggatgagagctctgggacgggaggagttaaggtggtgctcagtcgctgctgtgtgtgtctgttaccccggaagctcaccacaggcacatgtggggactgcatggctgtgctcagcaaagaaatgatgcctgagcagggtccttgcctccacttcttccccatttcctccacttcaggacaatgttctaatttgtccattctaaaaagtgtaatcttgatgcttttgggaatcaatgatggcacctacgggtaaacacagaacagacaaccccacaacacaaaaccatgacaagagctgacagcatccatcctgggtgtaagcttcttgctggagttgcaaaggatgtgtctttctattctcagaacttaaagaactggactttctggagtaaaagaaccacagaagaaaaaatagctgaaacctgaaccctgccaagacactgccacctgtcttttccatcattgcaaagattccttggcctcacctcccactttcaggcccctgggaagcttagggggttaggaaacaggtccccatgttatctttgaatgtagacacagcacgctttagggttgcaatagcaagagacttggttgcttgtaggtcattataacgaatgccccctggtcctctcagtttctcatcctgcttccctagatcctagaccccacttcctgggtctctactgagtcccaggtaaaccccactgccaaggaagggagccaggtctagtgagaggctgcagcagtgagtgtttcaggaaagcaagcagtggagtgggtgagagccagcaggggagaatctacttggcacctgtgccaggccccttggcattctcctcacgtttccagcccctgggaggattgatgcatctgcctttgagctgttgtgaaaacgcaggggctgagaaatcacttttgtgggctggggtagggggaagggctgaggcttctctgggagatgtccaatgagcggccagtgctgcaggcttggaatcttaatgaggcctgagccctgaccggagagagggaaggaaacatctgtgctggggcctgctccctctgccccagcactggggaatcactgccttcctggctgctgccacataccacagtggggccgacctctgggcctccctgtcacatgaaacatggaagccaacagcctggccccacagatctcctctgctggagaggagaggatgtgttcctcccaggccacggggcccctctgccttgccccaagcacagagctacctaagggactgacctttgccacactggagctcacctcccgggggcctctggcctacccttttccctgccagctgaagagactgccggcagcctggacgtcctggttagctgaaggcagctgaaatgtgggcctccctatgtggggtttagtactgaattatgtgtttgtggttttgcttctttttgtagacccacaaatatttttatttgaatagtaaagagctgtgctcagattgtagccatggttgggatttgggatcaacgaggctggttagctggactgggaggggaggcaggtgagatgggaatttggtgttggttttctcactgtgctgtgctcctagcccctgaggactttgcaggaaagctgtttggattgaccacctcaaggaactcctttgctgtcctatgacaattactcctcttgtctttccacctagaggaccgttatgccggggctgtgagttctggctgaagctgcacaatctggctttgatctgggtagctaagaacaggtgttcagggagggcatggtctggaggcgcctgagggaaaagtatcttctggttagaagtgaagcaccaatgctcctgttgcttcccttctcccatatcctctcctgaacaaacactgtgtggcttctgtcttcttggctgtgtgtttcaaaactgatagtcactgatgggctgggaagaagcaggagaagaactgggttggagatgggtggaaaactgggaataaggaccatgctatggttttatcttcagggcactgattcatcatggcctaatgaaagaaggtgattccttgggggaacaagaagcactgaaggagaccgcagttgtcagattgctgccaccttaggcccacgggctttcacgctgatcttggggtcatgttcttactgtgtccattgtaagcagctggctgtggaagagtgccagagagagaggggcagagcagggaagggtagcgagggaagatatattactttgatatattagccttggcagattaaaaaaaaaagatttattttggagagtaaaggaggcagaaaatagggccatggaaattgcagggtacactatggctctggggagtgtggcatgtactgggacacacctgtccctcctatattgggacaggactgataaggcttagtacactgtatcttaatttccccatctgcagaataaggggtacacctgctaatctctggccccttctagctctgagttcttagattctgacaacgttgtgtcactgtgccatttttctcaataccattctggaatggcaggacagccttgcataggtggggggcagttgccgctgaaggctcactgggcctgggcaaggctgagttccaataagacactcctctccttctttgccccttgtctcaacatttcggagtctgaagtgtctgagacgactgtaagccaggaggggagcatttggtcggcttcccatcctggcattcctacataccagaaaagcactgtcgacggtccatttatttatttatttatgaagagcctaatgtgatgaaatgtgttgtgaaatgacatcctattagccaatatggccactccagttcatttcaccctcattttcccataccaaagtccaccttttagaagaccatgtcacatcgcctctgagagcttagctttgaagtgagctcttttgtgtcttttgatgaacgctccagggcacccaggctggtctctctgccatgccaacctcaccggatcctctcctggactgagtgagagtgacctgctgttgggtgtccaccttggagtagggtgttgtggccatgggacctcccaattcttctttctttggatgtgaacctaactctgccctctaatgatatccctgaactctgaggggaccttgggcatcactgaacctcctctagtcaagagacaagaaaaaggaggtgctgcttcctccattcagcagatcatgcacaaattactcctagacagaggcttcccacttgaggcaaaggatctttggtccttctgggttcaggttgaacattaatgttcttctctagtgatgcctactttgcggaatacactagtgcaagtcattttggtgctaaatactgcagaaaccaacaccaacgggagagctgtcagccagcttctggggctgcacgagactcaactcagaagtagaagtgagtcagccatgccgggagagtacagggtgcattggaagttaagtagaaaagtggcagggtgaaaagaccagcaaaatttctgattttgctattagttatccacgtggccttggccaaatcagcttatttctcatttgagacgtttaagaggtttgtatagatatttttagagtccttttcatttttttaaagctaggttctggtcccacctgatatatatgtacttgcttgttaaaaataagagatgaagtgaaagataaggaaggagacgaagaaaagttaccaatggcaaaggattgattatagaagacaatgaaagaaggcctggtgactggtagatttgaggaatattgtacagggagccaggcagacaagccagagacttcatttttatgctcttcacaggaggtcactggcctagccacttgcctgcgtttcaccaggctgttcccctcagctagtcaaccttccctgttcaaaaccaacatgctaacacttggcagaaactattccaagatatggagagaggcctgcccatctctcatgcaggccactggaaatcaactctgggtcaaaaccgacaagatttttatcttttaatgcttattgtaagaggccgggatgtttgaaagagggcagcgtcttaagttgcagactattcaccaaagtccttttctattcaggaatgactaatgactttctttgttccccttttccaaaccaaacagcagcagtattaccttgactccaaagtacagtgattgcaatggacagtcttgagattaagggtctttttagtgctttgctcggaatctggagcaaggaaatgtctagggatgtttgttgaatgaacccacctgccagcagtccagagagctgtcccaaaggcccctgagatgtctattgtctgctttggtagggagccaacctgctagaagactcaagggcatttgccttagagagaaaaaaaaatggcacatttagttccgggcttgggtgctgaagtcactgtgtttttggctcctgaagctccactttcttttttcataccaaataaattgttttggccaatatttaaggatttaaatatttaaggttttgaaggaaaattccaagtttatgcatcattattaagacatcaatatgggcttgggaaaatgttttggtgttgcttaataatactgggagtgtcttctaaagaaagtatacatatatgtacgttgtgtgtttatttctatatattggattggaatggcttcaaagaaggcctgtttgaagtcagcaaattgatttcaatttgggattctcttttagccttaccgttcttggggataagaaatctggagatgaactggcctcagaaacattcaggaagattacgatgggaaatctgtcatcttgtaaagttggatcatttggtgctctctgaactaaattatgctgactaacattctgagcatctgaaaatcattcttcctggttgcctgatagatgaccacacaggagtcatttctatcacatcttatttgggggttggtatagacgccaccagcactgcagggaaaaccaccccctaggaggggtgagacagagagaatgtgagattttgatcattgattgctctttctgaaaggcactgctgaccatagtaggtgtggtgaatgtttgtgtcactgcattgggtattagaacttttgctctgagcatgggctgccctaccctgaagagctgaatcttggcttctgacagaagctctgcatgactcattctcctcaagcatattttccttattttcctatgaaagtatctatagtttgcaacaagaactgtctttggttcaagtgtcagtgtactagaatagatgcttttctgtgagacctgtggaggttggattggggtggggggtgttccttcaaagtacttggaattctttatcaaagaaataaaactcagattctcttctttctagtttacttctaagttccaaatttaaaacaagacccagtttgacatttgcagtaggagataatgtcttgaaccagtgagcttggctatctttctttcatcatagggaggcagggcagtaagttgtattaatttttcctaatcacccaggcaaatattgctttttgttcagtgtcgtgacccacattcatagcttgcaggggatacttctagctgacaaagctggaagtttctaatagaagatgtatcaagataacagaggtgaggaaaggcctgttgaacatctacttctgagaatcattgaggcactcaggtcttgcttttcagtttagtatcttacaaaaggagttggtttcttctcattttagcaatgataaatttagacatcttttttgaaaggccaaaggtagattgagagctcaaatacttacggtgtactctgacagtcttttattctatttttcaagacttattttatatatacatattttttatttccataggttattggggaacaggtagtgtttggttacatgagtaagttctttggtggtgatttgtgagattttggcacacccatcacccaggcagtatgcaccatatttgtagtcttttatccctcactcccttcccacactttccccctgagtccgcagagtccattgtgtcattcttataccttgacatcctcacagcttagctcccacatatcagtgagaacgtacgatgtttggttttctattcctgaattgcttcacttagaatagtggtctccaatctcacccaggccgcttcgaatgccattaattcattcctttttatggatgagtagtattccatcatacatatatatgtttgtgtgtgtgtgtgtgtgtgtgtgtgtgtgtgtgtgtaccacagtttctttatctgctcattgattgatgggcatttgggttgattccactttttgcaattgcaaattgtgctgctataaacatatgtgtgcaagtatctctatctcttatgtataatgacttcttttcctctgggcagatacccagtaatagggctgcgggattactgacattcttttacatgcaacatccctaaaacccaacaagacgtgtaaacactgaagtccaagtctgataattattctttcatttaagaagaacacatgggagtttcacatattatttttaaaaactaattttgtaatatgttaaatggtaggacacattcaattttttttcctcattggagatctgtccatgttctgtctcaatttaatagctacctggcatgtaactgatgcctctgagcctacctctggagtgaataaataaacgtggtccaggtgatttgatttctgccatttgtagtggagaagtaaatctcagtgaagaagattttttttcatatttcttcttttttctttttcttttggcttctatcctcctgcttccatgggactgacaatccagcttctcaaactccacgaaagtcttaatacaaggtgaatcccagagccacaaacagtttggacttcaaacagtgagaaaattggaccgaatgattcggcacacaaagcagaaaactttcatttcctgtaggcaggatggaattgactgttatattaagacttctgaagaagccctgccctggagtaatttcagtgctgtcagacgatgttgacatctagattaacctctgtgcagtgaattttactgcaaagctcattgagatgcatgtgatgtgtctccacaaatcccatctggaacccgtggtggcccaccctcagcaaccctcctctccccactgactctgctttctctgtccataaacttttagcctccaggagcagaggagccgtggctgagatctgaccctaggagagggagtttgggccttctctgaccatgatgctgagccaggctttctgcttagagtgacagagattgtcattgtattgggtttcccctgagggaactgcaaattaaggcatgaaaaacactctgctgatgacagtttcacagcatatcttaatgctgcacaaagtgaaatggtttaaaaggaattagtgtgtcctaagtcattgagccctgctctgggctctgcaatcctctagggattcaagatcccattgcaacaaggtcatgactgcgtctaggccggacagacggattctgaaggacaaggtgtttgtcttttgtgcaggtctttcctatggtccttacaagagaggcagccacagctcctgtttctcccaactgggacaaaagatgtagctgttttctaagcactggtttgtcatctctttaaggagtggttggctccagaatgagccagactcactctataattccacattgtggcacaacagccacatagaacctcaaagctgaaattgcaacttttctgcaaaatagggccattgattacatccaaagttttgagaaaaggccttcaggttagattcagtcaccttcagactccacagagttgctcagcatggttgcaggcgtctttggaaacttacctcagggttttagaatacgttaccctgaagtctggataacaattttctctgagattttactgattctaattccctttgaaattgtttttcctacttgcctcttgtctttcatttaaataataagaatctccttgcatattcttaggtatttctttaggagaattggaagtttccagccaaggacactgtgtttatgaattggaaagatatctttctgggctgaaaaaggtaaaaatcatacggaggcaatcttgatgactttatttaaaagcaattatcagcattgtgttttattctaggtgctcgtgctcatagtgcaaactacattcacgtttttaattatttatgttatgatacttattcgtgtgtacatatatgcgcacacacacacacaaaagcctccacaatcctactggtatacaaatccatatgccccatttgtgtcgcttccgttgatctttgtgtgcatgcatttgcaatgaagtgtgcatcacatgtgtgatttcttaaagtcatctatagctcatctttcctcttattatcgacacataccctttgactttctgcactttggagctcatgaaattaaggctttttcaagggagactacatcttgagtttctttaaaccttcatatttaaagataggactggttttcttcgagaaacaaaaacttgattcagattttatttaattaattaattttctgggggaaatggtactgaatttgttcctgagtaacaaatagtagtaagtgcaaatataaatgtgaagtagtttaagcaggttattgaacctatcttgtggctgaaacaagtagtcagatatgaagctcagacactcaaattgaattagaacgtgaaccaaaacggttagcatggggcaggagaagagcactgggctaggagcaagcctgctagattgtcacttgagtgtccttagcttaatgtgggcatctgacaactagttgcagtcctgtgcccctctaattccagagatggggcaagccaatctgtttctcatccattgctcaagttggttatcactgaaatattatataccatctcaagatcctgccctgaacaagtttgcaaacatcccccccacactgaggagtcagtcccatttttgttttatggcagtttatctcttgtgaccctcctttggaagctgtccaaactctgaacacttgctgtccattccctgctccccaccccaccctttaaggccagggaaaagccagggaaagccctattggctcatctccctctttcctagcctgcacatcaaccaagcttgggatttagcctaggttttcagagaggaaaacaagtgcgttctcccagccatcagtatctttccagccccctggaccaagtgattagtgcctcattagcgcctcatcagttgcttttgcacaagagactctgggacaacaagcctattataatgcctcagtctgtctttttgaagtcaaaccatccttctggcttggttaggaaaggggagcctttctccaaacacgttttcttttaatttcctctgttgttttctggctgttaactatatggataaatggtaaaacataattttcattttttctaggcccctatgagagtggatatgctatatttctatagatgaggagttccacatgtcagcaaagcatttatttatttagtgaagtgactataatattgacacacagatttgcagcagcatccaaatcttgagggggaaaaaaaagcctttttcaatcaaatgaaaccttttctgtgatggcaccagagaggtttatatcttaagcacagaactgaacctgataatatgcttcctttctagaaacaaagagatagaagaaattgctgaaggagctggaagaccaaaccatctattccccaaatcattatgtatcaatagatacttcaactctgttccttaaatggctctgaaaaacacaaataaccatatttactattaaaaacaaattcataatgtctaacccaatcccttattttaatatatatacatattaaaataatcccatgtatatatgattattattatatgggatatatatagtttacatatatataggggtgtgtgtgtatatatatatatatatttacatatatatgtaaatgtatactttttaatgctggggatagttccatattgtttcttttcttgattccttctaatatattcaatgagatttataatttttttcacttttatttctttaggaaagagttggggtctattatgaatgctttctaggataaaatggcaaaacagatggaaagtgtggtagagggaggataatgggaataggaggcagagaacttgcacatttctatttcctgctgagagatcttggaaaaacattgtctgccctttacctcagtttcttaacatacaaaatagaactactatcaaccccctgcatgccacagagggttatttgagccctaaatgagatcataactgttaacatactctgtgaactgtacagggccagacaaatagagcccactctctgggcagttgcatacagtcttgctgggtgctgagctgagtgactgagagccgcttttgagacacattttgaggcatatcttccctaaatttttgctggtgtctgttttttagaagttttgccattagcattttttgctttgagtatctagtgatggagaatgctctaaaaaatacaaggagacagggggattctttatctatttggcttagtcactggaaagggacaattagagtttgttttcaggcttggaactctgacattttgtagcaaaatggcatcttctgctgcatttaattccttgccatcaactcttgctttcataaggttctgtcaatttcaacataatgctttgtaaaaataggactctgtgaaactttctctataaatttggtggtataactaatttttcagggagcatctgaagggataatgatttttaagtggtaccctgttcatgccaggaccagcatttgtacatgggaaaataatcttaagatgagtgaagagcaaatgctagaccacacctttaataggcccaagtgccaacaaaaatacgtgtgagtgggattcccacggagaaatgtttctgaatgaaaatgaaacccttgataatgaatcctgaggccagaatgccaaagtcacagtattctgtcatttacagcccctaaaaaatcccaggtgtttgtgatgacaagccactattataagtactctgtattagtgactacttggcaagtaaaattttctcaaatgttatttttcatagatctccaagcaacaatcacttgcagtgtatctgacaaaatgctatacatgattttctaaaagtattttgatcactccagaaagaaatcagaataaagctgcataccccaaatcctagaaaattcacaaatattttgctcaagttcaggctctctcagcttctcttttctctttaacttttgcctcatacagaggcaatcttgatgacaaaagattgggctcaatttttgtttttcttgtgtagaaaagcaacacctctgtttcggactactgttcagtagacaaaaagcatcccagctttcgaaatgagagtccaaattgggttaccactgagattttaaattatgatgtgaaattggagaaggtgaactgtacagggccagacaaataatagagcccactctctgggcagttgcatgcagtcttgctgggtgctgagctgagagactgagagccgcttttgagacgtgttttgaggcatatcttccttaaatttttgctggtgtctgttttttagaagttttgccattagcatttttcactttgagtatctagccatggagaatgctctaaaaaatacaaggagacagggggatgctttatctatttggcttagtcactggttcagggtgaagtttcataatttattctgataatctggaggggatgacctccttcacagtactgttaaagccttccttgaacaagctcatttcactatataacttcctaaagacaaggctgattatttaaggatttcagagatatcatttggggaataactcaccaggtagagaaagtttgacctgatatgttaacaggcacaacaagaacttatttcaatgctttctcaccatctgccccttctctgagagtttagcctttcttaggatacttccttttccaggctagaaaagattgattgtattcatatcccaaagtaaaggctcctccaattctatcagatgtggaggcaacagaaaatgactcatgttatttttctctcacttcacagcatcaagaactcttcttgtttatgtgggggccatggtggattagcttgaggaactgtcaaattaaaatgaaaggaaagcaagttggggcaagaaaattctcactctgaatgtgaaagaaaatagcagtgttcttctctctggcaaagatttgtgtactgttgggggaagattcatgttatttctcaggtagactttactttttgagattctgtgtgtgtgtgtgtgtgtgtgtgtgtatgtgtgtgtgtgtgtgtgtattttgcctggtggggggttaaaggcagatagaatgtagttgtttatgagtttatactttctctttagcataatagatgccctgtttattttctcagaatgtgacaataaaattaggaaaggagaggaattcagaggcccatgttgcagttcatggcaaagttttacccaaatatttccttcagaaacatttagtcatagcaagccatataaattattgtctgcaactggtatcagaaaaagaaatcagtaggtggggactgtagaccccaatggtgcatctgtttacaatctttcttttccaaggttttaaggttcatgaataacatgagggaattttgggagagctaccacatcagtactttggcacgcattaactgttccacaggaaaactagggttgcttcagggctatttttgtttgaaatgacacacatacaaaaaaaaaaaaagacttttggaacatgttttgaggcctgtcacctgaaatcagcctttgctgaaagagtatttagtttgataagcgggatttcttgcttctcgcatcccagagtgtaaattctgcagcgaagcctgggtgtatgttttcagcaccagggacagcaaacaaccccctgaattgaaggggcccgctatcctgattgacctcccagactaggaagggcagttgttttgttttcctttatttcctatctaccggagcctcaacttggaagactttattcttaaggttcttacatctttcatttattaccaagcctagcttagctgtctctgtcacattcttgacctcctaacaaatgggtttggagtaacactcccagctctcctggctcagagaagtagaaagaaacacactagacttcatttgaagggcatgctttagagtaggctccccctacacaagtctttataacataccactgacttgtaaacccctaaatgaaggactgcttatacacaatcattttccttttaacatttctgcacatttctcacaatgtacaagtctatgtttgactcagtgtgcatctggaggcaatgtttcacagcagttgagcagaagctatgaagtttggggacagaaaaggtcccagtgatgctgctgttattcattttggagactcattgggggcacttctctgattctcagcatccacccttcaaccctgagtgctctttaacagctgtttttgccttaaacaatcacaataagaatgcaaatgactccttgtggcattcttcaggccagactgaagggtgtactgagaaaacaagggtagcagaagcacatgttgtcacattgctttgtgattggccagtttttatatagcacaatgacacacagatatcatcttagagtcatcattgtctactcttaactaattgtcagcatcttctccactcatgtcaggatgaatcctgtgttgggaaggcaagtcagtcactgcaggtttcatcataaatattgccttctatctgacagatgttagaaaacctccattttgaatgcctctgtaccatccaaggctctgaatttcccctatatcttatccctttctttccattccctttccatgttgcttatcataacatccataacatttagtggtgtaagaaaataaggggtttcttttaggaataaatgaggtggaaaaagtaggacagagccagatctcagaggagcaagttcagtggaacgatgatttctaacttgggagtgggtctaagatgtagccatccattccttccattccttgtcctcttgagtgaatctcattccagtgctcacacttgccctgcaaaaaaggccatggcacaagattctctagattatttgtatgaagactggaggtgagcagcatccaacacctctgttagagttctttagaatagaggagttttgtcttatattttaatccaggaaccttcccatatttaaaaagtttagaaataatatgtacagaaaggacctatggggataggaaagcttaataatacaagttattttcccttagaacagaggggaaggagctgcaaagtgactgctaatggaggaacgatctgctggctgggggaaatgtggcctgagactagagatgcagagcaggcatctctagaaggagagatgatgccaatagaagccccaggagggaactggccagcgaatacaattttataaagattctcagtactttttctgaaaaaattcctaaaattttcaagggcgcgtctagccctctgcctctttagaggtgagctcagtgctacttctgggttcttaaggcaaatgccctacaaacaccaaagtcttgtattttccactctcagccggcactggctaagtgggattttttttttcccttccctaagtgaaagggacttgttttcaggagaagtgtcctttaaacatccagctaaaactggaagtaattttttgcatgaatggctttttacagctaggtctacagcagaacaaaacttgtattaaactaagtgctactttacatatcttatttcaagggaaatgaaggatttattgtcgtgtaaatgatatacactgatttgtttcctactggacattacatgtccctttatctctaggctgatgttgtgcagcaaactttcaatatctggaaaatcagctggtacctacttcagtgcattattaggatattacgttccctgagaggaattttgccaatgaagtttaggtactgttgtaactgatttccatcccccctccctaagcttctgtactaagtatatgtaaagctattatatcttgtctgatcccatgtgttcatgtgtgtttcccaatgtttgcataaatatattggcaatctgaagggttagctggcttatggcaactgctgaacaatggtatgtttggcatgtgggacacctgatacctgggcacacctgttctgaggccaagctaattttacagattttggaaaagaaaaaacagcagcaacaacaacagtgtggcttgaattaaggaaagtttaacaatgaatacagtattgccaagtcatcatttatgggtggagacgttgaccacagcagttgaggtgctttcaactggatcttatgccctttattttaatcccaattgatagttcctcatttgtattttatgacttagaaatactcctacgtgatgctcgtttgtttttctcccaggcttctggaaagtgctgatcatgattgcttcctatcatttggatcccaaagttgataacttaacataagtgactgaggttttagtagaactgggttttactaatcgtatctcctccttgaggtgctgttcccttcattcctttcctttatgacaggtgaatgcattggctatgctgattttatttgcatcctggcgagtcttggagaatcaatctcgttaaaatgatgactgcatttgcctttggagaagaggtttattgtctaagtaaaactcttatgtgaaggtgtcatttatgtaaaaaaaaaaaaaaattctaacttgataagatggccacaggttagaggggtgtgttctcagttgatattaagggatatgttgaaaaggcaagtgcccaccctctattcttcatctcacccttcctttctccccaacctcaaaagaaaacaaaaaattatttttttaaattgtattagtaatttatgtttaatacaaaaggatgatttaatggtatcaccaaaggttgtttctcccattgtgcatgtccttcagtctcggccccatacccatcaccccattcttcaccctcatgcttccatcccaaggcaaacatgtgtcttcatcggaatctatgggtgttgaagttaaatgtgggggcagagatttaacaccatgacactaatacaaatcaaccattcttcactttcaaatggttaatcactacaggaaggcgaactcttttcttggtttttgtttaaaaacattttatacatatatatgtatatatgtgtgtgtatgtatggacataggtatgtatatgcacatgtacatgtatatatgtatatatccatcttcaatataaatatatcataagtgagagttgtaaatactccttggtcatatgtctgtctttctcatagtatcatatcttcaatgttatgttaacaactccatttattgattgatgaaatcgtgtgtagacctgtatcctcctgacatagtttatgtagggtctcttctcaaataaagtataaaacaacaa

SEQ ID NO: 80 - DNA, 4938 bp, Synthetic construct
  source 1..4938: mol_type = other DNA
gagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagggggtgcagcttttattttcccaaatacttcagtatatcctgaagctacgcaaagaattacaactaggccagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaa

SEQ ID NO: 81 - DNA, 4267 bp, Synthetic construct
  source 1..4267: mol_type = other DNA
gggcagctgcaatggcgctgtcgtgtacccttaacaggtatctgctcctcatggcgcaggagcatctggagttccgcctgccggtggtatttttgaatatgtggaatctggcccaatgggagctgaggaacttgcattcagatttgctgtgaacacaattaacagaaacagaacattgctacccaatactacccttacctatgatacccagaagataaacctttatgatagttttgaagcatccaagaaagcctgtgatcagctgtctcttggggtggctgccatcttcgggccttcacacagctcatcagcaaacgcagtgcagtccatctgcaatgctctgggagttccccacatacagacccgctggaagcaccaggtgtcagacaacaaagattccttctatgtcagtctctacccagacttctcttcactcagccgtgccattttagacctggtgcagtttttcaagtggaaaaccgtcacggttgtgtatgatgacagcactggtctcattcgtttgcaagagctcatcaaagctccatcaaggtataatcttcgactcaaaattcgtcagttacctgctgatacaaaggatgcaaaacccttactaaaagaaatgaaaagaggcaaggagtttcatgtaatctttgattgtagccatgaaatggcagcaggcattttaaaacaggcattagctatgggaatgatgacagaatactatcattatatctttaccactctggacctctttgctcttgatgttgagccctaccgatacagtggtgttaacatgacagggttcagaatattaaatacagaaaatacccaagtctcctccatcattgaaaagtggtcgatggaacgattgcaggcacctccgaaacccgattcaggtttgctggatggatttatgacgactgatgctgctctaatgtatgatgctgtgcatgtggtgtctgtggccgttcaacagtttccccagatgacagtcagttccttgcagtgtaatcgacataaaccctggcgcttcgggacccgctttatgagtctaattaaagaggcacattgggaaggcctcacaggcagaataactttcaacaaaaccaatggcttgagaacagattttgatttggatgtgatcagtctgaaggaagaaggtctagaaaagattggaacgtgggatccagccagtggcctgaatatgacagaaagtcaaaagggaaagccagcgaacatcacagattccttatccaatcgttctttgattgttaccaccattttggaagagccttatgtcctttttaagaagtctgacaaacctctctatggtaatgatcgatttgaaggctattgcattgatctcctcagagagttatctacaatccttggctttacatatgaaattagacttgtggaagatgggaaatatggagcccaggatgatgccaatggacaatggaatggaatggttcgtgaactaattgatcataaagctgaccttgcagttgctccactggctattacctatgttcgagagaaggtcatcgacttttccaagccctttatgacacttggaataagtattttgtaccgcaagcccaatggtacaaacccaggcgtcttctccttcctgaatcctctctcccctgatatctggatgtatattctgctggcttacttgggtgtcagttgtgtgctctttgtcatagccaggtttagtccttatgagtggtataatccacacccttgcaaccctgactcagacgtggtggaaaacaattttaccttgctaaatagtttctggtttggagttggagctctcatgcagcaaggttctgagctcatgcccaaagcactgtccaccaggatagtgggaggcatttggtggtttttcacacttatcatcatttcttcgtatactgctaacttagccgcctttctgacagtggaacgcatggaatcccctattgactctgctgatgatttagctaaacaaaccaagatagaatatggagcagtagaggatggtgcaaccatgacttttttcaagaaatcaaaaatctccacgtatgacaaaatgtgggcctttatgagtagcagaaggcagtcagtgctggtcaaaagtaatgaagaaggaatccagcgagtcctcacctctgattatgctttcctaatggagtcaacaaccatcgagtttgttacccagcggaactgtaacctgacacagattggcggccttatagactctaaaggttatggcgttggcactcccatgggttctccatatcgagacaaaattaccatagcaattcttcagctgcaagaggaaggcaaactgcatatgatgaaggagaaatggtggaggggcaatggttgcccagaagaggagagcaaagaggccagtgccctgggggttcagaatattggtggcatcttcattgttctggcagccggcttggtgctttcagtttttgtggcagtgggagaatttttatacaaatccaaaaaaaacgctcaattggaaaagaggtccttctgtagtgccatggtagaagaattgaggatgtccctgaagtgccagcgtcggttaaaacataagccacaggccccagttattgtgaaaacagaagaagttatcaacatgcacacatttaacgacagaaggttgccaggtaaagaaaccatggcataaagctgggaggccaaacacccaagcacaaactgtcgtctttttccaaacaatttagccagaatgtttcctgtggaaatatgcaacctgtgcaaaataaaatgagttacctcatgccgctgtgtctatgaactagagactctgtgatctaagcagttgcaatgatcagacttgatttacaagcatcatggatcaaccaagttacacggggttacactgttaatcatgggttcctcccttcttctgagtgaatgttaacatgcgcattttgtggctgatttcaaatgcagtccagtgagaaattacaggttccttttgaagctcaactgttgccaggagatggaatatcaatgcccaacagggcaaccaataaaagtgtcactaagaatataaatatttggaatcagcaaaaactgtagtgttacaggaaacagtacagtcttctgaacacccagatcatagaggtgatgatgttactagcccccaactactcagtataattattgtctgaatgcaaagtatgtgtttataggatgtgaaaaaatgtaatgcaaaacaaatttgaatcccatggcagttggaatataaagcagatgttcatcacttattttccttttttcttttcttatttttttttttgacagtctgtgtcactgattgagatagaaatgccaattatcaaggaaataatgttttcttaagttccctaaggcagaagatttaacatgcaattctaccagatcccttcctattcccccaacaccttttctctaacccccatatcccaaataataataataataataataataataataataataataataaaagcagttggttcagtgattctgaattaaaaggataatgttttgcaatgttcaagttgtaaaaactggccgagtattggctgtgtggaagactaaagctttcattctaacattcagacatagcaatccaaacccttgttcctgctgtaaatgaacttgatggagcatgggcagatttcagtgatacgagaaaggggactggtcatctatagaaaaatctgtgagagaacttggaagtggactgcgtttatcaatacagtcacaatgttaaatgaacaaaattcttgaacagttttttttcaaaaaatgttcaggtttatttgtggaaatgcaagatttctatgaaaatagtttttgtatggaaatttttgtaatactttttatcaacaaaacaagaacatgtgttcctgtcaggggtgtgatgtcaagcatgaatggtagtgcgtgtgcaccaccaacgtttggtgaaaactatttttatcaagaaaaaaggaatcatagaagagaaatattttcaagttagataatataaaagctaggtgcactaccaccactgcttaccatgccacacccctggtttccacgaggctgacaacatactgtaatgaacaattgtgtgtaaaatggtaaaagacacagacctcttgacaacattgtgataacagttgagtgcacacagtttgctgtttgaatccaatgcacaaaattaaaaaaaatcattaaaactatgttcattttactttca

SEQ ID NO: 82 - DNA, 3522 bp, Synthetic construct
  source 1..3522: mol_type = other DNA
agagcgcgtgtggcggccgagcacatgggcccgcgggccgggcgggctcggggcggccgggacgaggaggggcgacgacgagctgcgagcaaagatgtgccccgggacccccggcaccttccagtggatttccttgcggaaaggatgttggcggtccctgtgacctgtggagacacggccagatctgccctccacagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaatcttaagttgtgaatgttttgtttcgtttcttccccctcccaaccaccaccatccctgtttgttttcatcaattgccccttcagagggtggtcttaagaaaggcaagagttttcctctgttgaaatgggtctgggggccttaaggtctttaagttcttggaggttctaagatgcttcctggagactatgataacagccagagttgacagttagaaggaatggcagaaggcaggtgagaaggtgagaggtaggcaaaggagatacaagaggtcaaaggtagcagttaagtacacaaagaggcataaggactggggagttgggaggaaggtgaggaagaaactcctgttactttagttaaccagtgccagtcccctgctcactccaaacccaggaattctgcccagttgatggggacacggtgggaaccagcttctgctgccttcacaaccaggcgccagtcctgtccatgggttatctcgcaaaccccagaggatctctgggaggaatgctactattaaccctatttcacaaacaaggaaatagaagagctcaaagaggttatgtaacttatctgtagccacgcagataatacaaagcagcaatctggacccattctgttcaaaacacttaacccttcgctatcatgccttggttcatctgggtctaatgtgctgagatcaagaaggtttaggacctaatggacagactcaagtcataacaatgctaagctctatttgtgtcccaagcactcctaagcattttatccctaactctacatcaaccccatgaaggagatactgttgatttccccatattagaagtagagagggaagctgaggcacacaaagactcatccacatgcccaagattcactgatagggaaaagtggaagcgagatttgaacccaggctgtttactcctaacctgtccaagccacctctcagacgacggtaggaatcagctggctgcttgtgagtacaggagttacagtccagtgggttatgttttttaagtctcaacatctaagcctggtcaggcatcagttcccctttttttgtgatttattttgtttttattttgttgttcattgtttaatttttccttttacaatgagaaggtcaccatcttgactcctaccttagccatttgttgaatcagactcatgacggctcctgggaagaagccagttcagatcataaaataaaacatatttattctttgtcatgggagtcattattttagaaactacaaactctccttgcttccatccttttttacatactcatgacacatgctcatcctgagtccttgaaaaggtatttttgaacatgtgtattaattataagcctctgaaaacctatggcccaaaccagaaatgatgttgattatataggtaaatgaaggatgctattgctgttctaattacctcattgtctcagtctcaaagtaggtcttcagctccctgtactttgggattttaatctaccaccacccataaatcaataaataattactttctttga

SEQ ID NO: 83 - DNA, 1018 bp, Synthetic construct
  source 1..1018: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccagaggcccatcctcaccatcatcacactggaagactccaggccaaggcaggtggatcacctgaggtcaggagttcaagacagcctggccaacatagcgaaatcccatctctactaaaaatacaaaaattagcctggcagtggtggcccacgcctgtaatcccagctactaagggggctgaggcaggaggatctcttaaccagggaggaggaggttgcagtgagcagagattgtgccactgcactccagcctgtgcaacagagtgagactctgtctc

SEQ ID NO: 84 - DNA, 498 bp, Synthetic construct
  source 1..498: mol_type = other DNA
gcatcggggagcagggtgtgctggagcagcggggcttctctgcctccgcctttcccggggagttggcctcagccccagggaagggcaaggtggtcagggtacttctggactctccattactatcgacttccattttacgcaccagtctgcacaatccagactaaatcccagaagatgtcactgtacaatttccaataggtcccaagggaacacagacataataatttttctgttctactccaggtaatttttcattatatctggtacctgtagcaggcaagttctgttctctttcagcatcagactctctggtacataactttccatagtaattccagccctggaggcactagatacaatggcagtcagggcaatttgggaaggtgtgtatacaaggtaagcatccgtcaatgcaattctattaagaaagtcatcagctgttttcctcaaaatctctggattctccaatatgggatagcgggtctttacgtcgatgag

SEQ ID NO: 85 - DNA, 501 bp, Synthetic construct
  source 1..501: mol_type = other DNA
ccttttagatgtgtgttttcagttctgacagctttatatttcatatctgagaccttttgttttgctaacattttttcttttatttctaattccatttttaactgtttttccagaagggcagctttctttgtgacagttgctatagtgatctccttctggtggagctcttctgttaggtcagagatttcattcctcattctttcctgctcagagttatggtactcttccacctgttggagttagcttcttatttgcccttagtgggtcttaaaaggaacttccagtaagagggtctcaaagtctgaacttccatggcagctgcctggaagcaaattgctgtggagatggaatagatggtggtatctgcatgaggaaaatagggaaccttccctgcttcaatgcctttggaatacattgtctctaccaatttggacgctaaatacatggaaattgttgtgcttttataaaacatcattgatatacctgccaaaaatccagcta

SEQ ID NO: 86 - DNA, 501 bp, Synthetic construct
  source 1..501: mol_type = other DNA
tattcaggtctccatgcttcatgtattcaaagaccatggtgggggggtccccatcgccgcacactccatagaacttggcaatgtgctcatgctgcaggttggtgggcagctcggcctccctctggaaatccttccgggcagccagggtgggatccttcagggccttcacagccacaagcatcttgtccttagtcgggctggggttgtagcactcggccaggaagacctttccaaaggctccctcacccagttctcgcttcagcacgatgtctctcctcttactgtgctgcacatctgctattctcccaataggcatggcgtgctcttcaggcggggagacagagaccatggtgtggttcatgtaagccaggtcttcccgatgagagaggttggtgggcttcccttccctatgcagcccgtcctcggagagcctggactgtttggaatccacggagtgccgggggttcaggatcagagggtgcatggtggggctgggcatca

SEQ ID NO: 87 - DNA, 264 bp, Synthetic construct
  source 1..264: mol_type = other DNA
ttggaagtctgtccatggtcgctggaggaggacgcggtcatctctgtcttagccaggtgtggcgttccgtgggcacactcaaacaacgactagtcctcactcacaactgataaggcttctgagttcaaagcatcttgctgttatcaacagcatcgagtaaggataggtatctagaatgttcaatatgacctgccgcgctccaggcggcgctccccgcccctcgccctccgcctccgcctccgcctcctgcttagctcgcgccta

SEQ ID NO: 88 - DNA, 201 bp, Synthetic construct
  source 1..201: mol_type = other DNA
cttctgggtatcataggtaagggtagtattgggtggcaatgttctgtttctgttaattgtgttcacagcaaatctgaatgcaagttcctcagctcccattaggccagattccacatattcaaaaataccaccggcaggcggaactccagatgctcctgcgccaggaggagcagatacctgttaagggtacacgacagcgcc

SEQ ID NO: 89 - DNA, 498 bp, Synthetic construct
  source 1..498: mol_type = other DNA
gtctcctcccagcagctcggtcaccatctccagctggtcggccgtggagaagctcccgccaccgccgtcgttgtctccccgaagggagaagggtgtgaccgcaacgtaggagggcgagcagagcccggagcggcggctaggggacaggggcggggtgggcagcagctcgaatttcttccagatatcctcgctgggcgccgggggctgcagctcgctctgctgctgctgctggtagaagttctcctcctcgtcgcagtagaaatacggctgcaccgagtcgtagtcgaggtcatagttcctgttggtgaagctaacgttgaggggcatcgtcgcgggaggctgtagagggcagatctggccgtgtctccacaggtcacagggaccgccaacatcctttccgcaaggaaatccactggaaggtgccgggggtcccggggcacatctttgctcgcagctcgtcgtcgcccctcctcgtcccggccgccccgagcccgcccg

SEQ ID NO: 90 - DNA, 111 bp, Synthetic construct
  source 1..111: mol_type = other DNA
tccacctgccttggcctggagtcttccagtgtgatgatggtgaggatgggcctctagttcaggccgcccaggcaggaactgttacacaggtggttgtggtggagggtggta

SEQ ID NO: 91 - DNA, 2971 bp, Synthetic construct; identical to SEQ ID NO: 37
  source 1..2971: mol_type = other DNA

(19) United States
(12) Patent Application Publication
(10) Pub. No.: US 2026/0009038 A1
(43) Pub. Date: Jan. 8, 2026

(54) COMPOSITIONS AND METHODS RELATED TO NUCLEIC ACID SENSORS

(71) Applicants: Arizona Board of Regents on behalf of Arizona State University, Scottsdale, AZ (US); CONNECTICUT CHILDREN’S MEDICAL CENTER, Hartford, CT (US); The Jackson Laboratory, Bar Harbor, ME (US)

(72) Inventors: Albert Cheng, Gilbert, AZ (US); Ching Lau, West Hartford, CT (US); Nathaniel Jillette, Weatogue, CT (US); Jacqueline Jufen Zhu, Scottsdale, AZ (US)

(21) Appl. No.: 19/124,475

(22) PCT Filed: Oct. 27, 2023
(86) PCT No.: PCT/US2023/078011

§ 371 Date: Apr. 25, 2025

Related U.S. Application Data
(60) Provisional application No. 63/381,234, filed on Oct. 27, 2022.
(60) Provisional application No. 63/498,367, filed on Apr. 26, 2023.

(51) Int. Cl. C12N 15/115; C12Q 1/6886; C12Q 1/6897

(52) U.S. Cl. CPC C12N 15/115; C12Q 1/6886; C12Q 1/6897; C12N 2310/16; C12Q 2600/156; C12Q 2600/158

(57) ABSTRACT

The present disclosure provides compositions and methods related to nucleic acid sensors. In particular, the present disclosure provides nucleic acids molecules that target transcripts of a gene or chromosomal fusion and activate a downstream event, including the production of a detectable signal and/or exert a therapeutic function.

### PRIORITY STATEMENT

This application claims priority to U.S. Provisional Application No. 63/381,234, filed Oct. 27, 2022, and to U.S. Provisional Application No. 63/498,367, filed Apr. 26, 2023 the entire contents of each of which are incorporated herein by reference.

### GOVERNMENT FUNDING

This invention was made with government support under R01 HG990004 awarded by the National Institutes of Health. The government has certain rights in the invention.

### SEQUENCE LISTING

The text of the computer readable sequence listing filed herewith, titled “41317_601_SequenceListing.xml”, created Oct. 26, 2023, having a file size of 262,458 bytes, is hereby incorporated by reference in its entirety.

### FIELD

The present disclosure provides compositions and methods related to nucleic acid sensors. In particular, the present disclosure provides nucleic acids molecules that target chromosomal fusions, mutant genes, and/or viral transcripts, and activate a downstream therapeutic function, thereby reducing or preventing the adverse effects of the fusion, gene, or transcript.

### BACKGROUND

Cancer cells harbor unique genetic or transcript changes that set them apart from normal cells. These unique genetic signatures include endogenous gene or transcript mutations, deletions, fusions, as well as the presence of viral genomes or transcripts. Whereas previous approaches of cancer treatment use non-specific chemotherapy or radiotherapy, more recent efforts of cancer therapeutics development focus on targeting unique molecular pathways within cancer cells or programming the immune system to attack them. Though powerful, these existing targeted approaches require significant time and cost to develop. Many cancers harbor recurrent chromosomal translocations each leading to fusion of two genes. Well-known examples include infant AML such as those with CBFA2T3: GLIS2 fusion, supratentorial ependymoma (ST-EPN) with ZFTA/RELA fusion, and Ewing sarcoma (EWS) with EWS/FLI1 fusion. CBFA2T3: GLIS2-positive AML is one of the most aggressive forms of infant AML, having almost no survivors. ZFTA: RELA-positive ST-EPN still does not have any effective chemotherapy or targeted therapy. Although localized EWS with EWS:FLI1 fusion has a fairly good prognosis, metastatic or recurrent EWS is usually fatal. The challenge of many fusion-positive cancers is the lack of established targets downstream of the gene fusion despite years of intensive research to understand the biology of the gene fusion. In other cancers caused by a viral infection, cancer cells express viral transcripts not present in non-infected cells. In many cancers, mutations or other forms of genetic aberrations in endogenous genes are present and are unique to the cancer cells. Accordingly, methods for rapid and cost-effective development of precise targeted cancer therapeutics without reliance on the extensive knowledge of the molecular biology underlying the targeted cancer cells are needed.

### SUMMARY

Embodiments of the present disclosure provide a single-stranded nucleic acid sensor molecule that includes a target sensing region having a nucleic acid sequence that is substantially complementary to a target nucleic acid, wherein the target sensing region comprises a TAG or TGA stop codon opposite a corresponding CAA, CTA, CGA, ACA, TCA, GCA, CCA, CCT, or CCC triplet in the target nucleic acid positioned on at least one side of a junctional sequence in the target nucleic acid; and a response gene positioned downstream of the target sensing region, wherein the response gene is expressed when the TAG or TGA stop codon is converted to a TGG codon by adenosine deaminase acting on RNA (ADAR)-mediated RNA editing upon binding of the sensor molecule to the target nucleic acid.

In some embodiments, the junctional sequence of the target nucleic acid corresponds to at least a portion of a gene, transcript or chromosomal fusion. In some embodiments, the junctional sequence of the target nucleic acid comprises a CBFA2T3-GLIS2 fusion sequence, an EML4-ALK fusion sequence, a ZFTA-RELA fusion sequence, an EWSR1-FL1 fusion sequence, a CCNH-C5orf30 fusion sequence, a TMEM135-CCDC67 fusion sequence, an EVT6-NTRK3 fusion sequence, a TMPRSS2-ERG fusion sequence, a TRMT11-GRIK2 fusion sequence, or a PVT1-MYC fusion sequence. The junctional sequence of the target nucleic acid “comprising” a given fusion sequence does not necessitate that the junctional sequence comprises the entire fusion sequence, but rather only a portion of the fusion sequence (e.g. in some embodiments, the junctional sequence of the target nucleic acid comprises at least a portion of the full sequence encoding such a fusion protein). An exemplary CBFA2T3-GLIS2 fusion sequence is shown, for example, in SEQ ID NO: 2. In some embodiments the junctional sequence of the target nucleic acid comprises at least a portion of SEQ ID NO: 2. An exemplary EML4-ALK fusion sequence is shown in SEQ ID NO: 74. In some embodiments, the junctional sequence of the target nucleic acid comprises at least a portion of SEQ ID NO: 74. An exemplary ZFTA-RELA fusion sequence is shown in SEQ ID NO: 75. In some embodiments, the junctional sequence of the target nucleic acid comprises at least a portion of SEQ ID NO: 75. An exemplary EWSR1-FL1 fusion sequence is shown in SEQ ID NO: 76. In some embodiments, the junctional sequence of the target nucleic acid comprises at least a portion of SEQ ID NO: 76. An exemplary a CCNH-C5orf30 fusion sequence is shown in SEQ ID NO: 77. In some embodiments, the junctional sequence of the target nucleic acid comprises at least a portion of SEQ ID NO: 77. An exemplary a TMEM135-CCDC67 fusion sequence is shown in SEQ ID NO: 78. In some embodiments, the junctional sequence of the target nucleic acid comprises at least a portion of SEQ ID NO: 78. An exemplary a ETV6-NTRK3 fusion sequence is shown in SEQ ID NO: 79. In some embodiments, the junctional sequence of the target nucleic acid comprises at least a portion of SEQ ID NO: 79. An exemplary a TMPRSS2-ERG fusion sequence is shown in SEQ ID NO: 80. In some embodiments, the junctional sequence of the target nucleic acid comprises at least a portion of SEQ ID NO: 80. An exemplary a TRMT11-GRIK2 fusion sequence is shown in SEQ ID NO: 81. In some embodiments, the junctional sequence of the target nucleic acid comprises at least a portion of SEQ ID NO: 81. An exemplary a PVT1-MYC fusion sequence is shown in SEQ ID NO: 82. In some embodiments, the junctional sequence of the target nucleic acid comprises at least a portion of SEQ ID NO: 82.

In some embodiments, the junctional sequence comprises a TP53(R248Q) mutant transcript. An exemplary a TP53(R248Q) mutant transcript is shown in SEQ ID NO: 83. In some embodiments, the junctional sequence of the target nucleic acid comprises at least a portion of SEQ ID NO: 83.

In some embodiments, the junctional sequence of the target nucleic acid comprises a CBFA2T3-GLIS2 fusion sequence, and the target sensing region includes the nucleic acid sequence set forth in SEQ ID NO: 3. The target sensing region may include additional nucleic acids to those set forth in SEQ ID NO: 3. In some embodiments, the junctional sequence of the target nucleic acid comprises a CBFA2T3-GLIS2 fusion sequence, and the target sensing region comprises an nucleic acid sequence having at least 80% sequence identity to SEQ ID NO: 4 or SEQ ID NO: 5. For example, in some embodiments the target sensing region comprises an nucleic acid sequence having aet least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4 or to SEQ ID NO: 5.

In some embodiments, the junctional sequence of the target nucleic acid comprises an EML4-ALK fusion sequence, and the target sensing region includes the nucleic acid sequence set forth in SEQ ID NO: 28. The target sensing region may include additional nucleic acids to those set forth in SEQ ID NO: 28. In some embodiments, the junctional sequence of the target nucleic acid comprises an EML4-ALK fusion sequence, and the target sensing region comprises a nucleic acid sequence having at least 80% sequence identity to SEQ ID NO: 20 or SEQ ID NO: 29. For example, in some embodiments the target sensing region comprises an nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 20 or to SEQ ID NO: 29.

In some embodiments, the junctional sequence of the target nucleic acid comprises a ZFTA-RELA fusion sequence, and the target sensing region includes the nucleic acid sequence set forth in SEQ ID NO: 32. The target sensing region may include additional nucleic acids to those set forth in SEQ ID NO: 32. In some embodiments, the junctional sequence of the target nucleic acid comprises a ZFTA-RELA fusion sequence, and the target sensing region comprises an nucleic acid sequence having at least 80% sequence identity to SEQ ID NO: 31 or SEQ ID NO: 30. For example, in some embodiments the target sensing region comprises an nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 31 or to SEQ ID NO: 30.

In some embodiments, the junctional sequence of the target nucleic acid comprises an EWSR1-FL1 fusion sequence, and the target sensing region comprises an nucleic acid sequence having at least 80% sequence identity to SEQ ID NO: 33. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 33.

In some embodiments, the junctional sequence in the target nucleic acid comprises a CCNH-C5orf30 fusion sequence, and the target sensing region includes an nucleic acid sequence having at least 80% sequence identity to SEQ ID NO: 84. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 84.

In some embodiments, the junctional sequence in the target nucleic acid comprises a TMEM135-CCDC67 fusion sequence, and the target sensing region includes a nucleic acid sequence having at least 80% sequence identity to SEQ ID NO: 85. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 85.

In some embodiments, the junctional sequence in the target nucleic acid comprises a EVT6-NTRK3 fusion sequence, and the target sensing region includes a nucleic acid sequence having at least 80% sequence identity to SEQ ID NO: 86. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 86.

In some embodiments, the junctional sequence in the target nucleic acid comprises a TMPRSS2-ERG fusion sequence, and the target sensing region includes a nucleic acid sequence having at least 80% sequence identity to SEQ ID NO: 87. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 87.

In some embodiments, the junctional sequence in the target nucleic acid comprises a TRMT11-GRIK2 fusion sequence, and the target sensing region includes a nucleic acid sequence having at least 80% sequence identity to SEQ ID NO: 88. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 88.

In some embodiments, the junctional sequence in the target nucleic acid comprises a PVT1-MYC fusion sequence, and the target sensing region includes a nucleic acid sequence having at least 80% sequence identity to SEQ ID NO: 89. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 89.

In some embodiments, the junctional sequence in the target nucleic acid comprises a TP53(R248Q) mutant transcript. The junctional sequence of the target nucleic acid “comprising” a given mutant transcript indicates that the junctional sequence comprises at least a portion of the mutant transcript. In some embodiments, the junctional sequence comprises a TP53(R248Q) mutant transcript and the target sensing region includes an nucleic acid sequence having at least 80% sequence identity to SEQ ID NO: 90. For example, in some embodiments the target sensing region comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 90

In some embodiments, the junctional sequence of the target nucleic acid sequence comprises a viral transcript. The junctional sequence of the target nucleic acid “comprising” a given viral transcript indicates that the junctional sequence comprises at least a portion of the viral transcript. In some embodiments, the viral transcript is a transcript associated with cancer. In some embodiments, the viral transcript is an Epstein Barr Virus (EBV) transcript or a Kaposi's sarcoma-associated herpesvirus (KSHV) transcript. For example, in some embodiments the viral transcript is the Epstein Barr Virus transcript EBNA1 and the target sensing region comprises a nucleic acid sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% sequence identity) to SEQ ID NO: 34.

In some embodiments, the viral transcript is the KSHV transcript ORF71 and wherein the target sensing region comprises a nucleic acid sequence having at least 80% least 93%, at least 94%, at least 95%, at least 96%, at least 97%, at least 98%, at least 99%, or 100% sequence identity) to SEQ ID NO: 35.

In some embodiments, the target sensing region is at least about 50 nucleotides long. In some embodiments, the target sensing region is from about 50 nucleotides to about 1000 nucleotides long.

In some embodiments, the response gene encodes at least one of a reporter protein, a caspase, a prodrug-converting enzyme, or an enzyme catalyzing specific functions.

In some embodiments, the sensor molecule further comprises a control gene. In some embodiments, the control gene is constitutively expressed. For example, in some embodiments the control gene is a fluorescent protein.

In some embodiments, the sensor molecule comprises a linker region positioned upstream of the response gene but downstream of the TAG or TGA stop codon. In some embodiments, the linker region comprises a XTEN80 peptide. In some embodiments, the linker region comprises a 2A peptide.

In some embodiments, the sensor molecule comprises an RNA aptamer sequence capable of binding its cognate binding protein. In some embodiments, the RNA aptamer comprises a sequence capable of binding at least one of MS2, PP7, BoxB, or Pumilio. In some embodiments, the cognate binding protein is fused to an ADAR protein, including any mutants, derivatives, or variants thereof. In some embodiments, the cognate binding protein is fused to a domain of an ADAR protein. In some embodiments, the cognate binding protein is fused to an ADAR mutant protein. Exemplary ADAR mutant proteins include ADARdd(E488Q) and ADARddm(C377F,E488Q). In some embodiments, the cognitive binding protein is MCP. In some embodiments, the sensor molecule comprises MCP-ADARdd(E488Q) or MCP-ADARddm(C377F,E488Q).

Embodiments of the present disclosure also include an expression vector comprising a DNA sequence corresponding to any of the RNA sensor molecules described herein.

In some embodiments, provided herein is a sensor molecule wherein no exogenous ADAR is necessary to detect a target sequence. In some embodiments, the sensor molecule comprises a gene encoding an ADAR or an ADAR fusion, wherein the ADAR or ADAR fusion is constitutively expressed. In some embodiments, the ADAR fusion comprises an ADAR enzyme fused to a cognate aptamer-binding protein. In some embodiments, the ADAR fusion comprises a mutant ADAR protein. Exemplary ADAR mutant proteins include ADARdd(E488Q) and ADARddm(C377F,E488Q). In some embodiments, the cognitive binding protein is MCP. In some embodiments, the sensor molecule comprises MCP-ADARdd(E488Q) or MCP-ADARddm(C377F,E488Q). For example, in some embodiments the ADAR enzyme (or mutant ADAR enzyme) is fused to a cognate aptamer-binding protein (e.g. MS2 coat protein (MCP), PP7 coat protein (PCP), lambaN protein, or Pumilio/PUF-HD domains) to form a single recombinant protein. In some embodiments, the sensor molecule further comprises an RNA aptamer sequence that recruits the cognate aptamer-binding protein-ADAR fusion.

In some embodiments, the sensor molecule is an RNA molecule.

In some embodiments, the expression vector is selected from the group consisting of: a pCR8-mRuby2-P2A-ccdbCam-E2A-EGFP vector; a pCR8-mRuby2-P2A-ccdbCam-E2A-EGFP-BsaI(agat) vector; a pCR8-mRuby2-P2A-ccdbCam-E2A-EGFP-NxMS2 vector; a pCR8-mRuby2-P2A-Sensor-E2A-EGFP vector; a pCR8-mRuby2-P2A-Sensor-E2A-EGFP-NxMS2 vector; a pmax-mRuby2-P2A-Sensor-XTEN80-EGFP-NxMS2 vector; a MCP-ADARdd(E488Q) vector; a pmax-MCP-ADARdd(E488Q), a pmax-MCP-ADARdd(E488Q)-P2A-Sensor-XTEN80-NTR1.1-NxMS2 vector; a pmax-MCP-ADARdd(E488Q)-P2A-Sensor-E2A-NTR1.1-NxMS2 vector; a pmax-MCP-ADARddm(C377F,E488Q)-P2A-Sensor-XTEN80-NTR1.1-NxMS2 vector; a pmax-MCP-ADARddm(C377F,E488Q)-P2A-Sensor-E2A-NTR1.1-NxMS2 vector; a pmax-MCP-ADARdd(E488Q)-P2A-Sensor-XTEN80-DTA-NxMS2 vector; a pmax-MCP-ADARdd(E488Q)-P2A-Sensor-E2A-DTA-NxMS2 vector; a pmax-MCP-ADARddm(C377F,E488Q)-P2A-Sensor-XTEN80-DTA-NxMS2 vector; a pmax-MCP-ADARddm(C377F,E488Q)-P2A-Sensor-E2A-DTA-NxMS2 vector; a pmax-MCP-ADARdd(E488Q)-P2A-Sensor-XTEN80-BAX-NxMS2 vector; a pmax-MCP-ADARdd(E488Q)-P2A-Sensor-E2A-BAX-NxMS2 vector; a pmax-MCP-ADARddm(C377F,E488Q)-P2A-Sensor-XTEN80-BAX-NxMS2 vector; a and pmax-MCP-ADARddm(C377F,E488Q)-P2A-Sensor-E2A-BAX-NxMS2 vector.

Embodiments of the present disclosure also include a cell comprising any of the RNA sensor molecules described herein, or any of the vectors described herein.

Embodiments of the present disclosure also include a kit comprising any of the RNA sensor molecules described herein, any of the vectors described herein, and/or any of the cells described herein.

Embodiments of the present disclosure also include a method of treating a subject having cancer or suspected of having cancer. In accordance with these embodiments, the method includes administering any of the RNA sensor molecules described herein, any of the vectors described herein, and/or any of the cells described herein to the subject; and treating the subject. In some embodiments, the cancer is caused by a chromosomal translocation and/or gene fusion.

Embodiments of the present disclosure also include a method of detecting a gene fusion transcript in a cell. In accordance with these embodiments, the method includes transfecting a cell with any of the RNA sensor molecules described herein, or any of the vectors described herein, and assessing the cell for expression of a reporter protein.

### BRIEF DESCRIPTION OF THE DRAWINGS

FIGS. 1A-1E: Representative schematic diagrams of a target gene fusion and its transcript (FIG. 1A), and the nucleic acid sensors designed to target that gene fusion transcript (FIGS. 1B-1E), according to one embodiment of the present disclosure.

FIGS. 2A-2E: Representative schematic diagrams of exemplary vector systems to facilitate cloning and testing of the different sensor sequences and sensor architectures of the present disclosure. FIG. 2A: pCR8-mRuby2-P2A-ccdbCam-E2A-EGFP vector. FIG. 2B: pCR8-mRuby2-P2A-ccdbCam-E2A-EGFP-BsaI(agat) vector; and pCR8-mRuby2-P2A-ccdbCam-E2A-EGFP-NxMS2 vector (e.g., where N can be any number). FIG. 2C: pCR8-mRuby2-P2A-Sensor-E2A-EGFP vector (Gateway destination expression vector in FIG. 2D); and pCR8-mRuby2-P2A-Sensor-E2A-EGFP-NxMS2 vector (Gateway destination expression vector in FIG. 2E).

FIGS. 3A-3C: Representative schematic diagrams of four exemplary sensor sequences to detect CBFA2T3-GLIS2 fusion sequence, with lengths of 93 (CBFA2T3GLIS2_93_sensor), 351 (CBFA2T3GLIS2_351_sensor), 495 (CBFA2T3GLIS2_495_sensor), roughly centered at the fusion junction. A sensor consisting of four MS2 stem loops inserted within the sensor region was also designed (CBFA2T3GLIS2_avidity5) (FIG. 3A). Representative fluorescence detection data (using flow cytometry) in HEK293 cells transfected with the sensor-response vectors (pmax-mRuby2-P2A-Sensor-E2A-EGFP-9xMS2), MCP-ADARdd(E488Q) vector (pmax-MCP-ADARdd(E488Q)) and either a control empty vector or vector expressing test fusion gene (pmax-CBFA2T3-GLIS2_FL or pmax-CBFA2T3-GLIS3_mini750) (FIG. 3B). Representative fluorescence detection data (using flow cytometry) in HEK293 cells transfected with the sensor-response vectors (pmax-mRuby2-P2A-Sensor-E2A-EGFP-9xMS2 or pmax-mRuby2-P2A-Sensor-XTEN80-EGFP-9xMS2), MCP-ADARdd(E488Q) vector (pmax-MCP-ADARdd(E488Q)) and either a control empty vector or vector expressing test fusion gene (pmax-CBFA2T3-GLIS2_FL or pmax-CBFA2T3-GLIS3_mini750) (FIG. 3C). The presence of fusion transcript led to increase of GFP signal not seen in cells transfected with empty vector control, demonstrating the specific detection of fusion CBFA2T3-GLIS2 transcripts in vivo in HEK293T.

FIG. 4: Representative schematic diagram of a nucleic acid sensor-NTR designed to detect gene fusion transcript, according to one embodiment of the present disclosure

FIGS. 5A-5B: FIG. 5A includes a schematic diagram of an exemplary sensor-NTR to detect CBFA2T3-GLIS2 fusion sequence with a length of 495 (CBFAZT3GLIS2_495_sensor), roughly centered at the fusion junction. The CBFAZT3GLIS2_495_sensor is cloned upstream of XTEN80 protein linker, NTR1.1 coding sequence, and 9 copies of MS2 stem loops (9xMS2). Also a schematic of pmax-MCP-ADARdd(E488Q) vector expressing MCP-ADARdd(E488Q) that can be recruited to the 9xMS2. FIG. 5B includes representative cellular viability data (using CellTiter-Glo 2.0 assay) in HEK293T cells transfected with the sensor-NTR vector (pmax-mRuby2-P2A-Sensor (CBFA2T3GLIS2_495)-XTEN80-NTR1.1-9xMS2), MCP-ADARdd(E488Q) vector, and either a control empty vector, combination of vectors expressing unfused constituents (pmax-CBFA2T3 and pmax-GLIS2), or vector expressing CBFA2T3-GLIS2 fusion gene (pmax-CBFA2T3-GLIS2_FL). The presence of fusion transcript, but not empty vector or unfused constituent transcripts, led to decrease in cellular viability, demonstrating the specific detection and ablation of cell populations expressing CBFA2T3-GLIS2 transcripts in vivo in HEK293T.

FIGS. 6A-6B: FIG. 6A includes a schematic diagram of an exemplary sensor-NTR to detect EML4-ALK fusion sequence with a length of 501 (EML4ALK_501_sensor), roughly centered at the fusion junction. The EML4ALK_501_sensor is cloned upstream of E2A peptide, NTR1.1 coding sequence, and 9 copies of MS2 stem loops (9xMS2). Also a schematic of pmax-MCP-ADARdd(E488Q) vector expressing MCP-ADARdd(E488Q) that can be recruited to the 9xMS2.

FIG. 6B includes representative cellular viability data (using CellTiter-Glo 2.0 assay) in HEK293T cells transfected with the sensor-NTR vector (pmax-mRuby2-P2A-Sensor (EML4ALK_501)-E2A-NTR1.1-9xMS2), MCP-ADARdd(E488Q) vector, and either a control empty vector, or vector expressing EML4-ALK fusion gene (pmax-EML4-ALK). The presence of fusion transcript, but not empty vector, led to decrease in cellular viability, demonstrating the specific detection and ablation of cell populations expressing EML4-ALK transcripts in vivo in HEK293T.

FIG. 7: Representative schematic diagram of a nucleic acid ADAR-sensor-NTR designed to detect gene fusion transcript, according to one embodiment of the present disclosure.

FIG. 8A: Representative schematic diagram of an exemplary ADAR-sensor-NTR to detect CBFA2T3-GLIS2 fusion sequence with a length of 495 (CBFAZT3GLIS2_495_sensor), roughly centered at the fusion junction. The construct consists of coding sequence of MCP-ADARdd(E488Q), P2A peptide, CBFAZT3GLIS2_495_sensor, E2A peptide, NTR1.1 coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 8B: Representative cellular viability data (using CellTiter-Glo 2.0 assay) in HEK293T cells transfected with the ADAR-sensor-NTR vector (pmax-MCP_ADARdd(E488Q)-P2A-Sensor (CBFA2T3GLIS2_495)-E2A-NTR1.1-9xMS2), and either a control empty vector, combination of vectors expressing unfused constituents (pmax-CBFA2T3 and pmax-GLIS2), or vector expressing CBFA2T3-GLIS2 fusion gene (pmax-CBFA2T3-GLIS2_FL). The presence of fusion transcript, but not empty vector or unfused constituent transcripts, led to decrease in cellular viability, demonstrating the specific detection and ablation of cell populations expressing CBFA2T3-GLIS2 transcripts in vivo in HEK293T.

FIG. 9A: Representative schematic diagram of exemplary ADAR-sensor-NTR constructs to detect CBFAT3-GLIS2 fusion sequence with different sensor lengths (L_{S}). Each construct consists of coding sequence of MCP-ADARdd(E488Q), P2A peptide, CBFA2T3-GLIS2 sensors with sensor length (L_{S}) of 90 nt, 150 nt or 495 nt, E2A peptide, NTR1.1 coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 9B: Alignment of Target and 501nt Sensor sequence for CBFA2T3-GLIS2 fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The sensing stop codons are underlined. The right-pointing and left-pointing arrows denote the start and end of the sensor sequence for the indicated sensor length (L_{S}).

FIG. 9C: Representative cellular viability data (using CellTiter-Glo 2.0 assay) in HEK293T cells transfected with the ADAR-sensor-NTR vector with 90-nt, 150nt or 495-nt sensors, and either a control empty vector or vector expressing CBFA2T3-GLIS2 fusion gene (pmax-CBFA2T3-GLIS2_FL). ADAR mutant variants, single mutant MCP-ADARdd(E488Q) and double mutant MCP-ADARddm(C377F,E488Q), were included. Column plot shows normalized cellular viability of CBFA2T3-GLIS2 fusion-positive compared to fusion-negative cells for ADAR-sensor-NTR constructs with the indicated sensor lengths and ADAR mutant variants. Increase in sensor length leads generally to decreased normalized cellular viability (higher degree of cell ablation). In addition, double mutant MCP-ADARddm(C377F,E488Q) induces higher degree of cell ablation compared to single mutant MCP-ADARdd(E488Q).

FIG. 10A: Representative schematic diagram of exemplary ADAR-sensor-NTR constructs to detect EML4-ALK fusion sequence with different sensor lengths (L_{S}). The construct consists of coding sequence of MCP-ADARdd(E488Q), P2A peptide, EML4-ALK sensors with sensor length (L_{S}) of 90 nt, 150 nt or 501 nt, E2A peptide, NTR1.1 coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 10B: Alignment of Target and 50Int Sensor sequence for EML4-ALK fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codons are underlined. The fusion junction is indicated. The right-pointing and left-pointing arrows denote the start and end of the sensor sequence for the indicated sensor length (L_{S}).

FIG. 10C: Representative cellular viability data (using CellTiter-Glo 2.0 assay) in HEK293T cells transfected with the ADAR-sensor-NTR vector with 90-nt, 150nt or 501-nt sensors, and either a control empty vector or vector expressing EML4-ALK fusion gene (pmax-EML4-ALK). ADAR mutant variants, single mutant MCP-ADARdd(E488Q) and double mutant MCP-ADARddm(C377F,E488Q), were included. Column plot shows normalized cellular viability of EML4-ALK fusion-positive compared to fusion-negative cells for ADAR-sensor-NTR constructs with the indicated sensor lengths and ADAR mutant variants. Increase in sensor length leads generally to decreased normalized cellular viability (higher degree of cell ablation). In addition, double mutant MCP-ADARddm(C377F,E488Q) induces higher degree of cell ablation compared to single mutant MCP-ADARdd(E488Q).

FIG. 11A: Representative schematic diagram of exemplary ADAR-sensor-NTR constructs to detect ZFTA-RELA fusion sequence with different sensor lengths (L_{S}). The construct consists of coding sequence of MCP-ADARdd(E488Q), P2A peptide, ZFTA-RELA sensors with sensor length (L_{S}) of 90 nt, 150 nt or 501 nt, E2A peptide, NTR1.1 coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 11B: Alignment of Target and 501nt Sensor sequence for ZFTA-RELA fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 8-flag denotes a mutation to the sensor sequence to allow synthesis for cloning. The sensing stop codons are underlined. The right-pointing and left-pointing arrows denote the start and end of the sensor sequence for the indicated sensor length (L_{S}).

FIG. 11C: Representative cellular viability data (using CellTiter-Glo 2.0 assay) in HEK293T cells transfected with the ADAR-sensor-NTR vector with 90-nt, 150nt or 501-nt sensors, and either a control empty vector or vector expressing ZFTA-RELA fusion gene (pmax-ZFTA-RELA). Column plot shows normalized cellular viability of ZFTA-RELA fusion-positive compared to fusion-negative cells for ADAR-sensor-NTR constructs with the indicated sensor lengths. 501-nt sensor outperforms the shorter 90-nt and 150-nt sensors for inducing higher degree of cell ablation.

FIG. 11D: Representative schematic diagram of lentivirus carrying exemplary ADAR-sensor-NTR constructs to detect endogenous ZFTA-RELA fusion transcripts expressed in BDX-1425EPN cancer cells and induce cell ablation. The construct consists of coding sequence of MCP-ADARdd(E488Q), P2A peptide, ZFTA-RELA sensor with sensor length of 501 nt, XTEN80 linker or E2A peptide, NTR1.1 coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 11E: Representative cellular viability data (using CellTiter-Glo 2.0 assay) of non-transduced BDX-1425EPN cells or those transduced with lentiviruses carrying ADAR-sensor-NTR vector with 501-nt sensors, and either XTEN80 or E2A peptides between the sensor and NTR. Column plot shows cellular viability of non-transduced cells (No Tdx), cells transduced with lentiviral sensors with XTEN80 or E2A peptides.

FIG. 12A: Representative schematic diagram of an exemplary ADAR-sensor-NTR to detect EWSR1-FLI1 fusion sequence with a length of 501 (EWSR1_FLI1_501_sensor). The construct consists of coding sequence of MCP-ADARdd(E488Q), P2A peptide, EWSR1_FLI1_501_sensor, E2A peptide, NTR1.1 coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined. <97nt> indicates 97 nucleotides not shown in the schematic.

FIG. 12B: Alignment of Target and Sensor sequence for EWSR1-FLI1 fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codons are underlined. The fusion junction is indicated.

FIG. 12C: Representative cellular viability data (using CellTiter-Glo 2.0 assay) in HEK293T cells transfected with the ADAR-sensor-NTR vector (pmax-MCP_ADARdd(E488Q)-P2A-Sensor (EWSR1FLI1_501)-E2A-NTR1.1-9xMS2), and either a control empty vector or vector expressing EWSR1-FLI1 fusion minigene (pmax-EWSR1-FLI1). Line plot shows normalized cellular viability of EWSR1-FLI1 fusion-positive compared to fusion-negative cells on the indicated days post drug addition for two trials of the experiment. The presence of fusion transcript, but not empty vector, led to decrease in cellular viability, demonstrating the specific detection and ablation of cell populations expressing EWSR1-FLI1 transcripts in vivo in HEK293T.

FIG. 13: Representative schematic diagram of a nucleic acid ADAR-sensor-DTA designed to detect gene fusion transcript, according to one embodiment of the present disclosure.

FIG. 14A: Representative schematic diagram of an exemplary ADAR-sensor-DTA to detect CBFA2T3-GLIS2 fusion sequence with a length of 495 (CBFA2T3GLIS2_495_sensor). The construct consists of coding sequence of MCP-ADARdd(E488Q), P2A peptide, CBFA2T3GLIS2_495_sensor, E2A peptide, DTA coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 14B: Representative cellular viability data (using CellTiter-Glo 2.0 assay) in HEK293T cells transfected with the ADAR-sensor-DTA vector (pmax-MCP_ADARdd(E488Q)-P2A-Sensor (CBFA2T3GLIS2_495)-E2A-DTA-9xMS2), and either a control empty vector or vector expressing CBFA2T3-GLIS2 fusion gene (pmax-CBFA2T3-GLIS2_FL). Line plot shows normalized cellular viability of CBFA2T3-GLIS2 fusion-positive compared to fusion-negative cells on the indicated days post drug addition. The presence of fusion transcript, but not empty vector, led to decrease in cellular viability, demonstrating the specific detection and ablation of cell populations expressing CBFA2T3-GLIS2 transcripts in vivo in HEK293T.

FIG. 15: Representative schematic diagram of a nucleic acid ADAR-sensor-BAX designed to detect gene fusion transcript, according to one embodiment of the present disclosure.

FIG. 16A: Representative schematic diagram of an exemplary ADAR-sensor-BAX to detect CBFA2T3-GLIS2 fusion sequence with a length of 495 (CBFA2T3GLIS2_495_sensor). The construct consists of coding sequence of MCP-ADARdd(E488Q), P2A peptide, CBFA2T3GLIS2_495_sensor, E2A peptide or XTEN80 linker, DTA coding sequence, and 9 copies of MS2 stem loops (9xMS2). The 9xMS2 can recruit MCP-ADARdd(E488Q) expressed from the same RNA molecule. Center sequences show part of the fusion transcript sequence (top, 3′-5′) spanning the junction with CCA triplets bold and underlined, as well as the corresponding part of the sensor sequence (bottom, 5′-3′) with two sensing stop codons bold and underlined.

FIG. 16B: Representative cellular viability data (using CellTiter-Glo 2.0 assay) in HEK293T cells transfected with the ADAR-sensor-BAX vectors, and either a control empty vector or vector expressing CBFA2T3-GLIS2 fusion gene (pmax-CBFA2T3-GLIS2_FL). Column plot shows normalized cellular viability of CBFA2T3-GLIS2 fusion-positive compared to fusion-negative cells on Day 5 post drug addition for the ADAR-sensor-BAX constructs with the indicated E2A or XTEN80 linker.

FIG. 17: Representative schematic diagram of a nucleic acid ADAR-sensor-NTR designed to detect viral transcript, according to one embodiment of the present disclosure.

FIG. 18A: Representative schematic diagram of an exemplary ADAR-sensor-NTR to detect Epstain Barr Virus (EBV)-EBNA1 transcript with a sensor length of 501 (EBNA1_501_sensor). The construct consists of coding sequence of MCP-ADARdd(E488Q), P2A peptide, EBNA1_501_sensor, XTEN80 protein linker, NTR1.1 coding sequence, and 9 copies of MS2 stem loops (9xMS2). The 9xMS2 can recruit MCP-ADARdd(E488Q) expressed from the same RNA molecule. Center sequences show part of the viral transcript sequence (top, 3′-5′) with the target CCA triplet bold and underlined, as well as the corresponding part of the sensor sequence (bottom, 5′-3′) with the sensing stop codon bold and underlined.

FIG. 18B: Alignment of Target and Sensor sequence for EBV-EBNA1. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 5-flag and 6-flag denote a mutation to the sensor sequence to eliminate unwanted stop codons or unwanted start codons, respectively. The sensing stop codon is underlined.

FIG. 18C: Representative cellular viability data (using CellTiter-Glo 2.0 assay) in HEK293T cells transfected with the ADAR-sensor-NTR vector (pmax-MCP_ADARdd(E488Q)-P2A-Sensor (EBNA1_501)-XTEN80-NTR1.1-9xMS2), and either a control empty vector or vector expressing EBV-EBNA1 gene (pmax-EBNA1). The presence of viral transcript, but not empty vector, led to decrease in cellular viability, demonstrating the specific detection and ablation of cell populations expressing EBV-EBNA1 transcripts in vivo in HEK293T.

FIG. 19A: Representative schematic diagram of an exemplary ADAR-sensor-NTR to detect Kaposi's sarcoma-associated herpesvirus (KSHV)-ORF71 transcript with a sensor length of 501 (KSHV_ORF71_501_sensor). The construct consists of coding sequence of MCP-ADARdd(E488Q), P2A peptide, KSHV_ORF71_501_sensor, XTEN80 protein linker, NTR1.1 coding sequence, and 9 copies of MS2 stem loops (9xMS2). The 9xMS2 can recruit MCP-ADARdd(E488Q) expressed from the same RNA molecule. Center sequences show part of the viral transcript sequence (top, 3′-5′) with the target CCA triplet bold and underlined, as well as the corresponding part of the sensor sequence (bottom, 5′-3′) with the sensing stop codon bold and underlined.

FIG. 19B: Alignment of Target and Sensor sequence for KSHV-ORF71. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 5-flag and 6-flag denote a mutation to the sensor sequence to eliminate unwanted stop codons or unwanted start codons, respectively. The sensing stop codon is underlined.

FIG. 19C: Representative cellular viability data (using CellTiter-Glo 2.0 assay) in HEK293T cells transfected with the ADAR-sensor-NTR vector (pmax-MCP_ADARdd(E488Q)-P2A-Sensor (KSHV_ORF71_501)-XTEN80-NTR1.1-9xMS2), and either a control empty vector or vector expressing KSHV-ORF71 gene (pOME0343_ORF71tagged). The presence of viral transcript, but not empty vector, led to decrease in cellular viability, demonstrating the specific detection and ablation of cell populations expressing KSHV-ORF71 transcripts in vivo in HEK293T.

FIG. 20A: Representative schematic diagram of an exemplary ADAR-sensor-NTR with two sensing stop codons surrounding the fusion junction. The construct consists of coding sequence of MCP-ADARddm(C377F,E488Q), P2A peptide, sensor sequence, E2A peptide, NTR1.1 coding sequence, and 9 copies of MS2 stem loops (9xMS2). The 9xMS2 can recruit MCP-ADARddm(C377F,E488Q) expressed from the same RNA molecule.

FIG. 20B: Alignment of Target and Sensor sequence for CCNH-C5orf30 fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codons are underlined. The fusion junction is indicated.

FIG. 20C: Representative cellular viability data (using CellTiter-Glo 2.0 assay) in HEK293T cells transfected with the ADAR-sensor-NTR vector (pmax-MCP_ADARddm(C377F,E488Q)-P2A-Sensor (CCNH_C5orf30_sensor_501)-E2A-NTR1.1-9xMS2), and either a control empty vector or vector expressing CCNH-C5orf30 minigene. The presence of fusion transcript, but not empty vector, led to decrease in cellular viability, demonstrating the specific detection and ablation of cell populations expressing CCNH-C5orf30 transcripts in vivo in HEK293T.

FIG. 20D: Alignment of Target and Sensor sequence for TMEM135-CCDC67 fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codons are underlined. The fusion junction is indicated.

FIG. 20E: Representative cellular viability data (using CellTiter-Glo 2.0 assay) in HEK293T cells transfected with the ADAR-sensor-NTR vector (pmax-MCP_ADARddm(C377F,E488Q)-P2A-Sensor (TMEM135_CCDC67_sensor_501)-E2A-NTR1.1-9xMS2), and either a control empty vector or vector expressing TMEM135-CCDC67 minigene. The presence of fusion transcript, but not empty vector, led to decrease in cellular viability, demonstrating the specific detection and ablation of cell populations expressing TMEM135-CCDC67 transcripts in vivo in HEK293T.

FIG. 20F: Alignment of Target and Sensor sequence for ETV6-NTRK3 fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codons are underlined. The fusion junction is indicated.

FIG. 20G: Representative microscopy images of HEK293T cells transfected with the ADAR-sensor-NTR vector (pmax-MCP_ADARddm(C377F,E488Q)-P2A-Sensor (ETV6_NTRK3_sensor_501)-E2A-NTR1.1-9xMS2), and either a control empty vector or vector expressing ETV6-NTRK3 minigene. The presence of fusion transcript, but not empty vector, led to decrease in cellular viability, demonstrating the specific detection and ablation of cell populations expressing ETV6-NTRK3 transcripts in vivo in HEK293T.

FIG. 20H: Alignment of Target and Sensor sequence for TMPRSS2-ERG fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codons are underlined. The fusion junction is indicated.

FIG. 20I: Representative microscopy images of HEK293T cells transfected with the ADAR-sensor-NTR vector (pmax-MCP_ADARddm(C377F,E488Q)-P2A-Sensor (TMPRSS2_ERG_sensor_264)-E2A-NTR1.1-9xMS2), and either a control empty vector or vector expressing TMPRSS2-ERG minigene. The presence of fusion transcript, but not empty vector, led to decrease in cellular viability, demonstrating the specific detection and ablation of cell populations expressing TMPRSS2-ERG transcripts in vivo in HEK293T.

FIG. 21A: Representative schematic diagram of an exemplary ADAR-sensor-NTR with one sensing stop codon. The construct consists of coding sequence of MCP-ADARddm(C377F,E488Q), P2A peptide, sensor sequence, E2A peptide, NTR1.1 coding sequence, and 9 copies of MS2 stem loops (9xMS2). The 9xMS2 can recruit MCP-ADARddm(C377F,E488Q) expressed from the same RNA molecule.

FIG. 21B: Alignment of Target and Sensor sequence for TRMT11-GRIK2 fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 5-flag and 6-flag denote a mutation to the sensor sequence to eliminate unwanted stop codons or unwanted start codons, respectively. The sensing stop codon is underlined. The fusion junction is indicated.

FIG. 21C: Representative cellular viability data (using CellTiter-Glo 2.0 assay) in HEK293T cells transfected with the ADAR-sensor-NTR vector (pmax-MCP_ADARddm(C377F,E488Q)-P2A-Sensor (TRMT11_GRIK2ss_Sensor_201)-E2A-NTR1.1-9xMS2), and either a control empty vector or vector expressing TRMT11-GRIK2 minigene. The presence of fusion transcript, but not empty vector, led to decrease in cellular viability, demonstrating the specific detection and ablation of cell populations expressing TRMT11-GRIK2 transcripts in vivo in HEK293T.

FIG. 21D: Alignment of Target and Sensor sequence for PVT1-MYC fusion. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 4-flag denotes an insertion to the sensor sequence to preserve frames between sensor stop codons. The corresponding target position has a gap (-) with respect to the sensor. The 5-flag and 6-flag denote a mutation to the sensor sequence to eliminate unwanted stop codons or unwanted start codons, respectively. The 7-flag denotes a deletion at the sensor sequence to preserve frames between sensing stop codons. The sensing stop codon is underlined. The fusion junction is indicated.

FIG. 21E: Representative cellular viability data (using CellTiter-Glo 2.0 assay) in HEK293T cells transfected with the ADAR-sensor-NTR vector (pmax-MCP_ADARddm(C377F,E488Q)-P2A-Sensor (PVT1_MYC_sensor_498)-E2A-NTR1.1-9xMS2), and either a control empty vector or vector expressing PVT1-MYC minigene. The presence of fusion transcript, but not empty vector, led to decrease in cellular viability, demonstrating the specific detection and ablation of cell populations expressing PVT1-MYC transcripts in vivo in HEK293T.

FIG. 22: Representative schematic diagram of a nucleic acid ADAR-sensor-NTR designed to detect mutant transcripts, according to one embodiment of the present disclosure

FIG. 23A: Representative schematic diagram of an exemplary ADAR-sensor-NTR for the TP53(R248Q) mutant transcript. The construct consists of coding sequence of MCP-ADARddm(C377F,E488Q), P2A peptide, sensor sequence, XTEN80 linker peptide, NTR1.1 coding sequence, and 9 copies of MS2 stem loops (9xMS2). The 9xMS2 can recruit MCP-ADARddm(C377F,E488Q) expressed from the same RNA molecule.

FIG. 23B: Alignment of Target and Sensor sequence for the TP53(R248Q) mutant transcript. The alignment is displayed in groups of three lines. The top line displays the target sequence in reverse (3′->5′). The middle line displays number-encoded flags for the alignment. The bottom line displays the sensor sequence in 5′->3′ direction. The nucleotides are organized into codons encoded by the sensor. The 0-flag denotes that the target and sensor are aligned at that position. The 1-flag, 2-flag and 3-flag denote the first, second, and third nucleotide of a sensing stop codon, respectively. The 5-flag and 6-flag denote a mutation to the sensor sequence to eliminate unwanted stop codons or unwanted start codons, respectively. The sensing stop codon is underlined.

FIG. 23C: Representative cellular viability data (using CellTiter-Glo 2.0 assay) in HEK293T cells transfected with the ADAR-sensor-NTR vector (pmax-MCP_ADARddm(C377F,E488Q)-P2A-Sensor (TP53_R248Q_sensor111)-E2A-NTR1.1-9xMS2), and either plasmid expressing wid-type TP53 (pmax-TP53) or mutant TP53(R248Q) (pmax-TP53(R248Q)). The presence of mutant TP53(R248Q) transcript, but not wild-type TP53 transcript, led to decrease in cellular viability, demonstrating the specific detection and ablation of cell populations expressing TP53(R248Q) mutant transcripts in vivo in HEK293T.

### DETAILED DESCRIPTION

The present disclosure provides compositions and methods related to nucleic acid sensors. In particular, the present disclosure provides nucleic acids molecules that target transcripts of a gene fusion (or viral transcript, or mutant gene) and activate a downstream therapeutic function, thereby reducing or preventing the adverse effects of the gene fusion (or viral transcript, or mutant gene). In accordance with these embodiments, experiments were conducted to investigate the ability of nucleic acid sensors (e.g., RNA sensors) to target the genomic abnormality of a gene fusion (or viral transcript, or mutant gene) without prior knowledge of the function of the fusion gene (or viral transcript, or mutant gene). As described further herein, the nucleic acid sensors of the present disclosure target specific gene fusions (or viral transcript, or mutant gene) and trigger programmed events, such as cytotoxic events, the expression of immunostimulatory proteins, or other therapeutic functions upon binding to the fusion sequence on RNA transcripts expressed from fusion genes (or viral transcript, or mutant gene) in live cancer cells. For example, as long as cancer cells express the fusion transcript (or viral transcript, or mutant gene), the RNA-sensor approach of the present disclosure can seek out these cancer cells and destroy them by triggering the downstream event of apoptosis or activation of a prodrug, or the execution of other programmed therapeutic functions. This platform technology combines the advantages of targeting only cancer cells with the fusion (or viral transcript, or mutant gene) (specificity) and saving enormous amounts of time by eliminating the need to study the biology of the fusion before developing relevant therapeutics (rapid development). Additionally, this platform technology may be readily reprogrammed to target many different types of cancers harboring fusion genes and transcripts.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA, including, but not limited to, oligos of peptide nucleic acids (PNA). The term “RNA analog” or “RNA derivative” or “modified RNA” generally refer to a polymeric molecule, which in addition to containing ribonucleosides as its units, also contains at least one of the following: 2′-deoxy, 2′-halo (including 2′-fluoro), 2′-amino (preferably not substituted or mono- or disubstituted), 2′-mono-, di- or tri-halomethyl, 2′-O-alkyl, 2′-O-halo-substituted alkyl, 2′-alkyl, azido, phosphorothioate, sulfhydryl, methylphosphonate, fluorescein, rhodamine, pyrene, biotin, xanthine, hypoxanthine, 2,6-diamino purine, 2-hydroxy-6-mercaptopurine, N1-Methyl-Pseudouridine-5′-Triphosphate (N1meΨTP), and pyrimidine bases substituted at the 6-position with sulfur or 5 position with halo or C1-5 alkyl groups, a basic linkers, 3′-deoxy-adenosine as well as other available “chain terminator” or “non-extendible” analogs (at the 3′-end of the RNA), or labels such as ³²p, ³³P and the like. All of the foregoing can be incorporated into an RNA using the standard synthesis techniques disclosed herein.

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

“Sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

### 2. COMPOSITIONS AND METHODS

Embodiments of the present disclosure provide a single-stranded nucleic acid sensor molecule that includes a target sensing region having a nucleic acid sequence that is substantially complementary to a target nucleic acid, wherein the target sensing region comprises a TAG or TGA stop codon opposite a corresponding CAA, CTA, CGA, ACA, TCA, GCA, CCA, CCT, or CCC triplet in the target nucleic acid positioned on at least one side of a junctional sequence in the target nucleic acid; and a response gene positioned downstream of the target sensing region, wherein the response gene is expressed when the TAG or TGA stop codon is converted to a TGG codon by adenosine deaminase acting on RNA (ADAR)-mediated gene editing upon binding of the sensor molecule to the target nucleic acid. In some embodiments, the sensor molecule itself constitutively expresses an ADAR or an ADAR-fusion, which enables ADAR-mediated gene editing upon binding of the sensor molecule to the target nucleic acid. Such a sensor is referred to herein as an “all-in-one” sensor.

In some embodiments, the sensor molecules provided herein are used for detection of a fusion (e.g. a gene fusion, a chromosomal fusion). In some embodiments, the junctional sequence of the target nucleic acid corresponds to sequence spanning the junction between the constituents of a gene or chromosomal fusion. In other words, in some embodiments the junctional sequence is a sub-portion of a sequence of the gene or chromosomal fusion that comprises sequences on both sides of the junction of the fusion. In some embodiments, the sensor molecules provided herein are used for detection of mutant transcripts or viral transcripts wherein a fusion is not present. Although the junctional sequence is often used herein in reference to a fusion, the term “junctional sequence” does not necessitate that a fusion be present in the target nucleic acid. In some embodiments, for example when the target sequence is a mutant transcript or a viral transcript, the junctional sequence of the target nucleic acid refers to a portion of a mutant transcript or a portion of a viral transcript.

The sensor molecules are demonstrated herein be broadly applicable to a variety of gene or chromosomal fusion targets. In some embodiments, the gene or chromosomal fusion is associated with cancer. For example, in some embodiments the gene or chromosomal fusion is a CBFA2T3-GLIS2 fusion sequence, an EML4-ALK fusion sequence, a ZFTA-RELA fusion sequence, an EWSR1-FL1 fusion sequence, a CCNH-C5orf30 fusion sequence, a TMEM135-CCDC67 fusion sequence, an EVT6-NTRK3 fusion sequence, a TMPRSS2-ERG fusion sequence, a TRMT11-GRIK2 fusion sequence, or a PVT1-MYC fusion sequence.

In some embodiments, the junctional sequence of the target nucleic acid corresponds to a sequence spanning a portion of a TP53(R248Q) mutant transcript. In some embodiments, the junctional sequence comprises at least a portion of a sequence corresponding to one of the above listed chromosomal fusions or mutant transcripts. Exemplary junctional sequences and corresponding target sensing sequences are provided herein.

In some embodiments, the junctional sequence of the target nucleic acid corresponds to a sequence spanning a portion of a viral transcript. In some embodiments, the viral transcript is a transcript associated with cancer. In some embodiments, the viral transcript is an Epstein Barr Virus (EBV) transcript or a Kaposi's sarcoma-associated herpesvirus (KSHV) transcript. For example, in some embodiments the viral transcript is the Epstein Barr Virus transcript EBNA1 and the target sensing region comprises a nucleic acid sequence having at least 80% sequence identity (e.g., at least 80%, at least 85%, at least 90%, at least 91%, at least 92%, at least 93%, at least 94%, at least 95%, at least 96%, at least 97%, at least 98%, at least 99%, or 100% sequence identity) to SEQ ID NO: 34.

In some embodiments, the viral transcript is the KSHV transcript ORF71 and wherein the target sensing region comprises a nucleic acid sequence having at least 80% least 93%, at least 94%, at least 95%, at least 96%, at least 97%, at least 98%, at least 99%, or 100% sequence identity) to SEQ ID NO: 35.

In some embodiments, the embodiments of the present disclosure activate a downstream event, such as the production of a detectable signal corresponding to the target nucleic acid, or exerting a therapeutic function.

In some embodiments, the target sensing region is at least about 50 nucleotides long. In some embodiments, the target sensing region is from about 50 nucleotides to about 1000 nucleotides long.

In some embodiments, the response gene encodes at least one of a reporter protein, a caspase, a prodrug-converting enzyme, or an enzyme catalyzing a specific reaction.

In some embodiments, the sensor molecule further comprises a control gene. In some embodiments, the control gene is constitutively expressed.

In some embodiments, the sensor molecule comprises a linker region positioned upstream of the response gene but downstream of the TAG or TGA stop codon.

In some embodiments, the sensor molecule comprises an RNA aptamer sequence capable of binding its cognate binding protein. In some embodiments, the RNA aptamer comprises a sequence capable of binding at least one of MS2, PP7, BoxB, or Pumilio. In some embodiments, the cognate binding protein is fused to an ADAR protein.

In some embodiments, the sensor molecule is an RNA molecule.

Embodiments of the present disclosure also include an expression vector comprising a DNA sequence corresponding to any of the RNA sensor molecules described herein.

In some embodiments, the expression vector is selected from the group consisting of: a pCR8-mRuby2-P2A-ccdbCam-E2A-EGFP vector; a pCR8-mRuby2-P2A-ccdbCam-E2A-EGFP-BsaI(agat) vector; a pCR8-mRuby2-P2A-ccdbCam-E2A-EGFP-NxMS2 vector; a pCR8-mRuby2-P2A-Sensor-E2A-EGFP vector; a pCR8-mRuby2-P2A-Sensor-E2A-EGFP-NxMS2 vector; a pmax-mRuby2-P2A-Sensor-XTEN80-EGFP-NxMS2 vector; a MCP-ADARdd(E488Q) vector; a pmax-MCP-ADARdd(E488Q), a pmax-MCP-ADARdd(E488Q)-P2A-Sensor-XTEN80-NTR1.1-NxMS2 vector; a pmax-MCP-ADARdd(E488Q)-P2A-Sensor-E2A-NTR1.1-NxMS2 vector; a pmax-MCP-ADARddm(C377F,E488Q)-P2A-Sensor-XTEN80-NTR1.1-NxMS2 vector; a pmax-MCP-ADARddm(C377F,E488Q)-P2A-Sensor-E2A-NTR1.1-NxMS2 vector; a pmax-MCP-ADARdd(E488Q)-P2A-Sensor-XTEN80-DTA-NxMS2 vector; a pmax-MCP-ADARdd(E488Q)-P2A-Sensor-E2A-DTA-NxMS2 vector; a pmax-MCP-ADARddm(C377F,E488Q)-P2A-Sensor-XTEN80-DTA-NxMS2 vector; a pmax-MCP-ADARddm(C377F,E488Q)-P2A-Sensor-E2A-DTA-NxMS2 vector; a pmax-MCP-ADARdd(E488Q)-P2A-Sensor-XTEN80-BAX-NxMS2 vector; a pmax-MCP-ADARdd(E488Q)-P2A-Sensor-E2A-BAX-NxMS2 vector; a pmax-MCP-ADARddm(C377F,E488Q)-P2A-Sensor-XTEN80-BAX-NxMS2 vector; a and pmax-MCP-ADARddm(C377F,E488Q)-P2A-Sensor-E2A-BAX-NxMS2 vector.

Embodiments of the present disclosure also include a cell comprising any of the RNA sensor molecules described herein, or any of the vectors described herein.

Embodiments of the present disclosure also include a kit comprising any of the RNA sensor molecules described herein, any of the vectors described herein, and/or any of the cells described herein.

Embodiments of the present disclosure also include a method of treating a subject having cancer or suspected of having cancer. In accordance with these embodiments, the method includes administering any of the RNA sensor molecules described herein, any of the vectors described herein, and/or any of the cells described herein to the subject; and treating the subject. In some embodiments, the cancer genome contains a chromosomal translocation and/or gene fusion. In some embodiments, the cancer cell contains viral genomes and/or express viral transcripts. In some embodiments, the cancer cell contains mutations in endogenous genes. The RNA sensor, vector, and/or cells may be administered to the subject by any suitable route, including parenteral routes (e.g. injection, such as intravenous, intramuscular, intraarterial, subcutaneous, etc.). In some embodiments, delivery of the sensor to the subject is achieved by use of vectors (e.g. viral vectors such as AAV, viral-like particles) nanoparticles (e.g. mRNA-lipid nanoparticles), or other suitable means.

Embodiments of the present disclosure also include a method of detecting a gene fusion transcript in a cell. In accordance with these embodiments, the method includes transfecting a cell with any of the RNA sensor molecules described herein, or any of the vectors described herein, and assessing the cell for expression of a reporter protein.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

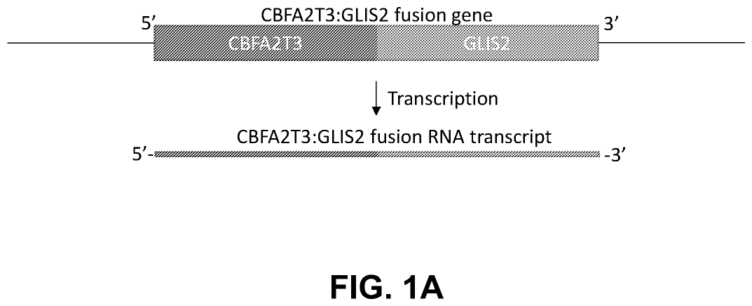

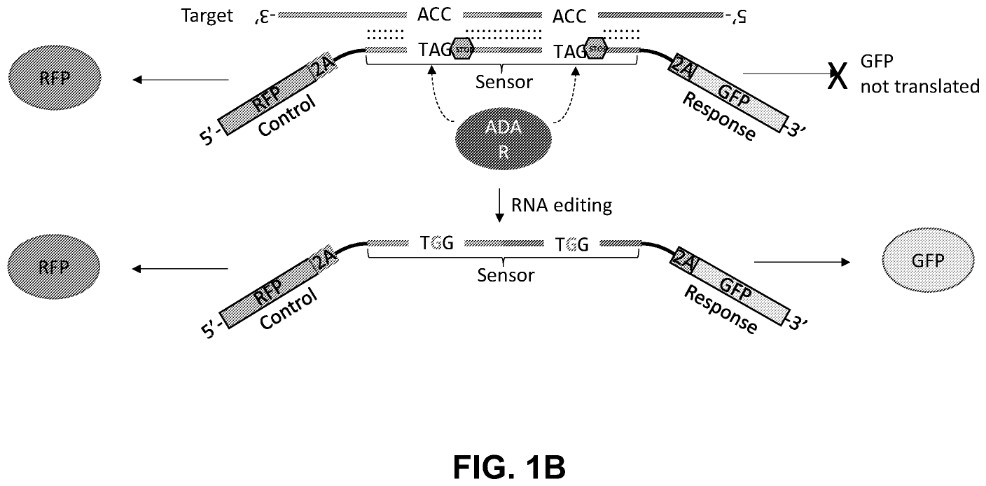

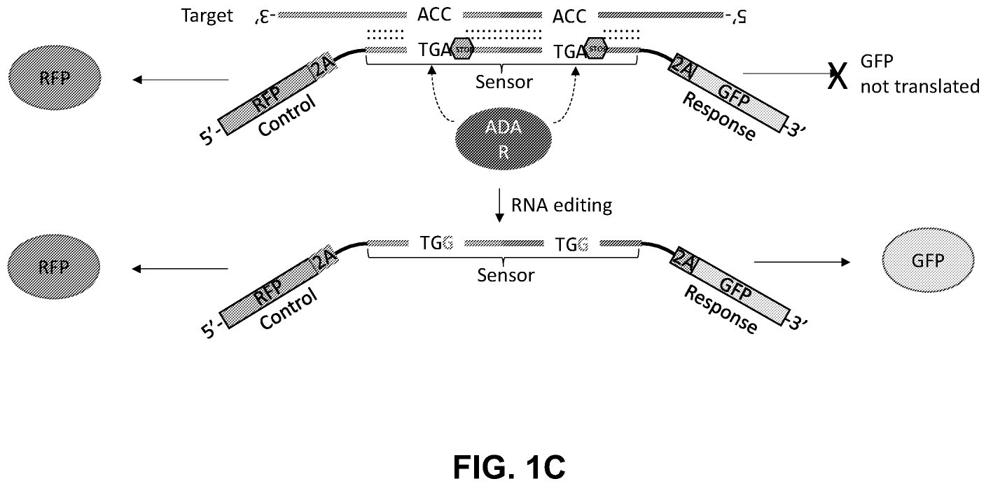

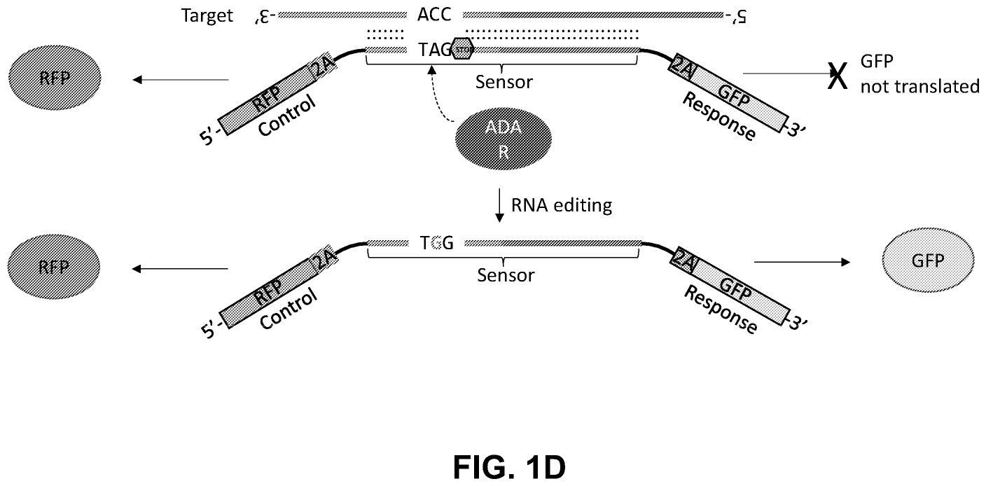

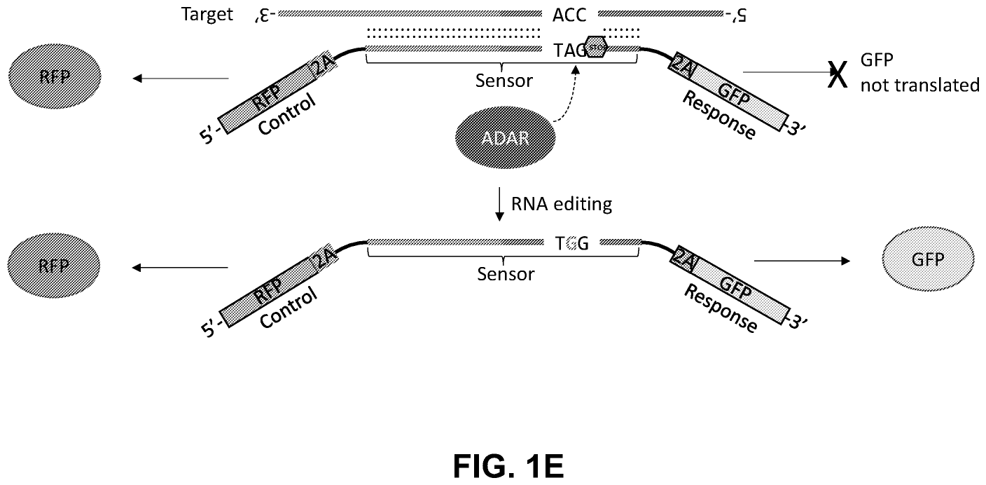

Design of fusion RNA sensor. Fusion genes arise from genomic rearrangement and/or chromosomal deletions placing two genes that are originally apart in a normal genome to close proximity, allowing transcription machinery to transcribe a chimeric RNA transcript (FIG. 1A). The chimeric transcript (fusion transcript) harbors unique junctional sequences as a result of the fusion or brings together on the same nucleic acid molecule sequences that are from two originally separate genes. These unique features serve as sensing targets for our RNA sensors.

Exemplary RNA sensors of the present disclosure (e.g., FIGS. 1B-1E) can be composed of, from 5′ to 3′: (i) optionally, a control sequence constitutively expressing a detectable gene product, such as a red fluorescent protein (RFP); (ii) optionally, a 2A peptide sequence that allows downstream peptides to be separated from upstream peptides; (iii) a sensor sequence reverse-complementary to the target sequence, with one or more 5′-TAG-3′ sensing triplets opposing 5′-CCA-3′ triplets (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) on the target sequence, surrounding the junctional sequence of the fusion transcript; (iv) optionally, a 2A peptide or protein linker sequence, that either allows downstream peptide to be separated or allows for the “spacing” of downstream peptide from the upstream peptides; (v) a response gene (e.g., green fluorescent proteins, caspase, or prodrug-converting enzymes) whose expression is depending on the activation of the upstream sensor fragment when the 5′-TAG-3′ stop codons are converted to 5′-TGG-3′ codon upon binding to target sequence with 5′-CCA-3′ triplet (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) and ADAR-mediated RNA editing;

In some embodiments, RNA aptamer sequences, such as MS2, PP7, BoxB, or Pumilio binding sites, can be inserted upstream of the control sequence, in the middle of sensor sequence, or downstream of the response sequence, that can recruit recombinant ADAR fused to cognate aptamer-binding proteins, such as MS2 coat protein (MCP), PP7 coat protein (PCP), lambaN protein, or Pumilio/PUF-HD domains.

The binding of the sensor fragment to the target sequence produces C: A (or A:A, G:A) mismatch within the context of mostly double-stranded RNA presents as a substrate for RNA editing by endogenous or exogenously supplied ADAR, converting the A in the sensor RNA to Inosine, which is read as G by the translation machinery. As the edited A is within the context of a designed in-frame stop coding upstream of the response coding sequence, the conversion of A-to-I (A-to-G) changes the stop codon to tryptophan-coding 5′-TGG-3′ codon allowing the downstream response gene to be translated. The response gene codes for a fluorescent protein (e.g., GFP), giving fluorescence upon detection of target transcript. Alternatively, the response gene codes for a cell death protein (e.g., caspases), in such case, apoptotic cell death is triggered upon target detection. Alternatively, the response gene codes for prodrug-converting enzymes, such as bacterial nitroreductase (NTR), that will be expressed upon target detection to render host cells and potentially neighboring cells sensitive to prodrugs such as CB1945 or MTZ. Alternatively, the response gene codes for an immunostimulatory or immunoattractive proteins, which will attract or recruit immune cells to the cellular microenvironment, in order to trigger local immune response and/or clearance of target and neighboring cells.

### Example 2

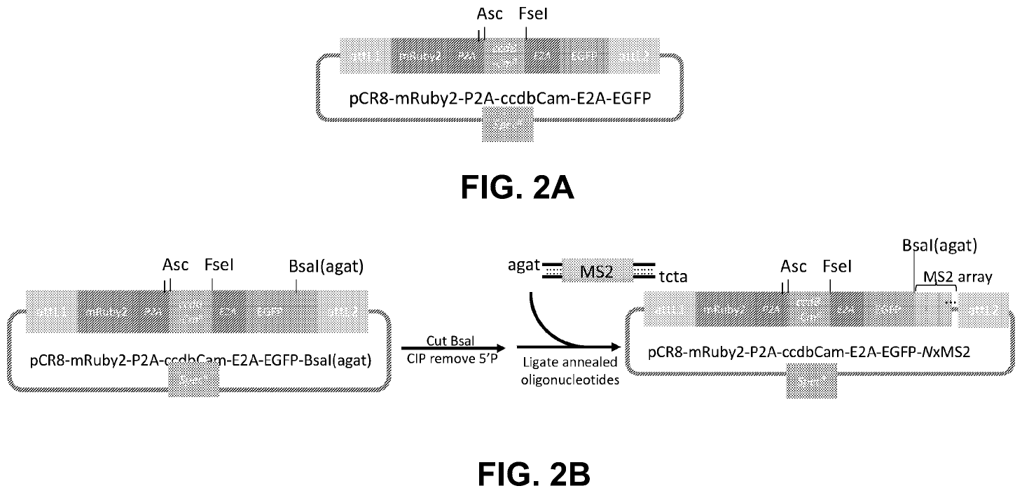

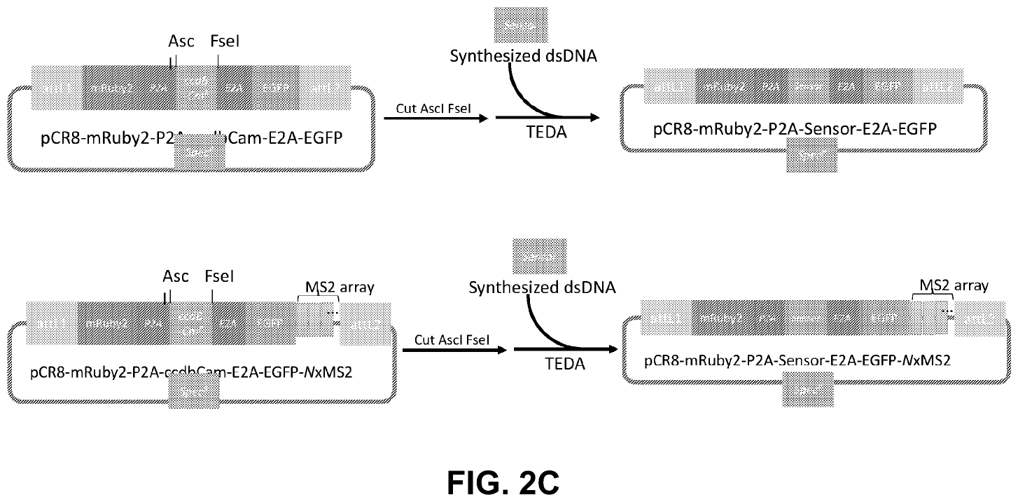

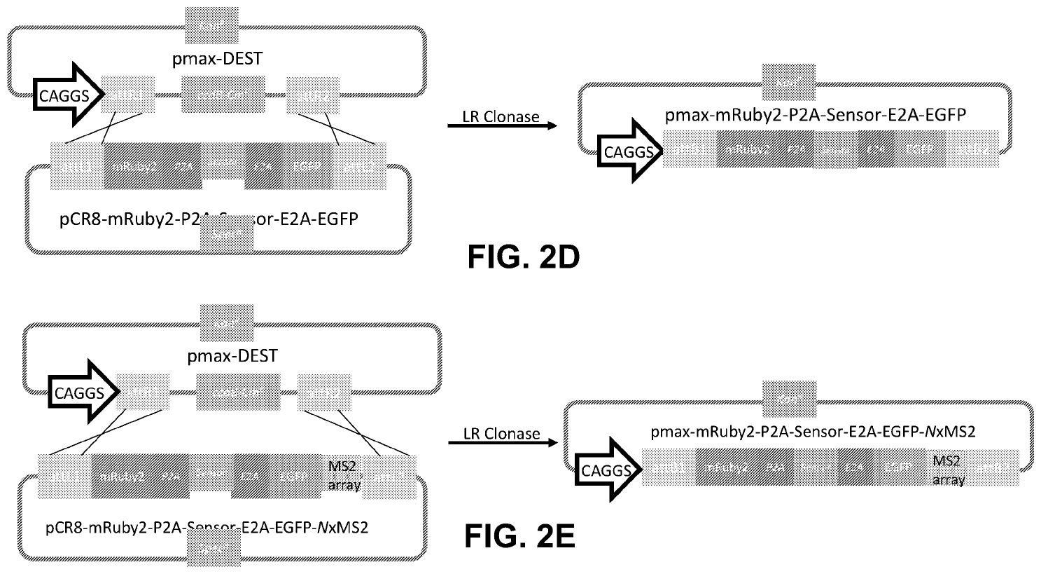

Cloning Vectors for fusion RNA Sensors. A vector system was created to facilitate cloning and testing of different sensor sequences and sensor architectures (FIG. 2). For example, mRuby2 (control sequence), P2A, AscI restriction site, ccdB-Cm^{R} negative-positive selection cassette, FseI restriction site, E2A, EGFP (response sequence) were assembled in 5′->3′ order via multiple PCR reactions, and cloned into pCR8/GW/TOPO Gateway donor vector via TEDA ligation-free cloning method, to create pCR8-mRuby2-P2A-ccdbCam-E2A-EGFP vector (FIG. 2A). mRuby2 (control sequence), P2A, AscI restriction site, ccdB-Cm^{R} negative-positive selection cassette, FseI restriction site, E2A, EGFP (response sequence), BsaI restriction site that can generate 5′-agat-3′ overhang, were assembled in 5′->3′ order via multiple PCR reactions, and cloned into pCR8/GW/TOPO Gateway donor vector via TEDA ligation-free cloning method, to create pCR8-mRuby2-P2A-ccdbCam-E2A-EGFP-BsaI(agat) vector, which was subsequently digested by BsaI enzyme, end dephosphorylated by Quick CIP, and ligated with annealed double stranded polyoligonucleotides encoding MS2 stem loop sequences to generate pCR8-mRuby2-P2A-ccdbCam-E2A-EGFP-NxMS2 vectors screened for varying number N (e.g., N=9) of MS2 stem loops (FIG. 2B).

To insert sensor sequence, the pCR8-mRuby2-P2A-ccdbCam-E2A-EGFP vector or pCR8-mRuby2-P2A-ccdbCam-E2A-EGFP-NxMS2 vectors were doubly digested by AscI and FseI restriction enzymes with sites flanking the ccdb-Cm^{R} selection cassette, with the intervening sequence replaced by sensor sequence provided as PCR or synthesized double stranded DNA fragment through TEDA method, resulting in pCR8-mRuby2-P2A-Sensor-E2A-EGFP or pCR8-mRuby2-P2A-Sensor-E2A-EGFP-NxMS2 vectors (FIG. 2C), which can be subsequently shuttled into Gateway destination expression vectors (FIGS. 2D and 2E), to create expression vectors for the sensor-response gene.

### Example 3

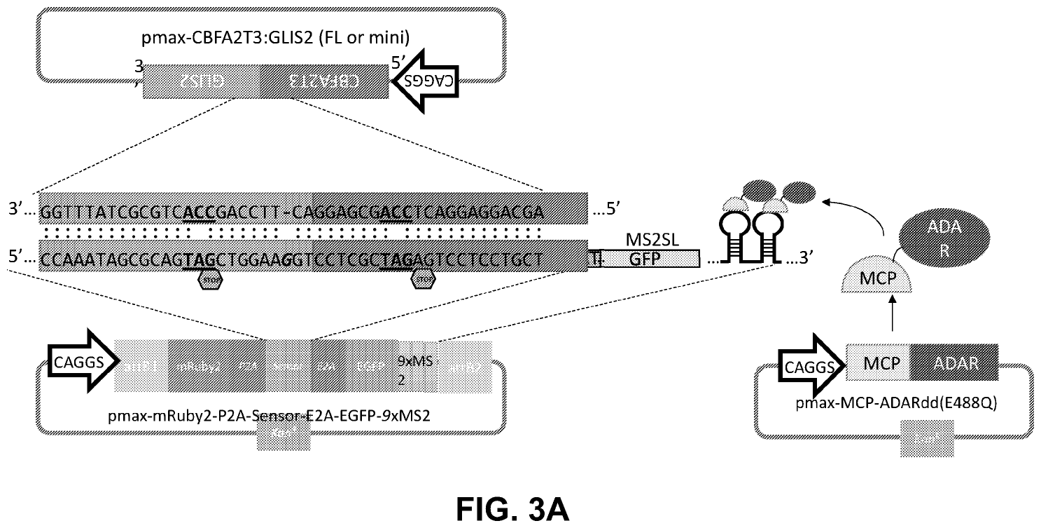

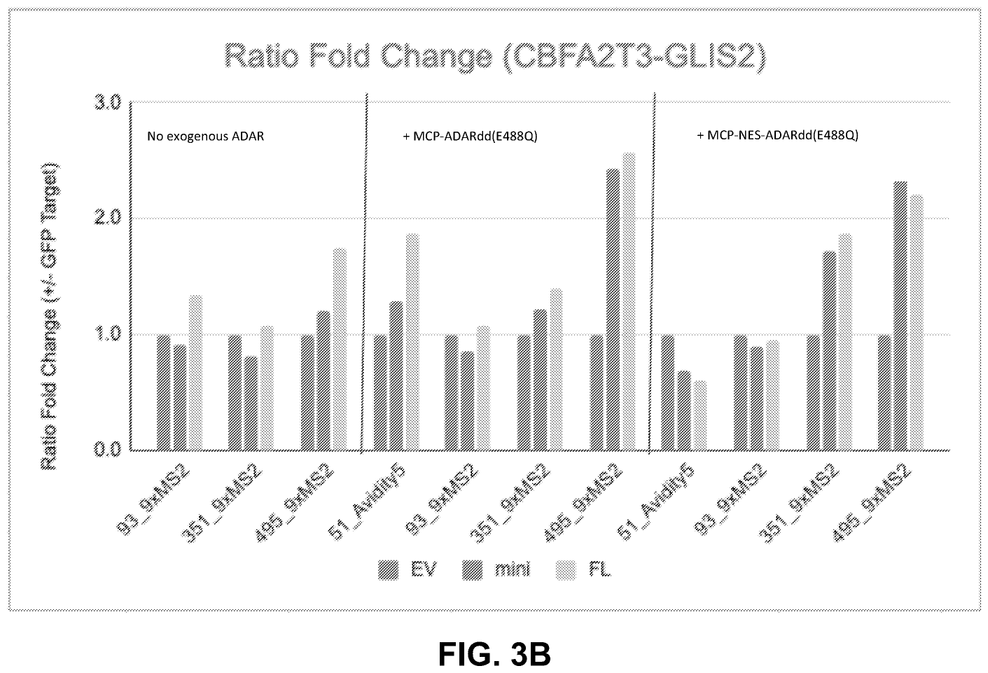

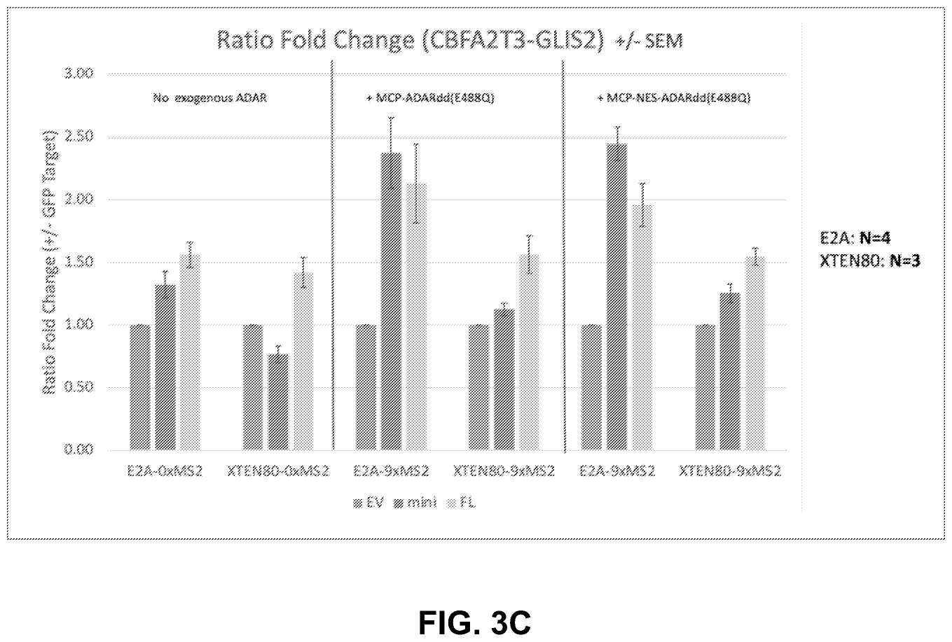

In vivo detection of CBFAT3-GLIS2 fusion transcripts. Four sensor sequences were designed to detect CBFA2T3-GLIS2 fusion sequence, with length of 93 (CBFA2T3GLIS2_93_sensor), 351 (CBFA2T3GLIS2_351_sensor), 495 (CBFA2T3GLIS2_495_sensor), roughly centered at the fusion junction. A sensor consisting of four MS2 stem loops inserted within the sensor region was also designed (CBFA2T3GLIS2_avidity5). Because the two sensor stop codons were not in frame with each other (distance between them is not a multiple of 3), an extra G was added to ensure the two sensor stop codons are in frame with respective to each other as well as upstream and downstream sequences (FIG. 3A, sensor sequence, italic G). All in-frame stops in the sensor region that are not part of the RNA editing substrates were mutated to a non-stop codon (TAG->TGG, TAA->TAC, TGA->TGG) and all in-frame start codons in the sensor regions (ATG) were mutated to ATC. Sensor-response expression vectors were constructed according to the workflow in example 2 (pmax-mRuby2-P2A-Sensor-E2A-EGFP-9xMS2). A minigene (375 bp sequences on both sides of the fusion transcript) and full-length CBFA2T3-GLIS were cloned into the pmax expression vector (pmax-CBFA2T3-GLIS2_FL or pmax-CBFA2T3-GLIS3_mini750) serving as test fusion genes. An expression vector was constructed for MS2 coat protein-hyperactive ADAR (E488Q) fusion (pmax-MCP-ADARdd(E488Q)). Cells were transfected with the sensor-response vectors (pmax-mRuby2-P2A-Sensor-XTEN80-EGFP-9xMS2), MCP-ADARdd(E488Q) vector (pmax-MCP-ADARdd(E488Q)) and either a control empty vector or vector expressing test fusion gene (pmax-CBFA2T3-GLIS2_FL or pmax-CBFA2T3-GLIS3_mini750) into HEK293T cells and analyzed for fluorescence by flow cytometry 48 hours post-transfection (FIG. 3B). The presence of fusion transcript led to increase of GFP signal not seen in cells transfected with empty vector control, demonstrating the specific detection of fusion CBFA2T3-GLIS2 transcripts in vivo in HEK293T. The reproducibility of the CBFA2T3-GLIS2_495 sensor probe with E2A linker (N=4) is also demonstrated (FIG. 3C).

### Example 4

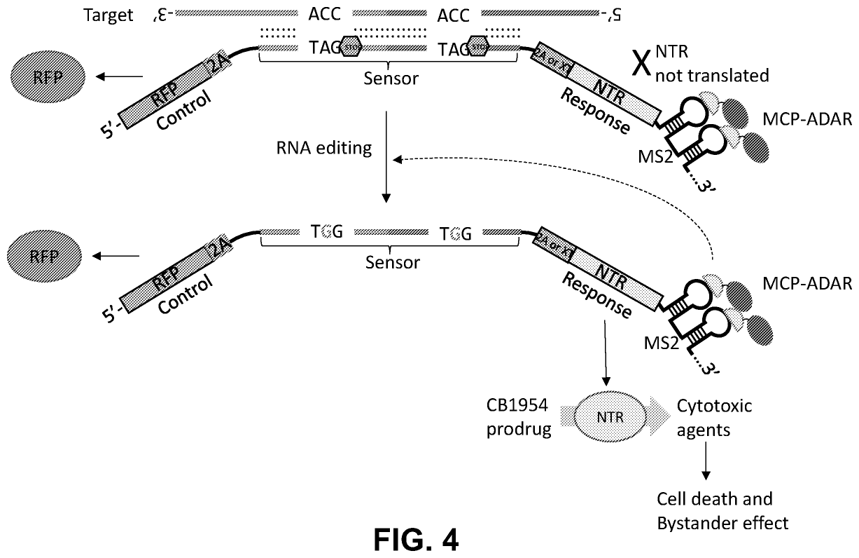

Design of fusion RNA sensor-NTR (nitroreductase) response. Chimeric transcripts (fusion transcript) arising from fusion genes harbor unique junctional sequences that can act as target for detection (FIG. 4). RNA sensors-NTR response can be constructed to express nitroreductase (NTR) upon fusion transcript detection. The expressed NTR as a result of fusion transcript detection converts prodrug such as CB1954 (Tretazicar) to cytotoxic agents that can lead to cell death and diffuse to neighboring cells to cause neighboring cell death (also known as Bystander effect). Alternatively, MTZ (Metronidazole) can be used to cause cell death in the cells expressing fusion transcript without bystander effect.

One exemplary RNA sensor-NTR response of the present disclosure (e.g., FIG. 4) is composed of, from 5′ to 3′: (i) optionally, a control sequence constitutively expressing a detectable gene product, such as a red fluorescent protein (RFP); (ii) optionally, a 2A peptide sequence that allows downstream peptides to be separated from upstream peptides; (iii) a sensor sequence reverse-complementary to the target sequence, with one or more 5′-TAG-3′ sensing triplet opposing 5′-CCA-3′ triplets (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) on the target sequence, surrounding the junctional sequence of the fusion transcript; (iv) optionally, a 2A peptide or protein linker sequence, that either allows downstream peptide to be separated or allows for the “spacing” of downstream peptide from the upstream peptides; (v) an nitroreductase (NTR) gene whose expression is depending on the activation of the upstream sensor fragment when the 5′-TAG-3′ stop codons are converted to 5′-TGG-3′ codon upon binding to target sequence with 5′-CCA-3′ triplet (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) and ADAR-mediated RNA editing; (vi) optionally, an array of binding sites such as MS2 stem loops that can recruit MS2 coat protein (MCP)-ADAR fusion.

### Example 5

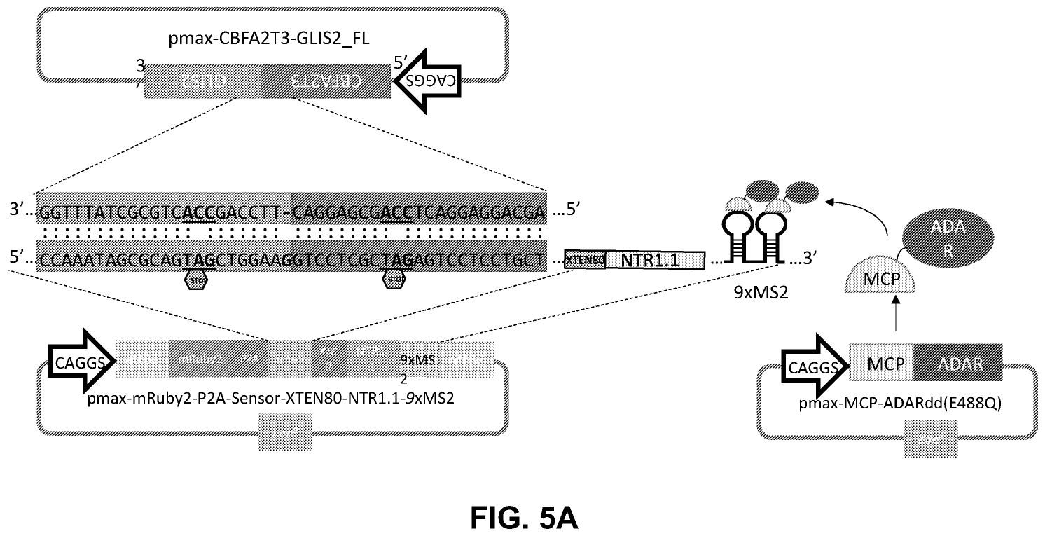

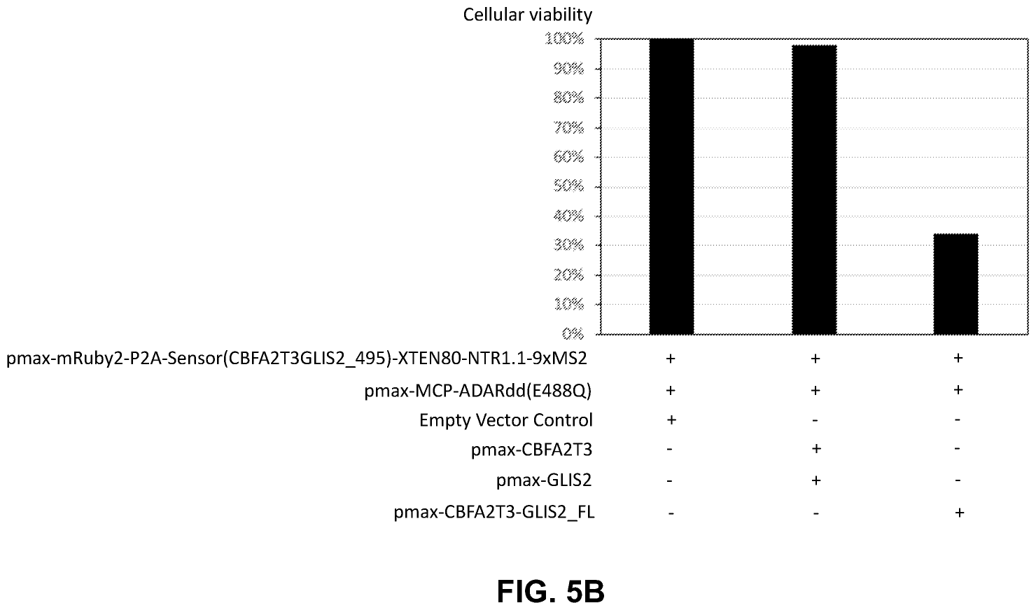

In vivo detection of CBFA2T3-GLIS2 fusion transcripts by sensor-NTR to trigger cell death in the presence of CB1945 prodrug. The CBFA2T3GLIS2_495_sensor was reprogrammed to express nitroreductase (NTR) upon CBFA2T3-GLIS2 fusion transcript detection by replacing the EGFP response gene with the coding sequence for NTR1.1, creating pmax-mRuby2-P2A-Sensor (CBFA2T3GLIS2_495)-XTEN80-NTR1.1-9xMS2. The sensor region contains two sensing stop codons. It is followed by XTEN80 protein linker, then by NTR1.1 coding sequence and 9 copies of MS2 stem loop (9xMS2). The 9xMS2 can recruit MCP-ADARdd(E488Q). The binding (detection) of sensor region to the fusion transcript triggers the editing of the two TAG stop codons to TGG codons, allowing the downstream NTR to be translated. The translated NTR can convert prodrugs (e.g., CB1954) to cytotoxic agents to achieve cell ablation (FIG. 5A). We co-transfected the sensor construct, pmax-MCP-ADARdd(E488Q)-expressing the MCP-ADARdd(E488Q) protein, with either empty vector (EV), pmax-CBFA2T3-GLIS2_FL-expressing fusion transcript, or a combination of unfused constituents pmax-CBFA2T3 and pmax-GLIS2 into HEK293T cells and added CB1954 prodrug 24 hours post-transfection (FIG. 5B). Cellular viability was measured using the CellTiter-Glo 2.0 Assay, a luminescent quantification of ATP indicating the presence of metabolically active cells, 7 days post prodrug addition. The presence of fusion transcript, but neither empty vector control nor the combination of unfused constituents, led to increase cell death in the presence of CB1954 prodrug, demonstrating the specific ablation of cells expressing fusion CBFA2T3-GLIS2 transcripts in vivo (FIG. 5B).

### Example 6

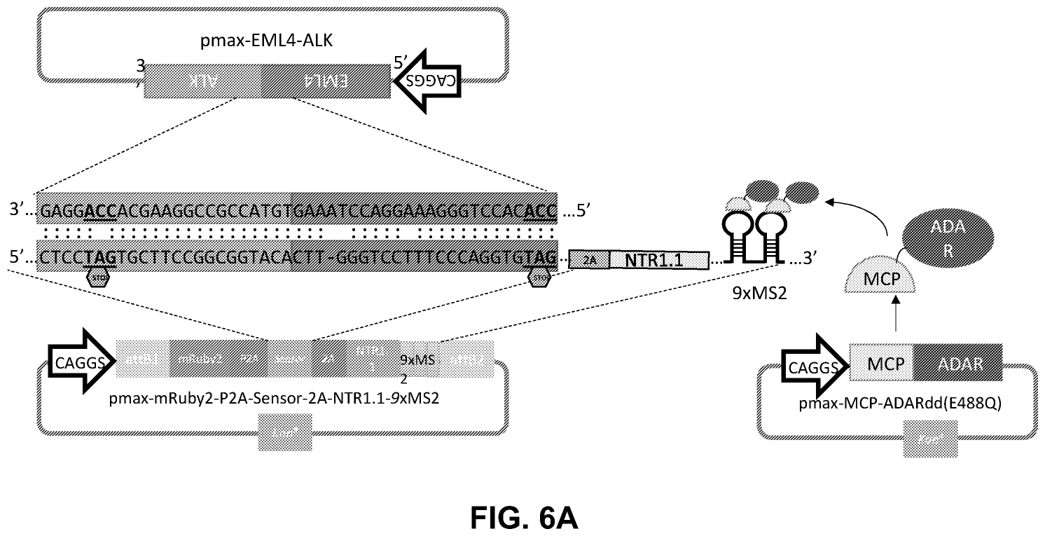

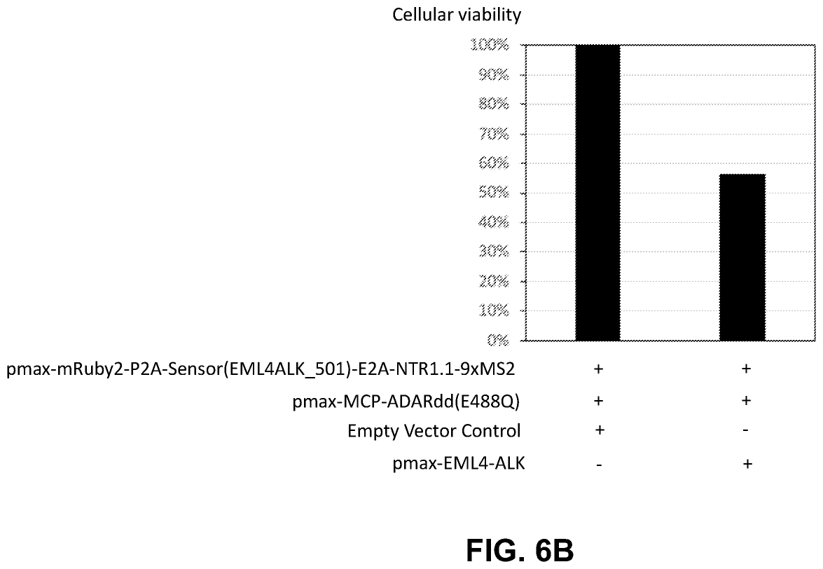

In vivo detection of EML4-ALK fusion transcripts by sensor-NTR to trigger cell death in the presence of CB1945 prodrug. The EML4ALK_501_sensor was reprogrammed to express nitroreductase (NTR) upon CBFA2T3-GLIS2 fusion transcript detection by replacing the EGFP response gene with the coding sequence for NTR1.1, creating pmax-mRuby2-P2A-Sensor (EML4ALK_501)-E2A-NTR1.1-9xMS2). The sensor region contains two sensing stop codons. It is followed by E2A peptide, NTR1.1 coding sequence, and 9 copies of MS2 stem loop (9xMS2). The 9xMS2 can recruit MCP-ADARdd(E488Q). The binding (detection) of sensor region to the fusion transcript triggers the editing of the two TAG stop codons to TGG codons, allowing the downstream NTR to be translated. The translated NTR can convert prodrugs (e.g., CB1954) to cytotoxic agents to achieve cell ablation (FIG. 6A). We co-transfected the sensor construct, pmax-MCP-ADARdd(E488Q)-expressing the MCP-ADARdd(E488Q) protein, with either empty vector (EV) or pmax-EML4-ALK (mini)-expressing 750 fragment surrounding the EML4-ALK fusion junction, into HEK293T cells and added CB1954 prodrug 24 hours post-transfection (FIG. 6B). Cellular viability was measured using the CellTiter-Glo 2.0 Assay, a luminescent quantification of ATP indicating the presence of metabolically active cells, 7 days post prodrug addition. The presence of fusion transcript, but not the empty vector control, led to increase cell death in the presence of CB1954 prodrug, demonstrating the specific ablation of cells expressing fusion EML4-ALK transcripts in vivo (FIG. 6B).

### Example 7

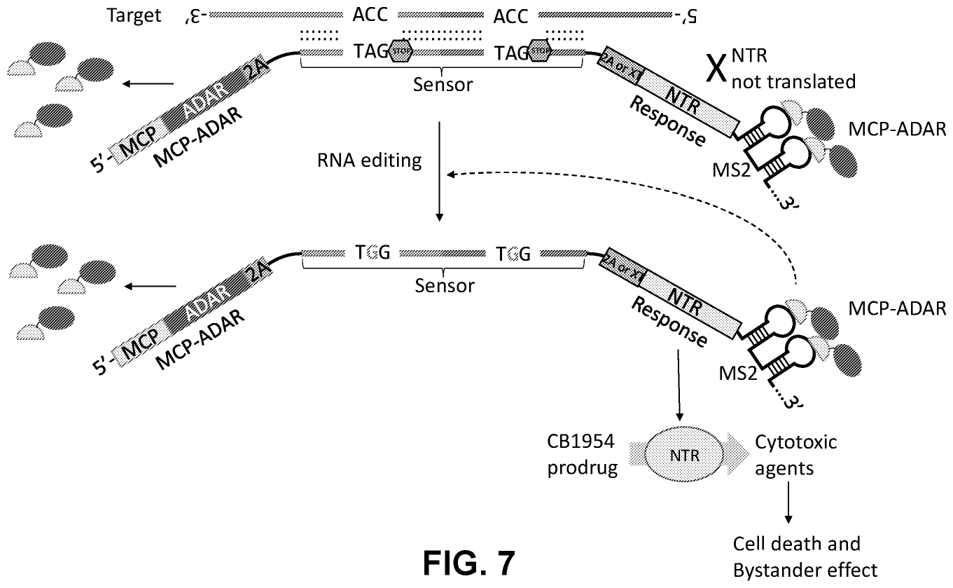

Design of all-in-one ADAR-sensor-NTR (nitroreductase). Chimeric transcripts (fusion transcript) arising from fusion genes harbor unique junctional sequences that can act as target for detection (FIG. 7). An all-in-one ADAR-sensor-NTR construct can be constructed to express constitutively a fusion protein containing ADAR enzyme, and RNA sensor fragment coupled to the coding sequence of nitroreductase (NTR) that get translated upon fusion transcript detection. The expressed NTR as a result of fusion transcript detection converts prodrug such as CB1954 (Tretazicar) to cytotoxic agents that can lead to cell death and diffuse to neighboring cells to cause neighboring cell death (also known as Bystander effect). Alternatively, MTZ (Metronidazole) can be used to cause cell death in the cells expressing fusion transcript without bystander effect.

One exemplary ADAR-sensor-NTR construct of the present disclosure (e.g., FIG. 7) is composed of, from 5′ to 3′: (i) coding sequence of ADAR fusion protein, such as the fusion of ADAR deaminase domain with MS2 coat protein (MCP); (ii) optionally, a 2A peptide sequence that allows downstream peptides to be separated from upstream peptides; (iii) a sensor sequence reverse-complementary to the target sequence, with one or more 5′-TAG-3′ sensing triplet opposing 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets on the target sequence, surrounding the junctional sequence of the fusion transcript; (iv) optionally, a 2A peptide or protein linker sequence, that either allows downstream peptide to be separated or allows for the “spacing” of downstream peptide from the upstream peptides; (v) an nitroreductase (NTR) gene whose expression is depending on the activation of the upstream sensor fragment when the 5′-TAG-3′ stop codons are converted to 5′-TGG-3′ codon upon binding to target sequence with 5′-CCA-3′ triplets (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) and ADAR-mediated RNA editing; (vi) optionally, an array of binding sites such as MS2 stem loops that can recruit MS2 coat protein (MCP)-ADAR fusion.

### Example 8

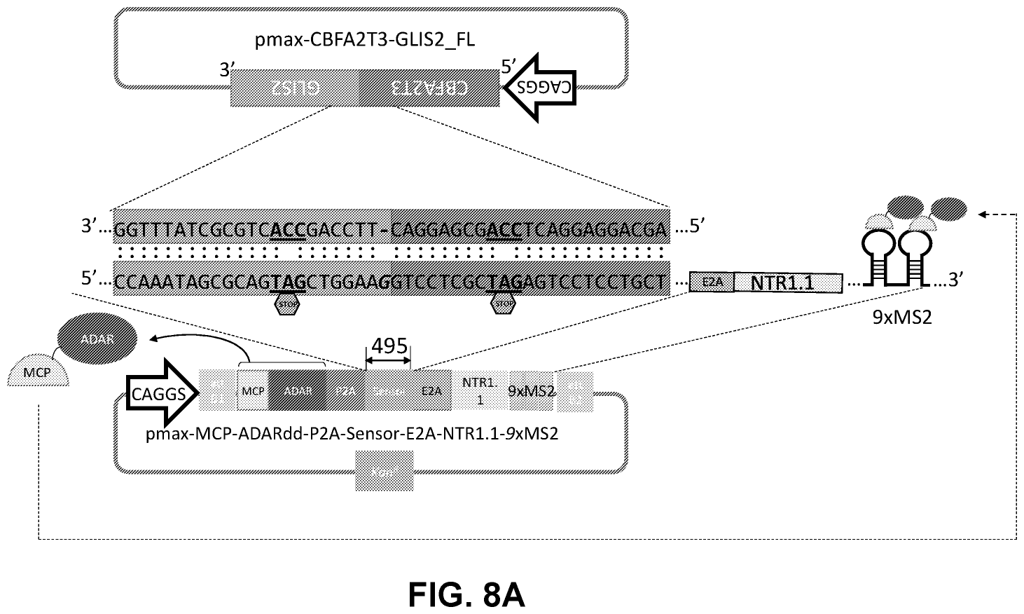

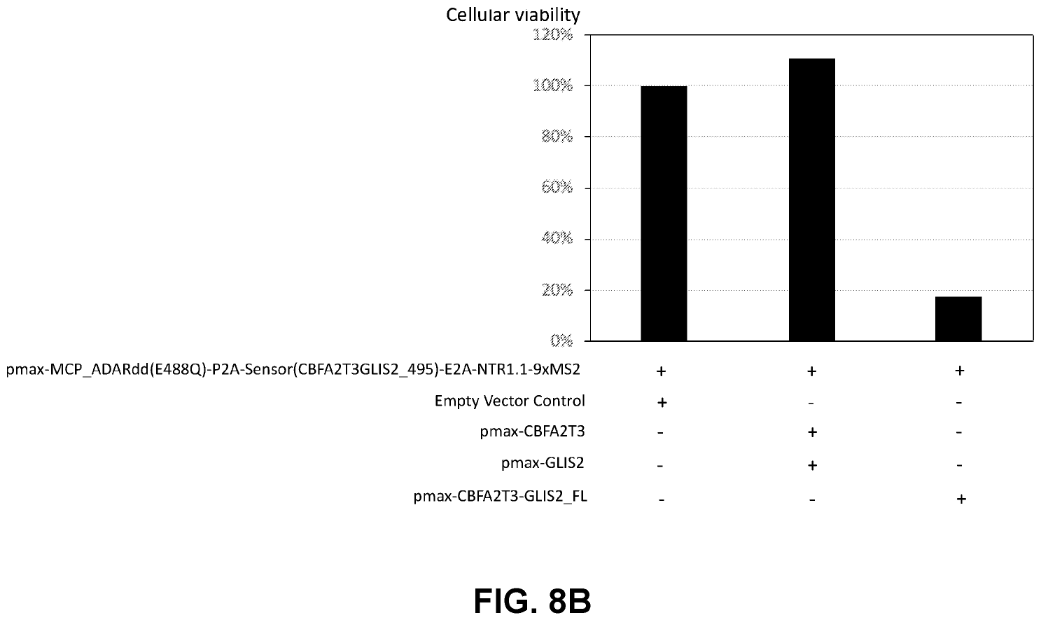

In vivo detection of CBFA2T3-GLIS2 fusion transcripts by all-in-one ADAR-sensor-NTR to trigger cell death in the presence of CB1945 prodrug. A pmax-MCP-ADAR-P2A-Sensor (CBFA2T3GLIS2_495)-E2A-NTR1.1-9xMS2 was constructed to express an RNA molecule containing coding sequence for ADAR deaminase domain fused with MCP (MCP-ADARdd(E488Q)), followed by that coding for P2A peptide, followed by a sensor region complementary to the target CBFA2T3-GLIS2 fusion transcripts and two sensing stop codons surrounding the fusion junction, followed by coding sequences of E2A peptide and NTR1.1, then by 9 copies of MS2 stem loop (9xMS2). The 9xMS2 can recruit MCP-ADARdd(E488Q) expressed from the same RNA molecule. The binding (detection) of sensor region to the fusion transcript triggers the editing of the two TAG stop codons to TGG codons, allowing the downstream NTR to be translated. The translated NTR can convert prodrugs (e.g., CB1954) to cytotoxic agents to achieve cell ablation (FIG. 8A). The all-in-one ADAR-sensor-NTR construct was co-transfected, with either empty vector (EV), pmax-CBFA2T3-GLIS2_FL-expressing fusion transcript, or a combination of unfused constituents pmax-CBFA2T3 and pmax-GLIS2 into HEK293T cells and added CB1954 prodrug 24 hours post-transfection (FIG. 8B). Cellular viability was measured using the CellTiter-Glo 2.0 Assay, a luminescent quantification of ATP indicating the presence of metabolically active cells, 5 days post prodrug addition. The presence of fusion transcript, but neither empty vector control nor the combination of unfused constituents, led to increase cell death in the presence of CB1954 prodrug, demonstrating the specific ablation of cells expressing fusion CBFA2T3-GLIS2 transcripts in vivo (FIG. 8B).

### Example 9

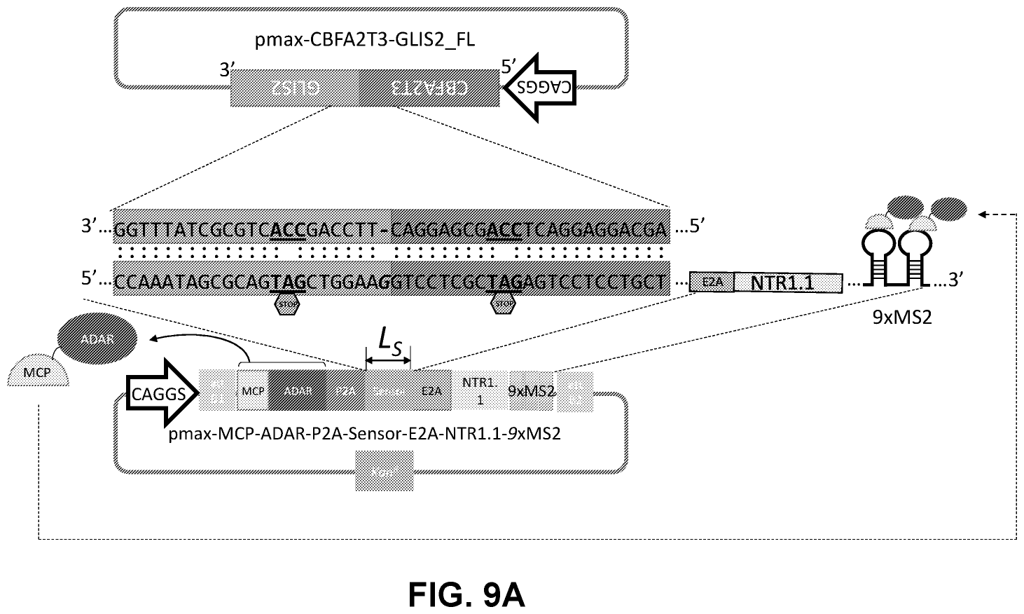

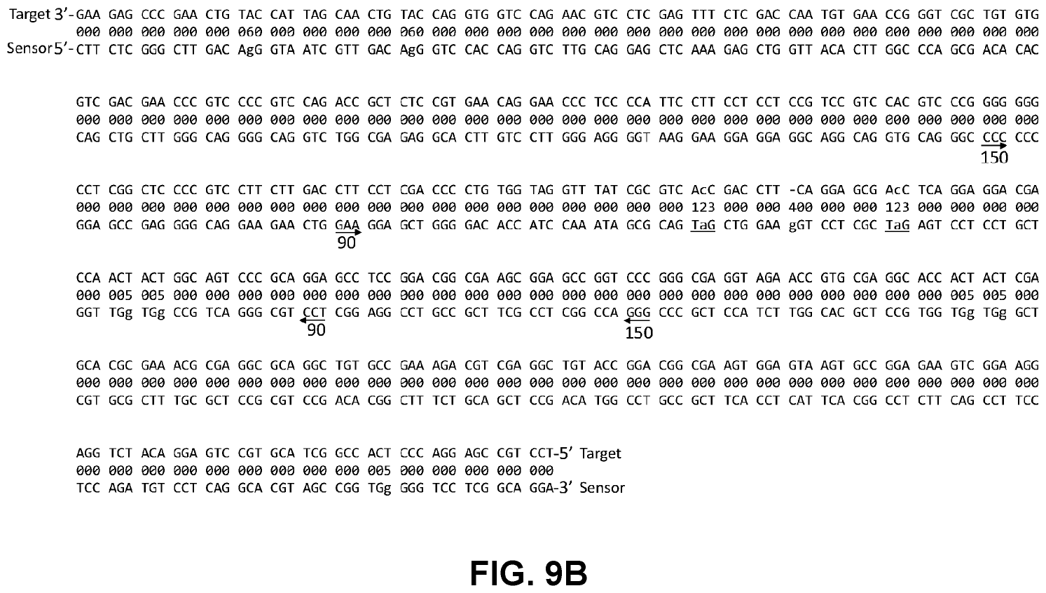

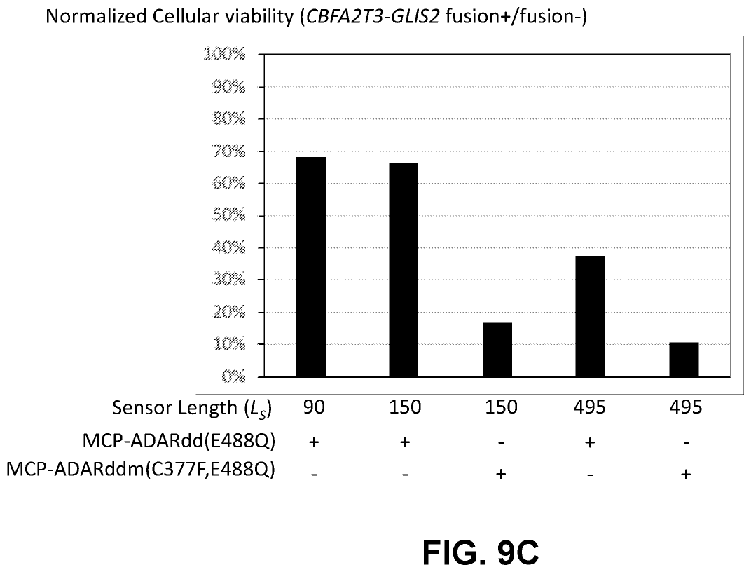

In vivo detection of CBFA2T3-GLIS2 fusion transcripts by all-in-one ADAR-sensor-NTR constructs with different sensor lengths and ADAR mutants to trigger cell death in the presence of CB1945 prodrug. ADAR-sensor-NTR constructs with different sensor lengths (L_{S}) complementary to CBFA2T3-GLIS2 fusion transcript were constructed to test the effect of sensor length on cell ablation efficacy (FIG. 9A-B). During the cloning of the ADAR-sensor-NTR constructs, we recovered an ADAR mutant with an additional C377F mutation, i.e., MCP-ADARddm(C377F,E488Q), and were thus included in the analysis. We co-transfected the all-in-one ADAR-sensor-NTR constructs, with either empty vector (EV) or pmax-CBFA2T3-GLIS2_FL into HEK293T cells and added CB1954 prodrug 24 hours post-transfection (FIG. 9C). Cellular viability of samples expressing CBFA2T3-GLIS2 fusion (fusion+) was measured using the CellTiter-Glo 2.0 Assay to quantify metabolically active cells normalized to that of samples receiving empty vector control (fusion−), 5 days post prodrug addition (FIG. 9C). We observed a general increase of cell ablation activity (decrease in normalized cellular viability) with increasing sensor length L_{S}. Additionally, we observed an increase of cell ablation activity (decrease in normalized cellular viability) of constructs expressing MCP-ADARddm(C377F,E488Q) double mutant compared to those expressing MCP-ADARdd(E488Q) single mutant (FIG. 9C).

### Example 10

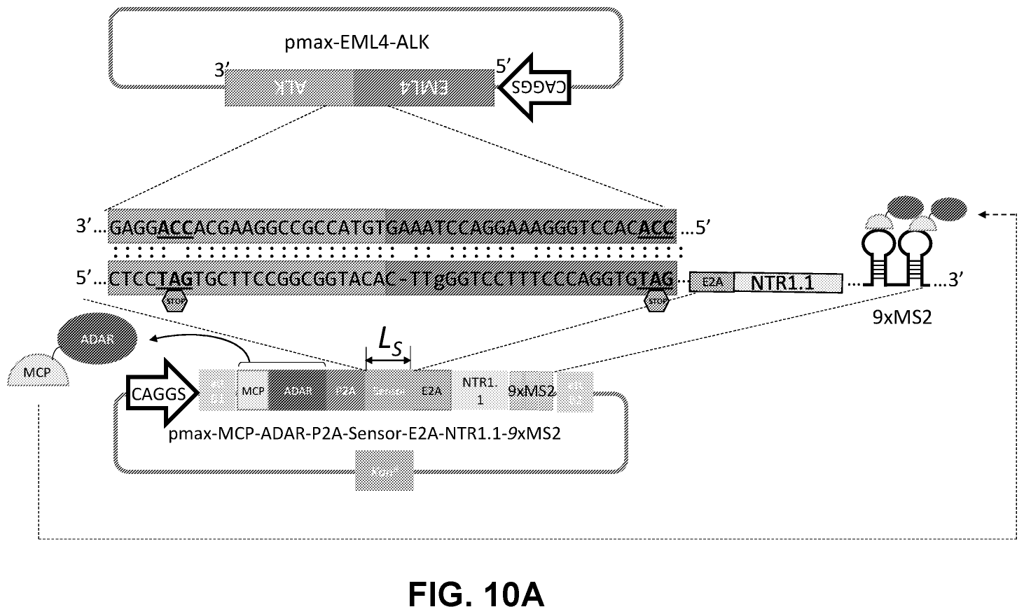

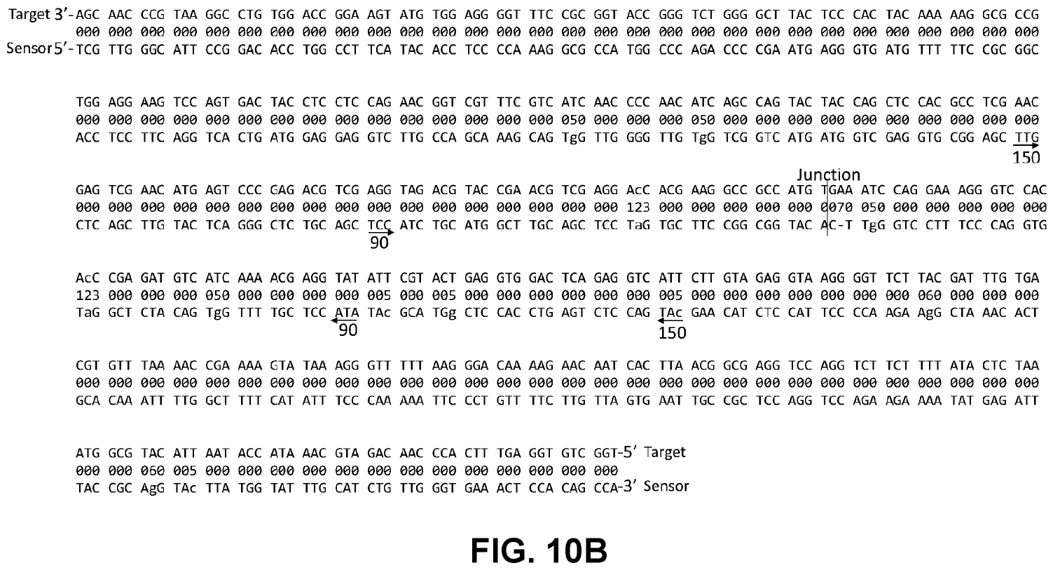

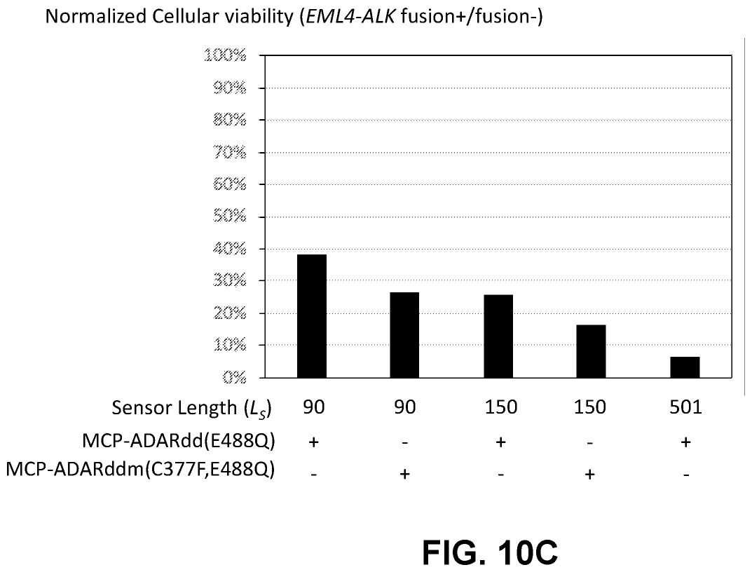

In vivo detection of EML4-ALK fusion transcripts by all-in-one ADAR-sensor-NTR constructs with different sensor lengths and ADAR mutants to trigger cell death in the presence of CB1945 prodrug. ADAR-sensor-NTR constructs with different sensor lengths (L_{S}) complementary to EML4-ALK fusion transcript and with single- or double-mutant ADAR were constructed to test the effect of sensor length on cell ablation efficacy (FIG. 10A-B). We co-transfected the all-in-one ADAR-sensor-NTR constructs, with either empty vector (EV) or pmax-EML4-ALK into HEK293T cells and added CB1954 prodrug 24 hours post-transfection (FIG. 10C). Cellular viability of samples expressing EML4-ALK fusion (fusion+) was measured using the CellTiter-Glo 2.0 Assay to quantify metabolically active cells normalized to that of samples receiving empty vector control (fusion−), 5 days post prodrug addition (FIG. 10C). We observed a general increase of cell ablation activity (decrease in normalized cellular viability) with increasing sensor length L_{S}. Additionally, we observed an increase of cell ablation activity (decrease in normalized cellular viability) of constructs expressing MCP-ADARddm(C377F,E488Q) double mutant compared to those expressing MCP-ADARdd(E488Q) single mutant (FIG. 10C).

### Example 11

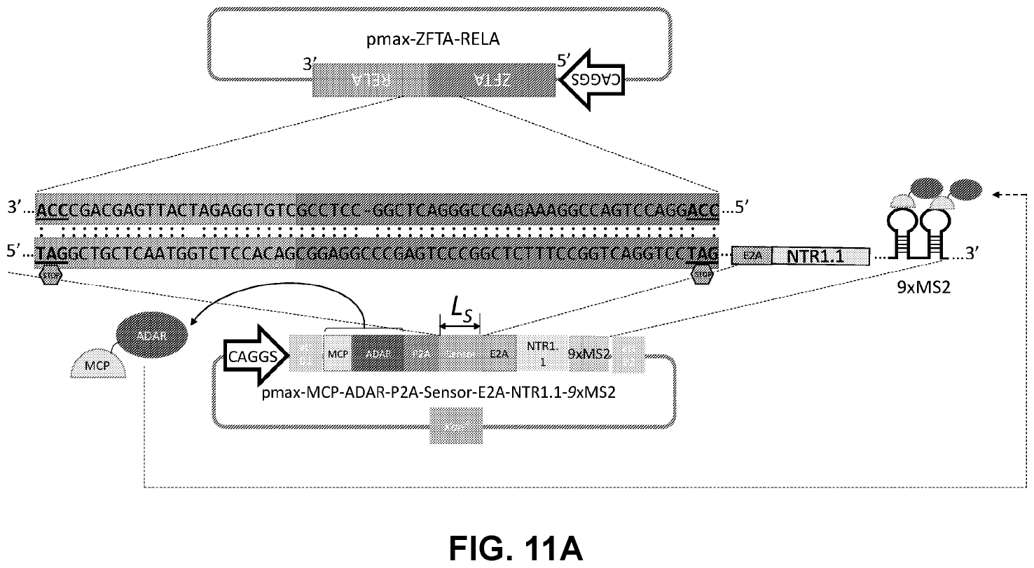

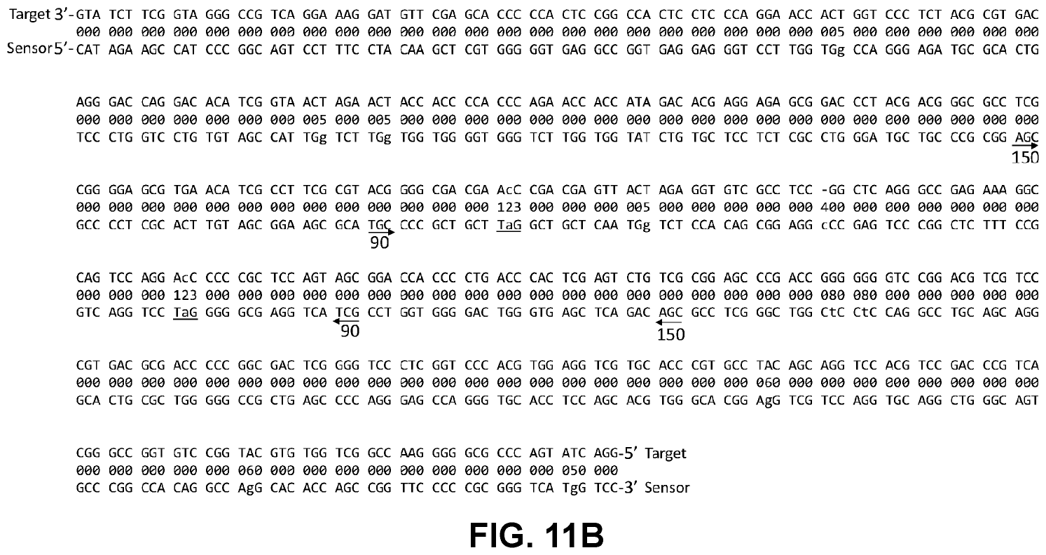

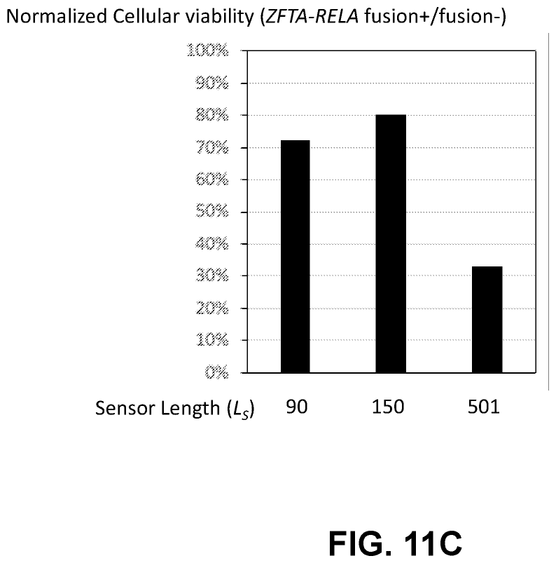

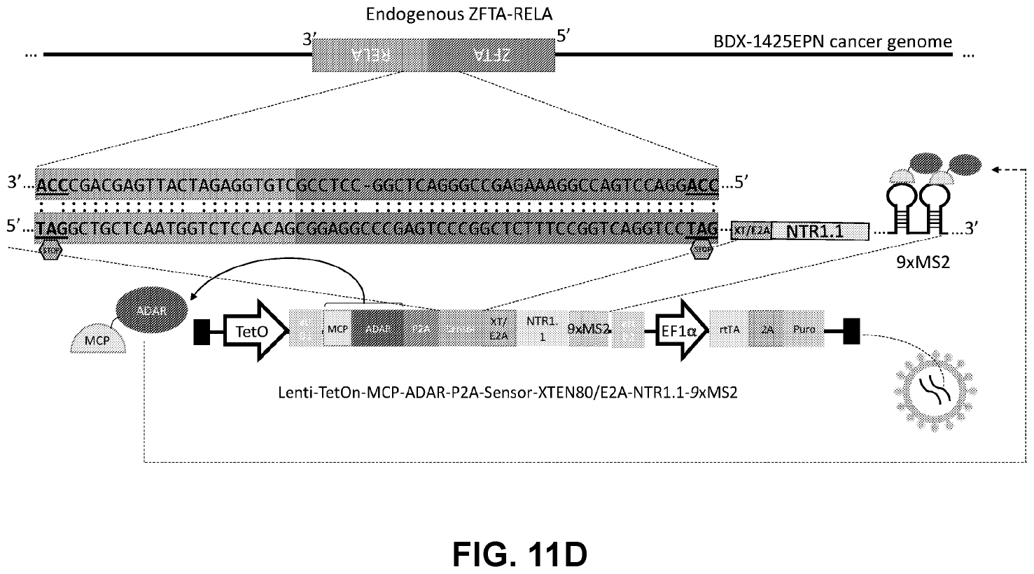

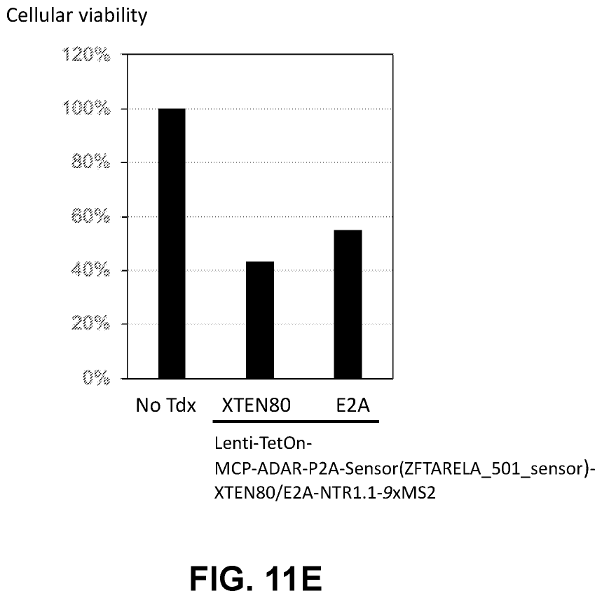

In vivo detection of ZFTA-RELA fusion transcripts by all-in-one ADAR-sensor-NTR constructs with different sensor lengths to trigger cell death in the presence of CB1945 prodrug. ADAR-sensor-NTR constructs with different sensor lengths (L_{S}) complementary to ZFTA-RELA fusion transcript were constructed to test the effect of sensor length on cell ablation efficacy (FIG. 11A-B). We co-transfected the all-in-one ADAR-sensor-NTR constructs, with either empty vector (EV) or pmax-ZFTA-RELA into HEK293T cells and added CB1954 prodrug 24 hours post-transfection (FIG. 11C). Cellular viability of samples expressing ZFTA-RELA fusion (fusion+) was measured using the CellTiter-Glo 2.0 Assay to quantify metabolically active cells normalized to that of samples receiving empty vector control (fusion−), 5 days post prodrug addition (FIG. 11C). We observed an increase of cell ablation activity (decrease in normalized cellular viability) with sensor length L_{S} of 501 compared to L_{S} of 90 or 150 (FIG. 11C). We further tested the sensor in ablating BDX-1425EPN cancer cells derived from a ST-EPN-RELA tumor expressing endogenous ZFTA-RELA fusion transcripts (FIG. 11D). We created lentiviral vectors to carry all-in-one ADAR-sensor-NTR against ZFTA-RELA. We produced virus particles by co-transfecting Lenti-X 293T cells with a mixture of pLP1, pLP2, and VSV-G, and the doxycyclineinducible lentiviral vectors carrying the ADAR-sensor-NTR. Virus was harvested from the Lenti-X 293T supernatant, filtered using 45 μM PES filters, concentrated 1:100 using Lenti-X Concentrator. The day prior to transduction, BXD-1425EPN cells were seeded into 24-well plates at a density of 1.0×10⁵ cells per well. Prior to transduction, media was changed to media containing 10 μg/mL polybrene, 0.5 mL per well. 25 μL of virus was added to each well and incubated overnight. Media was changed 24 h post-transduction. Two days post-transduction, media with 2 μg/mL Puromycin was added to each well. Antibiotic selection continued for 10 days before seeding for cell ablation experiment. The non-transduced (control) and transduced BXD-1425EPN cells were seeded into wells in a 96-well plate with media containing doxycycline (100 ng/mL) at concentrations of approximately 10,000 per well. CB1954 prodrug was added 24 hours post seeding and cellular viability of samples were measured using the CellTiter-Glo 2.0 Assay to quantify metabolically active cells five days after prodrug addition. BDX-1425EPN cells transduced with all-in-one ADAR-sensor-NTR targeting ZFTA-RELA in the presence of CB1954 prodrug displayed significant cell death compared to non-transduced cells, demonstrating the efficacy of all-in-one ADAR-sensor-NTR in ablating cancer cells expressing the endogenous fusion transcripts (FIG. 11E).

### Example 12

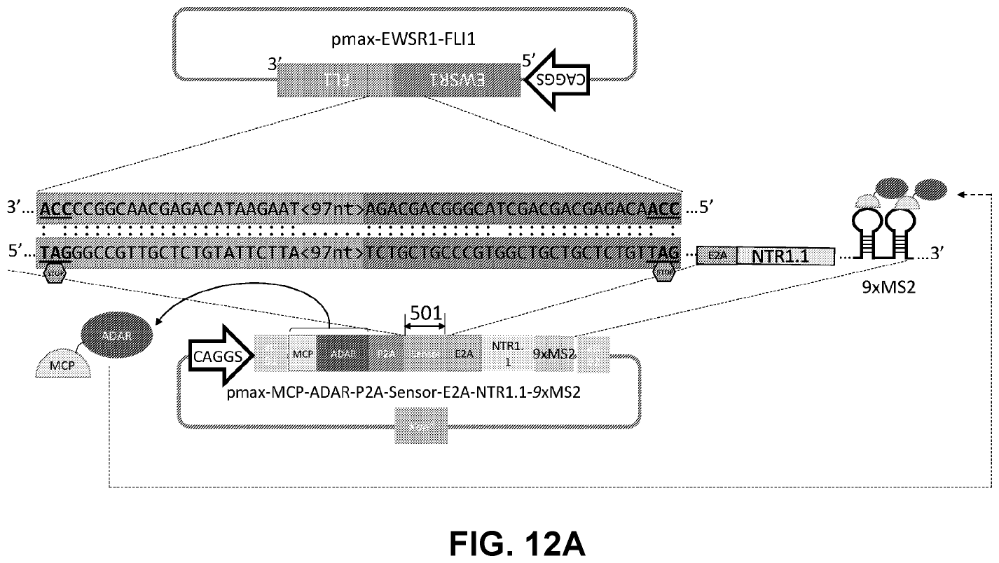

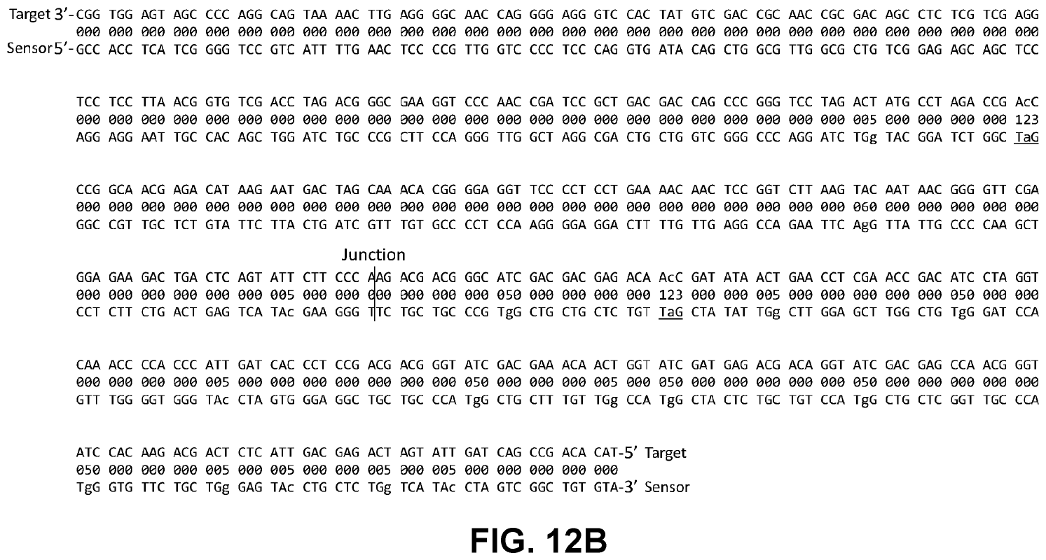

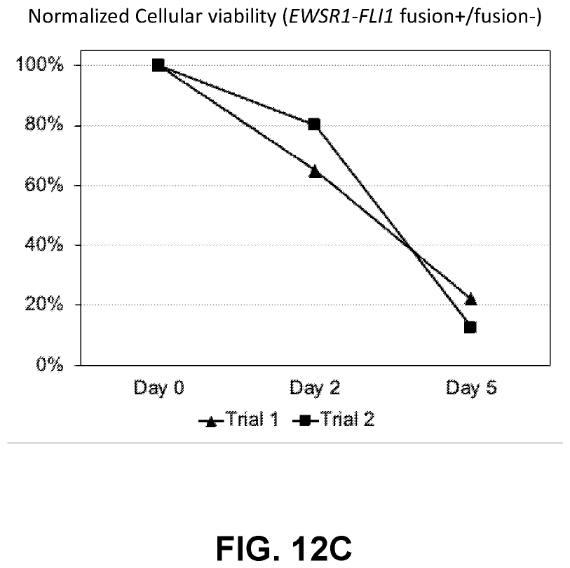

In vivo detection of EWSR1-FLI1 fusion transcripts by an all-in-one ADAR-sensor-NTR construct to trigger cell death in the presence of CB1945 prodrug. An ADAR-sensor-NTR construct with a 501-nt sensor complementary to EWSR1-FLI1 fusion transcript was constructed to ablate cells expressing EWSR1-FLI1 fusion transcript (FIG. 12A-B). We co-transfected the all-in-one ADAR-sensor-NTR construct, with either empty vector (EV) or pmax-EWSR1-FL1 into HEK293T cells and added CB1954 prodrug 24 hours post-transfection (FIG. 12C). Cellular viability of samples expressing EWSR1-FLI1 fusion (fusion+) was measured using the CellTiter-Glo 2.0 Assay to quantify metabolically active cells normalized to that of samples receiving empty vector control (fusion−), 2 days and 5 days post prodrug addition (FIG. 12C). The presence of fusion transcript, but not empty vector control, led to increased cell death in the presence of CB1954 prodrug on Day 2, and with more pronounced cell death on Day 5, demonstrating the specific ablation of cells expressing EWSR1-FLI1 fusion transcripts in vivo (FIG. 12C).

### Example 13

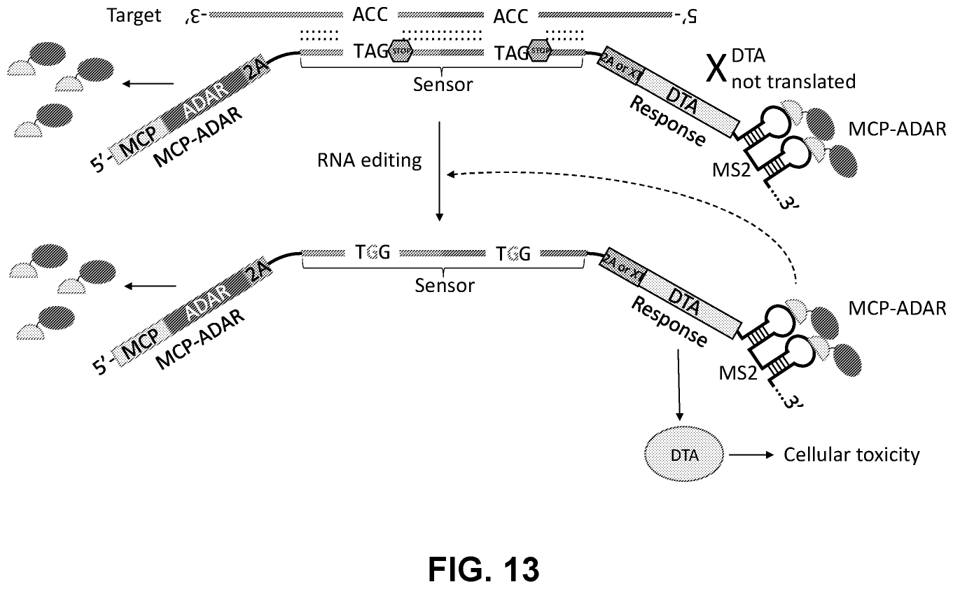

Design of sensor-DTA or ADAR-sensor-DTA (diphtheria toxin fragment A). Chimeric transcripts (fusion transcripts) arising from fusion genes harbor unique junctional sequences that can act as target for detection (FIG. 13). A sensor-DTA or ADAR-sensor-DTA construct can be constructed to express, optionally, a fusion protein containing ADAR enzyme or a fluorescent marker protein, and an RNA sensor fragment coupled to the coding sequence of diphtheria toxin fragment A (DTA) that get translated upon fusion transcript detection. The expressed DTA as a result of fusion transcript detection leads to cellular toxicity and target cell death.

One exemplary sensor-DTA or ADAR-sensor-DTA construct of the present disclosure (e.g., FIG. 13) is composed of, from 5′ to 3′: (i) optionally, the coding sequence of ADAR fusion protein, such as the fusion of ADAR deaminase domain with MS2 coat protein (MCP) or a fluorescent marker protein; (ii) optionally, a 2A peptide sequence that allows downstream peptides to be separated from upstream peptides; (iii) a sensor sequence reverse-complementary to the target sequence, with one or more 5′-TAG-3′ sensing triplet opposing 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets on the target sequence, surrounding the junctional sequence of the fusion transcript; (iv) optionally, a 2A peptide or protein linker sequence, that either allows downstream peptide to be separated or allows for the “spacing” of downstream peptide from the upstream peptides; (v) a diphtheria toxin fragment A (DTA) gene whose expression is dependent on the activation of the upstream sensor fragment when the 5′-TAG-3′ stop codons are converted to 5′-TGG-3′ codon upon binding to target sequence with 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets and ADAR-mediated RNA editing; (vi) optionally, an array of binding sites such as MS2 stem loops that can recruit MS2 coat protein (MCP)-ADAR fusion.

### Example 14

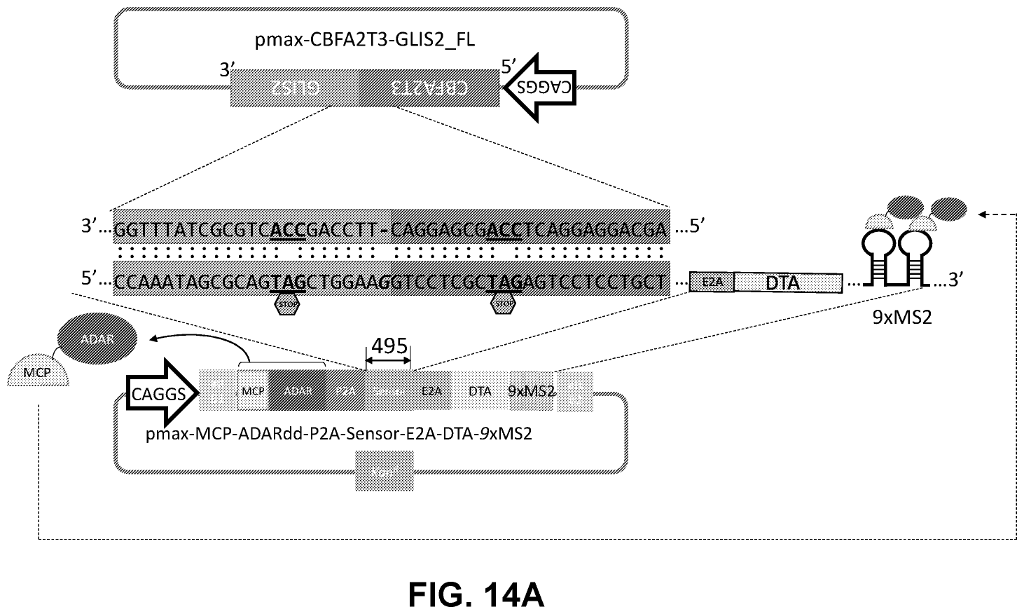

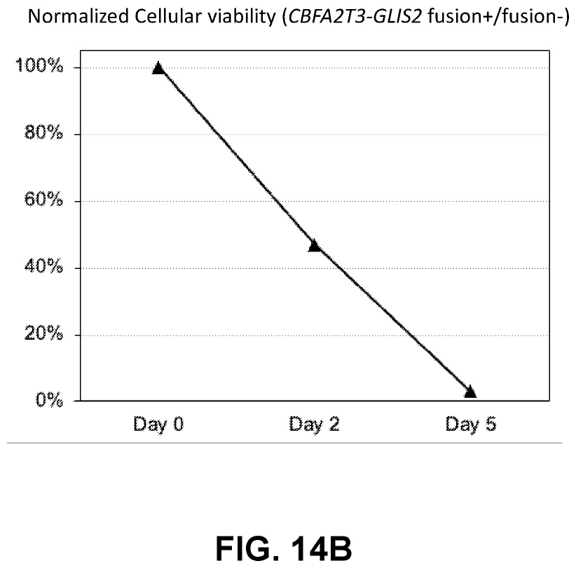

In vivo detection of CBFA2T3-GLIS2 fusion transcripts by all-in-one ADAR-sensor-DTA to trigger cellular toxicity and target cell death. A pmax-MCP-ADAR-P2A-Sensor (CBFA2T3GLIS2_495)-E2A-DTA.1-9xMS2 was constructed to express an RNA molecule containing coding sequence for ADAR deaminase domain fused with MCP (MCP-ADARdd(E488Q)), followed by that coding for P2A peptide, followed by a sensor region complementary to the target CBFA2T3-GLIS2 fusion transcripts and two sensing stop codons surrounding the fusion junction, followed by coding sequences of E2A peptide and DTA, then by 9 copies of MS2 stem loop (9xMS2). The 9xMS2 can recruit MCP-ADARdd(E488Q) expressed from the same RNA molecule. The binding (detection) of sensor region to the fusion transcript triggers the editing of the two TAG stop codons to TGG codons, allowing the downstream DTA to be translated. The target-dependent expression of DTA is cytotoxic and induces target cell death (FIG. 14A). The all-in-one ADAR-sensor-DTA construct was co-transfected with either empty vector (EV) or pmax-CBFA2T3-GLIS2 FL into HEK293T cells (FIG. 14B). Cellular viability was measured using the CellTiter-Glo 2.0 Assay, a luminescent quantification of ATP indicating the presence of metabolically active cells, 2 days and 5 days post transfection. The presence of fusion transcript, but not the empty vector control, led to increased cell death in the transfected cells on Day 2 which became more pronounced on Day 5, demonstrating the specific ablation of cells expressing fusion CBFA2T3-GLIS2 transcripts in vivo (FIG. 14B).

### Example 15

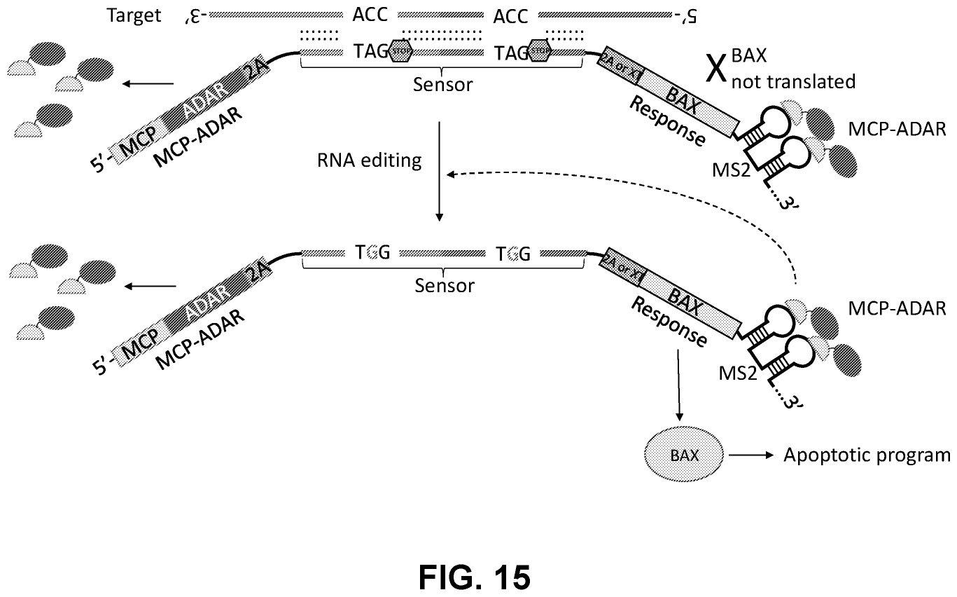

Design of sensor-BAX or ADAR-sensor-BAX (BCL2 associated X, apoptosis regulator). Chimeric transcripts (fusion transcript) arising from fusion genes harbor unique junctional sequences that can act as target for detection (FIG. 15). A sensor-BAX or ADAR-sensor-BAX construct can be constructed to express, optionally, a fusion protein containing ADAR enzyme or a fluorescent marker protein, and an RNA sensor fragment coupled to the coding sequence of an apoptosis regulator BCL2 associated X (BAX) that get translated upon fusion transcript detection. The expressed BAX protein induces or promotes apoptosis in target cells.

One exemplary sensor-BAX or ADAR-sensor-BAX construct of the present disclosure (e.g., FIG. 15) is composed of, from 5′ to 3′: (i) optionally, the coding sequence of ADAR fusion protein, such as the fusion of ADAR deaminase domain with MS2 coat protein (MCP) or a fluorescent marker protein; (ii) optionally, a 2A peptide sequence that allows downstream peptides to be separated from upstream peptides; (iii) a sensor sequence reverse-complementary to the target sequence, with one or more 5′-TAG-3′ sensing triplet opposing 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets on the target sequence, surrounding the junctional sequence of the fusion transcript; (iv) optionally, a 2A peptide or protein linker sequence, that either allows downstream peptide to be separated or allows for the “spacing” of downstream peptide from the upstream peptides; (v) BCL2 associated X (BAX) gene whose expression is depending on the activation of the upstream sensor fragment when the 5′-TAG-3′ stop codons are converted to 5′-TGG-3′ codon upon binding to target sequence with 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets and ADAR-mediated RNA editing; (vi) optionally, an array of binding sites such as MS2 stem loops that can recruit MS2 coat protein (MCP)-ADAR fusion.

### Example 16

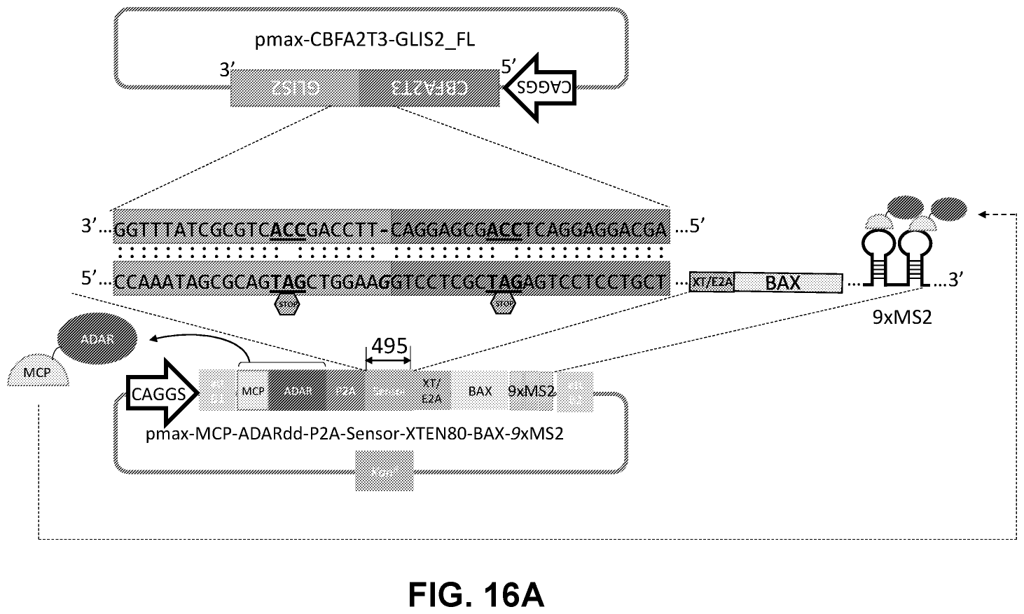

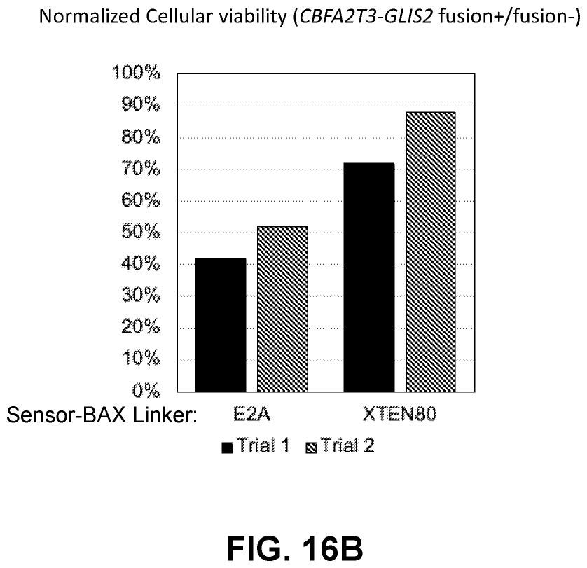

In vivo detection of CBFA2T3-GLIS2 fusion transcripts by all-in-one ADAR-sensor-BAX to induce apoptosis in target cells. A pmax-MCP-ADAR-P2A-Sensor (CBFA2T3GLIS2_495)-E2A-BAX-9xMS2 and a pmax-MCP-ADAR-P2A-Sensor (CBFAT3GLIS2_495)-XTEN80-BAX-9xMS2 were constructed to express an RNA molecule containing coding sequence for ADAR deaminase domain fused with MCP (MCP-ADARdd(E488Q)), followed by that coding for P2A peptide, followed by a sensor region complementary to the target CBFA2T3-GLIS2 fusion transcripts and two sensing stop codons surrounding the fusion junction, followed by coding sequences of E2A peptide (or XTEN80, respectively) and BAX, then by 9 copies of MS2 stem loop (9xMS2). The 9xMS2 can recruit MCP-ADARdd(E488Q) expressed from the same RNA molecule. The binding (detection) of sensor region to the fusion transcript triggers the editing of the two TAG stop codons to TGG codons, allowing the downstream BAX to be translated. The target-dependent expression of BAX induces apoptosis in target cells (FIG. 16A). The all-in-one ADAR-sensor-BAX constructs were co-transfected with either empty vector (EV) or pmax-CBFA2T3-GLIS2_FL into HEK293T cells (FIG. 16B). Cellular viability was measured using the CellTiter-Glo 2.0 Assay to quantify o metabolically active cells 2 days post transfection. The presence of fusion transcript, but not the empty vector control, led to increased cell death in the transfected cells, demonstrating the specific ablation of cells expressing fusion CBFA2T3-GLIS2 transcripts in vivo (FIG. 16B).

### Example 17

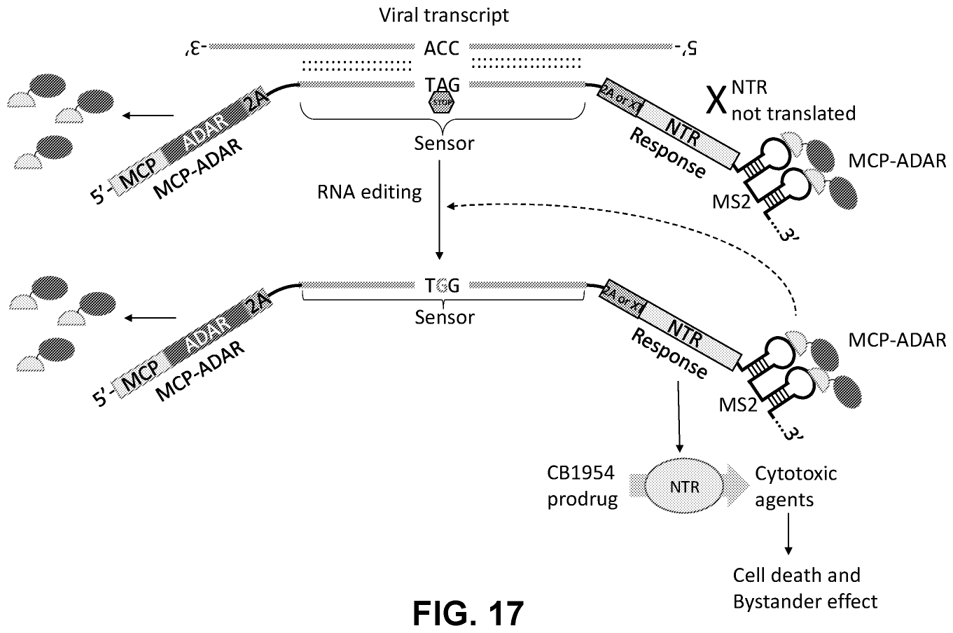

Design of sensor-NTR or ADAR-sensor-NTR for viral transcripts. Virus-infected cells and some cancer cells express viral transcripts that are not present in uninfected cells or normal cells. These viral transcripts serve as unique fingerprint for these cells (FIG. 17). A sensor-NTR or an ADAR-sensor-NTR construct can be constructed to express, optionally, a fusion protein containing ADAR enzyme or a fluorescent marker protein, and an RNA sensor fragment coupled to the coding sequence of nitroreductase (NTR) that get translated upon fusion transcript detection. The expressed NTR as a result of viral transcript detection converts prodrug such as CB1954 (Tretazicar) to cytotoxic agents that can lead to cell death and diffuse to neighboring cells to cause neighboring cell death (also known as Bystander effect). Alternatively, MTZ (Metronidazole) can be used to cause cell death in the cells expressing fusion transcript without bystander effect.

One exemplary sensor-NTR or ADAR-sensor-NTR construct of the present disclosure (e.g., FIG. 17) is composed of, from 5′ to 3′: (i) optionally, the coding sequence of ADAR fusion protein, such as the fusion of ADAR deaminase domain with MS2 coat protein (MCP), or a fluorescent protein; (ii) optionally, a 2A peptide sequence that allows downstream peptides to be separated from upstream peptides; (iii) a sensor sequence reverse-complementary to the target viral sequence, with one or more 5′-TAG-3′ sensing triplet opposing 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets on the target sequence on the target viral transcript; (iv) optionally, a 2A peptide or protein linker sequence, that either allows downstream peptide to be separated or allows for the “spacing” of downstream peptide from the upstream peptides; (v) an nitroreductase (NTR) gene whose expression is depending on the activation of the upstream sensor fragment when the 5′-TAG-3′ stop codons are converted to 5′-TGG-3′ codon upon binding to target sequence with 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets and ADAR-mediated RNA editing; (vi) optionally, an array of binding sites such as MS2 stem loops that can recruit MS2 coat protein (MCP)-ADAR fusion.

### Example 18

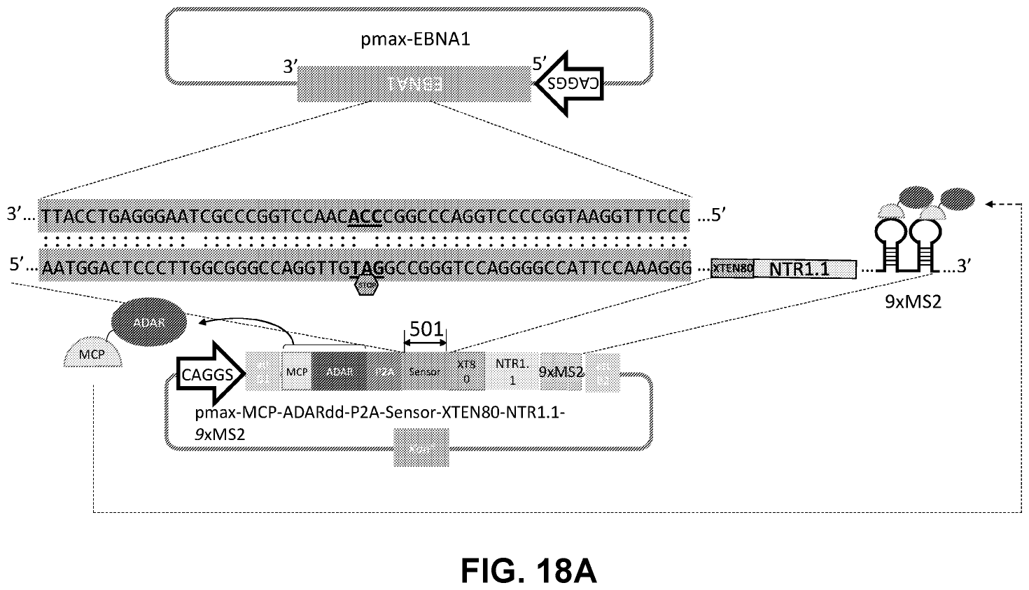

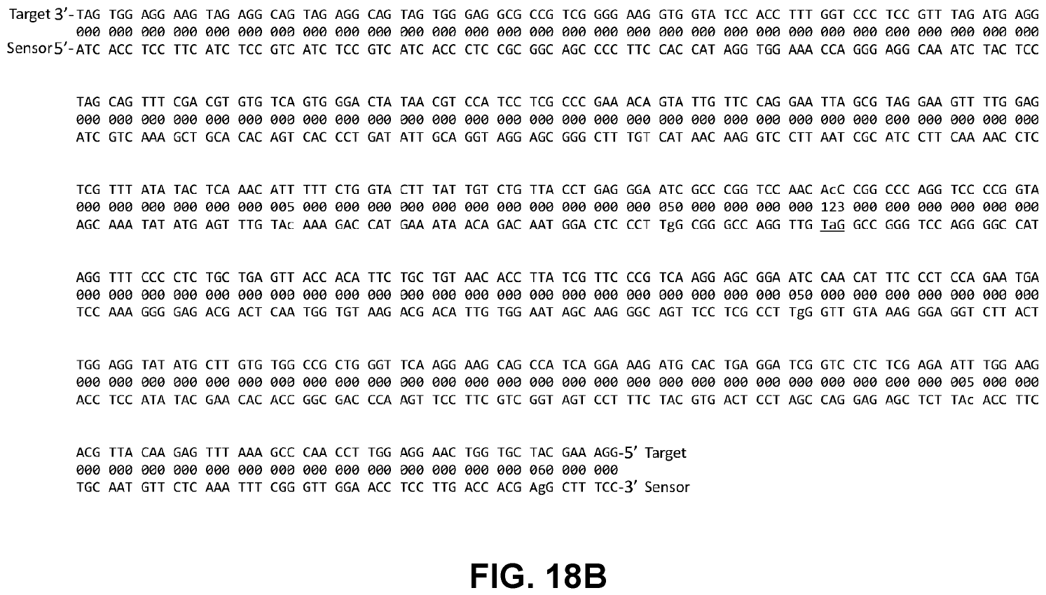

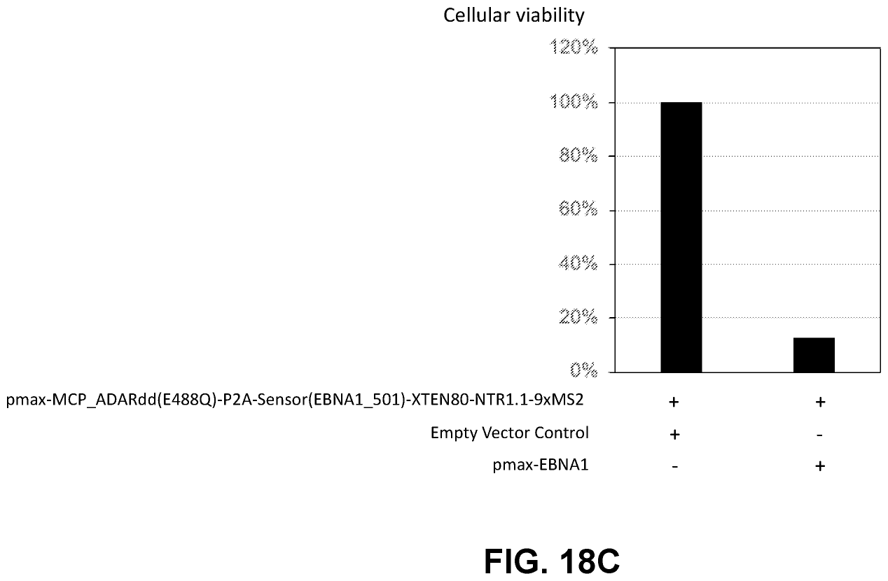

In vivo detection of Epstein Barr Virus (EBV)-EBNA1 transcripts by all-in-one ADAR-sensor-NTR to trigger cell death in the presence of CB1945 prodrug. Epstein Barr Virus (EBV) is present in nasopharyngeal carcinoma, some gastric cancers, and some lymphoma. The virus remains in latent cycle and expresses latent genes such as EBNA1. Plasmid pmax-MCP-ADAR-P2A-Sensor (EBNA1_501)-XTEN80-NTR1.1-9xMS2 was constructed to express an RNA molecule containing coding sequence for ADAR deaminase domain fused with MCP (MCP-ADARdd(E488Q)), followed by the coding sequence for P2A peptide, followed by a sensor region complementary to the target Epstein Barr Virus (EBV)-EBNA1 transcripts with a sensing stop codon complementary to a target CCA triplet on the EBNA1 transcript, followed by coding sequences of XTEN80 peptide and NTR1.1, then by 9 copies of MS2 stem loop (9xMS2). The 9xMS2 can recruit MCP-ADARdd(E488Q) expressed from the same RNA molecule. The binding (detection) of sensor region to the viral transcript triggers the editing of the TAG stop codon to TGG codon, allowing the downstream NTR to be translated. The translated NTR can convert prodrugs (e.g., CB1954) to cytotoxic agents to achieve cell ablation (FIG. 18A-B). We co-transfected the all-in-one ADAR-sensor-NTR construct, with either empty vector (EV), pmax-EBNA1-expressing EBV-EBNA1 transcript-into HEK293T cells and added CB1954 prodrug 24 hours post-transfection (FIG. 18C). Cellular viability was measured using the CellTiter-Glo 2.0 Assay to quantify metabolically active cells 7 days post prodrug addition. The presence of EBV-EBNA1 transcript, but not empty vector control, led to increase cell death in the presence of CB1954 prodrug, demonstrating the specific ablation of cells expressing EBV-EBNA1 transcripts in vivo (FIG. 18C).

### Example 19

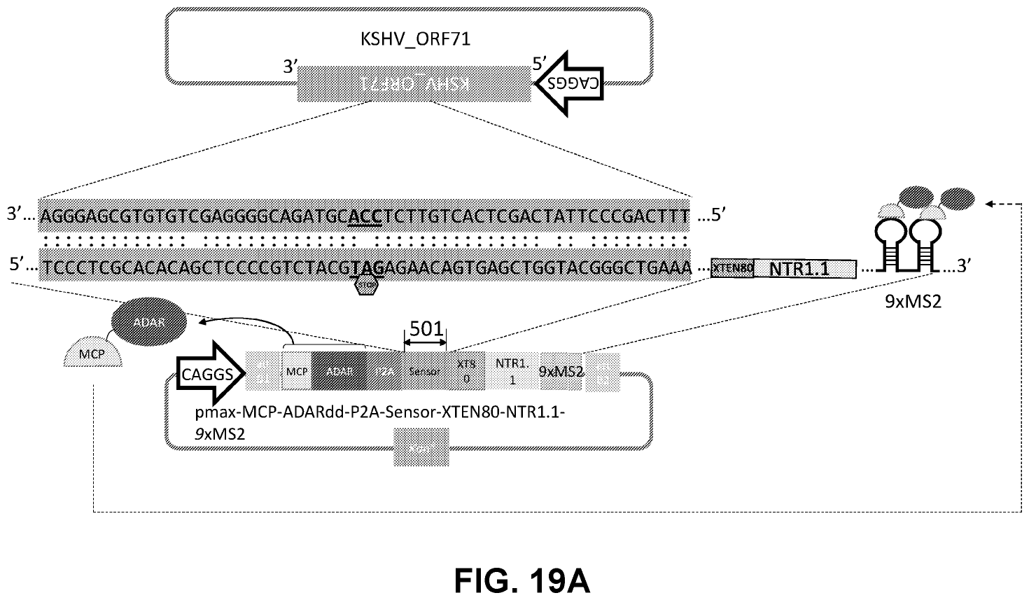

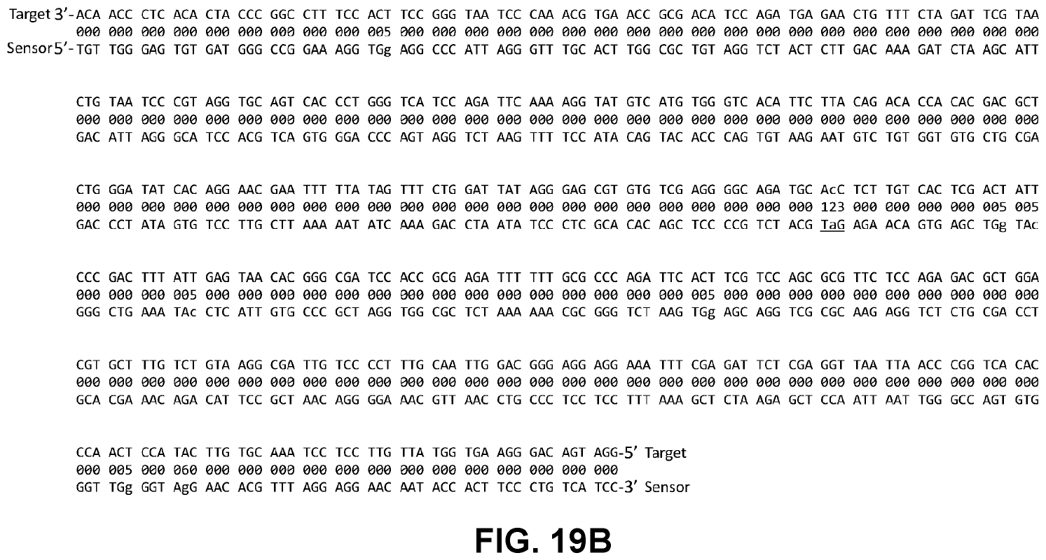

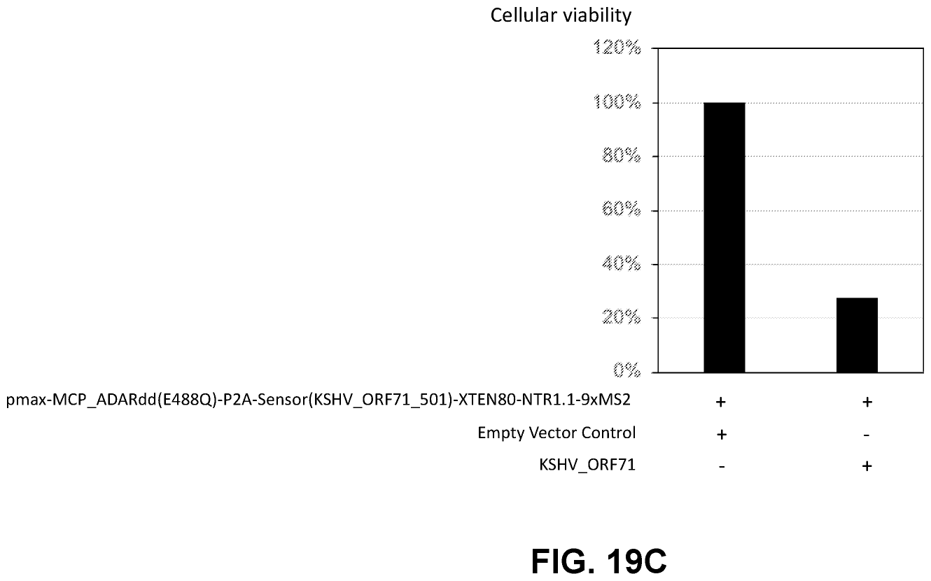

In vivo detection of Kaposi's sarcoma-associated herpesvirus (KSHV)-ORF71 transcripts by all-in-one ADAR-sensor-NTR to trigger cell death in the presence of CB1945 prodrug. Kaposi's sarcoma-associated herpesvirus (KSHV) is present in some sarcoma. The virus in the latent stage expresses latent genes such as ORF71. Plasmid pmax-MCP-ADAR-P2A-Sensor (KSHV_ORF71_501)-XTEN80-NTR1.1-9xMS2 was constructed to express an RNA molecule containing coding sequence for ADAR deaminase domain fused with MCP (MCP-ADARdd(E488Q)), followed by the coding sequence for P2A peptide, followed by a sensor region complementary to the target Kaposi's sarcoma-associated herpesvirus (KSHV)-ORF71 transcripts with a sensing stop codon complementary to a target CCA triplet on the KSHV-ORF71 transcript, followed by coding sequences of XTEN80 peptide and NTR1.1, then by 9 copies of MS2 stem loop (9xMS2). The 9xMS2 can recruit MCP-ADARdd(E488Q) expressed from the same RNA molecule. The binding (detection) of sensor region to the viral transcript triggers the editing of the TAG stop codon to TGG codon, allowing the downstream NTR to be translated. The translated NTR can convert prodrugs (e.g., CB1954) to cytotoxic agents to achieve cell ablation (FIG. 19A-B). The all-in-one ADAR-sensor-NTR construct was co-transfected with either empty vector (EV), pOME0343_ORF71tagged-expressing KSHV-ORF71 transcript-into HEK293T cells and added CB1954 prodrug 24 hours post-transfection (FIG. 19C). Cellular viability was measured using the CellTiter-Glo 2.0 Assay to quantify metabolically active cells 7 days post prodrug addition. The presence of KSHV-ORF71 transcript, but not empty vector control, led to increase cell death in the presence of CB1954 prodrug, demonstrating the specific ablation of cells expressing KSHV-ORF71 transcripts in vivo (FIG. 19C).

### Example 20

Exemplary software for designing sensor sequences. To facilitate the design of sensor sequences, a python program was created. The program accepts the target sequence in capital letters, with the core sensed nucleotide, such as the middle cytosine (C) within a CCA triplet in lower case (i.e., CcA). The program first creates a reverse complement of the target sequence as the initial sensor sequence. Then the nucleotide opposing the lowercase-marked sensed nucleotide of the target is converted to an adenosine (A) to allow ADAR to convert to Inosine upon target binding. The nucleotides immediately upstream and downstream are converted to T and G, respectively, for sensed triplets other than CCA, to create a sensing stop codon (TAG). Then for sensor with more than one sensing triplet, frame-correcting nucleotides are added, roughly in the middle between sensing stop codons on the sensor if the two sensing stop codons are not in frame, that is the nucleotides between the sensing stop codons are not multiples of three. The frame-correcting nucleotides ensure the sensing stop codons are in frame of each other. Alternatively, an option (activated with −deletePlus1 flag) is available for deleting nucleotides between sensing stop codons to ensure the stop codons are in frame. Then the sensor sequence is scanned for stop codons that are not involved in sensing and convert those unwanted stop codons as follows: TAA->TAc, TAG->TgG, TGA->TGg. The sensor sequence is also scanned for unwanted start codons after the first sensing stop codon and convert those from ATG to AgG. Alternatively, an option (activated with —removeAllATG) is available for converting all ATG to AgG. The program then outputs the target sequence, the sensor sequence, and target-sensor alignment. To further facilitate bulk design efforts over a database of fusion transcripts, we created another Python program (BulkSensorRNADesign.py Include CODE LISTING as TXT or FIG?) to scan through fusion transcript database and automate the design of sensor sequences over tens of thousands of fusion sequences. The default for the program is to design sensors with two sensing stop codons, but option (—singleSTOP) is available to instruct the program to design sensors with one sensing stop codon. The sensed triplet is defaulted to CCA, but option (—allowedTriplets) is available to include other triplets that can be used by ADAR (e.g., 9Triplets: CAA, CTA, CGA, ACA, TCA, GCA, CCA, CCT, or CCC). In addition, the program allows specifying the minimum (—minDist) and maximum (—maxDist) distance between sensing stop codons, padding size (—padding) which is the number of nucleotides before and after the sensing stop codons, number of sensors per target to be designed (—numOfSensorsPerTarget). The program accepts fusion transcript annotation from FusionGDB (e.g., ccsm.uth.edu/FusionGDB/tables/TCGA_ChiTaRS_combined_fusion_ORF_analyzed_gencod c_h19v19_In-frame_100k_check_cds_seq.txt). The program scans through each fusion transcript in the database, identifying sensing triplets upstream and downstream of the fusion junction (breakpoint). The program then generates a target design sequence, minimizing distance between sensed stop codons, formatted according to SensorRNADesigner.py requirement (i.e., lowercased sensed nucleotides in the context of uppercase sequence). Functions in the SensorRNADesigner.py is called to write input target design sequence, sensor sequence, and target-sensor alignment to a file for each design. If more than one sensor design is requested, the next sensor design with the next shortest distance between sensing stop codons is outputted, and so on, until the number of sensors to be designed is reached, or all possible designs have been exhausted according to the parameters.

### Example 21

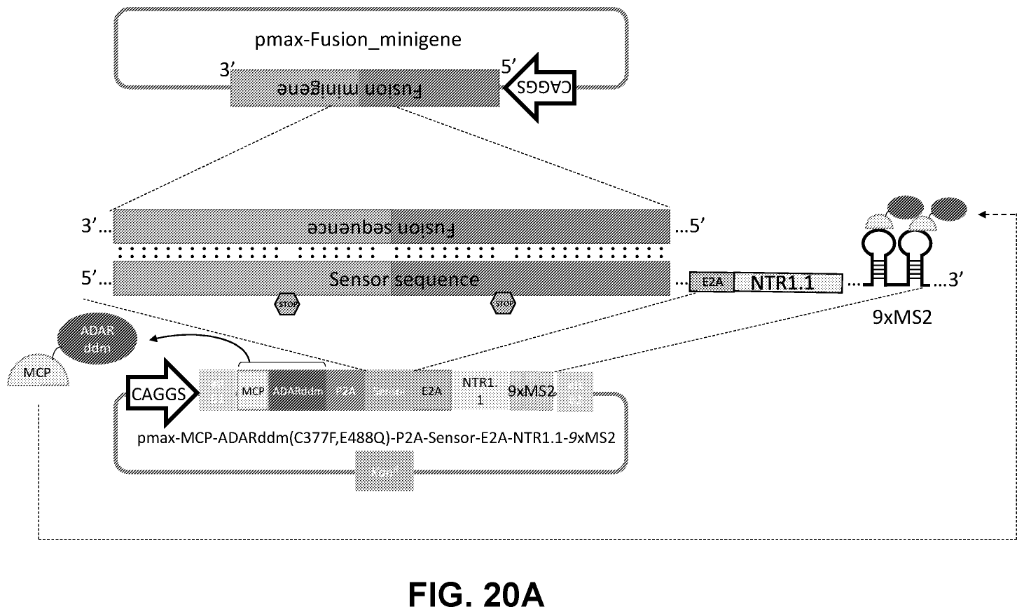

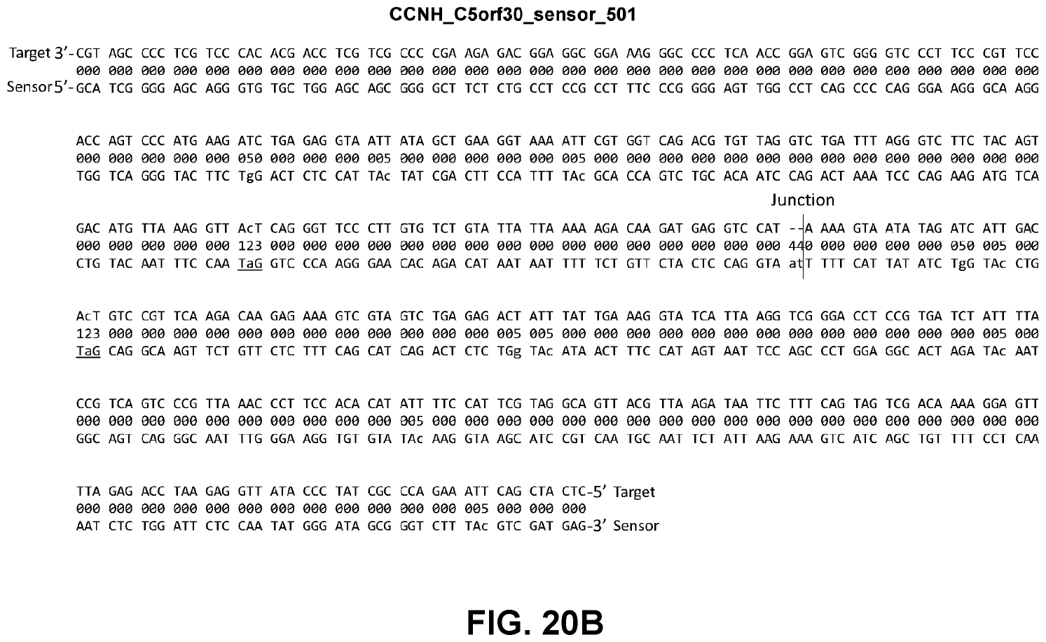

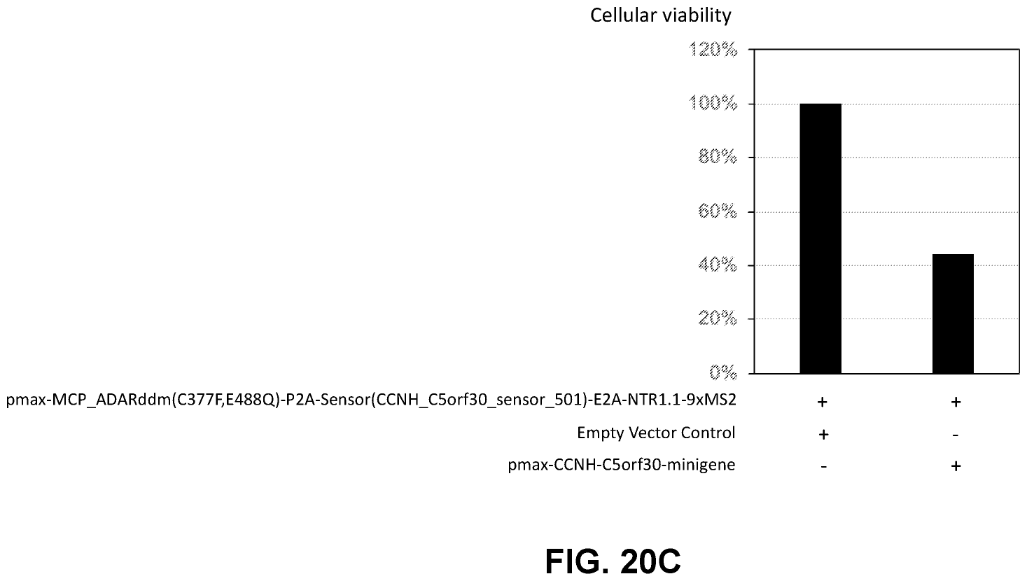

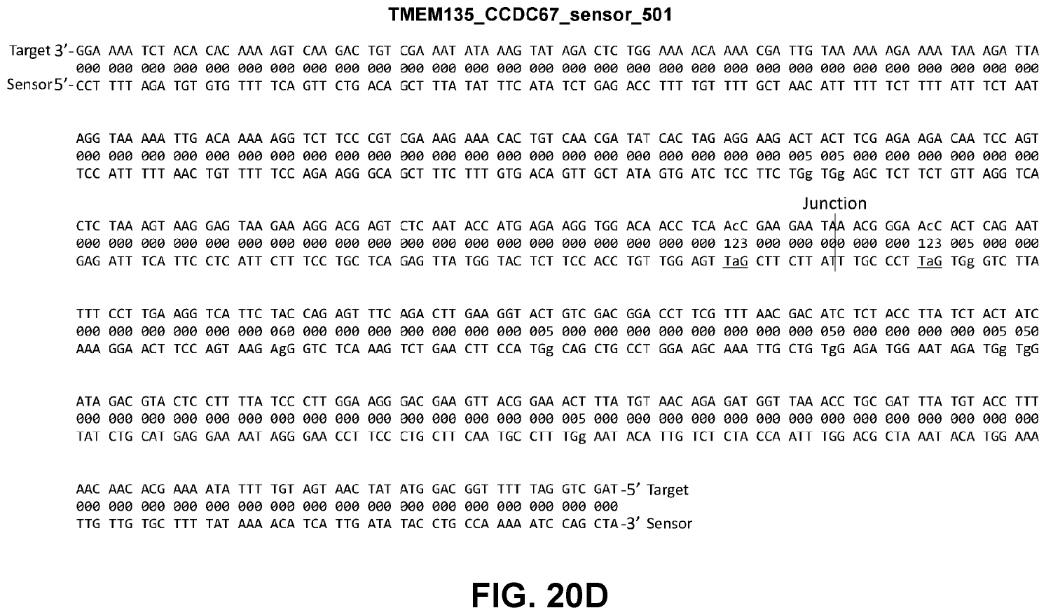

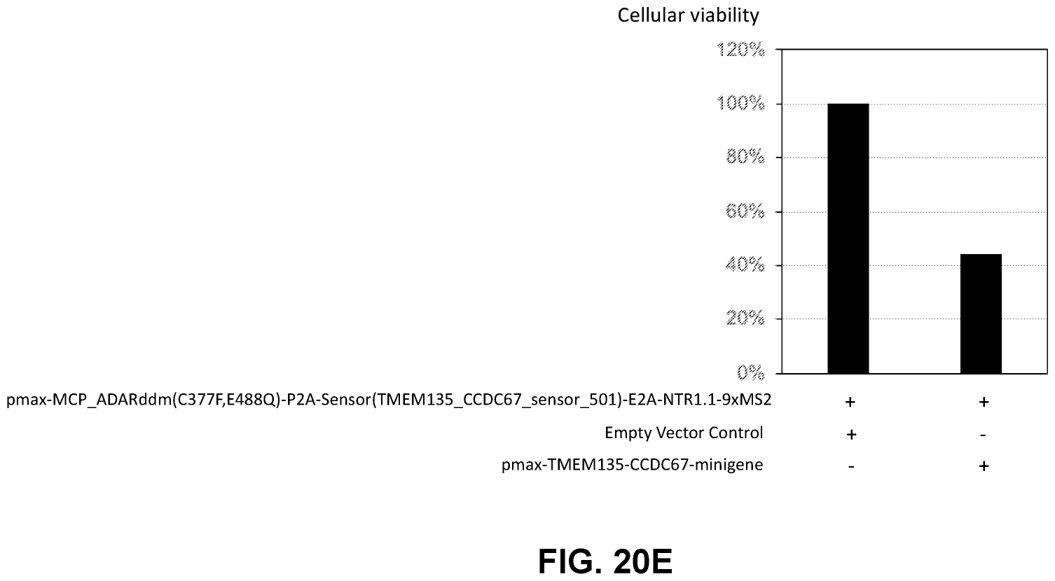

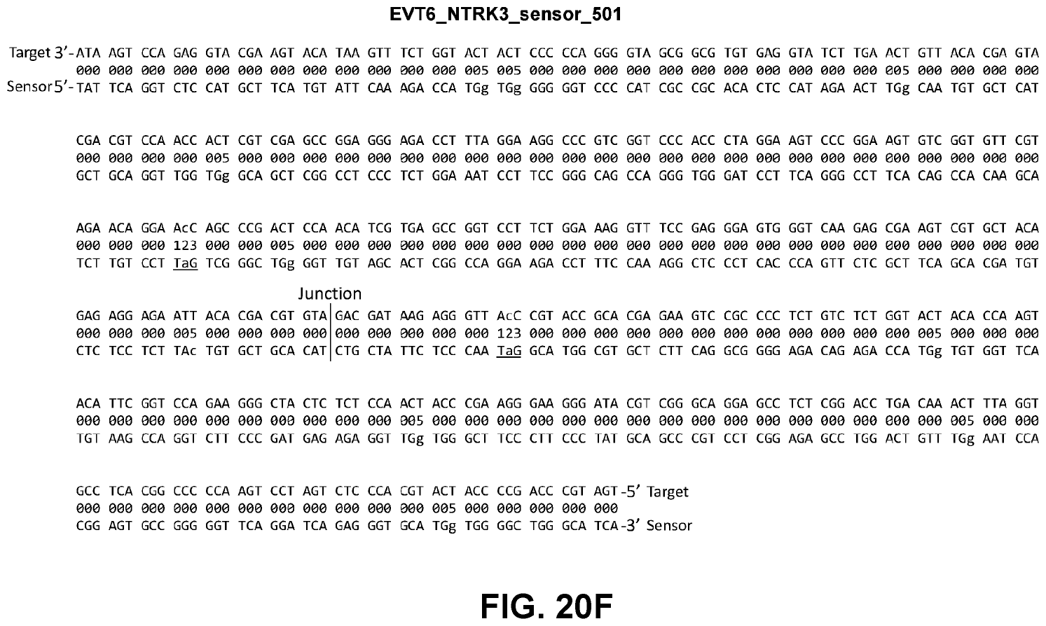

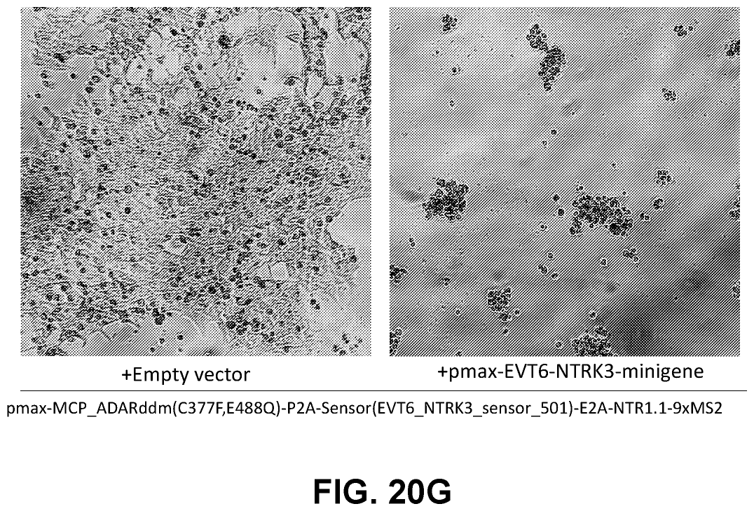

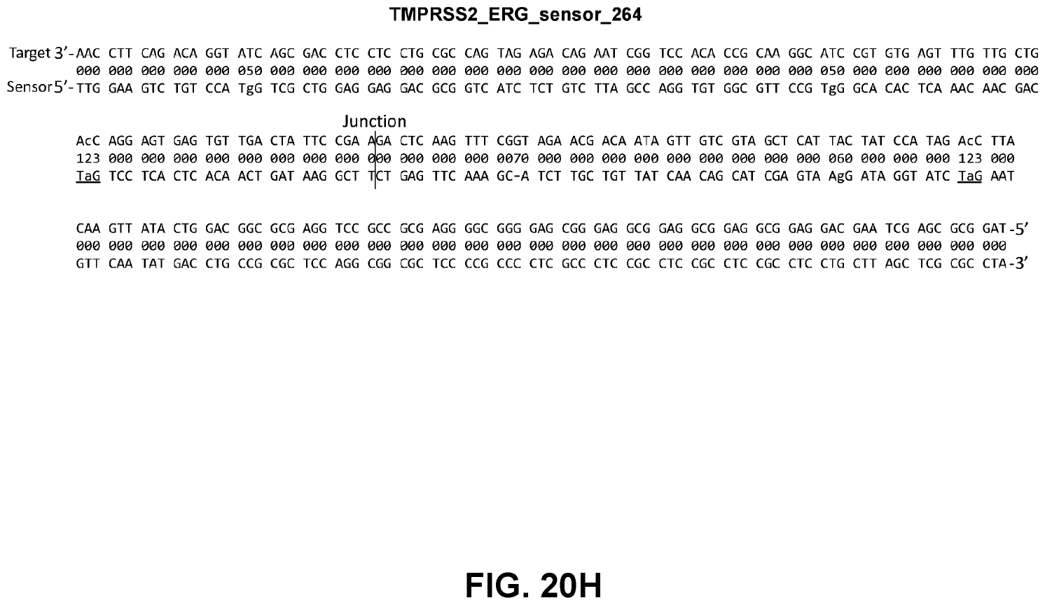

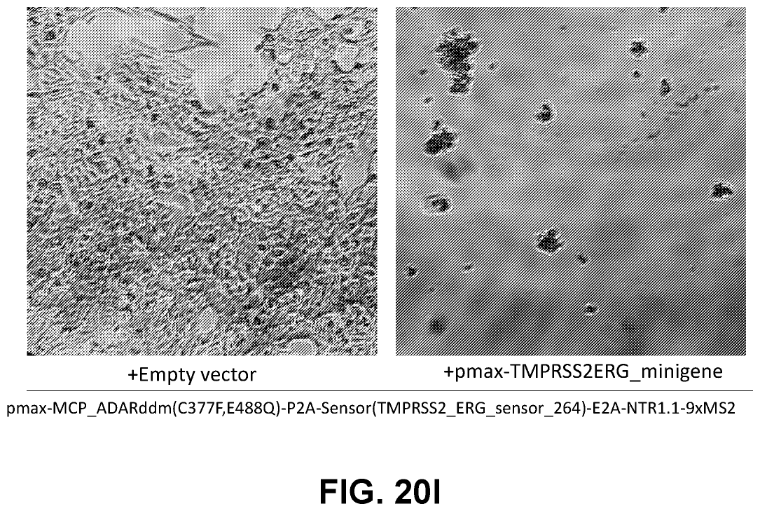

In vivo detection of CCNH-C5orf30, TMEM135-CCDC67, EVT6-NTRK3, and TMPRSS2-ERG fusion transcripts by all-in-one ADAR-sensor-NTR to trigger cell death in the presence of CB1945 prodrug. RNA sensors were designed to target CCNH-C5orf30, TMEM135-CCDC67, ETV6-NTRK3, and TMPRSS2-ERG fusion transcripts. The RNA sensors contain coding sequence for double-mutant ADAR deaminase domain fused with MCP (MCP-ADARddm(C377F,E488Q)), followed by the coding for P2A peptide, followed by a sensor region complementary to the target fusion transcripts and two sensing stop codons surrounding the fusion junction, followed by coding sequences of E2A peptide and NTR1.1, then by 9 copies of MS2 stem loop (9xMS2). The 9xMS2 can recruit MCP-ADARddm(C377F,E488Q) expressed from the same RNA molecule. The binding (detection) of sensor region to the fusion transcript triggers the editing of the two TAG stop codons to TGG codons, allowing the downstream NTR to be translated. The translated NTR can convert prodrugs (e.g., CB1954) to cytotoxic agents to achieve cell ablation (FIG. 20A). For each fusion target, the all-in-one ADAR-sensor-NTR construct was co-transfected with either empty vector (EV) or a fusion minigene into HEK293T cells and added CB1954 prodrug 24 hours post-transfection, and then measured cell viability using CellTiterGlo assay or microscopy seven days after drug addition (FIG. 20A). The ADAR-sensor-NTR charged with CCNH_C5orf30_sensor_501 (FIG. 20B) was able to specifically ablate cells in the presence of CCNH-C5orf30 fusion transcripts (FIG. 20C). The ADAR-sensor-NTR charged with TMEM135_CCDC67_sensor_501 (FIG. 20D) was able to specifically ablate cells in the presence of TMEM135-CCDC67 fusion transcripts (FIG. 20E). The ADAR-sensor-NTR charged with EVT6_NTRK3_sensor_501 (FIG. 20F) was able to specifically ablate cells in the presence of EVT6-NTRK3 fusion transcripts (FIG. 20G). The ADAR-sensor-NTR charged with TMPRSS2_ERG_sensor_264 (FIG. 20H) was able to specifically ablate cells in the presence of TMPRSS2-ERG fusion transcripts (FIG. 20I).

### Example 22

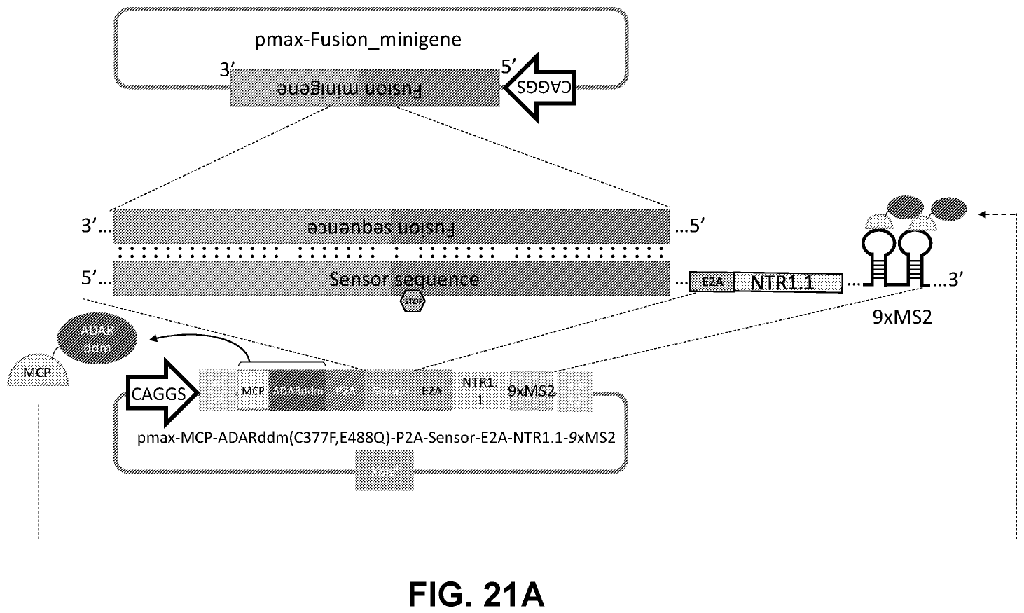

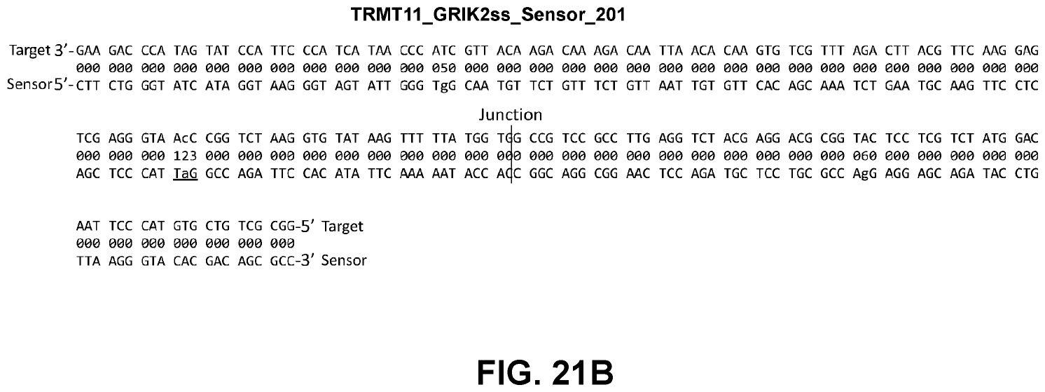

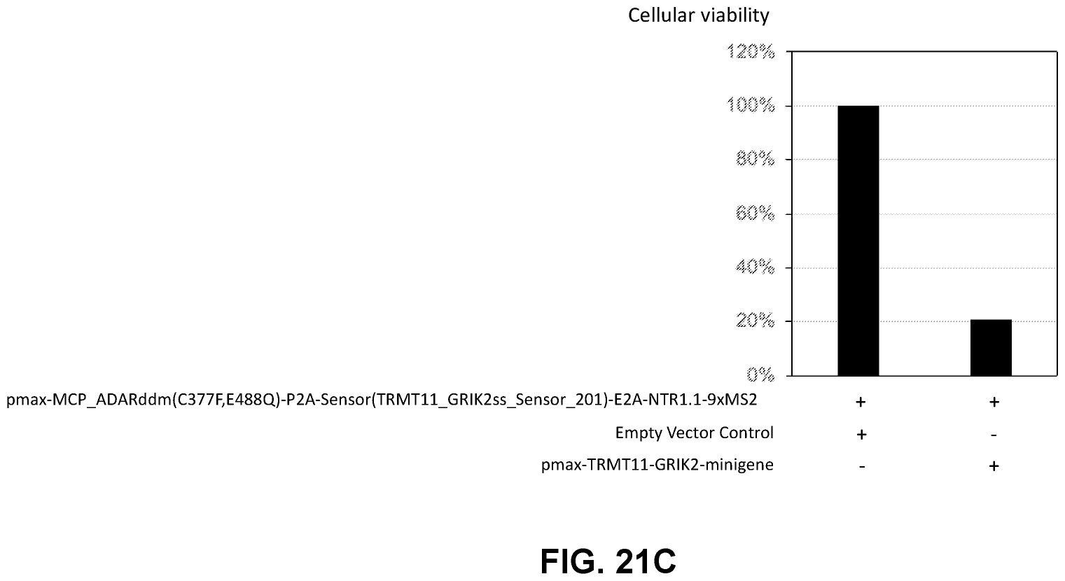

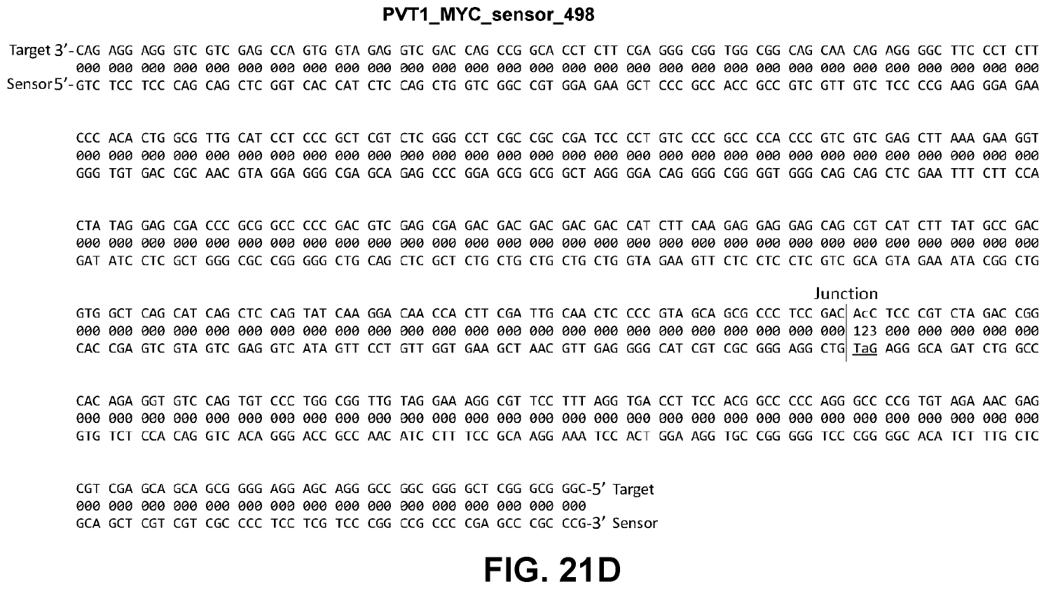

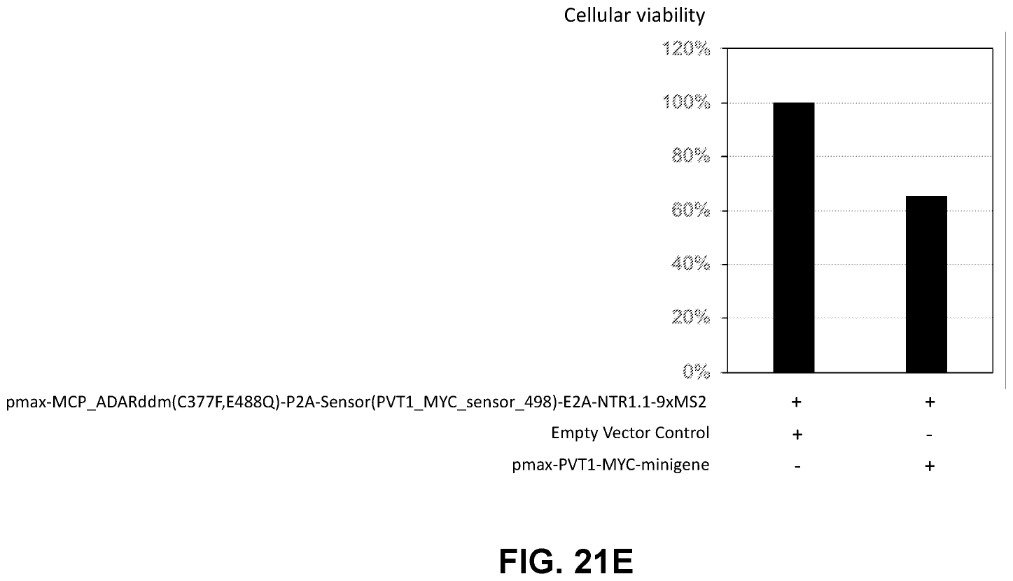

In vivo detection of TRMT11-GRIK2, and PVT1-MYC fusion transcripts by all-in-one ADAR-sensor-NTR to trigger cell death in the presence of CB1945 prodrug. RNA sensors were designed to target TRMT11-GRIK2 and PVT1-MYC fusion transcripts. The RNA sensors contain coding sequence for double-mutant ADAR deaminase domain fused with MCP (MCP-ADARddm(C377F,E488Q)), followed by the coding for P2A peptide, followed by a sensor region complementary to the target fusion transcripts and one sensing stop codon close to the fusion junction, followed by coding sequences of E2A peptide and NTR1.1, then by 9 copies of MS2 stem loop (9xMS2). The 9xMS2 can recruit MCP-MCP-ADARddm(C377F,E488Q) expressed from the same RNA molecule. The binding (detection) of sensor region to the fusion transcript triggers the editing of the TAG stop codon to TGG codon, allowing the downstream NTR to be translated. The translated NTR can convert prodrugs (e.g., CB1954) to cytotoxic agents to achieve cell ablation (FIG. 21A). For each fusion target, the all-in-one ADAR-sensor-NTR construct was co-transfected with either empty vector (EV) or a fusion minigene into HEK293T cells and added CB1954 prodrug 24 hours post-transfection, and then measured cell viability using CellTiterGlo assay 7 days post drug addition (FIG. 21A). The ADAR-sensor-NTR charged with TRMT11_GRIK2ss_Sensor_201 (FIG. 21B) was able to specifically ablate cells in the presence of TRMT11-GRIK2 fusion transcripts (FIG. 21C). The ADAR-sensor-NTR charged with PVT1_MYC_sensor_498 (FIG. 21D) was able to specifically ablate cells in the presence of PVT1-MYC fusion transcripts (FIG. 21E).

### Example 23

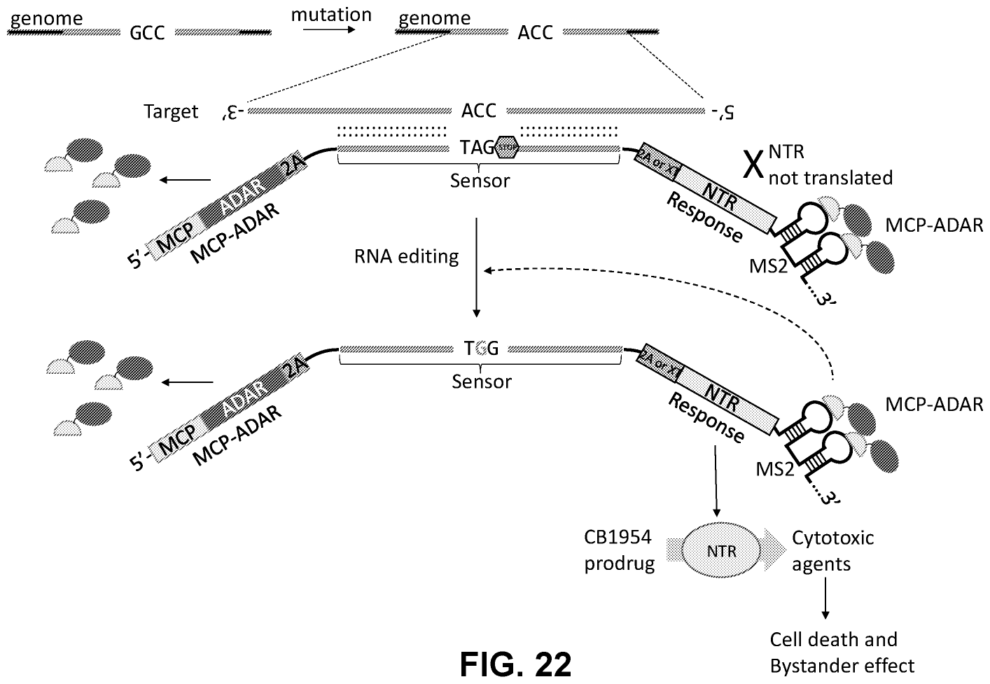

Design of sensor-NTR or ADAR-sensor-NTR for mutant transcripts. Somatic mutations may lead to diseases such as cancers. These mutant transcripts serve as unique fingerprint for these cells (FIG. 22). A sensor-NTR or an ADAR-sensor-NTR construct can be constructed to express, optionally, a fusion protein containing ADAR enzyme or a fluorescent marker protein, and an RNA sensor fragment coupled to the coding sequence of nitroreductase (NTR) that get translated upon mutant transcript detection. The expressed NTR as a result of fusion transcript detection converts prodrug such as CB1954 (Tretazicar) to cytotoxic agents that can lead to cell death and diffuse to neighboring cells to cause neighboring cell death (also known as Bystander effect). Alternatively, MTZ (Metronidazole) can be used to cause cell death in the cells expressing fusion transcript without bystander effect.

One exemplary sensor-NTR or ADAR-sensor-NTR construct of the present disclosure (e.g., FIG. 22) is composed of, from 5′ to 3′: (i) optionally, the coding sequence of ADAR fusion protein, such as the fusion of ADAR deaminase domain with MS2 coat protein (MCP), or a fluorescent protein; (ii) optionally, a 2A peptide sequence that allows downstream peptides to be separated from upstream peptides; (iii) a sensor sequence reverse-complementary to the target sequence, with one or more 5′-TAG-3′ sensing triplet opposing 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplets on the target sequence on the target viral transcript; (iv) optionally, a 2A peptide or protein linker sequence, that either allows downstream peptide to be separated or allows for the “spacing” of downstream peptide from the upstream peptides; (v) an nitroreductase (NTR) gene whose expression is depending on the activation of the upstream sensor fragment when the 5′-TAG-3′ stop codons are converted to 5′-TGG-3′ codon upon binding to target sequence with 5′-CCA-3′ (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplet and ADAR-mediated RNA editing; (vi) optionally, an array of binding sites such as MS2 stem loops that can recruit MS2 coat protein (MCP)-ADAR fusion.

### Example 24

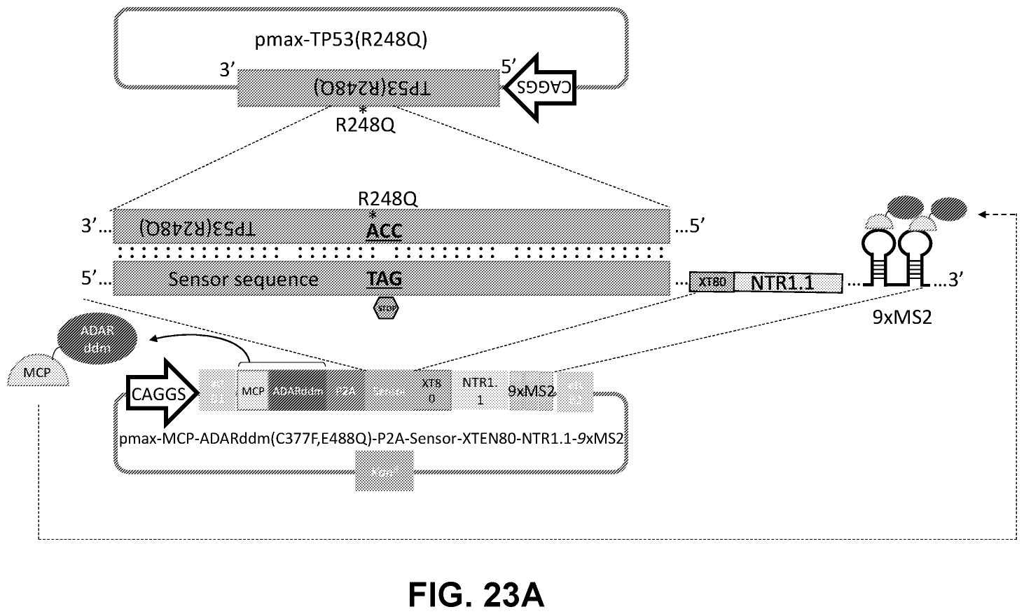

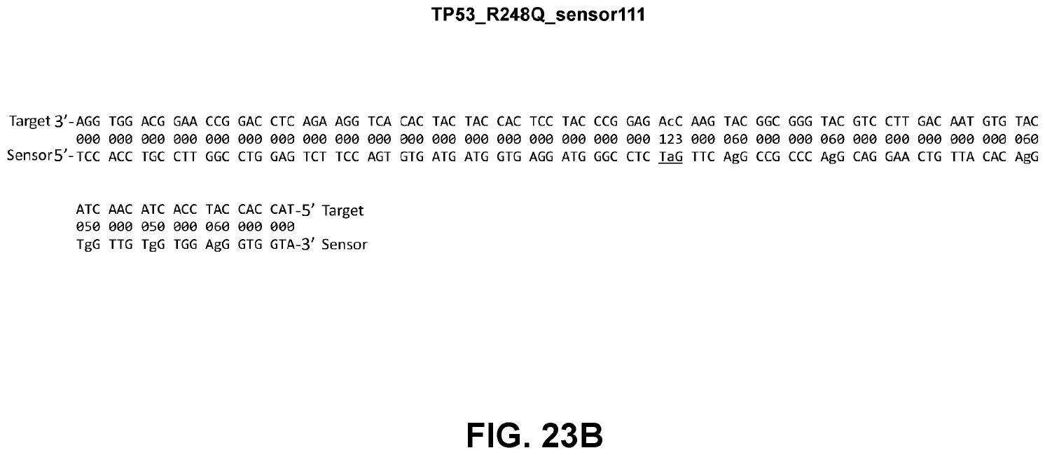

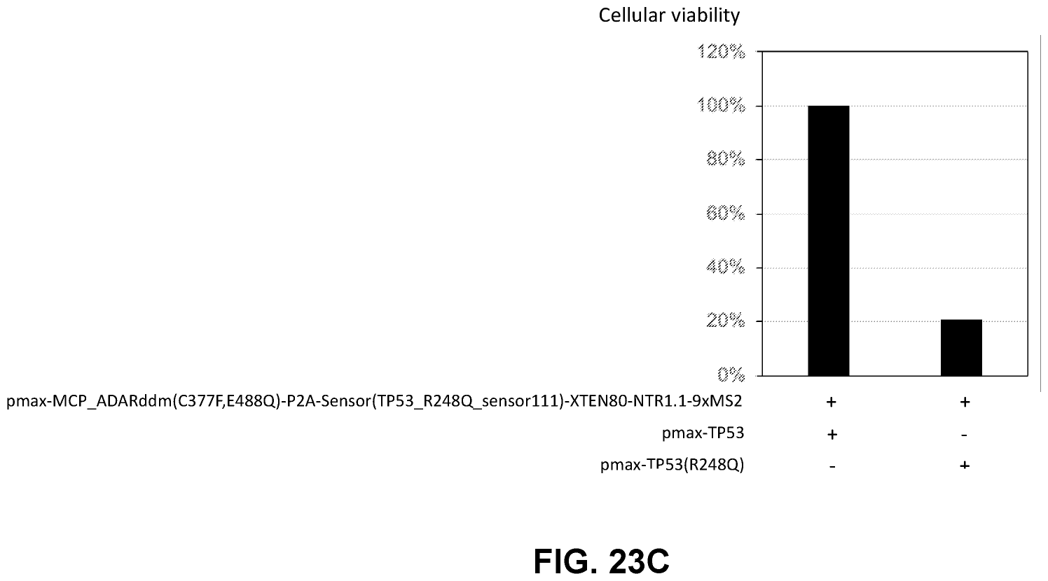

In vivo detection of TP53(R248Q) mutant transcripts by all-in-one ADAR-sensor-NTR to trigger cell death in the presence of CB1945 prodrug. RNA sensors were designed to detect TP53(R248Q) mutant transcript which harbors a CCA triplet not present in wild-type TP53. The RNA sensors contain coding sequence for double-mutant ADAR deaminase domain fused with MCP (MCP-ADARddm(C377F,E488Q)), followed by the coding for P2A peptide, followed by a sensor region complementary to the target TP53(R248Q) transcript and one sensing stop codon opposite the mutant-specific CCA triplet, followed by coding sequences of XTEN80 linker peptide and NTR1.1, then by 9 copies of MS2 stem loop (9xMS2). The 9xMS2 can recruit MCP-MCP-ADARddm(C377F,E488Q) expressed from the same RNA molecule. The binding (detection) of sensor region to the TP53(R248Q) mutant transcript triggers the editing of the TAG stop codon to TGG codon, allowing the downstream NTR to be translated. The translated NTR can convert prodrugs (e.g., CB1954) to cytotoxic agents to achieve cell ablation (FIG. 23A). The all-in-one ADAR-sensor-NTR construct was co-transfected with either empty vector (EV), plasmid expressing wild-type TP53, or mutant TP53(R248Q) into HEK293T cells and added CB1954 prodrug 24 hours post-transfection, and then measured cell viability using CellTiterGlo assay 7 days post drug addition (FIG. 23A). The ADAR-sensor-NTR charged with TP53_R248Q_sensor111 (FIG. 23B) was able to specifically ablate cells in the presence of TP53(R248Q) mutant transcript but not wild-type TP53 transcript (FIG. 23C).

### 4. SEQUENCES

The various embodiments of the present disclosure reference one or more of the nucleic acid sequences and amino acid sequences provided below.

CBFA2T3GLIS2_495_sensor (sensor stop codon lower case and underlined,
mismatches with target lower case):
CTTCTCGGGCTTGACAgGGTAATCGTTGACAgGGTCCACCAGGTCTTGCAGGAGC
TCAAAGAGCTGGTTACACTTGGCCCAGCGACACACCAGCTGCTTGGGCAGGGGC
AGGTCTGGCGAGAGGCACTTGTCCTTGGGAGGGGTAAGGAAGGAGGAGGCAGG
CAGGTGCAGGGCCCCCCCGGAGCCGAGGGGCAGGAAGAACTGGAAGGAGCTGG
GGACACCATCCAAATAGCGCAGtagCTGGAAGgTCCTCGCtagAGTCCTCCTGCTGG
TTGgTGgCCGTCAGGGCGTCCTCGGAGGCCTGCCGCTTCGCCTCGGCCAGGGCCC
GCTCCATCTTGGCACGCTCCGTGGTGgTGgGCTCGTGCGCTTTGCGCTCCGCGTCC
GACACGGCTTTCTGCAGCTCCGACATGGCCTGCCGCTTCACCTCATTCACGGCCT
CTTCAGCCTTCCTCCAGATGTCCTCAGGCACGTAGCCGGTGgGGGTCCTCGGCAG
GA (SEQ ID NO: 5)
CBFA2T3GLIS2_avidity5_sensor (sensor stop codon lower case and underlined,
MS2 stem loops lower case and italicized, mismatches with target lower case):
AGGAGGAGGCAGGCAGGTGCAGGGCCCacagaagcaccatcagggcttctgGAGCCGAGGGG
CAGGAAGAACTGGAAGatgacgcaggaccaccgcgtcGGGGACACCATCCAAATAGCGCA
GtagCTGGAAGgTCCTCGCtagAGTCCTCCTGCTGGTTGgTGgCCGagacatgaggatcacccat
gtGCGTCCTCGGAGGCCTGCCGCTTCGCCaagggtggaggaacaccccaccctCAGGGCCCGC
TCCATCTTGGCACG (SEQ ID NO: 6)
MS2SL (uppercase MS2 stem loop, lowercase spacers, agat: cloning overhang):
agatggccAACATGAGGATCACCCATGTCTGCAGggcc (SEQ ID NO: 7)
P2A amino acid sequence: GSGATNFSLLKQAGDVEENPGP (SEQ ID NO: 13)
E2A amino acid sequence: GSGQCTNYALLKLAGDVESNPGP (SEQ ID NO: 14)
XTEN80 amino acid sequence:
GGPSSGAPPPSGGSPAGSPTSTEEGTSESATPESGPGTSTEPSEGSAPGSPAGSPTSTEE
GTSTEPSEGSAPGTSTEPSE (SEQ ID NO: 15)
MCP-ADARdd(E488Q) amino acid sequence:
MNVGGGGSGGGGSGGGGSGRAMASNFTQFVLVDNGGTGDVTVAPSNFANGVAEW
ISSNSRSQAYKVTCSVRQSSAQKRKYTIKVEVPKVATQTVGGVELPVAAWRSYLNM
ELTIPIFATNSDCELIVKAMQGLLKDGNPIPSAIAANSGIYSAGGRGGGGSGGGGSGG
GGSGPAQLHLPQVLADAVSRLVLGKFGDLTDNFSSPHARRKVLAGVVMTTGTDVK
DAKVISVSTGTKCINGEYMSDRGLALNDCHAEIISRRSLLRFLYTQLELYLNNKDDQK
RSIFQKSERGGFRLKENVQFHLYISTSPCGDARIFSPHEPILEEPADRHPNRKARGQLR
TKIESGQGTIPVRSNASIQTWDGVLQGERLLTMSCSDKIARWNVVGIQGSLLSIFVEPI
YFSSIILGSLYHGDHLSRAMYQRISNIEDLPPLYTLNKPLLSGISNAEARQPGKAPNFS
VNWTVGDSAIEVINATTGKDELGRASRLCKHALYCRWMRVHGKVPSHLLRSKITKP
NVYHESKLAAKEYQAAKARLFTAFIKAGLGAWVEKPTEQDQFSLTNV (SEQ ID NO:
16)
MCP-NES-ADARdd(E488Q) amino acid sequence:
MNVGGGGSGGGGSGGGGSGRAMASNFTQFVLVDNGGTGDVTVAPSNFANGVAEW
ISSNSRSQAYKVTCSVRQSSAQKRKYTIKVEVPKVATQTVGGVELPVAAWRSYLNM
ELTIPIFATNSDCELIVKAMQGLLKDGNPIPSAIAANSGIYSAGGRGGGGSGGGGSGG
GGSGPALQLPPLERLTLGSGGGGSQLHLPQVLADAVSRLVLGKFGDLTDNFSSPHAR
RKVLAGVVMTTGTDVKDAKVISVSTGTKCINGEYMSDRGLALNDCHAEIISRRSLLR
FLYTQLELYLNNKDDQKRSIFQKSERGGFRLKENVQFHLYISTSPCGDARIFSPHEPIL
EEPADRHPNRKARGQLRTKIESGQGTIPVRSNASIQTWDGVLQGERLLTMSCSDKIAR
WNVVGIQGSLLSIFVEPIYFSSIILGSLYHGDHLSRAMYQRISNIEDLPPLYTLNKPLLS
GISNAEARQPGKAPNFSVNWTVGDSAIEVINATTGKDELGRASRLCKHALYCRWMR
VHGKVPSHLLRSKITKPNVYHESKLAAKEYQAAKARLFTAFIKAGLGAWVEKPTEQ
DQFSLTNV (SEQ ID NO: 17)
>NTR1.1 amino acid sequence:
VDIISVALKRHSTKAFDASKKLTPEQAEQIKTLLQYSPSSQNSQPWHFIVASTEEGKA
RVAKSAAGNYVFSERKMLDASHVVVFCAKTAMDDVWLKLVVDQEDADGRFATPE
AKAANDKGRKFTADMHRKDLHDDAEWMAKQVYLNVGNFLLGVAALGLDAVPIEG
FDAAILDAEFGLKEKGYTSLVVVPVGHHSVEDFNATLPKSRLPQNITLTEV (SEQ ID
NO: 18)
>mRuby2-P2A-Sensor(CBFA2T3GLIS2_495)-XTEN80-NTR1.1-9xMS2:
GCCGCCACCATGGTGCGGGGTTCTCATCATCATCATCATCATGGTATGGCTAGCA
TGACTGGTGGACAGCAAATGGGTCGGGATCTGTACGACGATGACGATAAGGATC
CGATGGTGTCTAAGGGCGAAGAGCTGATCAAGGAAAATATGCGTATGAAGGTGG
TCATGGAAGGTTCGGTCAACGGCCACCAATTCAAATGCACAGGTGAAGGAGAAG
GCAATCCGTACATGGGAACTCAAACCATGAGGATCAAAGTCATCGAGGGAGGAC
CCCTGCCATTTGCCTTTGACATTCTTGCCACGTCGTTCATGTATGGCAGCCGTACT
TTTATCAAGTACCCGAAAGGCATTCCTGATTTCTTTAAACAGTCCTTTCCTGAGGG
TTTTACTTGGGAAAGAGTTACGAGATACGAAGATGGTGGAGTCGTCACCGTCATG
CAGGACACCAGCCTTGAGGATGGCTGTCTCGTTTACCACGTCCAAGTCAGAGGG
GTAAACTTTCCCTCCAATGGTCCCGTGATGCAGAAGAAGACCAAGGGTTGGGAG
CCTAATACAGAGATGATGTATCCAGCAGATGGTGGTCTGAGGGGATACACTCAT
ATGGCACTGAAAGTTGATGGTGGTGGCCATCTGTCTTGCTCTTTCGTAACAACTT
ACAGGTCAAAAAAGACCGTCGGGAACATCAAGATGCCCGGTATCCATGCCGTTG
ATCACCGCCTGGAAAGGTTAGAGGAAAGTGACAATGAAATGTTCGTAGTACAAC
GCGAACACGCAGTTGCCAAGTTCGCCGGGCTTGGTGGTGGGATGGACGAGCTGT
ACAAGACTAGTGGCAGCGGCGCCACAAACTTCTCTCTGCTAAAGCAAGCAGGTG
ATGTTGAAGAAAACCCCGGGCCTGGCGCGCCACTTCTCGGGCTTGACAGGGTAA
TCGTTGACAGGGTCCACCAGGTCTTGCAGGAGCTCAAAGAGCTGGTTACACTTGG
CCCAGCGACACACCAGCTGCTTGGGCAGGGGCAGGTCTGGCGAGAGGCACTTGT
CCTTGGGAGGGGTAAGGAAGGAGGAGGCAGGCAGGTGCAGGGCCCCCCCGGAG
CCGAGGGGCAGGAAGAACTGGAAGGAGCTGGGGACACCATCCAAATAGCGCAG
TAGCTGGAAGGTCCTCGCTAGAGTCCTCCTGCTGGTTGGTGGCCGTCAGGGCGTC
CTCGGAGGCCTGCCGCTTCGCCTCGGCCAGGGCCCGCTCCATCTTGGCACGCTCC
GTGGTGGTGGGCTCGTGCGCTTTGCGCTCCGCGTCCGACACGGCTTTCTGCAGCT
CCGACATGGCCTGCCGCTTCACCTCATTCACGGCCTCTTCAGCCTTCCTCCAGATG
TCCTCAGGCACGTAGCCGGTGGGGGTCCTCGGCAGGAGGCCGGCCAGGCTCGGG
CCAGGGAGGGCCGTCATCTGGTGCTCCTCCTCCGTCAGGTGGCTCACCTGCTGGT
TCCCCGACATCAACTGAGGAAGGAACTAGCGAAAGTGCGACGCCTGAGAGTGGT
CCCGGTACTAGCACTGAACCGTCAGAGGGGAGTGCACCAGGCAGCCCCGCCGGC
TCTCCAACTTCCACGGAGGAGGGGACATCTACTGAGCCTTCTGAGGGTTCCGCAC
CTGGAACCAGTACTGAGCCCTCCGAGCCTAGGTTAATTAAGGTGGACATCATCAG
CGTGGCTCTGAAGAGGCACTCCACCAAGGCTTTCGACGCTTCCAAGAAACTGAC
CCCTGAACAGGCCGAGCAGATCAAGACCCTGCTCCAGTACAGCCCTAGCTCCCA
GAACAGCCAGCCTTGGCACTTCATCGTGGCTAGCACCGAGGAAGGCAAAGCTAG
GGTGGCTAAGAGCGCCGCTGGCAACTACGTGTTCAGCGAGAGGAAGATGCTGGA
TGCTAGCCACGTGGTGGTGTTCTGCGCTAAGACCGCCATGGACGATGTGTGGCTG
AAGCTGGTGGTGGATCAGGAAGATGCTGATGGCAGGTTCGCTACCCCTGAAGCT
AAGGCCGCTAACGACAAGGGCAGGAAGTTCACTGCCGACATGCACAGGAAGGAT
CTGCACGATGATGCTGAGTGGATGGCCAAGCAGGTGTACCTGAACGTGGGCAAC
TTCCTGCTCGGCGTGGCTGCCCTGGGCCTCGATGCTGTGCCCATCGAAGGCTTCG
ATGCTGCTATCCTGGATGCCGAGTTCGGCCTGAAGGAGAAAGGCTACACCAGCC
TGGTGGTGGTGCCTGTGGGCCACCACAGCGTGGAGGACTTCAACGCTACCCTGCC
TAAGAGCAGGCTGCCCCAGAACATCACCCTGACCGAGGTGTGATTAATTAAAAG
GGCGGATCCGGTCTCCAGATGGCCAACATGAGGATCACCCATGTCTGCAGGGCC
AGATGGCCAACATGAGGATCACCCATGTCTGCAGGGCCAGATGGCCAACATGAG
GATCACCCATGTCTGCAGGGCCAGATGGCCAACATGAGGATCACCCATGTCTGC
AGGGCCAGATGGCCAACATGAGGATCACCCATGTCTGCAGGGCCAGATGGCCAA
CATGAGGATCACCCATGTCTGCAGGGCCAGATGGCCAACATGAGGATCACCCAT
GTCTGCAGGGCCAGATGGCCAACATGAGGATCACCCATGTCTGCAGGGCCAGAT
GGCCAACATGAGGATCACCCATGTCTGCAGGGCCAGATAGATCTCAATTG (SEQ
ID NO: 19)
>EML4ALK_501_sensor (sensor stop codon lower case and underlined,
mismatches with target lower case, [del] deletion with respect to target):
TCGTTGGGCATTCCGGACACCTGGCCTTCATACACCTCCCCAAAGGCGCCATGGC
CCAGACCCCGAATGAGGGTGATGTTTTTCCGCGGCACCTCCTTCAGGTCACTGAT
GGAGGAGGTCTTGCCAGCAAAGCAGTgGTTGGGGTTGTgGTCGGTCATGATGGTC
GAGGTGCGGAGCTTGCTCAGCTTGTACTCAGGGCTCTGCAGCTCCATCTGCATGG
CTTGCAGCTCCtagTGCTTCCGGCGGTACACTT[del]gGGTCCTTTCCCAGGTGtagGCT
CTACAGTgGTTTTGCTCCATATAcGCATGgCTCCACCTGAGTCTCCAGTAcGAACAT
CTCCATTCCCCAAGAAgGCTAAACACTGCACAAATTTTGGCTTTTCATATTTCCCA
AAAATTCCCTGTTTTCTTGTTAGTGAATTGCCGCTCCAGGTCCAGAAGAAAATAT
GAGATTTACCGCAgGTAcTTATGGTATTTGCATCTGTTGGGTGAAACTCCACAGCC
A (SEQ ID NO: 20)
>mRuby2-P2A-Sensor(EML4ALK_501)-E2A-NTR1.1-9xMS2:
GCCGCCACCATGGTGCGGGGTTCTCATCATCATCATCATCATGGTATGGCTAGCA
TGACTGGTGGACAGCAAATGGGTCGGGATCTGTACGACGATGACGATAAGGATC
CGATGGTGTCTAAGGGCGAAGAGCTGATCAAGGAAAATATGCGTATGAAGGTGG
TCATGGAAGGTTCGGTCAACGGCCACCAATTCAAATGCACAGGTGAAGGAGAAG
GCAATCCGTACATGGGAACTCAAACCATGAGGATCAAAGTCATCGAGGGAGGAC
CCCTGCCATTTGCCTTTGACATTCTTGCCACGTCGTTCATGTATGGCAGCCGTACT
TTTATCAAGTACCCGAAAGGCATTCCTGATTTCTTTAAACAGTCCTTTCCTGAGGG
TTTTACTTGGGAAAGAGTTACGAGATACGAAGATGGTGGAGTCGTCACCGTCATG
CAGGACACCAGCCTTGAGGATGGCTGTCTCGTTTACCACGTCCAAGTCAGAGGG
GTAAACTTTCCCTCCAATGGTCCCGTGATGCAGAAGAAGACCAAGGGTTGGGAG
CCTAATACAGAGATGATGTATCCAGCAGATGGTGGTCTGAGGGGATACACTCAT
ATGGCACTGAAAGTTGATGGTGGTGGCCATCTGTCTTGCTCTTTCGTAACAACTT
ACAGGTCAAAAAAGACCGTCGGGAACATCAAGATGCCCGGTATCCATGCCGTTG
ATCACCGCCTGGAAAGGTTAGAGGAAAGTGACAATGAAATGTTCGTAGTACAAC
GCGAACACGCAGTTGCCAAGTTCGCCGGGCTTGGTGGTGGGATGGACGAGCTGT
ACAAGACTAGTGGCAGCGGCGCCACAAACTTCTCTCTGCTAAAGCAAGCAGGTG
ATGTTGAAGAAAACCCCGGGCCTGGCGCGCCATCGTTGGGCATTCCGGACACCT
GGCCTTCATACACCTCCCCAAAGGCGCCATGGCCCAGACCCCGAATGAGGGTGA
TGTTTTTCCGCGGCACCTCCTTCAGGTCACTGATGGAGGAGGTCTTGCCAGCAAA
GCAGTGGTTGGGGTTGTGGTCGGTCATGATGGTCGAGGTGCGGAGCTTGCTCAGC
TTGTACTCAGGGCTCTGCAGCTCCATCTGCATGGCTTGCAGCTCCTAGTGCTTCCG
GCGGTACACTTGGGTCCTTTCCCAGGTGTAGGCTCTACAGTGGTTTTGCTCCATAT
ACGCATGGCTCCACCTGAGTCTCCAGTACGAACATCTCCATTCCCCAAGAAGGCT
AAACACTGCACAAATTTTGGCTTTTCATATTTCCCAAAAATTCCCTGTTTTCTTGT
TAGTGAATTGCCGCTCCAGGTCCAGAAGAAAATATGAGATTTACCGCAGGTACTT
ATGGTATTTGCATCTGTTGGGTGAAACTCCACAGCCAGGCCGGCCAGGCTCGGGC
CAGTGTACTAATTATGCTCTCTTGAAATTGGCTGGAGATGTTGAGAGCAACCCAG
GTCCCTTAATTAAGGTGGACATCATCAGCGTGGCTCTGAAGAGGCACTCCACCAA
GGCTTTCGACGCTTCCAAGAAACTGACCCCTGAACAGGCCGAGCAGATCAAGAC
CCTGCTCCAGTACAGCCCTAGCTCCCAGAACAGCCAGCCTTGGCACTTCATCGTG
GCTAGCACCGAGGAAGGCAAAGCTAGGGTGGCTAAGAGCGCCGCTGGCAACTAC
GTGTTCAGCGAGAGGAAGATGCTGGATGCTAGCCACGTGGTGGTGTTCTGCGCTA
AGACCGCCATGGACGATGTGTGGCTGAAGCTGGTGGTGGATCAGGAAGATGCTG
ATGGCAGGTTCGCTACCCCTGAAGCTAAGGCCGCTAACGACAAGGGCAGGAAGT
TCACTGCCGACATGCACAGGAAGGATCTGCACGATGATGCTGAGTGGATGGCCA
AGCAGGTGTACCTGAACGTGGGCAACTTCCTGCTCGGCGTGGCTGCCCTGGGCCT
CGATGCTGTGCCCATCGAAGGCTTCGATGCTGCTATCCTGGATGCCGAGTTCGGC
CTGAAGGAGAAAGGCTACACCAGCCTGGTGGTGGTGCCTGTGGGCCACCACAGC
GTGGAGGACTTCAACGCTACCCTGCCTAAGAGCAGGCTGCCCCAGAACATCACC
CTGACCGAGGTGTGATTAATTAAAAGGGCGGATCCGGTCTCCAGATGGCCAACA
TGAGGATCACCCATGTCTGCAGGGCCAGATGGCCAACATGAGGATCACCCATGT
CTGCAGGGCCAGATGGCCAACATGAGGATCACCCATGTCTGCAGGGCCAGATGG
CCAACATGAGGATCACCCATGTCTGCAGGGCCAGATGGCCAACATGAGGATCAC
CCATGTCTGCAGGGCCAGATGGCCAACATGAGGATCACCCATGTCTGCAGGGCC
AGATGGCCAACATGAGGATCACCCATGTCTGCAGGGCCAGATGGCCAACATGAG
GATCACCCATGTCTGCAGGGCCAGATGGCCAACATGAGGATCACCCATGTCTGC
AGGGCCAGATAGATCTCAATTG (SEQ ID NO: 21)
>MCP-ADARddm(E488Q) amino acid sequence on all-in-one vectors
MASNFTQFVLVDNGGTGDVTVAPSNFANGVAEWISSNSRSQAYKVTCSVRQSSAQK
RKYTIKVEVPKVATQTVGGVELPVAAWRSYLNMELTIPIFATNSDCELIVKAMQGLL
KDGNPIPSAIAANSGIYSAGGRGGGGSGGGGSGGGGSQLHLPQVLADAVSRLVLGKF
GDLTDNFSSPHARRKVLAGVVMTTGTDVKDAKVISVSTGTKCINGEYMSDRGLALN
DCHAEIISRRSLLRFLYTQLELYLNNKDDQKRSIFQKSERGGFRLKENVQFHLYISTSP
CGDARIFSPHEPILEEPADRHPNRKARGQLRTKIESGQGTIPVRSNASIQTWDGVLQG
ERLLTMSCSDKIARWNVVGIQGSLLSIFVEPIYFSSIILGSLYHGDHLSRAMYQRISNIE
DLPPLYTLNKPLLSGISNAEARQPGKAPNFSVNWTVGDSAIEVINATTGKDELGRASR
LCKHALYCRWMRVHGKVPSHLLRSKITKPNVYHESKLAAKEYQAAKARLFTAFIKA
GLGAWVEKPTEQDQFSLT (SEQ ID NO: 22)
>MCP-ADARddm(C377F, E488Q) amino acid sequence on all-in-one vectors
MASNFTQFVLVDNGGTGDVTVAPSNFANGVAEWISSNSRSQAYKVTCSVRQSSAQK
RKYTIKVEVPKVATQTVGGVELPVAAWRSYLNMELTIPIFATNSDCELIVKAMQGLL
KDGNPIPSAIAANSGIYSAGGRGGGGSGGGGSGGGGSQLHLPQVLADAVSRLVLGKF
GDLTDNFSSPHARRKVLAGVVMTTGTDVKDAKVISVSTGTKFINGEYMSDRGLALN
DCHAEIISRRSLLRFLYTQLELYLNNKDDQKRSIFQKSERGGFRLKENVQFHLYISTSP
CGDARIFSPHEPILEEPADRHPNRKARGQLRTKIESGQGTIPVRSNASIQTWDGVLQG
ERLLTMSCSDKIARWNVVGIQGSLLSIFVEPIYFSSIILGSLYHGDHLSRAMYQRISNIE
DLPPLYTLNKPLLSGISNAEARQPGKAPNFSVNWTVGDSAIEVINATTGKDELGRASR
LCKHALYCRWMRVHGKVPSHLLRSKITKPNVYHESKLAAKEYQAAKARLFTAFIKA
GLGAWVEKPTEQDQFSLT (SEQ ID NO: 23)
>DTA amino acid sequence
DPDDVVDSSKSFVMENFSSYHGTKPGYVDSIQKGIQKPKSGTQGNYDDDWKGFYST
DNKYDAAGYSVDNENPLSGKAGGVVKVTYPGLTKVLALKVDNAETIKKELGLSLTE
PLMEQVGTEEFIKRFGDGASRVVLSLPFAEGSSSVEYINNWEQAKALSVELEINFETR
GKRGQDAMYEYMAQACAGNRVRRSLCEGTLLLWCDIIGQTTYRDLKL (SEQ ID
NO: 24)
>BAX amino acid sequence
DGSGEQPRGGGPTSSEQIMKTGALLLQGFIQDRAGRMGGEAPELALDPVPQDASTKK
LSECLKRIGDELDSNMELQRMIAAVDTDSPREVFFRVAADMFSDGNFNWGRVVALF
YFASKLVLKALCTKVPELIRTIMGWTLDFLRERLLGWIQDQGGWDGLLSYFGTPTW
QTVTIFVAGVLTASLTIWKKMG (SEQ ID NO: 25)
>ZFTARELA_501_sensor (sensor stop codon lower case and underlined,
mismatches with target lower case, [c] insertion of a cytosine with respect to target):
CATAGAAGCCATCCCGGCAGTCCTTTCCTACAAGCTCGTGGGGGTGAGGCCGGTG
AGGAGGGTCCTTGGTGgCCAGGGAGATGCGCACTGTCCCTGGTCCTGTGTAGCCA
TTGgTCTTGgTGGTGGGGTGGGTCTTGGTGGTATCTGTGCTCCTCTCGCCTGGGAT
GCTGCCCGCGGAGCGCCCCTCGCACTTGTAGCGGAAGCGCATGCCCCGCTGCTtag
GCTGCTCAATGgTCTCCACAGCGGAGGC[c]CGAGTCCCGGCTCTTTCCGGTCAGGT
CCtagGGGGCGAGGTCATCGCCTGGTGGGGACTGGGTGAGCTCAGACAGCGCCTC
GGGCTGGCtCCtCCAGGCCTGCAGCAGGGCACTGCGCTGGGGGCCGCTGAGCCCC
AGGGAGCCAGGGTGCACCTCCAGCACGTGGGCACGGAgGTCGTCCAGGTGCAGG
CTGGGCAGTGCCCGGCCACAGGCCAgGCACACCAGCCGGTTCCCCCGCGGGTCAT
gGTCC (SEQ ID NO: 30)
>EWSRIFLI1_501_sensor (sensor stop codon lower case and underlined,
mismatches with target lower case):
GCCACCTCATCGGGGTCCGTCATTTTGAACTCCCCGTTGGTCCCCTCCCAGGTGA
TACAGCTGGCGTTGGCGCTGTCGGAGAGCAGCTCCAGGAGGAATTGCCACAGCT
GGATCTGCCCGCTTCCAGGGTTGGCTAGGCGACTGCTGGTCGGGCCCAGGATCTG
gTACGGATCTGGCtagGGCCGTTGCTCTGTATTCTTACTGATCGTTTGTGCCCCTCC
AAGGGGAGGACTTTTGTTGAGGCCAGAATTCATGTTATTGCCCCAAGCTCCTCTT
CTGACTGAGTCATAcGAAGGGTTCTGCTGCCCGTgGCTGCTGCTCTGTtagCTATAT
TGgCTTGGAGCTTGGCTGTgGGATCCAGTTTGGGGTGGGTAcCTAGTGGGAGGCTG
CTGCCCATgGCTGCTTTGTTGgCCATgGCTACTCTGCTGTCCATgGCTGCTCGGTTG
CCCATgGGTGTTCTGCTGgGAGTAcCTGCTCTGgTCATAcCTAGTCGGCTGTGTA
(SEQ ID NO: 33)
>EBNA1_501_sensor (sensor stop codon lower case and underlined, mismatches
with target in lowercase):
ATCACCTCCTTCATCTCCGTCATCTCCGTCATCACCCTCCGCGGCAGCCCCTTCCA
CCATAGGTGGAAACCAGGGAGGCAAATCTACTCCATCGTCAAAGCTGCACACAG
TCACCCTGATATTGCAGGTAGGAGCGGGCTTTGTCATAACAAGGTCCTTAATCGC
ATCCTTCAAAACCTCAGCAAATATATGAGTTTGTAcAAAGACCATGAAATAACAG
ACAATGGACTCCCTTgGCGGGCCAGGTTGtagGCCGGGTCCAGGGGCCATTCCAAA
GGGGAGACGACTCAATGGTGTAAGACGACATTGTGGAATAGCAAGGGCAGTTCC
TCGCCTTgGGTTGTAAAGGGAGGTCTTACTACCTCCATATACGAACACACCGGCG
ACCCAAGTTCCTTCGTCGGTAGTCCTTTCTACGTGACTCCTAGCCAGGAGAGCTC
TTAcACCTTCTGCAATGTTCTCAAATTTCGGGTTGGAACCTCCTTGACCACGAgGC
TTTCC (SEQ ID NO: 34)
>KSHV_ORF71_501_sensor (sensor stop codon lower case and underlined,
mismatches with target in lowercase):
TGTTGGGAGTGTGATGGGCCGGAAAGGTGgAGGCCCATTAGGGTTTGCACTTGGC
GCTGTAGGTCTACTCTTGACAAAGATCTAAGCATTGACATTAGGGCATCCACGTC
AGTGGGACCCAGTAGGTCTAAGTTTTCCATACAGTACACCCAGTGTAAGAATGTC
TGTGGTGTGCTGCGAGACCCTATAGTGTCCTTGCTTAAAAATATCAAAGACCTAA
TATCCCTCGCACACAGCTCCCCGTCTACGtagAGAACAGTGAGCTGgTAcGGGCTG
AAATAcCTCATTGTGCCCGCTAGGTGGCGCTCTAAAAAACGCGGGTCTAAGTGgA
GCAGGTCGCGCAAGAGGTCTCTGCGACCTGCACGAAACAGACATTCCGCTAACA
GGGGAAACGTTAACCTGCCCTCCTCCTTTAAAGCTCTAAGAGCTCCAATTAATTG
GGCCAGTGTGGGTTGgGGTAgGAACACGTTTAGGAGGAACAATACCACTTCCCTG
TCATCC (SEQ ID NO: 35)
>MCP-ADARdd(E488Q)-Sensor(CBFA2T3GLIS2_495)-E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCACTTCTCGGGCTTGACAGGGTAATCGTTGACAGGGTCCACCAGGTCTT
GCAGGAGCTCAAAGAGCTGGTTACACTTGGCCCAGCGACACACCAGCTGCTTGG
GCAGGGGCAGGTCTGGCGAGAGGCACTTGTCCTTGGGAGGGGTAAGGAAGGAG
GAGGCAGGCAGGTGCAGGGCCCCCCCGGAGCCGAGGGGCAGGAAGAACTGGAA
GGAGCTGGGGACACCATCCAAATAGCGCAGTAGCTGGAAGGTCCTCGCTAGAGT
CCTCCTGCTGGTTGGTGGCCGTCAGGGCGTCCTCGGAGGCCTGCCGCTTCGCCTC
GGCCAGGGCCCGCTCCATCTTGGCACGCTCCGTGGTGGTGGGCTCGTGCGCTTTG
CGCTCCGCGTCCGACACGGCTTTCTGCAGCTCCGACATGGCCTGCCGCTTCACCT
CATTCACGGCCTCTTCAGCCTTCCTCCAGATGTCCTCAGGCACGTAGCCGGTGGG
GGTCCTCGGCAGGAGGCCGGCCAGGCTCGGGCCAGTGTACTAATTATGCTCTCTT
GAAATTGGCTGGAGATGTTGAGAGCAACCCAGGTCCCTTAATTAAGGTGGACAT
CATCAGCGTGGCTCTGAAGAGGCACTCCACCAAGGCTTTCGACGCTTCCAAGAA
ACTGACCCCTGAACAGGCCGAGCAGATCAAGACCCTGCTCCAGTACAGCCCTAG
CTCCCAGAACAGCCAGCCTTGGCACTTCATCGTGGCTAGCACCGAGGAAGGCAA
AGCTAGGGTGGCTAAGAGCGCCGCTGGCAACTACGTGTTCAGCGAGAGGAAGAT
GCTGGATGCTAGCCACGTGGTGGTGTTCTGCGCTAAGACCGCCATGGACGATGTG
TGGCTGAAGCTGGTGGTGGATCAGGAAGATGCTGATGGCAGGTTCGCTACCCCT
GAAGCTAAGGCCGCTAACGACAAGGGCAGGAAGTTCACTGCCGACATGCACAGG
AAGGATCTGCACGATGATGCTGAGTGGATGGCCAAGCAGGTGTACCTGAACGTG
GGCAACTTCCTGCTCGGCGTGGCTGCCCTGGGCCTCGATGCTGTGCCCATCGAAG
GCTTCGATGCTGCTATCCTGGATGCCGAGTTCGGCCTGAAGGAGAAAGGCTACAC
CAGCCTGGTGGTGGTGCCTGTGGGCCACCACAGCGTGGAGGACTTCAACGCTAC
CCTGCCTAAGAGCAGGCTGCCCCAGAACATCACCCTGACCGAGGTGTGATTAATT
AAAAGGGCGGATCCGGTCTCCAGATGGCCAACATGAGGATCACCCATGTCTGCA
GGGCCAGATGGCCAACATGAGGATCACCCATGTCTGCAGGGCCAGATGGCCAAC
ATGAGGATCACCCATGTCTGCAGGGCCAGATGGCCAACATGAGGATCACCCATG
TCTGCAGGGCCAGATGGCCAACATGAGGATCACCCATGTCTGCAGGGCCAGATG
GCCAACATGAGGATCACCCATGTCTGCAGGGCCAGATGGCCAACATGAGGATCA
CCCATGTCTGCAGGGCCAGATGGCCAACATGAGGATCACCCATGTCTGCAGGGC
CAGATGGCCAACATGAGGATCACCCATGTCTGCAGGGCCAGATAGATCTCAATT
G (SEQ ID NO: 38)
>MCP-ADARddm(C377F, E488Q)-Sensor(CBFA2T3GLIS2_495)-E2A-NTR1.1-
9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCACTTCTCGGGCTTGACAGGGTAATCGTTGACAGGGTCCACCAGGTCTTG
CAGGAGCTCAAAGAGCTGGTTACACTTGGCCCAGCGACACACCAGCTGCTTGGG
CAGGGGCAGGTCTGGCGAGAGGCACTTGTCCTTGGGAGGGGTAAGGAAGGAGG
AGGCAGGCAGGTGCAGGGCCCCCCCGGAGCCGAGGGGCAGGAAGAACTGGAAG
GAGCTGGGGACACCATCCAAATAGCGCAGTAGCTGGAAGGTCCTCGCTAGAGTC
CTCCTGCTGGTTGGTGGCCGTCAGGGCGTCCTCGGAGGCCTGCCGCTTCGCCTCG
GCCAGGGCCCGCTCCATCTTGGCACGCTCCGTGGTGGTGGGCTCGTGCGCTTTGC
GCTCCGCGTCCGACACGGCTTTCTGCAGCTCCGACATGGCCTGCCGCTTCACCTC
ATTCACGGCCTCTTCAGCCTTCCTCCAGATGTCCTCAGGCACGTAGCCGGTGGGG
GTCCTCGGCAGGAGGCCGGCCAGGCTCGGGCCAGTGTACTAATTATGCTCTCTTG
AAATTGGCTGGAGATGTTGAGAGCAACCCAGGTCCCTTAATTAAGGTGGACATC
ATCAGCGTGGCTCTGAAGAGGCACTCCACCAAGGCTTTCGACGCTTCCAAGAAA
CTGACCCCTGAACAGGCCGAGCAGATCAAGACCCTGCTCCAGTACAGCCCTAGC
TCCCAGAACAGCCAGCCTTGGCACTTCATCGTGGCTAGCACCGAGGAAGGCAAA
GCTAGGGTGGCTAAGAGCGCCGCTGGCAACTACGTGTTCAGCGAGAGGAAGATG
CTGGATGCTAGCCACGTGGTGGTGTTCTGCGCTAAGACCGCCATGGACGATGTGT
GGCTGAAGCTGGTGGTGGATCAGGAAGATGCTGATGGCAGGTTCGCTACCCCTG
AAGCTAAGGCCGCTAACGACAAGGGCAGGAAGTTCACTGCCGACATGCACAGGA
AGGATCTGCACGATGATGCTGAGTGGATGGCCAAGCAGGTGTACCTGAACGTGG
GCAACTTCCTGCTCGGCGTGGCTGCCCTGGGCCTCGATGCTGTGCCCATCGAAGG
CTTCGATGCTGCTATCCTGGATGCCGAGTTCGGCCTGAAGGAGAAAGGCTACACC
AGCCTGGTGGTGGTGCCTGTGGGCCACCACAGCGTGGAGGACTTCAACGCTACC
CTGCCTAAGAGCAGGCTGCCCCAGAACATCACCCTGACCGAGGTGTGATTAATTA
AAAGGGCGGATCCGGTCTCCAGATGGCCAACATGAGGATCACCCATGTCTGCAG
GGCCAGATGGCCAACATGAGGATCACCCATGTCTGCAGGGCCAGATGGCCAACA
TGAGGATCACCCATGTCTGCAGGGCCAGATGGCCAACATGAGGATCACCCATGT
CTGCAGGGCCAGATGGCCAACATGAGGATCACCCATGTCTGCAGGGCCAGATGG
CCAACATGAGGATCACCCATGTCTGCAGGGCCAGATGGCCAACATGAGGATCAC
CCATGTCTGCAGGGCCAGATGGCCAACATGAGGATCACCCATGTCTGCAGGGCC
AGATGGCCAACATGAGGATCACCCATGTCTGCAGGGCCAGATAGATCTCAATTG
(SEQ ID NO: 39)
>MCP-ADARdd(E488Q)-Sensor(EML4ALK_501)-E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCATCGTTGGGCATTCCGGACACCTGGCCTTCATACACCTCCCCAAAGGC
GCCATGGCCCAGACCCCGAATGAGGGTGATGTTTTTCCGCGGCACCTCCTTCAGG
TCACTGATGGAGGAGGTCTTGCCAGCAAAGCAGTGGTTGGGGTTGTGGTCGGTC
ATGATGGTCGAGGTGCGGAGCTTGCTCAGCTTGTACTCAGGGCTCTGCAGCTCCA
TCTGCATGGCTTGCAGCTCCTAGTGCTTCCGGCGGTACACTTGGGTCCTTTCCCAG
GTGTAGGCTCTACAGTGGTTTTGCTCCATATACGCATGGCTCCACCTGAGTCTCC
AGTACGAACATCTCCATTCCCCAAGAAGGCTAAACACTGCACAAATTTTGGCTTT
TCATATTTCCCAAAAATTCCCTGTTTTCTTGTTAGTGAATTGCCGCTCCAGGTCCA
GAAGAAAATATGAGATTTACCGCAGGTACTTATGGTATTTGCATCTGTTGGGTGA
AACTCCACAGCCAGGCCGGCCAGGCTCGGGCCAGTGTACTAATTATGCTCTCTTG
AAATTGGCTGGAGATGTTGAGAGCAACCCAGGTCCCTTAATTAAGGTGGACATC
ATCAGCGTGGCTCTGAAGAGGCACTCCACCAAGGCTTTCGACGCTTCCAAGAAA
CTGACCCCTGAACAGGCCGAGCAGATCAAGACCCTGCTCCAGTACAGCCCTAGC
TCCCAGAACAGCCAGCCTTGGCACTTCATCGTGGCTAGCACCGAGGAAGGCAAA
GCTAGGGTGGCTAAGAGCGCCGCTGGCAACTACGTGTTCAGCGAGAGGAAGATG
CTGGATGCTAGCCACGTGGTGGTGTTCTGCGCTAAGACCGCCATGGACGATGTGT
GGCTGAAGCTGGTGGTGGATCAGGAAGATGCTGATGGCAGGTTCGCTACCCCTG
AAGCTAAGGCCGCTAACGACAAGGGCAGGAAGTTCACTGCCGACATGCACAGGA
AGGATCTGCACGATGATGCTGAGTGGATGGCCAAGCAGGTGTACCTGAACGTGG
GCAACTTCCTGCTCGGCGTGGCTGCCCTGGGCCTCGATGCTGTGCCCATCGAAGG
CTTCGATGCTGCTATCCTGGATGCCGAGTTCGGCCTGAAGGAGAAAGGCTACACC
AGCCTGGTGGTGGTGCCTGTGGGCCACCACAGCGTGGAGGACTTCAACGCTACC
CTGCCTAAGAGCAGGCTGCCCCAGAACATCACCCTGACCGAGGTGTGATTAATTA
AAAGGGCGGATCCGGTCTCCAGATGGCCAACATGAGGATCACCCATGTCTGCAG
GGCCAGATGGCCAACATGAGGATCACCCATGTCTGCAGGGCCAGATGGCCAACA
TGAGGATCACCCATGTCTGCAGGGCCAGATGGCCAACATGAGGATCACCCATGT
CTGCAGGGCCAGATGGCCAACATGAGGATCACCCATGTCTGCAGGGCCAGATGG
CCAACATGAGGATCACCCATGTCTGCAGGGCCAGATGGCCAACATGAGGATCAC
CCATGTCTGCAGGGCCAGATGGCCAACATGAGGATCACCCATGTCTGCAGGGCC
AGATGGCCAACATGAGGATCACCCATGTCTGCAGGGCCAGATAGATCTCAATTG
(SEQ ID NO: 44)
>MCP-ADARdd(E488Q)-Sensor(ZFTARELA_501)-E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCACATAGAAGCCATCCCGGCAGTCCTTTCCTACAAGCTCGTGGGGGTG
AGGCCGGTGAGGAGGGTCCTTGGTGGCCAGGGAGATGCGCACTGTCCCTGGTCC
TGTGTAGCCATTGGTCTTGGTGGTGGGGTGGGTCTTGGTGGTATCTGTGCTCCTCT
CGCCTGGGATGCTGCCCGCGGAGCGCCCCTCGCACTTGTAGCGGAAGCGCATGC
CCCGCTGCTTAGGCTGCTCAATGGTCTCCACAGCGGAGGCCCGAGTCCCGGCTCT
TTCCGGTCAGGTCCTAGGGGGCGAGGTCATCGCCTGGTGGGGACTGGGTGAGCT
CAGACAGCGCCTCGGGCTGGCTCCTCCAGGCCTGCAGCAGGGCACTGCGCTGGG
GGCCGCTGAGCCCCAGGGAGCCAGGGTGCACCTCCAGCACGTGGGCACGGAGGT
CGTCCAGGTGCAGGCTGGGCAGTGCCCGGCCACAGGCCAGGCACACCAGCCGGT
TCCCCCGCGGGTCATGGTCCGGCCGGCCAGGCTCGGGCCAGTGTACTAATTATGC
TCTCTTGAAATTGGCTGGAGATGTTGAGAGCAACCCAGGTCCCTTAATTAAGGTG
GACATCATCAGCGTGGCTCTGAAGAGGCACTCCACCAAGGCTTTCGACGCTTCCA
AGAAACTGACCCCTGAACAGGCCGAGCAGATCAAGACCCTGCTCCAGTACAGCC
CTAGCTCCCAGAACAGCCAGCCTTGGCACTTCATCGTGGCTAGCACCGAGGAAG
GCAAAGCTAGGGTGGCTAAGAGCGCCGCTGGCAACTACGTGTTCAGCGAGAGGA
AGATGCTGGATGCTAGCCACGTGGTGGTGTTCTGCGCTAAGACCGCCATGGACG
ATGTGTGGCTGAAGCTGGTGGTGGATCAGGAAGATGCTGATGGCAGGTTCGCTA
CCCCTGAAGCTAAGGCCGCTAACGACAAGGGCAGGAAGTTCACTGCCGACATGC
ACAGGAAGGATCTGCACGATGATGCTGAGTGGATGGCCAAGCAGGTGTACCTGA
ACGTGGGCAACTTCCTGCTCGGCGTGGCTGCCCTGGGCCTCGATGCTGTGCCCAT
CGAAGGCTTCGATGCTGCTATCCTGGATGCCGAGTTCGGCCTGAAGGAGAAAGG
CTACACCAGCCTGGTGGTGGTGCCTGTGGGCCACCACAGCGTGGAGGACTTCAA
CGCTACCCTGCCTAAGAGCAGGCTGCCCCAGAACATCACCCTGACCGAGGTGTG
ATTAATTAAAAGGGCGGATCCGGTCTCCAGATGGCCAACATGAGGATCACCCAT
GTCTGCAGGGCCAGATGGCCAACATGAGGATCACCCATGTCTGCAGGGCCAGAT
GGCCAACATGAGGATCACCCATGTCTGCAGGGCCAGATGGCCAACATGAGGATC
ACCCATGTCTGCAGGGCCAGATGGCCAACATGAGGATCACCCATGTCTGCAGGG
CCAGATGGCCAACATGAGGATCACCCATGTCTGCAGGGCCAGATGGCCAACATG
AGGATCACCCATGTCTGCAGGGCCAGATGGCCAACATGAGGATCACCCATGTCT
GCAGGGCCAGATGGCCAACATGAGGATCACCCATGTCTGCAGGGCCAGATAGAT
CTCAATTG (SEQ ID NO: 45)
>MCP-ADARdd(E488Q)-Sensor(EWSRIFLI1_501)-E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCAGCCACCTCATCGGGGTCCGTCATTTTGAACTCCCCGTTGGTCCCCTC
CCAGGTGATACAGCTGGCGTTGGCGCTGTCGGAGAGCAGCTCCAGGAGGAATTG
CCACAGCTGGATCTGCCCGCTTCCAGGGTTGGCTAGGCGACTGCTGGTCGGGCCC
AGGATCTGGTACGGATCTGGCTAGGGCCGTTGCTCTGTATTCTTACTGATCGTTTG
TGCCCCTCCAAGGGGAGGACTTTTGTTGAGGCCAGAATTCATGTTATTGCCCCAA
GCTCCTCTTCTGACTGAGTCATACGAAGGGTTCTGCTGCCCGTGGCTGCTGCTCTG
TTAGCTATATTGGCTTGGAGCTTGGCTGTGGGATCCAGTTTGGGGTGGGTACCTA
GTGGGAGGCTGCTGCCCATGGCTGCTTTGTTGGCCATGGCTACTCTGCTGTCCAT
GGCTGCTCGGTTGCCCATGGGTGTTCTGCTGGGAGTACCTGCTCTGGTCATACCT
AGTCGGCTGTGTAGGCCGGCCAGGCTCGGGCCAGTGTACTAATTATGCTCTCTTG
AAATTGGCTGGAGATGTTGAGAGCAACCCAGGTCCCTTAATTAAGGTGGACATC
ATCAGCGTGGCTCTGAAGAGGCACTCCACCAAGGCTTTCGACGCTTCCAAGAAA
CTGACCCCTGAACAGGCCGAGCAGATCAAGACCCTGCTCCAGTACAGCCCTAGC
TCCCAGAACAGCCAGCCTTGGCACTTCATCGTGGCTAGCACCGAGGAAGGCAAA
GCTAGGGTGGCTAAGAGCGCCGCTGGCAACTACGTGTTCAGCGAGAGGAAGATG
CTGGATGCTAGCCACGTGGTGGTGTTCTGCGCTAAGACCGCCATGGACGATGTGT
GGCTGAAGCTGGTGGTGGATCAGGAAGATGCTGATGGCAGGTTCGCTACCCCTG
AAGCTAAGGCCGCTAACGACAAGGGCAGGAAGTTCACTGCCGACATGCACAGGA
AGGATCTGCACGATGATGCTGAGTGGATGGCCAAGCAGGTGTACCTGAACGTGG
GCAACTTCCTGCTCGGCGTGGCTGCCCTGGGCCTCGATGCTGTGCCCATCGAAGG
CTTCGATGCTGCTATCCTGGATGCCGAGTTCGGCCTGAAGGAGAAAGGCTACACC
AGCCTGGTGGTGGTGCCTGTGGGCCACCACAGCGTGGAGGACTTCAACGCTACC
CTGCCTAAGAGCAGGCTGCCCCAGAACATCACCCTGACCGAGGTGTGATTAATTA
AAAGGGCGGATCCGGTCTCCAGATGGCCAACATGAGGATCACCCATGTCTGCAG
GGCCAGATGGCCAACATGAGGATCACCCATGTCTGCAGGGCCAGATGGCCAACA
TGAGGATCACCCATGTCTGCAGGGCCAGATGGCCAACATGAGGATCACCCATGT
CTGCAGGGCCAGATGGCCAACATGAGGATCACCCATGTCTGCAGGGCCAGATGG
CCAACATGAGGATCACCCATGTCTGCAGGGCCAGATGGCCAACATGAGGATCAC
CCATGTCTGCAGGGCCAGATGGCCAACATGAGGATCACCCATGTCTGCAGGGCC
AGATGGCCAACATGAGGATCACCCATGTCTGCAGGGCCAGATAGATCTCAATTG
(SEQ ID NO: 48)
>MCP-ADARddm(C377F, E488Q)-Sensor(CBFA2T3GLIS2_495)-E2A-DTA-
9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCACTTCTCGGGCTTGACAGGGTAATCGTTGACAGGGTCCACCAGGTCTTG
CAGGAGCTCAAAGAGCTGGTTACACTTGGCCCAGCGACACACCAGCTGCTTGGG
CAGGGGCAGGTCTGGCGAGAGGCACTTGTCCTTGGGAGGGGTAAGGAAGGAGG
AGGCAGGCAGGTGCAGGGCCCCCCCGGAGCCGAGGGGCAGGAAGAACTGGAAG
GAGCTGGGGACACCATCCAAATAGCGCAGTAGCTGGAAGGTCCTCGCTAGAGTC
CTCCTGCTGGTTGGTGGCCGTCAGGGCGTCCTCGGAGGCCTGCCGCTTCGCCTCG
GCCAGGGCCCGCTCCATCTTGGCACGCTCCGTGGTGGTGGGCTCGTGCGCTTTGC
GCTCCGCGTCCGACACGGCTTTCTGCAGCTCCGACATGGCCTGCCGCTTCACCTC
ATTCACGGCCTCTTCAGCCTTCCTCCAGATGTCCTCAGGCACGTAGCCGGTGGGG
GTCCTCGGCAGGAGGCCGGCCAGGCTCGGGCCAGTGTACTAATTATGCTCTCTTG
AAATTGGCTGGAGATGTTGAGAGCAACCCAGGTCCCTTAATTAAGGACCCTGAT
GATGTTGTTGATTCTTCTAAATCTTTTGTGATGGAAAACTTTTCTTCGTACCACGG
GACTAAACCTGGTTATGTAGATTCCATTCAAAAAGGTATACAAAAGCCAAAATCT
GGTACACAAGGAAATTATGACGATGATTGGAAAGGGTTTTATAGTACCGACAAT
AAATACGACGCTGCGGGATACTCTGTAGATAATGAAAACCCGCTCTCTGGAAAA
GCTGGAGGCGTGGTCAAAGTGACGTATCCAGGACTGACGAAGGTTCTCGCACTA
AAAGTGGATAATGCCGAAACTATTAAGAAAGAGTTAGGTTTAAGTCTCACTGAA
CCGTTGATGGAGCAAGTCGGAACGGAAGAGTTTATCAAAAGGTTCGGTGATGGT
GCTTCGCGTGTAGTGCTCAGCCTTCCCTTCGCTGAGGGGAGTTCTAGCGTTGAAT
ATATTAATAACTGGGAACAGGCGAAAGCGTTAAGCGTAGAACTTGAGATTAATT
TTGAAACCCGTGGAAAACGTGGCCAAGATGCGATGTATGAGTATATGGCTCAAG
CCTGTGCAGGAAATCGTGTCAGGCGATCTCTTTGTGAAGGAACCTTACTTCTGTG
GTGTGACATAATTGGACAAACTACCTACAGAGATTTAAAGCTCTAATTAATTAAA
AGGGCGGATCCGGTCTCCAGATGGCCAACATGAGGATCACCCATGTCTGCAGGG
CCAGATGGCCAACATGAGGATCACCCATGTCTGCAGGGCCAGATGGCCAACATG
AGGATCACCCATGTCTGCAGGGCCAGATGGCCAACATGAGGATCACCCATGTCT
GCAGGGCCAGATGGCCAACATGAGGATCACCCATGTCTGCAGGGCCAGATGGCC
AACATGAGGATCACCCATGTCTGCAGGGCCAGATGGCCAACATGAGGATCACCC
ATGTCTGCAGGGCCAGATGGCCAACATGAGGATCACCCATGTCTGCAGGGCCAG
ATGGCCAACATGAGGATCACCCATGTCTGCAGGGCCAGATAGATCTCAATTG
(SEQ ID NO: 49)
>MCP-ADARdd(E488Q)-Sensor(CBFA2T3GLIS2_495)-E2A-BAX-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCACTTCTCGGGCTTGACAGGGTAATCGTTGACAGGGTCCACCAGGTCTT
GCAGGAGCTCAAAGAGCTGGTTACACTTGGCCCAGCGACACACCAGCTGCTTGG
GCAGGGGCAGGTCTGGCGAGAGGCACTTGTCCTTGGGAGGGGTAAGGAAGGAG
GAGGCAGGCAGGTGCAGGGCCCCCCCGGAGCCGAGGGGCAGGAAGAACTGGAA
GGAGCTGGGGACACCATCCAAATAGCGCAGTAGCTGGAAGGTCCTCGCTAGAGT
CCTCCTGCTGGTTGGTGGCCGTCAGGGCGTCCTCGGAGGCCTGCCGCTTCGCCTC
GGCCAGGGCCCGCTCCATCTTGGCACGCTCCGTGGTGGTGGGCTCGTGCGCTTTG
CGCTCCGCGTCCGACACGGCTTTCTGCAGCTCCGACATGGCCTGCCGCTTCACCT
CATTCACGGCCTCTTCAGCCTTCCTCCAGATGTCCTCAGGCACGTAGCCGGTGGG
GGTCCTCGGCAGGAGGCCGGCCAGGCTCGGGCCAGTGTACTAATTATGCTCTCTT
GAAATTGGCTGGAGATGTTGAGAGCAACCCAGGTCCCTTAATTAAGGACGGGTC
CGGGGAGCAGCCCAGAGGCGGGGGGCCCACCAGCTCTGAGCAGATCATGAAGA
CAGGGGCCCTTTTGCTTCAGGGTTTCATCCAGGATCGAGCAGGGCGAATGGGGG
GGGAGGCACCCGAGCTGGCCCTGGACCCGGTGCCTCAGGATGCGTCCACCAAGA
AGCTGAGCGAGTGTCTCAAGCGCATCGGGGACGAACTGGACAGTAACATGGAGC
TGCAGAGGATGATTGCCGCCGTGGACACAGACTCCCCCCGAGAGGTCTTTTTCCG
AGTGGCAGCTGACATGTTTTCTGACGGCAACTTCAACTGGGGCCGGGTTGTCGCC
CTTTTCTACTTTGCCAGCAAACTGGTGCTCAAGGCCCTGTGCACCAAGGTGCCGG
AACTGATCAGAACCATCATGGGCTGGACATTGGACTTCCTCCGGGAGCGGCTGTT
GGGCTGGATCCAAGACCAGGGTGGTTGGGACGGCCTCCTCTCCTACTTTGGGACG
CCCACGTGGCAGACCGTGACCATCTTTGTGGCGGGAGTGCTCACCGCCTCACTCA
CCATCTGGAAGAAGATGGGCTGATTAATTAAAAGGGCGGATCCGGTCTCCAGAT
GGCCAACATGAGGATCACCCATGTCTGCAGGGCCAGATGGCCAACATGAGGATC
ACCCATGTCTGCAGGGCCAGATGGCCAACATGAGGATCACCCATGTCTGCAGGG
CCAGATGGCCAACATGAGGATCACCCATGTCTGCAGGGCCAGATGGCCAACATG
AGGATCACCCATGTCTGCAGGGCCAGATGGCCAACATGAGGATCACCCATGTCT
GCAGGGCCAGATGGCCAACATGAGGATCACCCATGTCTGCAGGGCCAGATGGCC
AACATGAGGATCACCCATGTCTGCAGGGCCAGATGGCCAACATGAGGATCACCC
ATGTCTGCAGGGCCAGATAGATCTCAATTG (SEQ ID NO: 50)
>MCP-ADARdd(E488Q)-Sensor(CBFAT3GLIS2_495)-XTEN80-BAX-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCACTTCTCGGGCTTGACAGGGTAATCGTTGACAGGGTCCACCAGGTCTT
GCAGGAGCTCAAAGAGCTGGTTACACTTGGCCCAGCGACACACCAGCTGCTTGG
GCAGGGGCAGGTCTGGCGAGAGGCACTTGTCCTTGGGAGGGGTAAGGAAGGAG
GAGGCAGGCAGGTGCAGGGCCCCCCCGGAGCCGAGGGGCAGGAAGAACTGGAA
GGAGCTGGGGACACCATCCAAATAGCGCAGTAGCTGGAAGGTCCTCGCTAGAGT
CCTCCTGCTGGTTGGTGGCCGTCAGGGCGTCCTCGGAGGCCTGCCGCTTCGCCTC
GGCCAGGGCCCGCTCCATCTTGGCACGCTCCGTGGTGGTGGGCTCGTGCGCTTTG
CGCTCCGCGTCCGACACGGCTTTCTGCAGCTCCGACATGGCCTGCCGCTTCACCT
CATTCACGGCCTCTTCAGCCTTCCTCCAGATGTCCTCAGGCACGTAGCCGGTGGG
GGTCCTCGGCAGGAGGCCGGCCAGGCTCGGGCCAGGGAGGGCCGTCATCTGGTG
CTCCTCCTCCGTCAGGTGGCTCACCTGCTGGTTCCCCGACATCAACTGAGGAAGG
AACTAGCGAAAGTGCGACGCCTGAGAGTGGTCCCGGTACTAGCACTGAACCGTC
AGAGGGGAGTGCACCAGGCAGCCCCGCCGGCTCTCCAACTTCCACGGAGGAGGG
GACATCTACTGAGCCTTCTGAGGGTTCCGCACCTGGAACCAGTACTGAGCCCTCC
GAGCCTAGGTTAATTAAGGACGGGTCCGGGGAGCAGCCCAGAGGCGGGGGGCCC
ACCAGCTCTGAGCAGATCATGAAGACAGGGGCCCTTTTGCTTCAGGGTTTCATCC
AGGATCGAGCAGGGCGAATGGGGGGGGAGGCACCCGAGCTGGCCCTGGACCCG
GTGCCTCAGGATGCGTCCACCAAGAAGCTGAGCGAGTGTCTCAAGCGCATCGGG
GACGAACTGGACAGTAACATGGAGCTGCAGAGGATGATTGCCGCCGTGGACACA
GACTCCCCCCGAGAGGTCTTTTTCCGAGTGGCAGCTGACATGTTTTCTGACGGCA
ACTTCAACTGGGGCCGGGTTGTCGCCCTTTTCTACTTTGCCAGCAAACTGGTGCTC
AAGGCCCTGTGCACCAAGGTGCCGGAACTGATCAGAACCATCATGGGCTGGACA
TTGGACTTCCTCCGGGAGCGGCTGTTGGGCTGGATCCAAGACCAGGGTGGTTGGG
ACGGCCTCCTCTCCTACTTTGGGACGCCCACGTGGCAGACCGTGACCATCTTTGT
GGCGGGAGTGCTCACCGCCTCACTCACCATCTGGAAGAAGATGGGCTGATTAATT
AAAAGGGCGGATCCGGTCTCCAGATGGCCAACATGAGGATCACCCATGTCTGCA
GGGCCAGATGGCCAACATGAGGATCACCCATGTCTGCAGGGCCAGATGGCCAAC
ATGAGGATCACCCATGTCTGCAGGGCCAGATGGCCAACATGAGGATCACCCATG
TCTGCAGGGCCAGATGGCCAACATGAGGATCACCCATGTCTGCAGGGCCAGATG
GCCAACATGAGGATCACCCATGTCTGCAGGGCCAGATGGCCAACATGAGGATCA
CCCATGTCTGCAGGGCCAGATGGCCAACATGAGGATCACCCATGTCTGCAGGGC
CAGATGGCCAACATGAGGATCACCCATGTCTGCAGGGCCAGATAGATCTCAATT
G (SEQ ID NO: 51)
>MCP-ADARdd(E488Q)-Sensor(EBNA1_501)-XTEN80-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCAATCACCTCCTTCATCTCCGTCATCTCCGTCATCACCCTCCGCGGCAG
CCCCTTCCACCATAGGTGGAAACCAGGGAGGCAAATCTACTCCATCGTCAAAGC
TGCACACAGTCACCCTGATATTGCAGGTAGGAGCGGGCTTTGTCATAACAAGGTC
CTTAATCGCATCCTTCAAAACCTCAGCAAATATATGAGTTTGTACAAAGACCATG
AAATAACAGACAATGGACTCCCTTGGCGGGCCAGGTTGTAGGCCGGGTCCAGGG
GCCATTCCAAAGGGGAGACGACTCAATGGTGTAAGACGACATTGTGGAATAGCA
AGGGCAGTTCCTCGCCTTGGGTTGTAAAGGGAGGTCTTACTACCTCCATATACGA
ACACACCGGCGACCCAAGTTCCTTCGTCGGTAGTCCTTTCTACGTGACTCCTAGC
CAGGAGAGCTCTTACACCTTCTGCAATGTTCTCAAATTTCGGGTTGGAACCTCCTT
GACCACGAGGCTTTCCGGCCGGCCAGGCTCGGGCCAGGGAGGGCCGTCATCTGG
TGCTCCTCCTCCGTCAGGTGGCTCACCTGCTGGTTCCCCGACATCAACTGAGGAA
GGAACTAGCGAAAGTGCGACGCCTGAGAGTGGTCCCGGTACTAGCACTGAACCG
TCAGAGGGGAGTGCACCAGGCAGCCCCGCCGGCTCTCCAACTTCCACGGAGGAG
GGGACATCTACTGAGCCTTCTGAGGGTTCCGCACCTGGAACCAGTACTGAGCCCT
CCGAGCCTAGGTTAATTAAGGTGGACATCATCAGCGTGGCTCTGAAGAGGCACT
CCACCAAGGCTTTCGACGCTTCCAAGAAACTGACCCCTGAACAGGCCGAGCAGA
TCAAGACCCTGCTCCAGTACAGCCCTAGCTCCCAGAACAGCCAGCCTTGGCACTT
CATCGTGGCTAGCACCGAGGAAGGCAAAGCTAGGGTGGCTAAGAGCGCCGCTGG
CAACTACGTGTTCAGCGAGAGGAAGATGCTGGATGCTAGCCACGTGGTGGTGTT
CTGCGCTAAGACCGCCATGGACGATGTGTGGCTGAAGCTGGTGGTGGATCAGGA
AGATGCTGATGGCAGGTTCGCTACCCCTGAAGCTAAGGCCGCTAACGACAAGGG
CAGGAAGTTCACTGCCGACATGCACAGGAAGGATCTGCACGATGATGCTGAGTG
GATGGCCAAGCAGGTGTACCTGAACGTGGGCAACTTCCTGCTCGGCGTGGCTGCC
CTGGGCCTCGATGCTGTGCCCATCGAAGGCTTCGATGCTGCTATCCTGGATGCCG
AGTTCGGCCTGAAGGAGAAAGGCTACACCAGCCTGGTGGTGGTGCCTGTGGGCC
ACCACAGCGTGGAGGACTTCAACGCTACCCTGCCTAAGAGCAGGCTGCCCCAGA
ACATCACCCTGACCGAGGTGTGATTAATTAAAAGGGCGGATCCGGTCTCCAGAT
GGCCAACATGAGGATCACCCATGTCTGCAGGGCCAGATGGCCAACATGAGGATC
ACCCATGTCTGCAGGGCCAGATGGCCAACATGAGGATCACCCATGTCTGCAGGG
CCAGATGGCCAACATGAGGATCACCCATGTCTGCAGGGCCAGATGGCCAACATG
AGGATCACCCATGTCTGCAGGGCCAGATGGCCAACATGAGGATCACCCATGTCT
GCAGGGCCAGATGGCCAACATGAGGATCACCCATGTCTGCAGGGCCAGATGGCC
AACATGAGGATCACCCATGTCTGCAGGGCCAGATGGCCAACATGAGGATCACCC
ATGTCTGCAGGGCCAGATAGATCTCAATTG (SEQ ID NO: 52)
>MCP-ADARdd(E488Q)-Sensor(KSHV_ORF71_501)-XTEN80-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATGTATTAATGGT
GAATACATGAGTGATCGTGGCCTTGCATTAAATGACTGCCATGCAGAAATAATAT
CTCGGAGATCCTTGCTCAGATTTCTTTATACACAACTTGAGCTTTACTTAAATAAC
AAAGATGATCAAAAAAGATCCATCTTTCAGAAATCAGAGCGAGGGGGGTTTAGG
CTGAAGGAGAATGTCCAGTTTCATCTGTACATCAGCACCTCTCCCTGTGGAGATG
CCAGAATCTTCTCACCACATGAGCCAATCCTGGAAGAACCAGCAGATAGACACC
CAAATCGTAAAGCAAGAGGACAGCTACGGACCAAAATAGAGTCTGGTCAGGGG
ACGATTCCAGTGCGCTCCAATGCGAGCATCCAAACGTGGGACGGGGTGCTGCAA
GGGGAGCGGCTGCTCACCATGTCCTGCAGTGACAAGATTGCACGCTGGAACGTG
GTGGGCATCCAGGGATCACTGCTCAGCATTTTCGTGGAGCCCATTTACTTCTCGA
GCATCATCCTGGGCAGCCTTTACCACGGGGACCACCTTTCCAGGGCCATGTACCA
GCGGATCTCCAACATAGAGGACCTGCCACCTCTCTACACCCTCAACAAGCCTTTG
CTCAGTGGCATCAGCAATGCAGAAGCACGGCAGCCAGGGAAGGCCCCCAACTTC
AGTGTCAACTGGACGGTAGGCGACTCCGCTATTGAGGTCATCAACGCCACGACT
GGGAAGGATGAGCTGGGCCGCGCGTCCCGCCTGTGTAAGCACGCGTTGTACTGT
CGCTGGATGCGTGTGCACGGCAAGGTTCCCTCCCACTTACTACGCTCCAAGATTA
CCAAGCCCAACGTGTACCATGAGTCCAAGCTGGCGGCAAAGGAGTACCAGGCCG
CCAAGGCGCGTCTGTTCACAGCCTTCATCAAGGCGGGGCTGGGGGCCTGGGTGG
AGAAGCCCACCGAGCAGGACCAGTTCTCACTCACGACTAGTGGCAGCGGCGCCA
CAAACTTCTCTCTGCTAAAGCAAGCAGGTGATGTTGAAGAAAACCCCGGGCCTG
GCGCGCCATGTTGGGAGTGTGATGGGCCGGAAAGGTGGAGGCCCATTAGGGTTT
GCACTTGGCGCTGTAGGTCTACTCTTGACAAAGATCTAAGCATTGACATTAGGGC
ATCCACGTCAGTGGGACCCAGTAGGTCTAAGTTTTCCATACAGTACACCCAGTGT
AAGAATGTCTGTGGTGTGCTGCGAGACCCTATAGTGTCCTTGCTTAAAAATATCA
AAGACCTAATATCCCTCGCACACAGCTCCCCGTCTACGTAGAGAACAGTGAGCT
GGTACGGGCTGAAATACCTCATTGTGCCCGCTAGGTGGCGCTCTAAAAAACGCG
GGTCTAAGTGGAGCAGGTCGCGCAAGAGGTCTCTGCGACCTGCACGAAACAGAC
ATTCCGCTAACAGGGGAAACGTTAACCTGCCCTCCTCCTTTAAAGCTCTAAGAGC
TCCAATTAATTGGGCCAGTGTGGGTTGGGGTAGGAACACGTTTAGGAGGAACAA
TACCACTTCCCTGTCATCCGGCCGGCCAGGCTCGGGCCAGGGAGGGCCGTCATCT
GGTGCTCCTCCTCCGTCAGGTGGCTCACCTGCTGGTTCCCCGACATCAACTGAGG
AAGGAACTAGCGAAAGTGCGACGCCTGAGAGTGGTCCCGGTACTAGCACTGAAC
CGTCAGAGGGGAGTGCACCAGGCAGCCCCGCCGGCTCTCCAACTTCCACGGAGG
AGGGGACATCTACTGAGCCTTCTGAGGGTTCCGCACCTGGAACCAGTACTGAGCC
CTCCGAGCCTAGGTTAATTAAGGTGGACATCATCAGCGTGGCTCTGAAGAGGCA
CTCCACCAAGGCTTTCGACGCTTCCAAGAAACTGACCCCTGAACAGGCCGAGCA
GATCAAGACCCTGCTCCAGTACAGCCCTAGCTCCCAGAACAGCCAGCCTTGGCA
CTTCATCGTGGCTAGCACCGAGGAAGGCAAAGCTAGGGTGGCTAAGAGCGCCGC
TGGCAACTACGTGTTCAGCGAGAGGAAGATGCTGGATGCTAGCCACGTGGTGGT
GTTCTGCGCTAAGACCGCCATGGACGATGTGTGGCTGAAGCTGGTGGTGGATCA
GGAAGATGCTGATGGCAGGTTCGCTACCCCTGAAGCTAAGGCCGCTAACGACAA
GGGCAGGAAGTTCACTGCCGACATGCACAGGAAGGATCTGCACGATGATGCTGA
GTGGATGGCCAAGCAGGTGTACCTGAACGTGGGCAACTTCCTGCTCGGCGTGGCT
GCCCTGGGCCTCGATGCTGTGCCCATCGAAGGCTTCGATGCTGCTATCCTGGATG
CCGAGTTCGGCCTGAAGGAGAAAGGCTACACCAGCCTGGTGGTGGTGCCTGTGG
GCCACCACAGCGTGGAGGACTTCAACGCTACCCTGCCTAAGAGCAGGCTGCCCC
AGAACATCACCCTGACCGAGGTGTGATTAATTAAAAGGGCGGATCCGGTCTCCA
GATGGCCAACATGAGGATCACCCATGTCTGCAGGGCCAGATGGCCAACATGAGG
ATCACCCATGTCTGCAGGGCCAGATGGCCAACATGAGGATCACCCATGTCTGCA
GGGCCAGATGGCCAACATGAGGATCACCCATGTCTGCAGGGCCAGATGGCCAAC
ATGAGGATCACCCATGTCTGCAGGGCCAGATGGCCAACATGAGGATCACCCATG
TCTGCAGGGCCAGATGGCCAACATGAGGATCACCCATGTCTGCAGGGCCAGATG
GCCAACATGAGGATCACCCATGTCTGCAGGGCCAGATGGCCAACATGAGGATCA
CCCATGTCTGCAGGGCCAGATAGATCTCAATTG (SEQ ID NO: 53)
MCP_ADARddm(C377F, E488Q)-P2A-Sensor(CCNH_C5orf30_sensor_501)-
E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCAGCATCGGGGAGCAGGGTGTGCTGGAGCAGCGGGGCTTCTCTGCCTCC
GCCTTTCCCGGGGAGTTGGCCTCAGCCCCAGGGAAGGGCAAGGTGGTCAGGGTA
CTTCTGGACTCTCCATTACTATCGACTTCCATTTTACGCACCAGTCTGCACAATCC
AGACTAAATCCCAGAAGATGTCACTGTACAATTTCCAATAGGTCCCAAGGGAAC
ACAGACATAATAATTTTTCTGTTCTACTCCAGGTAATTTTTCATTATATCTGGTAC
CTGTAGCAGGCAAGTTCTGTTCTCTTTCAGCATCAGACTCTCTGGTACATAACTTT
CCATAGTAATTCCAGCCCTGGAGGCACTAGATACAATGGCAGTCAGGGCAATTT
GGGAAGGTGTGTATACAAGGTAAGCATCCGTCAATGCAATTCTATTAAGAAAGT
CATCAGCTGTTTTCCTCAAAATCTCTGGATTCTCCAATATGGGATAGCGGGTCTTT
ACGTCGATGAGGGCCGGCCAGGCTCGGGCCAGTGTACTAATTATGCTCTCTTGAA
ATTGGCTGGAGATGTTGAGAGCAACCCAGGTCCCTTAATTAAGGTGGACATCATC
AGCGTGGCTCTGAAGAGGCACTCCACCAAGGCTTTCGACGCTTCCAAGAAACTG
ACCCCTGAACAGGCCGAGCAGATCAAGACCCTGCTCCAGTACAGCCCTAGCTCC
CAGAACAGCCAGCCTTGGCACTTCATCGTGGCTAGCACCGAGGAAGGCAAAGCT
AGGGTGGCTAAGAGCGCCGCTGGCAACTACGTGTTCAGCGAGAGGAAGATGCTG
GATGCTAGCCACGTGGTGGTGTTCTGCGCTAAGACCGCCATGGACGATGTGTGGC
TGAAGCTGGTGGTGGATCAGGAAGATGCTGATGGCAGGTTCGCTACCCCTGAAG
CTAAGGCCGCTAACGACAAGGGCAGGAAGTTCACTGCCGACATGCACAGGAAGG
ATCTGCACGATGATGCTGAGTGGATGGCCAAGCAGGTGTACCTGAACGTGGGCA
ACTTCCTGCTCGGCGTGGCTGCCCTGGGCCTCGATGCTGTGCCCATCGAAGGCTT
CGATGCTGCTATCCTGGATGCCGAGTTCGGCCTGAAGGAGAAAGGCTACACCAG
CCTGGTGGTGGTGCCTGTGGGCCACCACAGCGTGGAGGACTTCAACGCTACCCTG
CCTAAGAGCAGGCTGCCCCAGAACATCACCCTGACCGAGGTGTGATTAATTAAA
AGGGCGGATCCGGTCTCCAGATGGCCAACATGAGGATCACCCATGTCTGCAGGG
CCAGATGGCCAACATGAGGATCACCCATGTCTGCAGGGCCAGATGGCCAACATG
AGGATCACCCATGTCTGCAGGGCCAGATGGCCAACATGAGGATCACCCATGTCT
GCAGGGCCAGATGGCCAACATGAGGATCACCCATGTCTGCAGGGCCAGATGGCC
AACATGAGGATCACCCATGTCTGCAGGGCCAGATGGCCAACATGAGGATCACCC
ATGTCTGCAGGGCCAGATGGCCAACATGAGGATCACCCATGTCTGCAGGGCCAG
ATGGCCAACATGAGGATCACCCATGTCTGCAGGGCCAGATAGATCTCAATTG
(SEQ ID NO: 59)
>MCP_ADARddm(C377F, E488Q)-P2A-
Sensor(TMEM135_CCDC67_sensor_501)-E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCACCTTTTAGATGTGTGTTTTCAGTTCTGACAGCTTTATATTTCATATCTG
AGACCTTTTGTTTTGCTAACATTTTTTCTTTTATTTCTAATTCCATTTTTAACTGTTT
TTCCAGAAGGGCAGCTTTCTTTGTGACAGTTGCTATAGTGATCTCCTTCTGGTGGA
GCTCTTCTGTTAGGTCAGAGATTTCATTCCTCATTCTTTCCTGCTCAGAGTTATGG
TACTCTTCCACCTGTTGGAGTTAGCTTCTTATTTGCCCTTAGTGGGTCTTAAAAGG
AACTTCCAGTAAGAGGGTCTCAAAGTCTGAACTTCCATGGCAGCTGCCTGGAAGC
AAATTGCTGTGGAGATGGAATAGATGGTGGTATCTGCATGAGGAAAATAGGGAA
CCTTCCCTGCTTCAATGCCTTTGGAATACATTGTCTCTACCAATTTGGACGCTAAA
TACATGGAAATTGTTGTGCTTTTATAAAACATCATTGATATACCTGCCAAAAATC
CAGCTAGGCCGGCCAGGCTCGGGCCAGTGTACTAATTATGCTCTCTTGAAATTGG
CTGGAGATGTTGAGAGCAACCCAGGTCCCTTAATTAAGGTGGACATCATCAGCGT
GGCTCTGAAGAGGCACTCCACCAAGGCTTTCGACGCTTCCAAGAAACTGACCCCT
GAACAGGCCGAGCAGATCAAGACCCTGCTCCAGTACAGCCCTAGCTCCCAGAAC
AGCCAGCCTTGGCACTTCATCGTGGCTAGCACCGAGGAAGGCAAAGCTAGGGTG
GCTAAGAGCGCCGCTGGCAACTACGTGTTCAGCGAGAGGAAGATGCTGGATGCT
AGCCACGTGGTGGTGTTCTGCGCTAAGACCGCCATGGACGATGTGTGGCTGAAG
CTGGTGGTGGATCAGGAAGATGCTGATGGCAGGTTCGCTACCCCTGAAGCTAAG
GCCGCTAACGACAAGGGCAGGAAGTTCACTGCCGACATGCACAGGAAGGATCTG
CACGATGATGCTGAGTGGATGGCCAAGCAGGTGTACCTGAACGTGGGCAACTTC
CTGCTCGGCGTGGCTGCCCTGGGCCTCGATGCTGTGCCCATCGAAGGCTTCGATG
CTGCTATCCTGGATGCCGAGTTCGGCCTGAAGGAGAAAGGCTACACCAGCCTGG
TGGTGGTGCCTGTGGGCCACCACAGCGTGGAGGACTTCAACGCTACCCTGCCTAA
GAGCAGGCTGCCCCAGAACATCACCCTGACCGAGGTGTGATTAATTAAAAGGGC
GGATCCGGTCTCCAGATGGCCAACATGAGGATCACCCATGTCTGCAGGGCCAGA
TGGCCAACATGAGGATCACCCATGTCTGCAGGGCCAGATGGCCAACATGAGGAT
CACCCATGTCTGCAGGGCCAGATGGCCAACATGAGGATCACCCATGTCTGCAGG
GCCAGATGGCCAACATGAGGATCACCCATGTCTGCAGGGCCAGATGGCCAACAT
GAGGATCACCCATGTCTGCAGGGCCAGATGGCCAACATGAGGATCACCCATGTC
TGCAGGGCCAGATGGCCAACATGAGGATCACCCATGTCTGCAGGGCCAGATGGC
CAACATGAGGATCACCCATGTCTGCAGGGCCAGATAGATCTCAATTG (SEQ ID
NO: 60)
>MCP_ADARddm(C377F, E488Q)-P2A-Sensor(EVT6_NTRK3_sensor_501)-
E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCATATTCAGGTCTCCATGCTTCATGTATTCAAAGACCATGGTGGGGGGGT
CCCCATCGCCGCACACTCCATAGAACTTGGCAATGTGCTCATGCTGCAGGTTGGT
GGGCAGCTCGGCCTCCCTCTGGAAATCCTTCCGGGCAGCCAGGGTGGGATCCTTC
AGGGCCTTCACAGCCACAAGCATCTTGTCCTTAGTCGGGCTGGGGTTGTAGCACT
CGGCCAGGAAGACCTTTCCAAAGGCTCCCTCACCCAGTTCTCGCTTCAGCACGAT
GTCTCTCCTCTTACTGTGCTGCACATCTGCTATTCTCCCAATAGGCATGGCGTGCT
CTTCAGGCGGGGAGACAGAGACCATGGTGTGGTTCATGTAAGCCAGGTCTTCCC
GATGAGAGAGGTTGGTGGGCTTCCCTTCCCTATGCAGCCCGTCCTCGGAGAGCCT
GGACTGTTTGGAATCCACGGAGTGCCGGGGGTTCAGGATCAGAGGGTGCATGGT
GGGGCTGGGCATCAGGCCGGCCAGGCTCGGGCCAGTGTACTAATTATGCTCTCTT
GAAATTGGCTGGAGATGTTGAGAGCAACCCAGGTCCCTTAATTAAGGTGGACAT
CATCAGCGTGGCTCTGAAGAGGCACTCCACCAAGGCTTTCGACGCTTCCAAGAA
ACTGACCCCTGAACAGGCCGAGCAGATCAAGACCCTGCTCCAGTACAGCCCTAG
CTCCCAGAACAGCCAGCCTTGGCACTTCATCGTGGCTAGCACCGAGGAAGGCAA
AGCTAGGGTGGCTAAGAGCGCCGCTGGCAACTACGTGTTCAGCGAGAGGAAGAT
GCTGGATGCTAGCCACGTGGTGGTGTTCTGCGCTAAGACCGCCATGGACGATGTG
TGGCTGAAGCTGGTGGTGGATCAGGAAGATGCTGATGGCAGGTTCGCTACCCCT
GAAGCTAAGGCCGCTAACGACAAGGGCAGGAAGTTCACTGCCGACATGCACAGG
AAGGATCTGCACGATGATGCTGAGTGGATGGCCAAGCAGGTGTACCTGAACGTG
GGCAACTTCCTGCTCGGCGTGGCTGCCCTGGGCCTCGATGCTGTGCCCATCGAAG
GCTTCGATGCTGCTATCCTGGATGCCGAGTTCGGCCTGAAGGAGAAAGGCTACAC
CAGCCTGGTGGTGGTGCCTGTGGGCCACCACAGCGTGGAGGACTTCAACGCTAC
CCTGCCTAAGAGCAGGCTGCCCCAGAACATCACCCTGACCGAGGTGTGATTAATT
AAAAGGGCGGATCCGGTCTCCAGATGGCCAACATGAGGATCACCCATGTCTGCA
GGGCCAGATGGCCAACATGAGGATCACCCATGTCTGCAGGGCCAGATGGCCAAC
ATGAGGATCACCCATGTCTGCAGGGCCAGATGGCCAACATGAGGATCACCCATG
TCTGCAGGGCCAGATGGCCAACATGAGGATCACCCATGTCTGCAGGGCCAGATG
GCCAACATGAGGATCACCCATGTCTGCAGGGCCAGATGGCCAACATGAGGATCA
CCCATGTCTGCAGGGCCAGATGGCCAACATGAGGATCACCCATGTCTGCAGGGC
CAGATGGCCAACATGAGGATCACCCATGTCTGCAGGGCCAGATAGATCTCAATT
G (SEQ ID NO: 61)
>MCP_ADARddm(C377F, E488Q)-P2A-Sensor(TMPRSS2_ERG_sensor_264)-
E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCATTGGAAGTCTGTCCATGGTCGCTGGAGGAGGACGCGGTCATCTCTGTC
TTAGCCAGGTGTGGCGTTCCGTGGGCACACTCAAACAACGACTAGTCCTCACTCA
CAACTGATAAGGCTTCTGAGTTCAAAGCATCTTGCTGTTATCAACAGCATCGAGT
AAGGATAGGTATCTAGAATGTTCAATATGACCTGCCGCGCTCCAGGCGGCGCTCC
CCGCCCCTCGCCCTCCGCCTCCGCCTCCGCCTCCTGCTTAGCTCGCGCCTAGGCCG
GCCAGGCTCGGGCCAGTGTACTAATTATGCTCTCTTGAAATTGGCTGGAGATGTT
GAGAGCAACCCAGGTCCCTTAATTAAGGTGGACATCATCAGCGTGGCTCTGAAG
AGGCACTCCACCAAGGCTTTCGACGCTTCCAAGAAACTGACCCCTGAACAGGCC
GAGCAGATCAAGACCCTGCTCCAGTACAGCCCTAGCTCCCAGAACAGCCAGCCT
TGGCACTTCATCGTGGCTAGCACCGAGGAAGGCAAAGCTAGGGTGGCTAAGAGC
GCCGCTGGCAACTACGTGTTCAGCGAGAGGAAGATGCTGGATGCTAGCCACGTG
GTGGTGTTCTGCGCTAAGACCGCCATGGACGATGTGTGGCTGAAGCTGGTGGTGG
ATCAGGAAGATGCTGATGGCAGGTTCGCTACCCCTGAAGCTAAGGCCGCTAACG
ACAAGGGCAGGAAGTTCACTGCCGACATGCACAGGAAGGATCTGCACGATGATG
CTGAGTGGATGGCCAAGCAGGTGTACCTGAACGTGGGCAACTTCCTGCTCGGCGT
GGCTGCCCTGGGCCTCGATGCTGTGCCCATCGAAGGCTTCGATGCTGCTATCCTG
GATGCCGAGTTCGGCCTGAAGGAGAAAGGCTACACCAGCCTGGTGGTGGTGCCT
GTGGGCCACCACAGCGTGGAGGACTTCAACGCTACCCTGCCTAAGAGCAGGCTG
CCCCAGAACATCACCCTGACCGAGGTGTGATTAATTAAAAGGGCGGATCCGGTC
TCCAGATGGCCAACATGAGGATCACCCATGTCTGCAGGGCCAGATGGCCAACAT
GAGGATCACCCATGTCTGCAGGGCCAGATGGCCAACATGAGGATCACCCATGTC
TGCAGGGCCAGATGGCCAACATGAGGATCACCCATGTCTGCAGGGCCAGATGGC
CAACATGAGGATCACCCATGTCTGCAGGGCCAGATGGCCAACATGAGGATCACC
CATGTCTGCAGGGCCAGATGGCCAACATGAGGATCACCCATGTCTGCAGGGCCA
GATGGCCAACATGAGGATCACCCATGTCTGCAGGGCCAGATGGCCAACATGAGG
ATCACCCATGTCTGCAGGGCCAGATAGATCTCAATTG (SEQ ID NO: 62)
>MCP_ADARddm(C377F, E488Q)-P2A-
Sensor(TRMT11_GRIK2ss_Sensor_201)-E2A-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCACTTCTGGGTATCATAGGTAAGGGTAGTATTGGGTGGCAATGTTCTGTT
TCTGTTAATTGTGTTCACAGCAAATCTGAATGCAAGTTCCTCAGCTCCCATTAGG
CCAGATTCCACATATTCAAAAATACCACCGGCAGGCGGAACTCCAGATGCTCCT
GCGCCAGGAGGAGCAGATACCTGTTAAGGGTACACGACAGCGCCGGCCGGCCAG
GCTCGGGCCAGTGTACTAATTATGCTCTCTTGAAATTGGCTGGAGATGTTGAGAG
CAACCCAGGTCCCTTAATTAAGGTGGACATCATCAGCGTGGCTCTGAAGAGGCA
CTCCACCAAGGCTTTCGACGCTTCCAAGAAACTGACCCCTGAACAGGCCGAGCA
GATCAAGACCCTGCTCCAGTACAGCCCTAGCTCCCAGAACAGCCAGCCTTGGCA
CTTCATCGTGGCTAGCACCGAGGAAGGCAAAGCTAGGGTGGCTAAGAGCGCCGC
TGGCAACTACGTGTTCAGCGAGAGGAAGATGCTGGATGCTAGCCACGTGGTGGT
GTTCTGCGCTAAGACCGCCATGGACGATGTGTGGCTGAAGCTGGTGGTGGATCA
GGAAGATGCTGATGGCAGGTTCGCTACCCCTGAAGCTAAGGCCGCTAACGACAA
GGGCAGGAAGTTCACTGCCGACATGCACAGGAAGGATCTGCACGATGATGCTGA
GTGGATGGCCAAGCAGGTGTACCTGAACGTGGGCAACTTCCTGCTCGGCGTGGCT
GCCCTGGGCCTCGATGCTGTGCCCATCGAAGGCTTCGATGCTGCTATCCTGGATG
CCGAGTTCGGCCTGAAGGAGAAAGGCTACACCAGCCTGGTGGTGGTGCCTGTGG
GCCACCACAGCGTGGAGGACTTCAACGCTACCCTGCCTAAGAGCAGGCTGCCCC
AGAACATCACCCTGACCGAGGTGTGATTAATTAAAAGGGCGGATCCGGTCTCCA
GATGGCCAACATGAGGATCACCCATGTCTGCAGGGCCAGATGGCCAACATGAGG
ATCACCCATGTCTGCAGGGCCAGATGGCCAACATGAGGATCACCCATGTCTGCA
GGGCCAGATGGCCAACATGAGGATCACCCATGTCTGCAGGGCCAGATGGCCAAC
ATGAGGATCACCCATGTCTGCAGGGCCAGATGGCCAACATGAGGATCACCCATG
TCTGCAGGGCCAGATGGCCAACATGAGGATCACCCATGTCTGCAGGGCCAGATG
GCCAACATGAGGATCACCCATGTCTGCAGGGCCAGATGGCCAACATGAGGATCA
CCCATGTCTGCAGGGCCAGATAGATCTCAATTG (SEQ ID NO: 63)
>MCP_ADARddm(C377F, E488Q)-P2A-Sensor(PVT1_MYC_sensor_498)-E2A-
NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCAGTCTCCTCCCAGCAGCTCGGTCACCATCTCCAGCTGGTCGGCCGTGGA
GAAGCTCCCGCCACCGCCGTCGTTGTCTCCCCGAAGGGAGAAGGGTGTGACCGC
AACGTAGGAGGGCGAGCAGAGCCCGGAGCGGCGGCTAGGGGACAGGGGCGGGG
TGGGCAGCAGCTCGAATTTCTTCCAGATATCCTCGCTGGGCGCCGGGGGCTGCAG
CTCGCTCTGCTGCTGCTGCTGGTAGAAGTTCTCCTCCTCGTCGCAGTAGAAATAC
GGCTGCACCGAGTCGTAGTCGAGGTCATAGTTCCTGTTGGTGAAGCTAACGTTGA
GGGGCATCGTCGCGGGAGGCTGTAGAGGGCAGATCTGGCCGTGTCTCCACAGGT
CACAGGGACCGCCAACATCCTTTCCGCAAGGAAATCCACTGGAAGGTGCCGGGG
GTCCCGGGGCACATCTTTGCTCGCAGCTCGTCGTCGCCCCTCCTCGTCCCGGCCG
CCCCGAGCCCGCCCGGGCCGGCCAGGCTCGGGCCAGTGTACTAATTATGCTCTCT
TGAAATTGGCTGGAGATGTTGAGAGCAACCCAGGTCCCTTAATTAAGGTGGACA
TCATCAGCGTGGCTCTGAAGAGGCACTCCACCAAGGCTTTCGACGCTTCCAAGAA
ACTGACCCCTGAACAGGCCGAGCAGATCAAGACCCTGCTCCAGTACAGCCCTAG
CTCCCAGAACAGCCAGCCTTGGCACTTCATCGTGGCTAGCACCGAGGAAGGCAA
AGCTAGGGTGGCTAAGAGCGCCGCTGGCAACTACGTGTTCAGCGAGAGGAAGAT
GCTGGATGCTAGCCACGTGGTGGTGTTCTGCGCTAAGACCGCCATGGACGATGTG
TGGCTGAAGCTGGTGGTGGATCAGGAAGATGCTGATGGCAGGTTCGCTACCCCT
GAAGCTAAGGCCGCTAACGACAAGGGCAGGAAGTTCACTGCCGACATGCACAGG
AAGGATCTGCACGATGATGCTGAGTGGATGGCCAAGCAGGTGTACCTGAACGTG
GGCAACTTCCTGCTCGGCGTGGCTGCCCTGGGCCTCGATGCTGTGCCCATCGAAG
GCTTCGATGCTGCTATCCTGGATGCCGAGTTCGGCCTGAAGGAGAAAGGCTACAC
CAGCCTGGTGGTGGTGCCTGTGGGCCACCACAGCGTGGAGGACTTCAACGCTAC
CCTGCCTAAGAGCAGGCTGCCCCAGAACATCACCCTGACCGAGGTGTGATTAATT
AAAAGGGCGGATCCGGTCTCCAGATGGCCAACATGAGGATCACCCATGTCTGCA
GGGCCAGATGGCCAACATGAGGATCACCCATGTCTGCAGGGCCAGATGGCCAAC
ATGAGGATCACCCATGTCTGCAGGGCCAGATGGCCAACATGAGGATCACCCATG
TCTGCAGGGCCAGATGGCCAACATGAGGATCACCCATGTCTGCAGGGCCAGATG
GCCAACATGAGGATCACCCATGTCTGCAGGGCCAGATGGCCAACATGAGGATCA
CCCATGTCTGCAGGGCCAGATGGCCAACATGAGGATCACCCATGTCTGCAGGGC
CAGATGGCCAACATGAGGATCACCCATGTCTGCAGGGCCAGATAGATCTCAATT
G (SEQ ID NO: 64)
>MCP_ADARddm(C377F,E488Q)-P2A-Sensor(TP53_R248Q_sensor111)-
XTEN80-NTR1.1-9xMS2
GCCGCCACCATGGCTTCAAACTTTACTCAGTTCGTGCTCGTGGACAATGGTGGGA
CAGGGGATGTGACAGTGGCTCCTTCTAATTTCGCTAATGGGGTGGCAGAGTGGAT
CAGCTCCAACTCACGGAGCCAGGCCTACAAGGTGACATGCAGCGTCAGGCAGTC
TAGTGCCCAGAAGAGAAAGTATACCATCAAGGTGGAGGTCCCCAAAGTGGCTAC
CCAGACAGTGGGCGGAGTCGAACTGCCTGTCGCCGCTTGGAGGTCCTACCTGAA
CATGGAGCTCACTATCCCAATTTTCGCTACCAATTCTGACTGTGAACTCATCGTG
AAGGCAATGCAGGGGCTCCTCAAAGACGGTAATCCTATCCCTTCCGCCATCGCCG
CTAACTCAGGTATCTACAGCGCTGGCGGCCGCGGGGGAGGCGGTTCCGGTGGCG
GCGGAAGCGGAGGTGGAGGATCACAGCTGCATTTACCGCAGGTTTTAGCTGACG
CTGTCTCACGCCTGGTCCTGGGTAAGTTTGGTGACCTGACCGACAACTTCTCCTCC
CCTCACGCTCGCAGAAAAGTGCTGGCTGGAGTCGTCATGACAACAGGCACAGAT
GTTAAAGATGCCAAGGTGATAAGTGTTTCTACAGGAACAAAATTTATTAATGGTG
AATACATGAGTGATCGTGGCCTTGCATTAAATGACTGCCATGCAGAAATAATATC
TCGGAGATCCTTGCTCAGATTTCTTTATACACAACTTGAGCTTTACTTAAATAACA
AAGATGATCAAAAAAGATCCATCTTTCAGAAATCAGAGCGAGGGGGGTTTAGGC
TGAAGGAGAATGTCCAGTTTCATCTGTACATCAGCACCTCTCCCTGTGGAGATGC
CAGAATCTTCTCACCACATGAGCCAATCCTGGAAGAACCAGCAGATAGACACCC
AAATCGTAAAGCAAGAGGACAGCTACGGACCAAAATAGAGTCTGGTCAGGGGA
CGATTCCAGTGCGCTCCAATGCGAGCATCCAAACGTGGGACGGGGTGCTGCAAG
GGGAGCGGCTGCTCACCATGTCCTGCAGTGACAAGATTGCACGCTGGAACGTGG
TGGGCATCCAGGGATCACTGCTCAGCATTTTCGTGGAGCCCATTTACTTCTCGAG
CATCATCCTGGGCAGCCTTTACCACGGGGACCACCTTTCCAGGGCCATGTACCAG
CGGATCTCCAACATAGAGGACCTGCCACCTCTCTACACCCTCAACAAGCCTTTGC
TCAGTGGCATCAGCAATGCAGAAGCACGGCAGCCAGGGAAGGCCCCCAACTTCA
GTGTCAACTGGACGGTAGGCGACTCCGCTATTGAGGTCATCAACGCCACGACTG
GGAAGGATGAGCTGGGCCGCGCGTCCCGCCTGTGTAAGCACGCGTTGTACTGTC
GCTGGATGCGTGTGCACGGCAAGGTTCCCTCCCACTTACTACGCTCCAAGATTAC
CAAGCCCAACGTGTACCATGAGTCCAAGCTGGCGGCAAAGGAGTACCAGGCCGC
CAAGGCGCGTCTGTTCACAGCCTTCATCAAGGCGGGGCTGGGGGCCTGGGTGGA
GAAGCCCACCGAGCAGGACCAGTTCTCACTCACGACTAGTGGCAGCGGCGCCAC
AAACTTCTCTCTGCTAAAGCAAGCAGGTGATGTTGAAGAAAACCCCGGGCCTGG
CGCGCCATCCACCTGCCTTGGCCTGGAGTCTTCCAGTGTGATGATGGTGAGGATG
GGCCTCTAGTTCAGGCCGCCCAGGCAGGAACTGTTACACAGGTGGTTGTGGTGG
AGGGTGGTAGGCCGGCCAGGCTCGGGCCAGTGTACTAATTATGCTCTCTTGAAAT
TGGCTGGAGATGTTGAGAGCAACCCAGGTCCCTTAATTAAGGTGGACATCATCA
GCGTGGCTCTGAAGAGGCACTCCACCAAGGCTTTCGACGCTTCCAAGAAACTGA
CCCCTGAACAGGCCGAGCAGATCAAGACCCTGCTCCAGTACAGCCCTAGCTCCC
AGAACAGCCAGCCTTGGCACTTCATCGTGGCTAGCACCGAGGAAGGCAAAGCTA
GGGTGGCTAAGAGCGCCGCTGGCAACTACGTGTTCAGCGAGAGGAAGATGCTGG
ATGCTAGCCACGTGGTGGTGTTCTGCGCTAAGACCGCCATGGACGATGTGTGGCT
GAAGCTGGTGGTGGATCAGGAAGATGCTGATGGCAGGTTCGCTACCCCTGAAGC
TAAGGCCGCTAACGACAAGGGCAGGAAGTTCACTGCCGACATGCACAGGAAGGA
TCTGCACGATGATGCTGAGTGGATGGCCAAGCAGGTGTACCTGAACGTGGGCAA
CTTCCTGCTCGGCGTGGCTGCCCTGGGCCTCGATGCTGTGCCCATCGAAGGCTTC
GATGCTGCTATCCTGGATGCCGAGTTCGGCCTGAAGGAGAAAGGCTACACCAGC
CTGGTGGTGGTGCCTGTGGGCCACCACAGCGTGGAGGACTTCAACGCTACCCTGC
CTAAGAGCAGGCTGCCCCAGAACATCACCCTGACCGAGGTGTGATTAATTAAAA
GGGCGGATCCGGTCTCCAGATGGCCAACATGAGGATCACCCATGTCTGCAGGGC
CAGATGGCCAACATGAGGATCACCCATGTCTGCAGGGCCAGATGGCCAACATGA
GGATCACCCATGTCTGCAGGGCCAGATGGCCAACATGAGGATCACCCATGTCTG
CAGGGCCAGATGGCCAACATGAGGATCACCCATGTCTGCAGGGCCAGATGGCCA
ACATGAGGATCACCCATGTCTGCAGGGCCAGATGGCCAACATGAGGATCACCCA
TGTCTGCAGGGCCAGATGGCCAACATGAGGATCACCCATGTCTGCAGGGCCAGA
TGGCCAACATGAGGATCACCCATGTCTGCAGGGCCAGATAGATCTCAATTG (SEQ
ID NO: 65)
>CCNH_C5orf30_sensor_501 (sensor stop codon lower case and underlined,
mismatches/insertion/deletion with target in lowercase):
GCATCGGGGAGCAGGGTGTGCTGGAGCAGCGGGGCTTCTCTGCCTCCGCCTTTCC
CGGGGAGTTGGCCTCAGCCCCAGGGAAGGGCAAGGTGGTCAGGGTACTTCTgGA
CTCTCCATTAcTATCGACTTCCATTTTAcGCACCAGTCTGCACAATCCAGACTAAA
TCCCAGAAGATGTCACTGTACAATTTCCAAtagGTCCCAAGGGAACACAGACATAA
TAATTTTTCTGTTCTACTCCAGGTAatTTTTCATTATATCTgGTAcCTGtagCAGGCAA
GTTCTGTTCTCTTTCAGCATCAGACTCTCTGgTACATAACTTTCCATAGTAATTCCA
GCCCTGGAGGCACTAGATAcAATGGCAGTCAGGGCAATTTGGGAAGGTGTGTAT
AcAAGGTAAGCATCCGTCAATGCAATTCTATTAAGAAAGTCATCAGCTGTTTTCC
TCAAAATCTCTGGATTCTCCAATATGGGATAGCGGGTCTTTAcGTCGATGAG (SEQ
ID NO: 84)
>TMEM135_CCDC67_sensor_501 (sensor stop codon lower case and underlined,
mismatches/insertion/deletion with target in lowercase):
CCTTTTAGATGTGTGTTTTCAGTTCTGACAGCTTTATATTTCATATCTGAGACCTTT
TGTTTTGCTAACATTTTTTCTTTTATTTCTAATTCCATTTTTAACTGTTTTTCCAGA
AGGGCAGCTTTCTTTGTGACAGTTGCTATAGTGATCTCCTTCTGgTGgAGCTCTTCT
GTTAGGTCAGAGATTTCATTCCTCATTCTTTCCTGCTCAGAGTTATGGTACTCTTC
CACCTGTTGGAGTtagCTTCTTATTTGCCCTtagTGgGTCTTAAAAGGAACTTCCAGT
AAGAgGGTCTCAAAGTCTGAACTTCCATGgCAGCTGCCTGGAAGCAAATTGCTGT
gGAGATGGAATAGATGgTgGTATCTGCATGAGGAAAATAGGGAACCTTCCCTGCT
TCAATGCCTTTGgAATACATTGTCTCTACCAATTTGGACGCTAAATACATGGAAAT
TGTTGTGCTTTTATAAAACATCATTGATATACCTGCCAAAAATCCAGCTA (SEQ ID
NO: 85)
>EVT6_NTRK3_sensor_501
TATTCAGGTCTCCATGCTTCATGTATTCAAAGACCATGgTGgGGGGGTCCCCATCG
CCGCACACTCCATAGAACTTGgCAATGTGCTCATGCTGCAGGTTGGTGgGCAGCT
CGGCCTCCCTCTGGAAATCCTTCCGGGCAGCCAGGGTGGGATCCTTCAGGGCCTT
CACAGCCACAAGCATCTTGTCCTtagTCGGGCTGgGGTTGTAGCACTCGGCCAGGA
AGACCTTTCCAAAGGCTCCCTCACCCAGTTCTCGCTTCAGCACGATGTCTCTCCTC
TTAcTGTGCTGCACATCTGCTATTCTCCCAAtagGCATGGCGTGCTCTTCAGGCGGG
GAGACAGAGACCATGTGTGGTTCATGTAAGCCAGGTCTTCCCGATGAGAGAGGT
TGgTGGGCTTCCCTTCCCTATGCAGCCCGTCCTCGGAGAGCCTGGACTGTTTGgAA
TCCACGGAGTGCCGGGGGTTCAGGATCAGAGGGTGCATGgTGGGGCTGGGCATC
A (SEQ ID NO: 86)
>TMPRSS2_ERG_sensor_264
TTGGAAGTCTGTCCATgGTCGCTGGAGGAGGACGCGGTCATCTCTGTCTTAGCCA
GGTGTGGCGTTCCGTgGGCACACTCAAACAACGACtagTCCTCACTCACAACTGAT
AAGGCTTCTGAGTTCAAAGCATCTTGCTGTTATCAACAGCATCGAGTAAgGATAG
GTATCtagAATGTTCAATATGACCTGCCGCGCTCCAGGCGGCGCTCCCCGCCCCTC
GCCCTCCGCCTCCGCCTCCGCCTCCTGCTTAGCTCGCGCCTA (SEQ ID NO: 87)
>TRMT11_GRIK2ss_Sensor_201
CTTCTGGGTATCATAGGTAAGGGTAGTATTGGGTgGCAATGTTCTGTTTCTGTTAA
TTGTGTTCACAGCAAATCTGAATGCAAGTTCCTCAGCTCCCATtagGCCAGATTCC
ACATATTCAAAAATACCACCGGCAGGCGGAACTCCAGATGCTCCTGCGCCAgGA
GGAGCAGATACCTGTTAAGGGTACACGACAGCGCC (SEQ ID NO: 88)
>PVT1_MYC_sensor_498
GTCTCCTCCCAGCAGCTCGGTCACCATCTCCAGCTGGTCGGCCGTGGAGAAGCTC
CCGCCACCGCCGTCGTTGTCTCCCCGAAGGGAGAAGGGTGTGACCGCAACGTAG
GAGGGCGAGCAGAGCCCGGAGCGGCGGCTAGGGGACAGGGGGGGGGTGGGCAG
CAGCTCGAATTTCTTCCAGATATCCTCGCTGGGCGCCGGGGGCTGCAGCTCGCTC
TGCTGCTGCTGCTGGTAGAAGTTCTCCTCCTCGTCGCAGTAGAAATACGGCTGCA
CCGAGTCGTAGTCGAGGTCATAGTTCCTGTTGGTGAAGCTAACGTTGAGGGGCAT
CGTCGCGGGAGGCTGtagAGGGCAGATCTGGCCGTGTCTCCACAGGTCACAGGGA
CCGCCAACATCCTTTCCGCAAGGAAATCCACTGGAAGGTGCCGGGGGTCCCGGG
GCACATCTTTGCTCGCAGCTCGTCGTCGCCCCTCCTCGTCCCGGCCGCCCCGAGC
CCGCCCG (SEQ ID NO: 89)
>TP53_R248Q_sensor111
TCCACCTGCCTTGGCCTGGAGTCTTCCAGTGTGATGATGGTGAGGATGGGCCTCta
gTTCAgGCCGCCCAgGCAGGAACTGTTACACAgGTgGTTGTgGTGGAgGGTGGTA
(SEQ ID NO: 90)

### 5. CLAUSES

The invention is further described by the following clauses:

Clause 1: A single-stranded nucleic acid sensor molecule comprising:
a target sensing region having a nucleic acid sequence that is substantially complementary to a target nucleic acid, wherein the target sensing region comprises a TAG or TGA stop codon opposite a corresponding CAA, CTA, CGA, ACA, TCA, GCA, CCA, CCT, or CCC triplet in the target nucleic acid positioned on at least one side of a junctional sequence in the target nucleic acid; and
a response gene positioned downstream of the target sensing region, wherein the response gene is expressed when the TAG or TGA stop codon is converted to a TGG codon by adenosine deaminase acting on RNA (ADAR)-mediated gene editing upon binding of the sensor molecule to the target nucleic acid.
   Clause 2: The sensor of clause 1, wherein the target sensing region comprises a TAG or TGA stop codon opposite a corresponding CCA (or 5′-CAA-3′, 5′-CTA-3′, 5′-CGA-3′, 5′-ACA-3′, 5′-TCA-3′, 5′-GCA-3′, 5′-CCT-3′, or 5′-CCC-3′) triplet in the target nucleic acid.
   Clause 3: The sensor molecule according to Clause 1 or Clause 2, wherein the junctional sequence in the target nucleic acid corresponds to at least a portion of a gene or chromosomal fusion.
   Clause 4: The sensor molecule according to Clause 3, wherein the junctional sequence in the target nucleic acid comprises at least a portion of a gene or chromosomal fusion associated with cancer.
   Clause 5: The sensor molecule according to any one of Clauses 1-4, wherein the junctional sequence in the target nucleic acid comprises a CBFA2T3-GLIS2 fusion sequence, an EML4-ALK fusion sequence, a ZFTA-RELA fusion sequence, an EWSR1-FL1 fusion sequence, a CCNH-C5orf30 fusion sequence, a TMEM135-CCDC67 fusion sequence, a EVT6-NTRK3 fusion sequence, a TMPRSS2-ERG fusion sequence, a TRMT11-GRIK2 fusion sequence, or a PVT1-MYC fusion sequence.
   Clause 6: The sensor molecule according to Clause 1 or Clause 2, wherein the junctional sequence in the target nucleic acid comprises a TP53(R248Q) mutant transcript.
   Clause 7: The sensor molecule according to Clause 5, wherein the junctional sequence in the target nucleic acid comprises a CBFA2T3-GLIS2 fusion sequence, and wherein the target sensing region includes the amino acid sequence set forth in SEQ ID NO: 3.
   Clause 8: The sensor molecule according to Clause 5, wherein the junctional sequence in the target nucleic acid comprises a CBFA2T3-GLIS2 fusion sequence, and wherein the target sensing region comprises an amino acid sequence having at least 80% sequence identity to SEQ ID NO: 4 or SEQ ID NO: 5.
   Clause 9: The sensor molecule according to Clause 5, wherein the junctional sequence in the target nucleic acid comprises an EML4-ALK fusion sequence, and wherein the target sensing region includes the amino acid sequence set forth in SEQ ID NO: 28.
   Clause 10: The sensor molecule according to Clause 5, wherein the junctional sequence in the target nucleic acid comprises an EML4-ALK fusion sequence, and wherein the target sensing region comprises an amino acid sequence having at least 80% sequence identity to SEQ ID NO: 20 or SEQ ID NO: 29.
   Clause 11: The sensor molecule according to Clause 5, wherein the junctional sequence in the target nucleic acid comprises a ZFTA-RELA fusion sequence, and wherein the target sensing region includes the amino acid sequence set forth in SEQ ID NO: 32.
   Clause 12: The sensor molecule according to Clause 5, wherein the junctional sequence in the target nucleic acid comprises a ZFTA-RELA fusion sequence, and wherein the target sensing region comprises an amino acid sequence having at least 80% sequence identity to SEQ ID NO: 31 or SEQ ID NO: 30.
   Clause 13: The sensor molecule according to Clause 5, wherein the junctional sequence of the target nucleic acid comprises an EWSR1-FL1 fusion sequence, and wherein the target sensing region comprises an amino acid sequence having at least 80% sequence identity to SEQ ID NO: 33.
   Clause 14: The sensor molecule according to Clause 5, wherein the junctional sequence in the target nucleic acid comprises a CCNH-C5orf30 fusion sequence, and wherein the target sensing region includes an amino acid sequence having at least 80% sequence identity to SEQ ID NO: 84.
   Clause 15: The sensor molecule according to Clause 5, wherein the junctional sequence in the target nucleic acid comprises a TMEM135-CCDC67 fusion sequence, and wherein the target sensing region includes an amino acid sequence having at least 80% sequence identity to SEQ ID NO: 85.
   Clause 16: The sensor molecule according to Clause 5, wherein the junctional sequence in the target nucleic acid comprises an EVT6-NTRK3 fusion sequence, and wherein the target sensing region includes an amino acid sequence having at least 80% sequence identity to SEQ ID NO: 86.
   Clause 17: The sensor molecule according to Clause 5, wherein the junctional sequence in the target nucleic acid comprises a TMPRSS2-ERG fusion sequence, and wherein the target sensing region includes an amino acid sequence having at least 80% sequence identity to SEQ ID NO: 87.
   Clause 18: The sensor molecule according to Clause 5, wherein the junctional sequence in the target nucleic acid comprises a TRMT11-GRIK2 fusion sequence, and wherein the target sensing region includes an amino acid sequence having at least 80% sequence identity to SEQ ID NO: 88.
   Clause 19: The sensor molecule according to Clause 5, wherein the junctional sequence in the target nucleic acid comprises a PVT1-MYC fusion sequence, and wherein the target sensing region includes an amino acid sequence having at least 80% sequence identity to SEQ ID NO: 89.
   Clause 20: The sensor molecule according to Clause 5, wherein the junctional sequence in the target nucleic acid comprises a TP53(R248Q) mutant transcript, and wherein the target sensing region includes an amino acid sequence having at least 80% sequence identity to SEQ ID NO: 90.
   Clause 21: The sensor molecule according to Clause 1 or Clause 2, wherein the junctional sequence of the target nucleic acid corresponds to a viral transcript.
   Clause 22: The sensor molecule according to Clause 21, wherein the viral transcript is an Epstein Barr Virus (EBV) transcript or a Kaposi's sarcoma-associated herpesvirus (KSHV) transcript.
   Clause 23: The sensor molecule according to Clause 21, wherein the viral transcript is the Epstein Barr Virus transcript EBNA1 and wherein the target sensing region comprises an amino acid sequence having at least 80% sequence identity to SEQ ID NO: 34.
   Clause 24: The sensor molecule according to Clause 21, wherein the viral transcript the KSHV transcript ORF71 and wherein the target sensing region comprises an amino acid sequence having at least 80% sequence identity to SEQ ID NO: 35.
   Clause 25: The sensor molecule according to any one of Clauses 1 to 24, wherein the target sensing region is at least about 50 nucleotides long.
   Clause 26: The sensor molecule according to Clause 25, wherein the target sensing region is from about 50 nucleotides to about 1000 nucleotides long.
   Clause 27: The sensor molecule according to any one of Clauses 1 to 26, wherein the response gene encodes at least one of a reporter protein, a caspase, a prodrug-converting enzyme, or an enzyme catalyzing other reactions.
   Clause 28: The sensor molecule according to Clause 27, wherein the response gene encodes nitroreductase (NTR), diptheria toxin fragment A (DTA), or BCL2 associated X (BAX).
   Clause 29: The sensor molecule according to any one of Clauses 1 to 28, wherein the sensor molecule further comprises a control gene.
   Clause 30: The sensor molecule according to Clause 29, wherein the control gene is constitutively expressed.
   Clause 31: The sensor of Clause 20 or Clause 30, wherein the control gene encodes a fluorescent protein.
   Clause 32: The sensor molecule according to any one of Clauses 1 to 31, wherein the sensor molecule comprises a linker region positioned upstream of the response gene but downstream of the TAG or TGA stop codon.
   Clause 33: The sensor molecule of Clause 32, wherein the linker region comprises a 2A peptide or an XTEN80 linker.
   Clause 34: The sensor molecule of Clause 33, wherein the linker region comprises SEQ ID NO: 13, SEQ ID NO: 14, or SEQ ID NO: 15.
   Clause 35: The sensor molecule according to any one of Clauses 1 to 34, wherein the sensor molecule comprises an RNA aptamer sequence capable of binding its cognate binding protein.
   Clause 36: The sensor molecule according to Clause 35, wherein the RNA aptamer sequence comprises a sequence capable of binding at least one of MS2, PP7, BoxB, or Pumilio.
   Clause 37: The sensor molecule according to Clause 36 or Clause 37, wherein the cognate binding protein is fused to an ADAR protein.
   Clause 38: The sensor molecule according to any one of Clauses 1 to 37, wherein the sensor molecule further comprises a gene encoding an ADAR or an ADAR fusion, wherein the ADAR or ADAR fusion is constitutively expressed.
   Clause 39: The sensor molecule according to Clause 38, wherein the ADAR fusion comprises an ADAR enzyme fused to a cognate aptamer-binding protein.
   Clause 40: The sensor molecule according to Clause 39, wherein the sensor molecule further comprises an RNA aptamer sequence that recruits the cognate aptamer-binding protein upon expression of the ADAR fusion.
   Clause 41: The sensor molecule according to any one of Clauses 1 to 40, wherein the sensor molecule is an RNA molecule.
   Clause 42: An expression vector comprising a DNA sequence corresponding to any of the sensor molecules of any one of Clauses 1 to 41.
   Clause 43: The expression vector of Clause 42, selected from the group consisting of:
(a) a pCR8-mRuby2-P2A-ccdbCam-E2A-EGFP vector;
(b) a pCR8-mRuby2-P2A-ccdbCam-E2A-EGFP-BsaI(agat) vector;
(c) a pCR8-mRuby2-P2A-ccdbCam-E2A-EGFP-NxMS2 vector;
(d) a pCR8-mRuby2-P2A-Sensor-E2A-EGFP vector;
(e) a pCR8-mRuby2-P2A-Sensor-E2A-EGFP-NxMS2 vector;
(f) a pmax-mRuby2-P2A-Sensor-XTEN80-EGFP-NxMS2 vector;
(g) an MCP-ADARdd(E488Q) vector;
(h) a pmax-MCP-ADARdd(E488Q) vector;
(i) a pmax-MCP-ADARdd(E488Q)-P2A-Sensor-XTEN80-NTR1.1-NxMS2 vector;
(j) a pmax-MCP-ADARdd(E488Q)-P2A-Sensor-E2A-NTR1.1-NxMS2 vector;
(k) a pmax-MCP-ADARddm(C377F,E488Q)-P2A-Sensor-XTEN80-NTR1.1-NxMS2 vector;
(l) a pmax-MCP-ADARddm(C377F,E488Q)-P2A-Sensor-E2A-NTR1.1-NxMS2 vector;
(m) a pmax-MCP-ADARdd(E488Q)-P2A-Sensor-XTEN80-DTA-NxMS2 vector;
(n) a pmax-MCP-ADARdd(E488Q)-P2A-Sensor-E2A-DTA-NxMS2 vector;
(o) a pmax-MCP-ADARddm(C377F,E488Q)-P2A-Sensor-XTEN80-DTA-NxMS2 vector;
(p) a pmax-MCP-ADARddm(C377F,E488Q)-P2A-Sensor-E2A-DTA-NxMS2 vector;
(q) a pmax-MCP-ADARdd(E488Q)-P2A-Sensor-XTEN80-BAX-NxMS2 vector;
(r) a pmax-MCP-ADARdd(E488Q)-P2A-Sensor-E2A-BAX-NxMS2 vector;
(s) a pmax-MCP-ADARddm(C377F,E488Q)-P2A-Sensor-XTEN80-BAX-NxMS2 vector; and
(t) a pmax-MCP-ADARddm(C377F,E488Q)-P2A-Sensor-E2A-BAX-NxMS2 vector.
   Clause 44: A cell comprising any of the sensor molecules of any one of Clauses 1 to 41, or any of the vectors of Clauses 42 or 43.
   Clause 45: A kit comprising:
any of the sensor molecules of any one of Clauses 1 to 41;
any of the vectors of Clause 42 or 43; and/or the cell of Clause 44.
   Clause 46: A method of treating a subject having cancer or suspected of having cancer, the method comprising administering any of the sensor molecules of any one of Clauses 1 to 41, any of the vectors of Clause 42 or 43, and/or the cell of Clause 44 to the subject.
   Clause 47: The method of Clause 46, further comprising administering a prodrug to the subject, wherein the sensor molecule converts the prodrug to a cytotoxic agent, thereby treating cancer in the subject.
   Clause 48: The method of Clause 46 or Clause 47, wherein the cancer contains a chromosomal translocation and/or gene fusion.
   Clause 49: The method of Clause 46 or Clause 47, wherein the cancer contains a viral genome or expresses viral transcripts.
   Clause 50: The method of Clause 46 or Clause 47, wherein the cancer contains one or more gene mutations or expresses one or more mutant gene transcripts.
   Clause 51: A method of detecting a transcript in a cell, the method comprising:
transfecting a cell with any of the sensor molecules of any one of Clauses 1 to 41, or any of the vectors of Clause 42 or 43; and
assessing the cell for expression of a reporter protein.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A single-stranded nucleic acid sensor molecule comprising:
a target sensing region having a nucleic acid sequence that is substantially complementary to a target nucleic acid, wherein the target sensing region comprises a TAG or TGA stop codon opposite a corresponding CAA, CTA, CGA, ACA, TCA, GCA, CCA, CCT, or CCC triplet in the target nucleic acid positioned on at least one side of a junctional sequence in the target nucleic acid; and
a response gene positioned downstream of the target sensing region, wherein the response gene is expressed when the TAG or TGA stop codon is converted to a TGG codon by adenosine deaminase acting on RNA (ADAR)-mediated gene editing upon binding of the sensor molecule to the target nucleic acid.

2. The sensor of claim 1, wherein the target sensing region comprises a TAG or TGA stop codon opposite a corresponding CCA triplet in the target nucleic acid.

3. The sensor molecule according to claim 1, wherein the junctional sequence in the target nucleic acid corresponds to at least a portion of a gene or chromosomal fusion.

4. The sensor molecule according to claim 3, wherein the junctional sequence in the target nucleic acid comprises at least a portion of a gene or chromosomal fusion associated with cancer.

5. The sensor molecule according to claim 1, wherein the junctional sequence in the target nucleic acid comprises a CBFA2T3-GLIS2 fusion sequence, an EML4-ALK fusion sequence, a ZFTA-RELA fusion sequence, an EWSR1-FL1 fusion sequence, a CCNH-C5orf30 fusion sequence, a TMEM135-CCDC67 fusion sequence, an EVT6-NTRK3 fusion sequence, a TMPRSS2-ERG fusion sequence, a TRMT11-GRIK2 fusion sequence, or a PVT1-MYC fusion sequence.

6. The sensor molecule according to claim 1, wherein the junctional sequence in the target nucleic acid comprises a TP53(R248Q) mutant transcript.

7. The sensor molecule according to claim 5, wherein the junctional sequence in the target nucleic acid comprises a CBFA2T3-GLIS2 fusion sequence, and wherein the target sensing region includes the nucleic acid sequence set forth in SEQ ID NO: 3.

8. The sensor molecule according to claim 5, wherein the junctional sequence in the target nucleic acid comprises a CBFA2T3-GLIS2 fusion sequence, and wherein the target sensing region comprises a nucleic acid sequence having at least 80% sequence identity to SEQ ID NO: 4 or SEQ ID NO: 5.

9. The sensor molecule according to claim 5, wherein the junctional sequence in the target nucleic acid comprises an EMLA-ALK fusion sequence, and wherein the target sensing region includes the nucleic acid set forth in SEQ ID NO: 28.

10. The sensor molecule according to claim 5, wherein the junctional sequence in the target nucleic acid comprises an EMLA-ALK fusion sequence, and wherein the target sensing region comprises a nucleic acid having at least 80% sequence identity to SEQ ID NO: 20 or SEQ ID NO: 29.

11. The sensor molecule according to claim 5, wherein the junctional sequence in the target nucleic acid comprises a ZFTA-RELA fusion sequence, and wherein the target sensing region includes the nucleic acid sequence set forth in SEQ ID NO: 32.

12. The sensor molecule according to claim 5, wherein the junctional sequence in the target nucleic acid comprises a ZFTA-RELA fusion sequence, and wherein the target sensing region comprises a nucleic acid sequence having at least 80% sequence identity to SEQ ID NO: 31 or SEQ ID NO: 30.

13. The sensor molecule according to claim 5, wherein the junctional sequence of the target nucleic acid comprises an EWSR1-FL1 fusion sequence, and wherein the target sensing region comprises a nucleic acid sequence having at least 80% sequence identity to SEQ ID NO: 33.

14. The sensor molecule according to claim 5, wherein the junctional sequence in the target nucleic acid comprises a CCNH-C5orf30 fusion sequence, and wherein the target sensing region includes a nucleic acid sequence having at least 80% sequence identity to SEQ ID NO: 84.

15. The sensor molecule according to claim 5, wherein the junctional sequence in the target nucleic acid comprises a TMEM135-CCDC67 fusion sequence, and wherein the target sensing region includes a nucleic acid sequence having at least 80% sequence identity to SEQ ID NO: 85.

16. The sensor molecule according to claim 5, wherein the junctional sequence in the target nucleic acid comprises a EVT6-NTRK3 fusion sequence, and wherein the target sensing region includes a nucleic acid sequence having at least 80% sequence identity to SEQ ID NO: 86.

17. The sensor molecule according to claim 5, wherein the junctional sequence in the target nucleic acid comprises a TMPRSS2-ERG fusion sequence, and wherein the target sensing region includes a nucleic acid sequence having at least 80% sequence identity to SEQ ID NO: 87.

18. The sensor molecule according to claim 5, wherein the junctional sequence in the target nucleic acid comprises a TRMT11-GRIK2 fusion sequence, and wherein the target sensing region includes a nucleic acid sequence having at least 80% sequence identity to SEQ ID NO: 88.

19. The sensor molecule according to claim 5, wherein the junctional sequence in the target nucleic acid comprises a PVT1-MYC fusion sequence, and wherein the target sensing region includes a nucleic acid having at least 80% sequence identity to SEQ ID NO: 89.

20. The sensor molecule according to claim 5, wherein the junctional sequence in the target nucleic acid comprises a TP53(R248Q) mutant transcript, and wherein the target sensing region includes a nucleic acid sequence having at least 80% sequence identity to SEQ ID NO: 90.

21. The sensor molecule according to claim 1, wherein the junctional sequence of the target nucleic acid corresponds to a viral transcript.

22. The sensor molecule according to claim 21, wherein the viral transcript is an Epstein Barr Virus (EBV) transcript or a Kaposi's sarcoma-associated herpesvirus (KSHV) transcript.

23. The sensor molecule according to claim 21, wherein the viral transcript is the Epstein Barr Virus transcript EBNA1 and wherein the target sensing region comprises a nucleic acid sequence having at least 80% sequence identity to SEQ ID NO: 34.

24. The sensor molecule according to claim 21, wherein the viral transcript the KSHV transcript ORF71 and wherein the target sensing region comprises a nucleic acid sequence having at least 80% sequence identity to SEQ ID NO: 35.

25. The sensor molecule according to claim 1, wherein the target sensing region is at least about 50 nucleotides long.

26. The sensor molecule according to claim 1, wherein the target sensing region is from about 50 nucleotides to about 1000 nucleotides long.

27. The sensor molecule according to claim 1, wherein the response gene encodes at least one of a reporter protein, a caspase, a prodrug-converting enzyme, or an enzyme catalyzing other reactions.

28. The sensor molecule according to claim 27, wherein the response gene encodes nitroreductase (NTR), diptheria toxin fragment A (DTA), or BCL2 associated X (BAX).

29. The sensor molecule according to claim 1, wherein the sensor molecule further comprises a control gene.

30. The sensor molecule according to claim 29, wherein the control gene is constitutively expressed.

31. The sensor molecule according to claim 29, wherein the control gene encodes a fluorescent protein.

32. The sensor molecule according to claim 1, wherein the sensor molecule comprises a linker region positioned upstream of the response gene but downstream of the TAG or TGA stop codon.

33. The sensor molecule according to claim 32, wherein the linker region comprises a 2A peptide or an XTEN80 peptide.

34. The sensor molecule according to claim 33, wherein the linker region comprises SEQ ID NO: 13,SEQ ID NO: 14, or SEQ ID NO: 15.

35. The sensor molecule according to claim 1, wherein the sensor molecule comprises an RNA aptamer sequence capable of binding its cognate binding protein.

36. The sensor molecule according to claim 35, wherein the RNA aptamer sequence comprises a sequence capable of binding at least one of MS2, PP7, BoxB, or Pumilio.

37. The sensor molecule according to claim 35, wherein the cognate binding protein is fused to an ADAR protein.

38. The sensor molecule according to claim 1, wherein the sensor molecule further comprises a gene encoding an ADAR or an ADAR fusion, wherein the ADAR or ADAR fusion is constitutively expressed.

39. The sensor molecule according to claim 38, wherein the ADAR fusion comprises an ADAR enzyme fused to a cognate aptamer-binding protein.

40. The sensor molecule according to claim 39, wherein the sensor molecule further comprises an RNA aptamer sequence that recruits the cognate aptamer-binding protein upon expression of the ADAR fusion.

41. The sensor molecule according to claim 1, wherein the sensor molecule is an RNA molecule.

42. An expression vector comprising a DNA sequence corresponding to the sensor molecule of claim 1.

43. The expression vector of claim 39, selected from the group consisting of:
(a) a pCR8-mRuby2-P2A-ccdbCam-E2A-EGFP vector;
(b) a pCR8-mRuby2-P2A-ccdbCam-E2A-EGFP-BsaI(agat) vector;
(c) a pCR8-mRuby2-P2A-ccdbCam-E2A-EGFP-NxMS2 vector;
(d) a pCR8-mRuby2-P2A-Sensor-E2A-EGFP vector;
(e) a pCR8-mRuby2-P2A-Sensor-E2A-EGFP-NxMS2 vector;
(f) a pmax-mRuby2-P2A-Sensor-XTEN80-EGFP-NxMS2 vector;
(g) an MCP-ADARdd(E488Q) vector;
(h) a pmax-MCP-ADARdd(E488Q) vector;
(i) a pmax-MCP-ADARdd(E488Q)-P2A-Sensor-XTEN80-NTR1.1-NxMS2 vector;
(j) a pmax-MCP-ADARdd(E488Q)-P2A-Sensor-E2A-NTR1.1-NxMS2 vector;
(k) a pmax-MCP-ADARddm(C377F,E488Q)-P2A-Sensor-XTEN80-NTR1.1-NxMS2 vector;
(l) a pmax-MCP-ADARddm(C377F,E488Q)-P2A-Sensor-E2A-NTR1.1-NxMS2 vector;
(m) a pmax-MCP-ADARdd(E488Q)-P2A-Sensor-XTEN80-DTA-NxMS2 vector;
(n) a pmax-MCP-ADARdd(E488Q)-P2A-Sensor-E2A-DTA-NxMS2 vector;
(o) a pmax-MCP-ADARddm(C377F,E488Q)-P2A-Sensor-XTEN80-DTA-NxMS2 vector;
(p) a pmax-MCP-ADARddm(C377F,E488Q)-P2A-Sensor-E2A-DTA-NxMS2 vector;
(q) a pmax-MCP-ADARdd(E488Q)-P2A-Sensor-XTEN80-BAX-NxMS2 vector;
(r) a pmax-MCP-ADARdd(E488Q)-P2A-Sensor-E2A-BAX-NxMS2 vector;
(s) a pmax-MCP-ADARddm(C377F,E488Q)-P2A-Sensor-XTEN80-BAX-NxMS2 vector; and
(t) a pmax-MCP-ADARddm(C377F,E488Q)-P2A-Sensor-E2A-BAX-NxMS2 vector.

44. A cell comprising any of the sensor molecules of claim 1 or the vector of claim 42.

45. (canceled)

46. (canceled)

47. (canceled)

48. (canceled)

49. (canceled)

50. (canceled)

51. A method of detecting a gene fusion transcript in a cell, the method comprising:
transfecting a cell with the sensor molecule of claim 1 or the vector of claim 42, and assessing the cell for expression of a reporter protein.
